(19) United States
(12) Patent Application Publication
(10) Pub. No.: US 2023/0159922 A1
(43) Pub. Date: May 25, 2023

(54) MODIFIED OLIGOMERIC COMPOUNDS AND USES THEREOF

(71) Applicant: Ionis Pharmaceuticals, Inc., Carlsbad, CA (US)

(72) Inventors: Punit P. Seth, Carlsbad, CA (US); Michael T. Migawa, Carlsbad, CA (US); Graeme C. Freestone, La Jolla, CA (US)

(73) Assignee: Ionis Pharmaceuticals, Inc., Carlsbad, CA (US)

(21) Appl. No.: 17/635,096

(22) PCT Filed: Aug. 14, 2020
(86) PCT No.: PCT/US2020/046540

§ 371 Date: Feb. 14, 2022

Related U.S. Application Data
(60) Provisional application No. 62/887,534, filed on Aug. 15, 2019.

(51) Int. Cl. C12N 15/113

(52) U.S. Cl. CPC C12N 15/113; C12N 2310/11; C12N 2310/351

(57) ABSTRACT

The present disclosure provides oligomeric compounds comprising a modified oligonucleotide having at least one stereo-non-standard nucleoside. An oligomeric compound comprising a modified oligonucleotide consisting of 12-30 linked nucleosides, wherein at least one nucleoside of the modified oligonucleotide is a stereo-non-standard nucleoside; and wherein the oligomeric compound is selected from among an RNAi compound, a modified CRISPR compound, and an artificial mRNA compound.

### SEQUENCE LISTING

The present application is being filed along with a Sequence Listing in electronic format. The Sequence Listing is provided as a file entitled CORE0156WOSEQ_ST25.txt created Aug. 13, 2020 which is 24 kb in size. The information in the electronic format of the sequence listing is incorporated herein by reference in its entirety.

### FIELD

The present disclosure provides oligomeric compounds comprising a modified oligonucleotide having at least one stereo-non-standard nucleoside.

### BACKGROUND

The principle behind antisense technology is that an antisense compound hybridizes to a target nucleic acid and modulates the amount, activity, and/or function of the target nucleic acid. For example, in certain instances, antisense compounds result in altered transcription or translation of a target. Such modulation of expression can be achieved by, for example, target RNA degradation or occupancy-based inhibition. An example of modulation of RNA target function by degradation is RNase H-based degradation of the target RNA upon hybridization with a DNA-like antisense compound.

Another example of modulation of gene expression by target degradation is RNA interference (RNAi). RNAi refers to antisense-mediated gene silencing through a mechanism that utilizes the RNA-induced silencing complex (RISC). An additional example of modulation of RNA target function is by an occupancy-based mechanism such as is employed naturally by microRNA. MicroRNAs are small non-coding RNAs that regulate the expression of protein-coding RNAs. The binding of an antisense compound to a microRNA prevents that microRNA from binding to its messenger RNA targets, and thus interferes with the function of the microRNA. MicroRNA mimics can enhance native microRNA function. Certain antisense compounds alter splicing of pre-mRNA. Another example of modulation of gene expression is the use of antisense compounds in a CRISPR system. Regardless of the specific mechanism, sequence-specificity makes antisense compounds attractive as tools for target validation and gene functionalization, as well as therapeutics to selectively modulate the expression of genes involved in the pathogenesis of disease.

Antisense technology is an effective means for modulating the expression of one or more specific gene products and can therefore prove to be uniquely useful in a number of therapeutic, diagnostic, and research applications. Chemically modified nucleosides may be incorporated into antisense compounds to enhance one or more properties, such as nuclease resistance, pharmacokinetics, or affinity for a target nucleic acid.

### SUMMARY

The present disclosure provides oligomeric compounds comprising a modified oligonucleotide, wherein at least one nucleoside of the modified oligonucleotide is a stereo-non-standard nucleoside having a structure selected from Formula I, Formula II, Formula III, Formula IV, Formula V, Formula VI, and Formula VII:

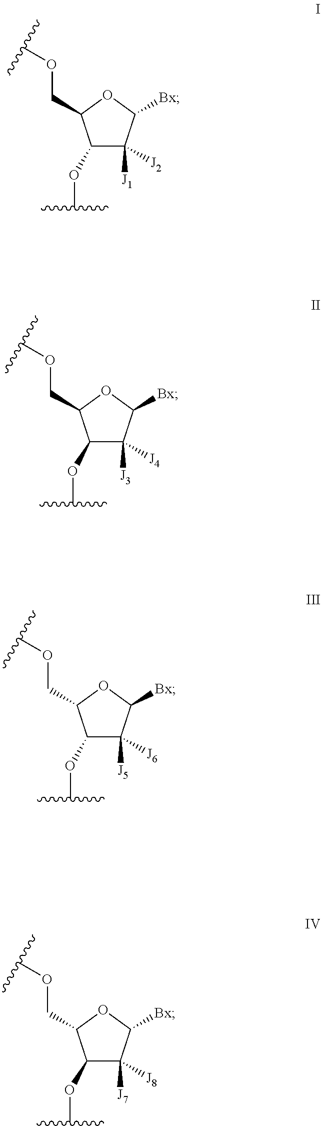

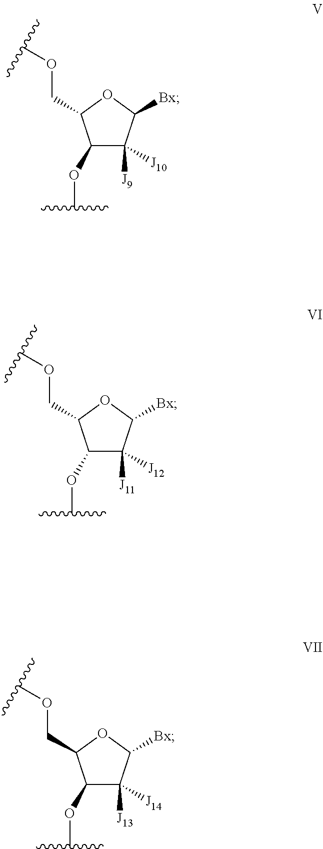

wherein

one of J₁ and J₂ is H and the other of J₁ and J₂ is selected from H, OH, F, OCH₃, OCH₂CH₂OCH₃, O—C₁-C₆ alkoxy, and SCH₃;

one of J₃ and J₄ is H and the other of J₃ and J₄ is selected from H, OH, F, OCH₃, OCH₂CH₂OCH₃, O—C₁-C₆ alkoxy, and SCH₃; and wherein

one of J₅ and J₆ is H and the other of J₅ and J₆ is selected from H, OH, F, OCH₃, OCH₂CH₂OCH₃, O—C₁-C₆ alkoxy, and SCH₃; and wherein

one of J₇ and J₈ is H and the other of J₇ and J₈ is selected from H, OH, F, OCH₃, OCH₂CH₂OCH₃, O—C₁-C₆ alkoxy, and SCH₃; and wherein

one of J₉ and J₁₀ is H and the other of J₉ and J₁₀ is selected from H, OH, F, OCH₃, OCH₂CH₂OCH₃, O—C₁-C₆ alkoxy, and SCH₃; and wherein

one of J₁₁ and J₁₂ is H and the other of J₁₁ and J₁₂ is selected from H, OH, F, OCH₃, OCH₂CH₂OCH₃, O—C₁-C₆ alkoxy, and SCH₃; and wherein

one of J₁₃ and J₁₄ is H and the other of J₁₃ and J₁₄ is selected from H, OH, F, OCH₃, OCH₂CH₂OCH₃, O—C₁-C₆ alkoxy, and SCH₃;

Bx is a is a heterocyclic base moiety; and

wherein the oligomeric compound is selected from among an RNAi compound, a modified CRISPR compound, and an artificial mRNA compound.

In certain embodiments, the modified oligonucleotides having at least one stereo-non-standard nucleoside provided herein have an increased maximum tolerated dose when administered to an animal compared to an otherwise identical oligomeric compound except that the otherwise identical oligomeric compound lacks the at least one stereo-non-standard nucleoside.

In certain embodiments, the modified oligonucleotides having at least one stereo-non-standard nucleoside provided herein have an increased therapeutic index compared to an otherwise identical oligomeric compound except that the otherwise identical oligomeric compound lacks the at least one stereo-non-standard nucleoside.

### BRIEF DESCRIPTION OF THE DRAWINGS

FIG. 1 depicts isomers of 2′-deoxyfuranosyl sugar moieties having formulas I-VII.

FIG. 2 depicts isomers of 2′-O-methyl furanosyl sugar moieties having formulas I-VII.

FIG. 3 depicts isomers of 2′-fluoro furanosyl sugar moieties having formulas I-VII.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH(H) sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

As used herein, “2′-substituted” in reference to a furanosyl sugar moiety or nucleoside comprising a furanosyl sugar moiety means the furanosyl sugar moiety or nucleoside comprising the furanosyl sugar moiety comprises a substituent other than H or OH at the 2′-position and is a non-bicyclic furanosyl sugar moiety. 2′-substituted furanosyl sugar moieties do not comprise additional substituents at other positions of the furanosyl sugar moiety other than a nucleobase and/or internucleoside linkage(s) when in the context of an oligonucleotide.

As used herein, “4′-substituted” in reference to a furanosyl sugar moiety or nucleoside comprising a furanosyl sugar moiety means the furanosyl sugar moiety or nucleoside comprising the furanosyl sugar moiety comprises a substituent other than H at the 4′-position and is a non-bicyclic furanosyl sugar moiety. 4′-substituted furanosyl sugar moieties do not comprise additional substituents at other positions of the furanosyl sugar moiety other than a nucleobase and/or internucleoside linkage(s) when in the context of an oligonucleotide.

As used herein, “5′-substituted” in reference to a furanosyl sugar moiety or nucleoside comprising a furanosyl sugar moiety means the furanosyl sugar moiety or nucleoside comprising the furanosyl sugar moiety comprises a substituent other than H at the 5′-position and is a non-bicyclic furanosyl sugar moiety. 5′-substituted furanosyl sugar moieties do not comprise additional substituents at other positions of the furanosyl sugar moiety other than a nucleobase and/or internucleoside linkage(s) when in the context of an oligonucleotide.

As used herein, “administration” or “administering” refers to routes of introducing a compound or composition provided herein to a subject. Examples of routes of administration that can be used include, but are not limited to, administration by inhalation, subcutaneous injection, intrathecal injection, and oral administration.

As used herein, “artificial mRNA compound” is an oligonucleotide or portion thereof that, when contacted with a cell, encodes a protein.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety, and the bicyclic sugar moiety is a modified furanosyl sugar moiety. In certain embodiments, the bicyclic sugar moiety does not comprise a furanosyl moiety.

As used herein, “cEt” or “constrained ethyl” means a bicyclic sugar moiety, wherein the first ring of the bicyclic sugar moiety is a ribosyl sugar moiety, the second ring of the bicyclic sugar is formed via a bridge connecting the 4′-carbon and the 2′-carbon, the bridge has the formula 4′-CH(CH₃)—O-2′, and the methyl group of the bridge is in the S configuration. A cEt bicyclic sugar moiety is in the $-D configuration.

As used herein, “complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases are nucleobase pairs that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms that is directly or indirectly attached to an oligonucleotide. Conjugate groups may comprise a conjugate moiety and a conjugate linker that attaches the conjugate moiety to the oligonucleotide.

As used herein, “conjugate linker” means a bond or a group of atoms comprising at least one bond that connects a conjugate moiety to an oligonucleotide.

As used herein, “conjugate moiety” means a group of atoms that is attached to an oligonucleotide via a conjugate linker.

As used herein, “CRISPR compound” means a modified oligonucleotide that comprises a DNA recognition portion and a tracrRNA recognition portion. As used herein, “DNA recognition portion” is nucleobase sequence that is complementary to a DNA target. As used herein, “tracrRNA recognition portion” is a nucleobase sequence that is bound to or is capable of binding to tracrRNA. The tracrRNA recognition portion of crRNA may bind to tracrRNA via hybridization or covalent attachment.

As used herein, “cytotoxic” or “cytotoxicity” in the context of an effect of an oligomeric compound or a parent oligomeric compound on cultured cells means an at least 2-fold increase in caspase activation following administration of 10 μM or less of the oligomeric compound or parent oligomeric compound to the cultured cells relative to cells cultured under the same conditions but that are not administered the oligomeric compound or parent oligomeric compound. In certain embodiments, cytotoxicity is measured using a standard in vitro cytotoxicity assay.

As used herein, “deoxy region” means a region of 5-12 contiguous nucleotides, wherein at least 70% of the nucleosides are stereo-standard DNA nucleosides. In certain embodiments, each nucleoside is selected from a stereo-standard DNA nucleoside (a nucleoside comprising a β-D-2′-deoxyribosyl sugar moiety), a stereo-non-standard nucleoside of Formula I-VII, a bicyclic nucleoside, and a substituted stereo-standard nucleoside. In certain embodiments, a deoxy region supports RNase H activity. In certain embodiments, a deoxy region is the gap of a gapmer.

As used herein, “double-stranded antisense compound” means an antisense compound comprising two oligomeric compounds that are complementary to each other and form a duplex, and wherein one of the two said oligomeric compounds comprises an antisense oligonucleotide.

As used herein, “expression” includes all the functions by which a gene's coded information is converted into structures present and operating in a cell. Such structures include, but are not limited to, the products of transcription and translation. As used herein, “modulation of expression” means any change in amount or activity of a product of transcription or translation of a gene. Such a change may be an increase or a reduction of any amount relative to the expression level prior to the modulation.

As used herein, “gapmer” means an oligonucleotide having a central region comprising a plurality of nucleosides that support RNase H cleavage positioned between a 5′-region and a 3′-region. In certain embodiments, the nucleosides of the 5′-region and 3′-region each comprise a 2′-substituted furanosyl sugar moiety or a bicyclic sugar moiety, and the 3′- and 5′-most nucleosides of the central region each comprise a sugar moiety independently selected from a 2′-deoxyfuranosyl sugar moiety or a sugar surrogate. The positions of the central region refer to the order of the nucleosides of the central region and are counted starting from the 5′-end of the central region. Thus, the 5′-most nucleoside of the central region is at position 1 of the central region. The “central region” may be referred to as a “gap”, and the “5′-region” and “3′-region” may be referred to as “wings”. Gaps of gapmers are deoxy regions. As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “inhibiting the expression or activity” refers to a reduction or blockade of the expression or activity relative to the expression or activity in an untreated or control sample and does not necessarily indicate a total elimination of expression or activity.

As used herein, the terms “internucleoside linkage” means a group of atoms or bond that forms a covalent linkage between adjacent nucleosides in an oligonucleotide. As used herein “modified internucleoside linkage” means any internucleoside linkage other than a naturally occurring, phosphodiester internucleoside linkage. “Phosphorothioate linkage” means a modified internucleoside linkage in which one of the non-bridging oxygen atoms of a phosphodiester is replaced with a sulfur atom. Modified internucleoside linkages may or may not contain a phosphorus atom. A “neutral internucleoside linkage” is a modified internucleoside linkage that does not have a negatively charged phosphate in a buffered aqueous solution at pH=7.0.

As used herein, “abasic nucleoside” means a sugar moiety in an oligonucleotide or oligomeric compound that is not directly connected to a nucleobase. In certain embodiments, an abasic nucleoside is adjacent to one or two nucleosides in an oligonucleotide.

As used herein, “linked nucleosides” are nucleosides that are connected in a continuous sequence (i.e. no additional nucleosides are present between those that are linked).

As used herein, “maximum tolerated dose” means the highest dose of a compound that does not cause unacceptable side effects. In certain embodiments, the maximum tolerated dose is the highest dose of a modified oligonucleotide that does not cause an ALT elevation of three times the upper limit of normal as measured by a standard assay, e.g. the assay of Example 4.

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “modulating” refers to changing or adjusting a feature in a cell, tissue, organ or organism.

As used herein, “MOE” means methoxyethyl. “2′-MOE” or “2′-O-methoxyethyl” means a 2′-OCH₂CH₂OCH₃ group at the 2′-position of a furanosyl ring. In certain embodiments, the 2′-OCH₂CH₂OCH₃ group is in place of the 2′-OH group of a ribosyl ring or in place of a 2′-H in a 2′-deoxyribosyl ring.

As used herein, “motif” means the pattern of unmodified and/or modified sugar moieties, nucleobases, and/or internucleoside linkages, in an oligonucleotide.

As used herein, “naturally occurring” means found in nature.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a modified nucleobase is a group of atoms capable of pairing with at least one unmodified nucleobase. A universal base is a nucleobase that can pair with any one of the five unmodified nucleobases. 5-methylcytosine (^{m}C) is one example of a modified nucleobase.

As used herein, “nucleobase sequence” means the order of contiguous nucleobases in a nucleic acid or oligonucleotide independent of any sugar moiety or internucleoside linkage modification.

As used herein, “nucleoside” means a moiety comprising a nucleobase and a sugar moiety. The nucleobase and sugar moiety are each, independently, unmodified or modified. As used herein, “modified nucleoside” means a nucleoside comprising a modified nucleobase and/or a modified sugar moiety.

As used herein, “oligomeric compound” means a compound consisting of (1) an oligonucleotide (a single-stranded oligomeric compound) or two oligonucleotides hybridized to one another (a double-stranded oligomeric compound); and (2) optionally one or more additional features, such as a conjugate group or terminal group which may be bound to the oligonucleotide of a single-stranded oligomeric compound or to one or both oligonucleotides of a double-stranded oligomeric compound.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 12-3000 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, liquids, powders, or suspensions that can be aerosolized or otherwise dispersed for inhalation by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds, i.e., salts that retain the desired biological activity of the compound and do not impart undesired toxicological effects thereto.

As used herein “pharmaceutical composition” means a mixture of substances suitable for administering to a subject. For example, a pharmaceutical composition may comprise an antisense compound and an aqueous solution.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense oligonucleotides that act through RNase H.

As used herein, the term “single-stranded” in reference to an antisense compound means such a compound consists of one oligomeric compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex, in which case the compound would no longer be single-stranded.

As used herein, “stereo-standard nucleoside” means a nucleoside comprising a non-bicyclic furanosyl sugar moiety having the configuration of naturally occurring DNA and RNA as shown below. A “stereo-standard DNA nucleoside” is a nucleoside comprising a β-D-2′-deoxyribosyl sugar moiety. A “stereo-standard RNA nucleoside” is a nucleoside comprising a β-D-ribosyl sugar moiety. A “substituted stereo-standard nucleoside” is a stereo-standard nucleoside other than a stereo-standard DNA or stereo-standard RNA nucleoside. In certain embodiments, R₁ is a 2′-substituent and R₂-R₅ are each H. In certain embodiments, the 2′-substituent is selected from OMe, F, OCH₂CH₂OCH₃, O-alkyl, SMe, or NMA. In certain embodiments, R₁-R₄ are H and R₅ is a 5′-substituent selected from methyl, allyl, or ethyl. In certain embodiments, the heterocyclic base moiety Bx is selected from uracil, thymine, cytosine, 5-methyl cytosine, adenine or guanine. In certain embodiments, the heterocyclic base moiety Bx is other than uracil, thymine, cytosine, 5-methyl cytosine, adenine or guanine.

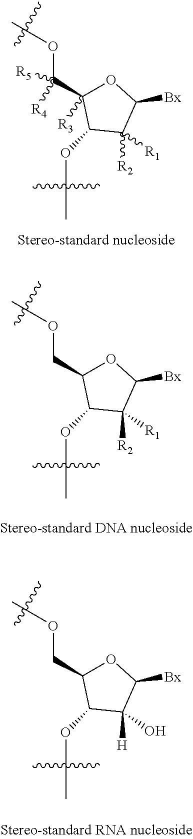

As used herein, “stereo-non-standard nucleoside” means a nucleoside comprising a non-bicyclic furanosyl sugar moiety having a configuration other than that of a stereo-standard sugar moiety. In certain embodiments, a “stereo-non-standard nucleoside” is represented by Formulas I-VII below. In certain embodiments, J₁-J₁₄ are independently selected from H, OH, F, OCH₃, OCH₂CH₂OCH₃, O—C₁-C₆ alkoxy, and SCH₃. A “stereo-non-standard RNA nucleoside” has one of formulas I-VII below, wherein each of J₁, J₃, J₅, J₇, J₉, J₁₁, and J₁₃ is H, and each of J₂, J₄, J₆, J₈, J₁₀, J₁₂, and J₁₄ is OH. An “stereo-non-standard DNA nucleoside” has one of formulas I-VII below, wherein each J is H. A “2′-substituted stereo-non-standard nucleoside” has one of formulas I-VII below, wherein either J₁, J₃, J₅, J₇, J₉, J₁₁, and J₁₃ is other than H and/or or J₂, J₄, J₆, J₈, J₁₀, J₁₂, and J₁₄ is other than H or OH. In certain embodiments, the heterocyclic base moiety Bx is selected from uracil, thymine, cytosine, 5-methyl cytosine, adenine or guanine. In certain embodiments, the heterocyclic base moiety Bx is other than uracil, thymine, cytosine, 5-methyl cytosine, adenine or guanine.

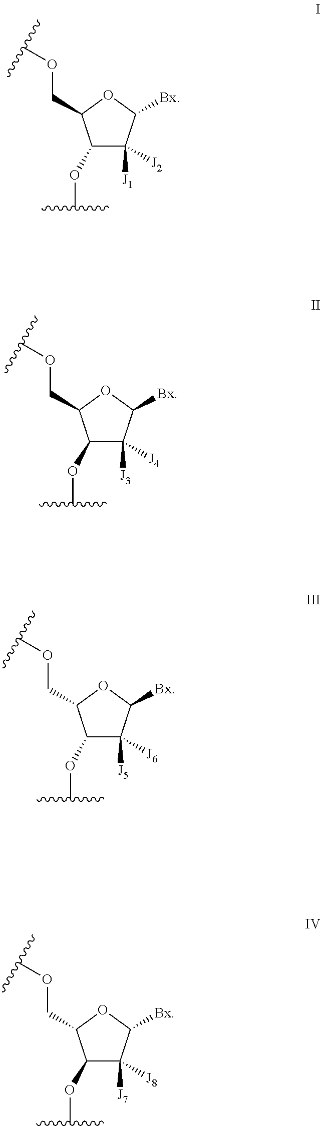

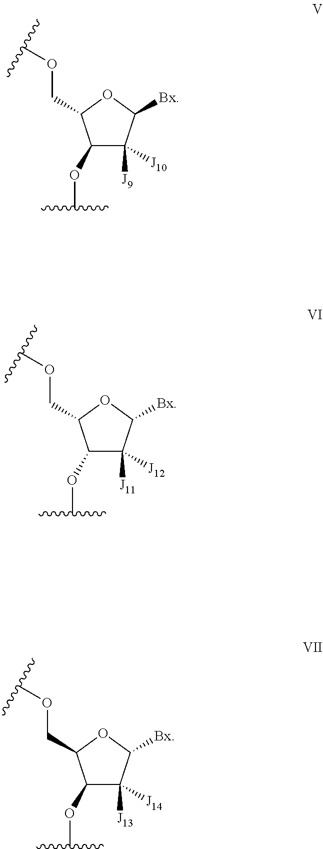

As used herein, “stereo-standard sugar moiety” means the sugar moiety of a stereo-standard nucleoside.

As used herein, “stereo-non-standard sugar moiety” means the sugar moiety of a stereo-non-standard nucleoside.

As used herein, “substituted stereo-non-standard nucleoside” means a stereo-non-standard nucleoside comprising a substituent other than the substituent corresponding to natural RNA or DNA. Substituted stereo-non-standard nucleosides include but are not limited to nucleosides of Formula I-VII wherein the J groups are other than: (1) both H or (2) one H and the other OH.

As used herein, “subject” means a human or non-human animal selected for treatment or therapy.

As used herein, “sugar moiety” means an unmodified sugar moiety or a modified sugar moiety. As used herein, “unmodified sugar moiety” means a β-D-ribosyl moiety, as found in naturally occurring RNA, or a β-D-2′-deoxyribosyl sugar moiety as found in naturally occurring DNA. As used herein, “modified sugar moiety” or “modified sugar” means a sugar surrogate or a furanosyl sugar moiety other than a β-D-ribosyl or a β-D-2′-deoxyribosyl. Modified furanosyl sugar moieties may be modified or substituted at a certain position(s) of the sugar moiety, or unsubstituted, and they may or may not be stereo-non-standard sugar moieties. Modified furanosyl sugar moieties include bicyclic sugars and non-bicyclic sugars. As used herein, “sugar surrogate” means a modified sugar moiety that does not comprise a furanosyl or tetrahydrofuranyl ring (is not a “furanosyl sugar moiety”) and that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid,” “target RNA,” “target RNA transcript” and “nucleic acid target” means a nucleic acid that an oligomeric compound, such as an antisense compound, is designed to affect. In certain embodiments, an oligomeric compound comprises an oligonucleotide having a nucleobase sequence that is complementary to more than one RNA, only one of which is the target RNA of the oligomeric compound. In certain embodiments, the target RNA is an RNA present in the species to which an oligomeric compound is administered.

As used herein, “therapeutic index” means a comparison of the amount of a compound that causes a therapeutic effect to the amount that causes toxicity. Compounds having a high therapeutic index have strong efficacy and low toxicity. In certain embodiments, increasing the therapeutic index of a compound increases the amount of the compound that can be safely administered.

As used herein, “treat” refers to administering a compound or pharmaceutical composition to an animal in order to effect an alteration or improvement of a disease, disorder, or condition in the animal.

### Certain Embodiments

The present disclosure provides the following non-limiting embodiments:
Embodiment 1. An oligomeric compound comprising a modified oligonucleotide consisting of 12-30 linked nucleosides, wherein at least one nucleoside of the modified oligonucleotide is a stereo-non-standard nucleoside; and wherein the oligomeric compound is selected from among an RNAi compound, a modified CRISPR compound, and an artificial mRNA compound.
Embodiment 2. The oligomeric compound of embodiment 1 comprising at least one stereo-non-standard DNA nucleoside.
Embodiment 3. The oligomeric compound of embodiment 1 or 2 comprising at least one stereo-non-standard RNA nucleoside.
Embodiment 4. The oligomeric compound of any of embodiments 1-3 comprising at least one substituted stereo-non-standard nucleoside.
Embodiment 5. The oligomeric compound of any of embodiments 1-4 comprising at least one 2′-substituted stereo-non-standard nucleoside.
Embodiment 6. The oligomeric compound of any of embodiment 1-5, wherein at least one stereo-non-standard nucleoside has a structure selected from Formula I, Formula II, Formula III, Formula IV, Formula V, Formula VI, and Formula VII:

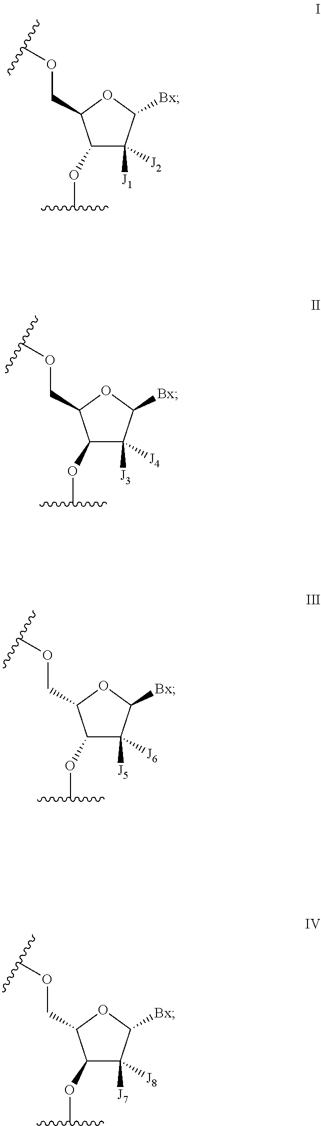

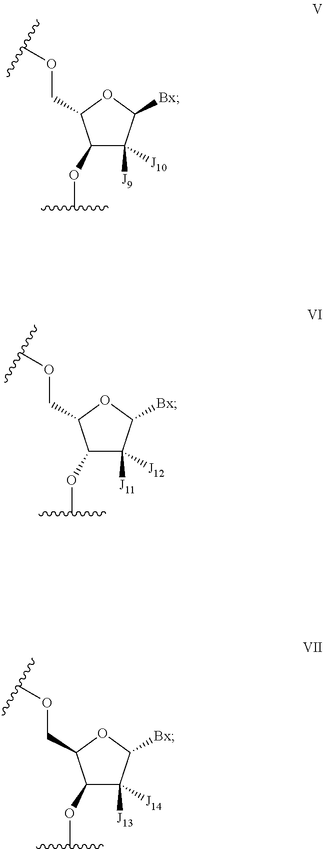

one of J₁ and J₂ is H and the other of J₁ and J₂ is selected from H, OH, F, OCH₃, OCH₂CH₂OCH₃, O—C₁-C₆ alkoxy, and SCH₃;

one of J₃ and J₄ is H and the other of J₃ and J₄ is selected from H, OH, F, OCH₃, OCH₂CH₂OCH₃, O—C₁-C₆ alkoxy, and SCH₃; and wherein

one of J₅ and J₆ is H and the other of J₅ and J₆ is selected from H, OH, F, OCH₃, OCH₂CH₂OCH₃, O—C₁-C₆ alkoxy, and SCH₃; and wherein

one of J₇ and J₈ is H and the other of J₇ and J₈ is selected from H, OH, F, OCH₃, OCH₂CH₂OCH₃, O—C₁-C₆ alkoxy, and SCH₃; and wherein

one of J₉ and J₁₀ is H and the other of J₉ and J₁₀ is selected from H, OH, F, OCH₃, OCH₂CH₂OCH₃, O—C₁-C₆ alkoxy, and SCH₃; and wherein

one of J₁₁ and J₁₂ is H and the other of J₁₁ and J₁₂ is selected from H, OH, F, OCH₃, OCH₂CH₂OCH₃, O—C₁-C₆ alkoxy, and SCH₃; and wherein

one of J₁₃ and J₁₄ is H and the other of J₁₃ and J₁₄ is selected from H, OH, F, OCH₃, OCH₂CH₂OCH₃, O—C₁-C₆ alkoxy, and SCH₃; and

Bx is a is a heterocyclic base moiety.
Embodiment 7. The oligomeric compound of embodiment 6, wherein:

one of J₁ and J₂ is H and the other of J₁ and J₂ is selected from OH, F, OCH₃, OCH₂CH₂OCH₃, O—C₁-C₆ alkoxy, and SCH₃;

one of J₃ and J₄ is H and the other of J₃ and J₄ is selected from OH, F, OCH₃, OCH₂CH₂OCH₃, O—C₁-C₆ alkoxy, and SCH₃;

one of J₅ and J₆ is H and the other of J₅ and J₆ is selected from OH, F, OCH₃, OCH₂CH₂OCH₃, O—C₁-C₆ alkoxy, and SCH₃;

one of J₇ and J₈ is H and the other of J₇ and J₈ is selected from OH, F, OCH₃, OCH₂CH₂OCH₃, O—C₁-C₆ alkoxy, and SCH₃;

one of J₉ and J₁₀ is H and the other of J₉ and J₁₀ is selected from OH, F, OCH₃, OCH₂CH₂OCH₃, O—C₁-C₆ alkoxy, and SCH₃;

one of J₁₁ and J₁₂ is H and the other of J₁₁ and J₁₂ is selected from OH, F, OCH₃, OCH₂CH₂OCH₃, O—C₁-C₆ alkoxy, and SCH₃;

one of J₁₃ and J₁₄ is H and the other of J₁₃ and J₁₄ is selected from OH, F, OCH₃, OCH₂CH₂OCH₃, O—C₁-C₆ alkoxy, and SCH₃.
Embodiment 8. The oligomeric compound of embodiment 6 or 7 comprising at least one stereo-non-standard nucleoside having a structure of Formula I.
Embodiment 9. The oligomeric compound of embodiment 8, wherein J₁ is H.
Embodiment 10. The oligomeric compound of embodiment 8, wherein J₁ is OH
Embodiment 11. The oligomeric compound of embodiment 8, wherein J₁ is F.
Embodiment 12. The oligomeric compound of embodiment 8, wherein J₁ is OCH₃.
Embodiment 13. The oligomeric compound of embodiment 8, wherein J₁ is OCH₂CH₂OCH₃.
Embodiment 14. The oligomeric compound of embodiment 8, wherein J₁ is O—C₁-C₆ alkoxy.
Embodiment 15. The oligomeric compound of embodiment 8, wherein J₁ is SCH₃.
Embodiment 16. The oligomeric compound of any of embodiments 8-15, wherein J₂ is H.
Embodiment 17. The oligomeric compound of embodiments 8-15, wherein J₂ is OH.
Embodiment 18. The oligomeric compound of embodiments 8-15, wherein J₂ is F.
Embodiment 19. The oligomeric compound of embodiments 8-15, wherein J₂ is OCH₃.
Embodiment 20. The oligomeric compound of embodiments 8-15, wherein J₂ is OCH₂CH₂OCH₃.
Embodiment 21. The oligomeric compound of embodiments 8-15, wherein J₂ is O—C₁-C₆ alkoxy.
Embodiment 22. The oligomeric compound of embodiments 8-15, wherein J₂ is SCH₃.
Embodiment 23. The oligomeric compound of any of embodiments 6-22 comprising at least one stereo-non-standard nucleoside having a structure of Formula II.
Embodiment 24. The oligomeric compound of embodiment 23, wherein J₃ is H.
Embodiment 25. The oligomeric compound of embodiment 23, wherein J₃ is OH
Embodiment 26. The oligomeric compound of embodiment 23, wherein J₃ is F.
Embodiment 27. The oligomeric compound of embodiment 23, wherein J₃ is OCH₃.
Embodiment 28. The oligomeric compound of embodiment 23, wherein J₃ is OCH₂CH₂OCH₃.
Embodiment 29. The oligomeric compound of embodiment 23, wherein J₃ is O—C₁-C₆ alkoxy.
Embodiment 30. The oligomeric compound of embodiment 23, wherein J₃ is SCH₃.
Embodiment 31. The oligomeric compound of any of embodiments 23-30, wherein J₄ is H.
Embodiment 32. The oligomeric compound of any of embodiments 23-30, wherein J₄ is OH
Embodiment 33. The oligomeric compound of any of embodiments 23-30, wherein J₄ is F.
Embodiment 34. The oligomeric compound of any of embodiments 23-30, wherein J₄ is OCH₃.
Embodiment 35. The oligomeric compound of any of embodiments 23-30, wherein J₄ is OCH₂CH₂OCH₃.
Embodiment 36. The oligomeric compound of any of embodiments 23-30, wherein J₄ is O—C₁-C₆ alkoxy.
Embodiment 37. The oligomeric compound of any of embodiments 23-30, wherein J₄ is SCH₃.
Embodiment 38. The oligomeric compound of any of embodiments 6-37 comprising at least one stereo-non-standard nucleoside having a structure of Formula III.
Embodiment 39. The oligomeric compound of embodiment 38, wherein J₅ is H.
Embodiment 40. The oligomeric compound of embodiment 38, wherein J₅ is OH
Embodiment 41. The oligomeric compound of embodiment 38, wherein J₅ is F.
Embodiment 42. The oligomeric compound of embodiment 38, wherein J₅ is OCH₃.
Embodiment 43. The oligomeric compound of embodiment 38, wherein J₅ is OCH₂CH₂OCH₃.
Embodiment 44. The oligomeric compound of embodiment 38, wherein J₅ is O—C₁-C₆ alkoxy.
Embodiment 45. The oligomeric compound of embodiment 38, wherein J₅ is SCH₃.
Embodiment 46. The oligomeric compound of any of embodiments 38-45, wherein J₆ is H.
Embodiment 47. The oligomeric compound of any of embodiments 38-45, wherein J₆ is OH
Embodiment 48. The oligomeric compound of any of embodiments 38-45, wherein J₆ is F.
Embodiment 49. The oligomeric compound of any of embodiments 38-45, wherein J₆ is OCH₃.
Embodiment 50. The oligomeric compound of any of embodiments 38-45, wherein J₆ is OCH₂CH₂OCH₃.
Embodiment 51. The oligomeric compound of any of embodiments 38-45, wherein J₆ is O—C₁-C₆ alkoxy.
Embodiment 52. The oligomeric compound of any of embodiments 38-45, wherein J₆ is SCH₃.
Embodiment 53. The oligomeric compound of any of embodiments 6-52 comprising at least one stereo-non-standard nucleoside having a structure of Formula IV.
Embodiment 54. The oligomeric compound of embodiment 53, wherein J₇ is H.
Embodiment 55. The oligomeric compound of embodiment 53, wherein J₇ is OH
Embodiment 56. The oligomeric compound of embodiment 53, wherein J₇ is F.
Embodiment 57. The oligomeric compound of embodiment 48, wherein J₇ is OCH₃.
Embodiment 58. The oligomeric compound of embodiment 53, wherein J₇ is OCH₂CH₂OCH₃.
Embodiment 59. The oligomeric compound of embodiment 53, wherein J₇ is O—C₁-C₆ alkoxy.
Embodiment 60. The oligomeric compound of embodiment 53, wherein J₇ is SCH₃.
Embodiment 61. The oligomeric compound of any of embodiments 53-60, wherein J₈ is H.
Embodiment 62. The oligomeric compound of any of embodiments 53-60, wherein J₈ is OH
Embodiment 63. The oligomeric compound of any of embodiments 53-60, wherein J₈ is F.
Embodiment 64. The oligomeric compound of any of embodiments 53-60, wherein J₈ is OCH₃.
Embodiment 65. The oligomeric compound of any of embodiments 53-60, wherein J₈ is OCH₂CH₂OCH₃.
Embodiment 66. The oligomeric compound of any of embodiments 53-60, wherein J₈ is O—C₁-C₆ alkoxy.
Embodiment 67. The oligomeric compound of any of embodiments 53-60, wherein J₈ is SCH₃.
Embodiment 68. The oligomeric compound of any of embodiments 6-67 comprising at least one stereo-non-standard nucleoside having a structure of Formula V.
Embodiment 69. The oligomeric compound of embodiment 68, wherein J₉ is H.
Embodiment 70. The oligomeric compound of embodiment 68, wherein J₉ is OH
Embodiment 71. The oligomeric compound of embodiment 68, wherein J₉ is F.
Embodiment 72. The oligomeric compound of embodiment 68, wherein J₉ is OCH₃.
Embodiment 73. The oligomeric compound of embodiment 68, wherein J₉ is OCH₂CH₂OCH₃.
Embodiment 74. The oligomeric compound of embodiment 68, wherein J₉ is O—C₁-C₆ alkoxy.
Embodiment 75. The oligomeric compound of embodiment 68, wherein J₉ is SCH₃.
Embodiment 76. The oligomeric compound of any of embodiments 68-75, wherein J₁₀ is H.
Embodiment 77. The oligomeric compound of any of embodiments 68-75, wherein J₁₀ is OH.
Embodiment 78. The oligomeric compound of any of embodiments 68-75, wherein J₁₀ is F.
Embodiment 79. The oligomeric compound of any of embodiments 68-75, wherein J₁₀ is OCH₃.
Embodiment 80. The oligomeric compound of any of embodiments 68-75, wherein J₁₀ is OCH₂CH₂OCH₃.
Embodiment 81. The oligomeric compound of any of embodiments 68-75, wherein J₁₀ is O—C₁-C₆ alkoxy.
Embodiment 82. The oligomeric compound of any of embodiments 68-75, wherein J₁₀ is SCH₃.
Embodiment 83. The oligomeric compound of any of embodiments 6-82 comprising at least one stereo-non-standard nucleoside having a structure of Formula VI.
Embodiment 84. The oligomeric compound of embodiment 83, wherein J₁₁ is H.
Embodiment 85. The oligomeric compound of embodiment 83, wherein J₁₁ is OH.
Embodiment 86. The oligomeric compound of embodiment 83, wherein J₁₁ is F.
Embodiment 87. The oligomeric compound of embodiment 83, wherein J₁₁ is OCH₃.
Embodiment 88. The oligomeric compound of embodiment 83, wherein J₁₁ is OCH₂CH₂OCH₃.
Embodiment 89. The oligomeric compound of embodiment 83, wherein J₁₁ is O—C₁-C₆ alkoxy.
Embodiment 90. The oligomeric compound of embodiment 83, wherein J₁₁ is SCH₃.
Embodiment 91. The oligomeric compound of any of embodiments 83-90, wherein J₁₂ is H.
Embodiment 92. The oligomeric compound of any of embodiments 83-90, wherein J₁₂ is OH.
Embodiment 93. The oligomeric compound of any of embodiments 83-90, wherein J₁₂ is F.
Embodiment 94. The oligomeric compound of any of embodiments 83-90, wherein J₁₂ is OCH₃.
Embodiment 95. The oligomeric compound of any of embodiments 83-90, wherein J₁₂ is OCH₂CH₂OCH₃.
Embodiment 96. The oligomeric compound of any of embodiments 83-90, wherein J₁₂ is O—C₁-C₆ alkoxy.
Embodiment 97. The oligomeric compound of any of embodiments 83-90, wherein J₁₂ is SCH₃.
Embodiment 98. The oligomeric compound of any of embodiments 6-97 comprising at least one stereo-non-standard nucleoside having a structure of Formula VII.
Embodiment 99. The oligomeric compound of embodiment 97, wherein J₁₃ is H.
Embodiment 100. The oligomeric compound of embodiment 97, wherein J₁₃ is OH.
Embodiment 101. The oligomeric compound of embodiment 97, wherein J₁₃ is F.
Embodiment 102. The oligomeric compound of embodiment 97, wherein J₁₃ is OCH₃.
Embodiment 103. The oligomeric compound of embodiment 97, wherein J₁₃ is OCH₂CH₂OCH₃.
Embodiment 104. The oligomeric compound of embodiment 97, wherein J₁₃ is O—C₁-C₆ alkoxy.
Embodiment 105. The oligomeric compound of embodiment 97, wherein J₁₃ is SCH₃.
Embodiment 106. The oligomeric compound of any of embodiments 97-105, wherein J₁₄ is H.
Embodiment 107. The oligomeric compound of any of embodiments 97-105, wherein J₁₄ is OH.
Embodiment 108. The oligomeric compound of any of embodiments 97-105, wherein J₁₄ is F.
Embodiment 109. The oligomeric compound of any of embodiments 97-105, wherein J₁₄ is OCH₃.
Embodiment 110. The oligomeric compound of any of embodiments 97-105, wherein J₁₄ is OCH₂CH₂OCH₃.
Embodiment 111. The oligomeric compound of any of embodiments 97-105, wherein J₁₄ is O—C₁-C₆ alkoxy.
Embodiment 112. The oligomeric compound of any of embodiments 97-105, wherein J₁₄ is SCH₃.
Embodiment 113. The oligomeric compound of any of embodiments 6-112, wherein Bx is selected from uracil, thymine, cytosine, 5-methyl cytosine, adenine and guanine.
Embodiment 114. The oligomeric compound any of embodiments 1-113, wherein exactly one nucleoside of the modified oligonucleotide is a stereo-non-standard nucleoside.
Embodiment 115. The oligomeric compound any of embodiments 1-113, wherein exactly two nucleosides of the modified oligonucleotide are stereo-non-standard nucleosides.
Embodiment 116. The oligomeric compound any of embodiments 1-113, wherein exactly three nucleosides of the modified oligonucleotide are stereo-non-standard nucleosides.
Embodiment 117. The oligomeric compound any of embodiments 1-113, wherein exactly four nucleosides of the modified oligonucleotide are stereo-non-standard nucleosides.
Embodiment 118. The oligomeric compound any of embodiments 1-113, wherein exactly five nucleosides of the modified oligonucleotide are stereo-non-standard nucleosides.
Embodiment 119. The oligomeric compound any of embodiments 1-113, wherein at least six nucleosides of the modified oligonucleotide are stereo-non-standard nucleosides.
Embodiment 120. The oligomeric compound of any of embodiments 1-113, wherein each nucleoside of the modified oligonucleotide is a stereo-non-standard nucleoside.
Embodiment 121. The oligomeric compound of any of embodiments 1-113 or 115-120, wherein the modified oligonucleotide has at least two stereo-non-standard nucleosides that are the same type of stereo-non-standard nucleoside as one another.
Embodiment 122. The oligomeric compound of any of embodiments 1-121, wherein the oligomeric compound is an RNAi compound.
Embodiment 123. The RNAi compound of embodiment 122, wherein the RNAi compound is an siRNA compound comprising an antisense siRNA oligonucleotide and a sense siRNA oligonucleotide, wherein at least one of the antisense siRNA oligonucleotide and the sense siRNA oligonucleotide is a modified oligonucleotide according to any of embodiments 1-121.
Embodiment 124. The siRNA compound of embodiment 123, wherein the antisense siRNA oligonucleotide consists of 17-30 linked nucleosides.
Embodiment 125. The siRNA compound of embodiment 123 or 124, wherein the antisense siRNA oligonucleotide is a modified oligonucleotide of any of embodiments 1-121.
Embodiment 126. The siRNA compound of embodiment 125, wherein at least one of the first 5 nucleosides from the 5′-end of the antisense siRNA oligonucleotide is a stereo-non-standard nucleoside.
Embodiment 127. The siRNA compound of any of embodiments 125-126, wherein at least one of the last 5 nucleosides counting back from the 3′-end of the antisense siRNA modified oligonucleotide is a stereo-non-standard nucleoside.
Embodiment 128. The siRNA compound of any of embodiments 125-127, wherein at least one nucleoside within the seed region of the antisense siRNA modified oligonucleotide is a stereo-non-standard nucleoside.
Embodiment 129. The siRNA compound of any of embodiments 123-128, wherein at least one nucleoside of the antisense siRNA oligonucleotide is a stereo-standard nucleoside or a bicyclic nucleoside.
Embodiment 130. The siRNA compound of embodiment 129, wherein at least one nucleoside of the antisense siRNA oligonucleotide is a substituted stereo-standard nucleoside or a bicyclic nucleoside.
Embodiment 131. The siRNA compound of embodiment 129 or 130, wherein at least one stereo-standard or bicyclic nucleoside of the antisense siRNA oligonucleotide is selected from: an LNA nucleoside, a cEt nucleoside, an ENA nucleoside, a 2′-MOE nucleoside, a 2′-OMe nucleoside, a 2′-F nucleoside, a 2′-NMA nucleoside, a 5′-Me nucleoside, a DNA nucleoside, and a RNA nucleoside.
Embodiment 132. The siRNA compound of any of embodiments 129-131, wherein each stereo-standard or bicyclic nucleoside of the antisense siRNA oligonucleotide is selected from: an LNA nucleoside, a cEt nucleoside, an ENA nucleoside, a 2′-MOE nucleoside, a 2′-OMe nucleoside, a 2′-F nucleoside, a 2′-NMA nucleoside, a 5′-Me nucleoside, a DNA nucleoside, and a RNA nucleoside.
Embodiment 133. The siRNA compound of any of embodiments 129-132, wherein at least one nucleoside of the antisense siRNA oligonucleotide is selected from: a 2′-OMe nucleoside, a 2′-F nucleoside, and a stereo-standard RNA nucleoside.
Embodiment 134. The siRNA compound of any of embodiments 129-133, wherein at least one nucleoside of the antisense siRNA oligonucleotide is a 2′-OMe nucleoside, and at least one nucleoside of the modified oligonucleotide is a stereo-standard RNA nucleoside.
Embodiment 135. The siRNA compound of any of embodiments 129-131 or 133-134, wherein at least one nucleoside of the antisense siRNA oligonucleotide is a (S)-GNA.
Embodiment 136. The siRNA compound of embodiment 135, wherein the (S)-GNA is at position 7 of the antisense strand as counted from the 5′ end.
Embodiment 137. The siRNA compound of any of embodiments 123-136 wherein the antisense siRNA oligonucleotide has at least one region of alternating nucleoside types having the motif ABABA wherein each A is a stereo-standard nucleoside having a sugar moiety of a first type and each B is a stereo-standard nucleoside having a sugar moiety of a second type, wherein the first type and the second type are different from one another.
Embodiment 138. The siRNA compound of embodiment 137 wherein A and B are selected from 2′-F nucleosides, 2′-OMe nucleosides, and RNA nucleosides.
Embodiment 139. The siRNA compound of any of embodiments 123-138, wherein the 5′-end of the antisense siRNA oligonucleotide comprises a stabilized phosphate group.
Embodiment 140. The siRNA compound of embodiment 139, wherein the stabilized phosphate group is 5′-vinyl phosphonate.
Embodiment 141. The siRNA compound of embodiment 139, wherein the stabilized phosphate group is 5′-cyclopropyl phosphonate.
Embodiment 142. The siRNA compound of any of embodiments 139-141, wherein the stabilized phosphate group is linked to the remainder of the antisense siRNA oligonucleotide through a 2′-5′ internucleoside linkage.
Embodiment 143. The siRNA compound of any of embodiments 123-142, wherein the sense siRNA oligonucleotide consists of 17-30 linked nucleosides.
Embodiment 144. The siRNA compound of any of embodiments 123-143, wherein the sense siRNA oligonucleotide is a modified oligonucleotide of any of embodiments 1-121.
Embodiment 145. The siRNA compound of any of embodiments 143-144, wherein at least one of the first 5 nucleosides from the 5′-end of the sense siRNA oligonucleotide is a stereo-non-standard nucleoside.
Embodiment 146. The siRNA compound of any of embodiments 143-145, wherein at least one of the last 5 nucleosides counting back from the 3′-end of the sense siRNA modified oligonucleotide is a stereo-non-standard nucleoside.
Embodiment 147. The siRNA compound of any of embodiments 143-146, wherein at least one nucleoside within the seed region of the sense siRNA modified oligonucleotide is a stereo-non-standard nucleoside.
Embodiment 148. The siRNA compound of any of embodiments 123-147, wherein at least one nucleoside of the sense siRNA oligonucleotide is a stereo-standard nucleoside or a bicyclic nucleoside
Embodiment 149. The siRNA compound of embodiment 148, wherein at least one nucleoside of the sense siRNA oligonucleotide is a substituted stereo-standard nucleoside or a bicyclic nucleoside.
Embodiment 150. The siRNA compound of embodiment 148 or 149 wherein at least one stereo-standard or bicyclic nucleoside of the sense siRNA oligonucleotide is selected from: an LNA nucleoside, a cEt nucleoside, an ENA nucleoside, a 2′-MOE nucleoside, a 2′-OMe nucleoside, a 2′-F nucleoside, a 2′-NMA nucleoside, a 5′-Me nucleoside, a DNA nucleoside, and a RNA nucleoside.
Embodiment 151. The siRNA compound of any of embodiments 148-150 wherein each stereo-standard or bicyclic nucleoside of the sense siRNA oligonucleotide is selected from: an LNA nucleoside, a cEt nucleoside, an ENA nucleoside, a 2′-MOE nucleoside, a 2′-OMe nucleoside, a 2′-F nucleoside, a 2′-NMA nucleoside, a 5′-Me nucleoside, a DNA nucleoside, and a RNA nucleoside.
Embodiment 152. The siRNA compound of any of embodiments 148-151, wherein at least one nucleoside of the sense siRNA oligonucleotide is selected from: a 2′-OMe nucleoside, a 2′-F nucleoside, and a stereo-standard RNA nucleoside.
Embodiment 153. The siRNA compound of any of embodiments 148-152 wherein at least one nucleoside of the sense siRNA oligonucleotide is a 2′-OMe nucleoside, and at least one nucleoside of the sense siRNA oligonucleotide is a stereo-standard RNA nucleoside.
Embodiment 154. The siRNA compound of any of embodiments 148-152, wherein at least one nucleoside of the sense siRNA oligonucleotide is an unlocked nucleic acid.
Embodiment 155. The siRNA compound of any of embodiments 148-154 wherein the sense siRNA oligonucleotide has at least one region of alternating nucleoside types having the motif ABABA wherein each A is a stereo-standard nucleoside having a sugar moiety of a first type and each B is a stereo-standard nucleoside having a sugar moiety of a second type, wherein the first type and the second type are different from one another.
Embodiment 156. The siRNA compound of embodiment 155 wherein A and B are selected from 2′-F nucleosides, 2′-OMe nucleosides, and RNA nucleosides.
Embodiment 157. The siRNA compound of any of embodiments 123-156, wherein the 5′-end of the sense siRNA oligonucleotide comprises a stabilized phosphate group.
Embodiment 158. The siRNA compound of any of embodiments 123-157, wherein at least one nucleoside of the antisense siRNA oligonucleotide and at least one nucleoside of the sense siRNA oligonucleotide is a stereo-non-standard nucleoside.
Embodiment 159. The siRNA compound of any of embodiments 123-158, wherein the antisense siRNA oligonucleotide has a nucleobase sequence comprising a targeting region comprising at least 15 contiguous nucleobases, wherein the nucleobase sequence of targeting region is at least 85% complementary to an equal length portion of the nucleobase sequence of a target RNA.
Embodiment 160. The siRNA compound of embodiment 159, wherein the nucleobase sequence of the targeting region is at least 90% complementary to the target RNA.
Embodiment 161. The siRNA compound of embodiment 160, wherein the nucleobase sequence of the targeting region is at least 95% complementary to the target RNA.
Embodiment 162. The siRNA compound of embodiment 160, wherein the nucleobase sequence of the targeting region is 100% complementary to the target RNA.
Embodiment 163. The siRNA compound of any of embodiments 159-162, wherein the targeting region comprises at least 18 contiguous nucleobases.
Embodiment 164. The siRNA of any of embodiments 159-163, wherein no more than 6 nucleobases of the antisense siRNA oligonucleotide are outside the targeting region.
Embodiment 165. The siRNA compound of any of embodiments 159-164, wherein the target RNA is a target mRNA, a target pre-mRNA, or a target microRNA.
Embodiment 166. The siRNA of compound 165, wherein the target RNA is a target mRNA.
Embodiment 167. The siRNA compound of any of embodiments 123-166, wherein the nucleobase sequence of the sense siRNA oligonucleotide comprises a duplexing region comprising at least 15 contiguous nucleobases, wherein the nucleobase sequence of the duplexing region of the sense siRNA oligonucleotide is at least 85% complementary to an equal length region portion of the nucleobase sequence of the antisense siRNA oligonucleotide.
Embodiment 168. The siRNA compound of embodiment 167, wherein the nucleobase sequence of the duplexing region is at least 90% complementary to the antisense siRNA oligonucleotide.
Embodiment 169. The siRNA compound of embodiment 167, wherein the nucleobase sequence of the duplexing region is at least 95% complementary to the antisense siRNA oligonucleotide.
Embodiment 170. The siRNA compound of embodiment 169, wherein the nucleobase sequence of the duplexing region is 100% complementary to the antisense siRNA oligonucleotide.
Embodiment 171. The siRNA compound of any of embodiments 167-170, wherein the duplexing region comprises at least 18 contiguous nucleobases.
Embodiment 172. The siRNA compound of any of embodiments 167-171, wherein no more than 6 nucleobases of the sense siRNA oligonucleotide are outside the duplexing region.
Embodiment 173. The siRNA compound of any of embodiments 123-172 comprising a conjugate.
Embodiment 174. The siRNA compound of embodiment 173, wherein a conjugate is attached to the antisense siRNA oligonucleotide.
Embodiment 175. The siRNA compound of embodiment 173 or 174, wherein a conjugate is attached to the sense siRNA oligonucleotide.
Embodiment 176. The siRNA compound of any of embodiments 173-175, wherein the conjugate comprises a GalNAc moiety.
Embodiment 177. The oligomeric compound of any of embodiments 173-176, wherein the conjugate group comprises 1-5 linker-nucleosides.
Embodiment 178. The siRNA compound of any of embodiments 122-177, wherein at least one stereo non-standard nucleoside is an independently a stereo-non-standard nucleoside of any of embodiments 6-121.
Embodiment 179. The siRNA compound of any of embodiments 122-177, wherein each stereo non-standard nucleoside is an independently a stereo-non-standard nucleoside of any of embodiments 6-121.
Embodiment 180. The siRNA compound of any of embodiments 122-177, wherein at least one stereo non-standard nucleoside is an independently a stereo-non-standard nucleoside of Formula I-VII.
Embodiment 181. The siRNA compound of any of embodiments 122-177, wherein each stereo non-standard nucleoside is an independently a stereo-non-standard nucleoside of Formula I-VII.
Embodiment 182. A pharmaceutical composition comprising the oligomeric compound, the RNAi compound, or the siRNA compound of any of embodiments 1-181.
Embodiment 183. The pharmaceutical composition of embodiment 182 comprising a pharmaceutically acceptable diluent.
Embodiment 184. A method comprising contacting a cell with the oligomeric compound, the RNAi compound, or the siRNA compound of any of embodiments 1-181 or the pharmaceutical composition of embodiment 182 or 183.
Embodiment 185. A method of administering to an animal the oligomeric compound, the RNAi compound, or the siRNA compound of any of embodiments 1-181 or the pharmaceutical composition of embodiment 182 or 183.
Embodiment 186. The RNAi compound of embodiment 122, wherein the RNAi compound is a single-stranded RNAi compound comprising a single-stranded RNAi oligonucleotide, wherein the single-stranded RNAi oligonucleotide is a modified oligonucleotide according to any of embodiments 1-121.
Embodiment 187. The single-stranded RNAi compound of embodiment 186, wherein the RNAi compound comprises a single-stranded RNAi oligonucleotide consisting of 12 to 30 linked nucleosides.
Embodiment 188. The single-stranded RNAi compound of any of embodiments 186-187, wherein at least one of the first 5 nucleosides from the 5′-end of the single-stranded RNAi oligonucleotide is a stereo-non-standard nucleoside.
Embodiment 189. The single-stranded RNAi compound of any of embodiments 186-188, wherein at least one of the last 5 nucleosides counting back from the 3′-end of the antisense siRNA modified oligonucleotide is a stereo-non-standard nucleoside.
Embodiment 190. The single-stranded RNAi compound of any of embodiments 186-189, wherein at least one nucleoside within the seed region of the single-stranded RNAi oligonucleotide is a stereo-non-standard nucleoside.
Embodiment 191. The single-stranded RNAi compound of any of embodiments 186-189, wherein at least one nucleoside of the single-stranded RNAi oligonucleotide is a stereo-standard nucleoside or a bicyclic nucleoside.
Embodiment 192. The single-stranded RNAi compound of embodiment 191, wherein at least one nucleoside of the single-stranded RNAi oligonucleotide is a substituted stereo-standard nucleoside or a bicyclic nucleoside.
Embodiment 193. The single-stranded RNAi compound of embodiment 191 or 192, wherein at least one stereo-standard or bicyclic nucleoside of the single-stranded RNAi oligonucleotide is selected from: an LNA nucleoside, a cEt nucleoside, an ENA nucleoside, a 2′-MOE nucleoside, a 2′-OMe nucleoside, a 2′-F nucleoside, a 2′-NMA nucleoside, a 5′-Me nucleoside, a DNA nucleoside, and a RNA nucleoside.
Embodiment 194. The single-stranded RNAi compound of any of embodiments 191-193, wherein each stereo-standard or bicyclic nucleoside of the single-stranded RNAi oligonucleotide is selected from: an LNA nucleoside, a cEt nucleoside, an ENA nucleoside, a 2′-MOE nucleoside, a 2′-OMe nucleoside, a 2′-F nucleoside, a 2′-NMA nucleoside, a 5′-Me nucleoside, a DNA nucleoside, and a RNA nucleoside.
Embodiment 195. The single-stranded RNAi compound of any of embodiments 191-194, wherein at least one nucleoside of the single-stranded RNAi oligonucleotide is selected from: a 2′-OMe nucleoside, a 2′-F nucleoside, and a stereo-standard RNA nucleoside.
Embodiment 196. The single-stranded RNAi compound of any of embodiments 191-195, wherein at least one nucleoside of the single-stranded RNAi oligonucleotide is a 2′-OMe nucleoside, and at least one nucleoside of the modified oligonucleotide is a stereo-standard RNA nucleoside.
Embodiment 197. The single-stranded RNAi compound of any of embodiments 191-196 wherein the single-stranded RNAi oligonucleotide has at least one region of alternating nucleoside types having the motif ABABA wherein each A is a stereo-standard nucleoside having a sugar moiety of a first type and each B is a stereo-standard nucleoside having a sugar moiety of a second type, wherein the first type and the second type are different from one another.
Embodiment 198. The single-stranded RNAi compound of embodiment 197 wherein A and B are selected from 2′-F nucleosides, 2′-OMe nucleosides, and RNA nucleosides.
Embodiment 199. The single-stranded RNAi compound of any of embodiments 186-198, wherein the 5′-end of the single-stranded RNAi oligonucleotide comprises a stabilized phosphate group.
Embodiment 200. The single-stranded RNAi compound of embodiment 199, wherein the stabilized phosphate group is 5′-vinyl phosphonate.
Embodiment 201. The single-stranded RNAi compound of embodiment 200, wherein the stabilized phosphate group is 5′-cyclopropyl phosphonate.
Embodiment 202. The single-stranded RNAi compound of any of embodiments 199-201, wherein the stabilized phosphate group is linked to the remainder of the single-stranded RNAi oligonucleotide through a 2′-5′ internucleoside linkage.
Embodiment 203. The single-stranded RNAi compound of any of embodiments 186-202, wherein the single-stranded RNAi oligonucleotide has a nucleobase sequence comprising a targeting region comprising at least 15 contiguous nucleobases, wherein the nucleobase sequence of targeting region is at least 85% complementary to an equal length portion of the nucleobase sequence of a target RNA.
Embodiment 204. The single-stranded RNAi compound of embodiment 203, wherein the nucleobase sequence of the targeting region is at least 90% complementary to the target RNA.
Embodiment 205. The single-stranded RNAi compound of embodiment 203, wherein the nucleobase sequence of the targeting region is at least 95% complementary to the target RNA.
Embodiment 206. The single-stranded RNAi compound of embodiment 203, wherein the nucleobase sequence of the targeting region is 100% complementary to the target RNA.
Embodiment 207. The single-stranded RNAi compound of any of embodiments 203-206, wherein the targeting region comprises at least 18 contiguous nucleobases.
Embodiment 208. The single-stranded RNAi compound of any of embodiments 203-207, wherein no more than 6 nucleobases are outside the targeting region.
Embodiment 209. The single-stranded RNAi compound of any of embodiments 203-208, wherein the target RNA is a target mRNA, a target pre-mRNA, or a target microRNA.
Embodiment 210. The single-stranded RNAi compound of compound 209, wherein the target RNA is a target mRNA.
Embodiment 211. The single-stranded RNAi compound of any of embodiments 186-210 comprising a conjugate.
Embodiment 212. The single-stranded RNAi compound of embodiment 211, wherein a conjugate is attached to the single-stranded RNAi oligonucleotide.
Embodiment 213. The single-stranded RNAi compound of any of embodiments 211-212, wherein the conjugate comprises a GalNAc moiety.
Embodiment 214. The oligomeric compound of any of embodiments 211-213, wherein the conjugate group comprises 1-5 linker-nucleosides.
Embodiment 215. The single-stranded RNAi compound of any of embodiments 186-214, wherein at least one stereo non-standard nucleoside is an independently a stereo-non-standard nucleoside of any of embodiments 6-177.
Embodiment 216. The single-stranded RNAi compound of any of embodiments 186-214, wherein each stereo non-standard nucleoside is an independently a stereo-non-standard nucleoside of any of embodiments 6-177.
Embodiment 217. The single-stranded RNAi compound of any of embodiments 186-214, wherein at least one stereo non-standard nucleoside is an independently a stereo-non-standard nucleoside of Formula I-VII.
Embodiment 218. The single-stranded RNAi compound of any of embodiments 186-214, wherein each stereo non-standard nucleoside is an independently a stereo-non-standard nucleoside of Formula I-VII.
Embodiment 219. A pharmaceutical composition comprising the single-stranded RNAi compound of any of embodiments 186-218.
Embodiment 220. The pharmaceutical composition of embodiment 219 comprising a pharmaceutically acceptable diluent.
Embodiment 221. A method comprising contacting a cell with the single-stranded RNAi compound of any of embodiments 185-216 or the pharmaceutical composition of embodiment 219 or 220.
Embodiment 222. A method of administering to an animal the single-stranded RNAi compound of any of embodiments 185-216 or the pharmaceutical composition of embodiment 219 or 220.
Embodiment 223. The oligomeric compound of any of embodiments 1-121, wherein the oligomeric compound is a CRISPR compound according to any of embodiments 1-121.
Embodiment 224. The CRISPR compound of embodiment 223 comprising a CRISPR oligonucleotide consisting of 20-120 linked nucleosides, wherein the CRISPR oligonucleotide is a modified oligonucleotide according to any of embodiments 1-121.
Embodiment 225. The CRISPR compound of embodiment 223, wherein the CRISPR oligonucleotide consists of 50-120 linked nucleosides.
Embodiment 226. The CRISPR compound of embodiment 223, wherein the CRISPR oligonucleotide consists of 20-50 linked nucleosides.
Embodiment 227. The CRISPR compound of embodiment 223, wherein the CRISPR oligonucleotide consists of 29-32 linked nucleosides.
Embodiment 228. The CRISPR compound of embodiment 223, wherein the CRISPR oligonucleotide consists of 20-28 linked nucleosides.
Embodiment 229. The CRISPR compound of embodiment 223, wherein the CRISPR oligonucleotide consists of 29 linked nucleosides.
Embodiment 230. The CRISPR compound of embodiment 223, wherein the CRISPR oligonucleotide consists of 32 linked nucleosides.
Embodiment 231. The CRISPR oligonucleotide of any of embodiments 223-230, wherein at least one of the first 5 nucleosides from the 5′-end of the CRISPR oligonucleotide is a stereo-non-standard nucleoside.
Embodiment 232. The CRISPR oligonucleotide of any of embodiments 223-231, wherein at least one of the last 5 nucleosides counting back from the 3′-end of the CRISPR oligonucleotide is a stereo-non-standard nucleoside.
Embodiment 233. The CRISPR oligonucleotide of any of embodiments 223-232, wherein at least one nucleoside of the CRISPR oligonucleotide is a stereo-standard nucleoside or a bicyclic nucleoside.
Embodiment 234. The CRISPR oligonucleotide of embodiment 233, wherein at least one nucleoside of the CRISPR oligonucleotide is a substituted stereo-standard nucleoside.
Embodiment 235. The CRISPR oligonucleotide of embodiment 233 or 234, wherein at least one nucleoside of the CRISPR oligonucleotide is a bicyclic nucleoside.
Embodiment 236. The CRISPR oligonucleotide of any of embodiments 233-235, wherein at least one stereo-standard or bicyclic nucleoside of the CRISPR oligonucleotide is selected from: an LNA nucleoside, a cEt nucleoside, an ENA nucleoside, a 2′-MOE nucleoside, a 2′-OMe nucleoside, a 2′-F nucleoside, a 2′-NMA nucleoside, a 5′-Me nucleoside, a DNA nucleoside, and a RNA nucleoside.
Embodiment 237. The CRISPR oligonucleotide of any of embodiments 233-236, wherein each stereo-standard or bicyclic nucleoside of the modified CRISPR oligonucleotide is selected from: an LNA nucleoside, a cEt nucleoside, an ENA nucleoside, a 2′-MOE nucleoside, a 2′-OMe nucleoside, a 2′-F nucleoside, a 2′-NMA nucleoside, a 5′-Me nucleoside, a DNA nucleoside, and a RNA nucleoside.
Embodiment 238. The CRISPR oligonucleotide of any of embodiments 233-237, wherein at least one nucleoside of the modified CRISPR oligonucleotide is selected from: a 2′-OMe nucleoside, a 2′-F nucleoside, and a stereo-standard RNA nucleoside.
Embodiment 239. The CRISPR oligonucleotide of any of embodiments 233-238 wherein at least one nucleoside of the modified CRISPR oligonucleotide is a 2′-OMe nucleoside, and at least one nucleoside of the modified oligonucleotide is a stereo-standard RNA nucleoside.
Embodiment 240. The CRISPR oligonucleotide of any of embodiments 233-239 wherein the CRISPR oligonucleotide has at least one region of alternating nucleoside types having the motif ABABA wherein each A is a stereo-standard nucleoside having a sugar moiety of a first type and each B is a stereo-standard nucleoside having a sugar moiety of a second type, wherein the first type and the second type are different from one another.
Embodiment 241. The CRISPR oligonucleotide of embodiment 240 wherein A and B are selected from 2′-F nucleosides, 2′-OMe nucleosides, and RNA nucleosides.
Embodiment 242. The CRISPR oligonucleotide of embodiment any of embodiments 233-241, wherein the CRISPR oligonucleotide is a modified crRNA oligonucleotide.
Embodiment 243. The CRISPR oligonucleotide of embodiment 242, wherein the tracrRNA recognition portion of the crRNA consists of 12 or fewer linked nucleosides.
Embodiment 244. The CRISPR oligonucleotide of any of embodiments 233-243, wherein the DNA recognition portion of the CRISPR oligonucleotide consists of 17 or fewer linked nucleosides.
Embodiment 245. The CRISPR oligonucleotide of any of embodiments 233-244, wherein at least one stereo non-standard nucleoside is an independently a stereo-non-standard nucleoside of any of embodiments 6-121.
Embodiment 246. The CRISPR oligonucleotide of any of embodiments 233-244, wherein each stereo non-standard nucleoside is an independently a stereo-non-standard nucleoside of any of embodiments 6-121.
Embodiment 247. The CRISPR oligonucleotide of any of embodiments 233-244, wherein at least one stereo non-standard nucleoside is an independently a stereo-non-standard nucleoside of Formula I-VII.
Embodiment 248. The CRISPR oligonucleotide of any of embodiments 233-244, wherein each stereo non-standard nucleoside is an independently a stereo-non-standard nucleoside of Formula I-VII.
Embodiment 249. A method comprising contacting a cell with the CRISPR oligonucleotide of any of embodiments 233-248.
Embodiment 250. The method of embodiment 249 comprising contacting the cell with a plasmid that encodes Cas9 or Cpf1.
Embodiment 251. The compound of embodiment 250, wherein the plasmid encodes a tracrRNA.
Embodiment 252. The method of embodiment 249, comprising contacting the cell with an mRNA that encodes Cas9 or Cpf1.
Embodiment 253. The method of embodiment 252 comprising contacting the cell with a plasmid that encodes a tracrRNA.
Embodiment 254. The method of any of embodiments 249-253 wherein a target gene is edited.
Embodiment 255. A pharmaceutical composition comprising the CRISPR compound or the CRISPR oligonucleotide of any of embodiments 223-254.
Embodiment 256. The pharmaceutical composition of embodiment 255 comprising a pharmaceutically acceptable diluent.
Embodiment 257. A method comprising contacting a cell with the CRISPR compound or the CRISPR oligonucleotide of any of embodiments 233-248 or the pharmaceutical composition of embodiment 255 or 256.
Embodiment 258. A method of administering to an animal the CRISPR compound or the CRISPR oligonucleotide of any of embodiments 233-248 or the pharmaceutical composition of embodiment 255 or 256.
Embodiment 259. The oligomeric compound of any of embodiments 1-121, wherein the oligomeric compound is an artificial mRNA compound.
Embodiment 260. The artificial mRNA compound of embodiment 259, wherein the oligomeric compound is an artificial mRNA oligonucleotide consisting of 17-3000 linked nucleosides, and wherein the artificial mRNA oligonucleotide is a modified oligonucleotide according to any of embodiments 1-121.
Embodiment 261. The artificial mRNA oligonucleotide of any of embodiments 259-260, wherein at least one of the first 5 nucleosides from the 5′-end of the artificial mRNA oligonucleotide is a stereo-non-standard nucleoside.
Embodiment 262. The artificial mRNA oligonucleotide of any of embodiments 259-261, wherein at least one of the last 5 nucleosides counting back from the 3′-end of the artificial mRNA oligonucleotide is a stereo-non-standard nucleoside.
Embodiment 263. The artificial mRNA oligonucleotide of any of embodiments 259-262, wherein at least one nucleoside of the artificial mRNA oligonucleotide is a stereo-standard nucleoside.
Embodiment 264. The artificial mRNA oligonucleotide of any of embodiments 259-263, wherein at least one nucleoside of the 5′-UTR of the artificial mRNA oligonucleotide is a stereo-non-standard nucleoside.
Embodiment 265. The artificial mRNA oligonucleotide of any of embodiments 259-264, wherein at least one nucleoside of the 3′-UTR of the artificial mRNA oligonucleotide is a stereo-non-standard nucleoside.
Embodiment 266. The artificial mRNA oligonucleotide of any of embodiments 259-265, wherein at least one nucleoside of the poly-A tail region of the artificial mRNA oligonucleotide is a stereo-non-standard nucleoside.
Embodiment 267. The artificial mRNA oligonucleotide of any of embodiments 259-266, wherein at least one nucleoside of the coding region of the artificial mRNA oligonucleotide is a stereo-non-standard nucleoside.
Embodiment 268. The artificial mRNA oligonucleotide of any of embodiments 259-267, wherein at least one stereo non-standard nucleoside is an independently a stereo-non-standard nucleoside of any of embodiments 6-177.
Embodiment 269. The artificial mRNA oligonucleotide of any of embodiments 259-267, wherein each stereo non-standard nucleoside is an independently a stereo-non-standard nucleoside of any of embodiments 6-177.
Embodiment 270. The artificial mRNA oligonucleotide of any of embodiments 259-267, wherein at least one stereo non-standard nucleoside is an independently a stereo-non-standard nucleoside of Formula I-VII.
Embodiment 271. The artificial mRNA oligonucleotide of any of embodiments 259-267, wherein each stereo non-standard nucleoside is an independently a stereo-non-standard nucleoside of Formula I-VII.
Embodiment 272. A pharmaceutical composition comprising the artificial mRNA oligonucleotide of any of embodiments 259-271, and a pharmaceutically acceptable carrier or diluent.
Embodiment 273. A pharmaceutical composition comprising the artificial mRNA oligonucleotide of any of embodiments 259-272 and a lipid nanoparticle.
Embodiment 274. A method comprising contacting a cell with the artificial mRNA compound or the artificial mRNA oligonucleotide of any of embodiments 259-271 or the pharmaceutical composition of embodiment 272 or 273.
Embodiment 275. A method of administering to an animal the artificial mRNA compound or the artificial mRNA oligonucleotide of any of embodiments 259-271 or the pharmaceutical composition of embodiment 272 or 273.
Embodiment 276. An oligomeric compound comprising a modified oligonucleotide consisting of 12-3000 linked nucleosides, wherein at least one nucleoside of the modified oligonucleotide is a stereo-non-standard nucleoside; and wherein the oligomeric compound is selected from among an RNAi compound, a modified CRISPR compound, and an artificial mRNA.
Embodiment 277. The oligomeric compound of embodiment 276 comprising at least one stereo-non-standard DNA nucleoside.
Embodiment 278. The oligomeric compound of embodiment 276 or 277 comprising at least one stereo-non-standard RNA nucleoside.
Embodiment 279. The oligomeric compound of any of embodiments 276-278 comprising at least one substituted stereo-non-standard nucleoside.
Embodiment 280. The oligomeric compound of any of embodiments 276-279 comprising at least one 2′-substituted stereo-non-standard nucleoside.
Embodiment 281. The oligomeric compound of any of embodiment 276-280, wherein at least one stereo-non-standard nucleoside has a structure selected from Formula I, Formula II, Formula III, Formula IV, Formula V, Formula VI, and Formula VII:

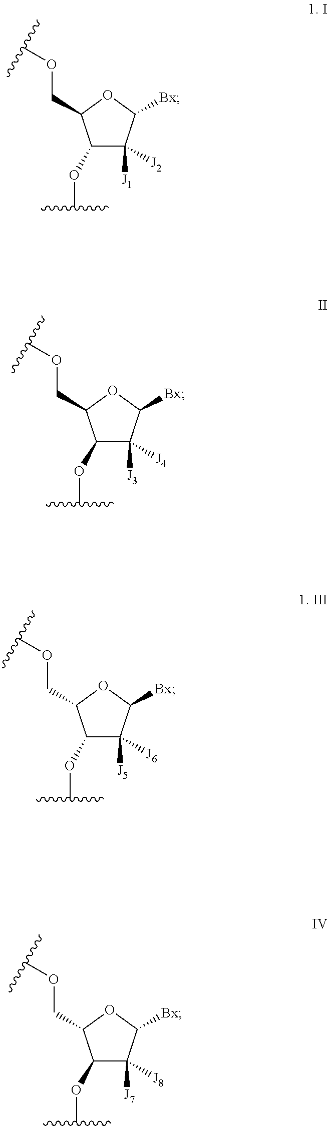

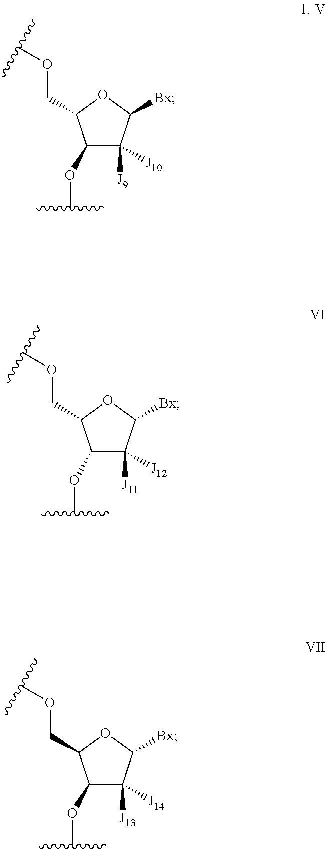

wherein
one of J₁ and J₂ is H and the other of J₁ and J₂ is selected from H, OH, F, OCH₃, OCH₂CH₂OCH₃, O—C₁-C₆ alkoxy, and SCH₃;
one of J₃ and J₄ is H and the other of J₃ and J₄ is selected from H, OH, F, OCH₃, OCH₂CH₂OCH₃, O—C₁-C₆ alkoxy, and SCH₃; and wherein
one of J₅ and J₆ is H and the other of J₅ and J₆ is selected from H, OH, F, OCH₃, OCH₂CH₂OCH₃, O—C₁-C₆ alkoxy, and SCH₃; and wherein
one of J₇ and J₈ is H and the other of J₇ and J₈ is selected from H, OH, F, OCH₃, OCH₂CH₂OCH₃, O—C₁-C₆ alkoxy, and SCH₃; and wherein
one of J₉ and J₁₀ is H and the other of J₉ and J₁₀ is selected from H, OH, F, OCH₃, OCH₂CH₂OCH₃, O—C₁-C₆ alkoxy, and SCH₃; and wherein
one of J₁₁ and J₁₂ is H and the other of J₁₁ and J₁₂ is selected from H, OH, F, OCH₃, OCH₂CH₂OCH₃, O—C₁-C₆ alkoxy, and SCH₃; and wherein
one of J₁₃ and J₁₄ is H and the other of J₁₃ and J₁₄ is selected from H, OH, F, OCH₃, OCH₂CH₂OCH₃, O—C₁-C₆ alkoxy, and SCH₃; and
Bx is a is a heterocyclic base moiety.
Embodiment 282. The oligomeric compound of embodiment 281, wherein:
   one of J₁ and J₂ is H and the other of J₁ and J₂ is selected from OH, F, OCH₃, OCH₂CH₂OCH₃, O—C₁-C₆ alkoxy, and SCH₃;
   one of J₃ and J₄ is H and the other of J₃ and J₄ is selected from OH, F, OCH₃, OCH₂CH₂OCH₃, O—C₁-C₆ alkoxy, and SCH₃;
   one of J₅ and J₆ is H and the other of J₅ and J₆ is selected from OH, F, OCH₃, OCH₂CH₂OCH₃, O—C₁-C₆ alkoxy, and SCH₃;
   one of J₇ and J₈ is H and the other of J₇ and J₈ is selected from OH, F, OCH₃, OCH₂CH₂OCH₃, O—C₁-C₆ alkoxy, and SCH₃;
   one of J₉ and J₁₀ is H and the other of J₉ and J₁₀ is selected from OH, F, OCH₃, OCH₂CH₂OCH₃, O—C₁-C₆ alkoxy, and SCH₃;
   one of J₁₁ and J₁₂ is H and the other of J₁₁ and J₁₂ is selected from OH, F, OCH₃, OCH₂CH₂OCH₃, O—C₁-C₆ alkoxy, and SCH₃;
   one of J₁₃ and J₁₄ is H and the other of J₁₃ and J₁₄ is selected from OH, F, OCH₃, OCH₂CH₂OCH₃, O—C₁-C₆ alkoxy, and SCH₃.
Embodiment 283. The oligomeric compound of embodiment 281 or 282 comprising at least one stereo-non-standard nucleoside having a structure of Formula I.
Embodiment 284. The oligomeric compound of embodiment 283, wherein J₁ is H.
Embodiment 285. The oligomeric compound of embodiment 283, wherein J₁ is OH.
Embodiment 286. The oligomeric compound of embodiment 283, wherein J₁ is F.
Embodiment 287. The oligomeric compound of embodiment 283, wherein J₁ is OCH₃.
Embodiment 288. The oligomeric compound of embodiment 283, wherein J₁ is OCH₂CH₂OCH₃.
Embodiment 289. The oligomeric compound of embodiment 283, wherein J₁ is O—C₁-C₆ alkoxy.
Embodiment 290. The oligomeric compound of embodiment 283, wherein J₁ is SCH₃.
Embodiment 291. The oligomeric compound of any of embodiments 283-290, wherein J₂ is H.
Embodiment 292. The oligomeric compound of embodiments 283-290, wherein J₂ is OH.
Embodiment 293. The oligomeric compound of embodiments 283-290, wherein J₂ is F.
Embodiment 294. The oligomeric compound of embodiments 283-290, wherein J₂ is OCH₃.
Embodiment 295. The oligomeric compound of embodiments 283-290, wherein J₂ is OCH₂CH₂OCH₃.
Embodiment 296. The oligomeric compound of embodiments 283-290, wherein J₂ is O—C₁-C₆ alkoxy.
Embodiment 297. The oligomeric compound of embodiments 283-290, wherein J₂ is SCH₃.
Embodiment 298. The oligomeric compound of any of embodiments 281-297 comprising at least one stereo-non-standard nucleoside having a structure of Formula II.
Embodiment 299. The oligomeric compound of embodiment 298, wherein J₃ is H.
Embodiment 300. The oligomeric compound of embodiment 298, wherein J₃ is OH.
Embodiment 301. The oligomeric compound of embodiment 298, wherein J₃ is F.
Embodiment 302. The oligomeric compound of embodiment 298, wherein J₃ is OCH₃.
Embodiment 303. The oligomeric compound of embodiment 298, wherein J₃ is OCH₂CH₂OCH₃.
Embodiment 304. The oligomeric compound of embodiment 298, wherein J₃ is O—C₁-C₆ alkoxy.
Embodiment 305. The oligomeric compound of embodiment 298, wherein J₃ is SCH₃.
Embodiment 306. The oligomeric compound of any of embodiments 298-305, wherein J₄ is H.
Embodiment 307. The oligomeric compound of any of embodiments 298-305, wherein J₄ is OH.
Embodiment 308. The oligomeric compound of any of embodiments 298-305, wherein J₄ is F.
Embodiment 309. The oligomeric compound of any of embodiments 298-305, wherein J₄ is OCH₃.
Embodiment 310. The oligomeric compound of any of embodiments 298-305, wherein J₄ is OCH₂CH₂OCH₃.
Embodiment 311. The oligomeric compound of any of embodiments 298-305, wherein J₄ is O—C₁-C₆ alkoxy.
Embodiment 312. The oligomeric compound of any of embodiments 298-305, wherein J₄ is SCH₃.
Embodiment 313. The oligomeric compound of any of embodiments 281-312 comprising at least one stereo-non-standard nucleoside having a structure of Formula III.
Embodiment 314. The oligomeric compound of embodiment 313, wherein J₅ is H.
Embodiment 315. The oligomeric compound of embodiment 313, wherein J₅ is OH.
Embodiment 316. The oligomeric compound of embodiment 313, wherein J₅ is F.
Embodiment 317. The oligomeric compound of embodiment 313, wherein J₅ is OCH₃.
Embodiment 318. The oligomeric compound of embodiment 313, wherein J₅ is OCH₂CH₂OCH₃.
Embodiment 319. The oligomeric compound of embodiment 313, wherein J₅ is O—C₁-C₆ alkoxy.
Embodiment 320. The oligomeric compound of embodiment 313, wherein J₅ is SCH₃.
Embodiment 321. The oligomeric compound of any of embodiments 313-320, wherein J₆ is H.
Embodiment 322. The oligomeric compound of any of embodiments 313-320, wherein J₆ is OH.
Embodiment 323. The oligomeric compound of any of embodiments 313-320, wherein J₆ is F.
Embodiment 324. The oligomeric compound of any of embodiments 313-320, wherein J₆ is OCH₃.
Embodiment 325. The oligomeric compound of any of embodiments 313-320, wherein J₆ is OCH₂CH₂OCH₃.
Embodiment 326. The oligomeric compound of any of embodiments 313-320, wherein J₆ is O—C₁-C₆ alkoxy.
Embodiment 327. The oligomeric compound of any of embodiments 313-320, wherein J₆ is SCH₃.
Embodiment 328. The oligomeric compound of any of embodiments 281-327 comprising at least one stereo-non-standard nucleoside having a structure of Formula IV.
Embodiment 329. The oligomeric compound of embodiment 328, wherein J₇ is H.
Embodiment 330. The oligomeric compound of embodiment 328, wherein J₇ is OH.
Embodiment 331. The oligomeric compound of embodiment 328, wherein J₇ is F.
Embodiment 332. The oligomeric compound of embodiment 328, wherein J₇ is OCH₃.
Embodiment 333. The oligomeric compound of embodiment 328, wherein J₇ is OCH₂CH₂OCH₃.
Embodiment 334. The oligomeric compound of embodiment 328, wherein J₇ is O—C₁-C₆ alkoxy.
Embodiment 335. The oligomeric compound of embodiment 328, wherein J₇ is SCH₃.
Embodiment 336. The oligomeric compound of any of embodiments 328-335, wherein J₈ is H.
Embodiment 337. The oligomeric compound of any of embodiments 328-335, wherein J₈ is OH.
Embodiment 338. The oligomeric compound of any of embodiments 328-335, wherein J₈ is F.
Embodiment 339. The oligomeric compound of any of embodiments 328-335, wherein J₈ is OCH₃.
Embodiment 340. The oligomeric compound of any of embodiments 328-335, wherein J₈ is OCH₂CH₂OCH₃.
Embodiment 341. The oligomeric compound of any of embodiments 328-335, wherein J₈ is O—C₁-C₆ alkoxy.
Embodiment 342. The oligomeric compound of any of embodiments 328-335, wherein J₈ is SCH₃.
Embodiment 343. The oligomeric compound of any of embodiments 281-342 comprising at least one stereo-non-standard nucleoside having a structure of Formula V.
Embodiment 344. The oligomeric compound of embodiment 343, wherein J₉ is H.
Embodiment 345. The oligomeric compound of embodiment 343, wherein J₉ is OH.
Embodiment 346. The oligomeric compound of embodiment 343, wherein J₉ is F.
Embodiment 347. The oligomeric compound of embodiment 343, wherein J₉ is OCH₃.
Embodiment 348. The oligomeric compound of embodiment 343, wherein J₉ is OCH₂CH₂OCH₃.
Embodiment 349. The oligomeric compound of embodiment 343, wherein J₉ is O—C₁-C₆ alkoxy.
Embodiment 350. The oligomeric compound of embodiment 343, wherein J₉ is SCH₃.
Embodiment 351. The oligomeric compound of any of embodiments 343-350, wherein J₁₀ is H.
Embodiment 352. The oligomeric compound of any of embodiments 343-350, wherein J₁₀ is OH.
Embodiment 353. The oligomeric compound of any of embodiments 343-350, wherein J₁₀ is F.
Embodiment 354. The oligomeric compound of any of embodiments 343-350, wherein J₁₀ is OCH₃.
Embodiment 355. The oligomeric compound of any of embodiments 343-350, wherein J₁₀ is OCH₂CH₂OCH₃.
Embodiment 356. The oligomeric compound of any of embodiments 343-350, wherein J₁₀ is O—C₁-C₆ alkoxy.
Embodiment 357. The oligomeric compound of any of embodiments 343-350, wherein J₁₀ is SCH₃.
Embodiment 358. The oligomeric compound of any of embodiments 281-357 comprising at least one stereo-non-standard nucleoside having a structure of Formula VI.
Embodiment 359. The oligomeric compound of embodiment 358, wherein J₁₁ is H.
Embodiment 360. The oligomeric compound of embodiment 358, wherein J₁₁ is OH.
Embodiment 361. The oligomeric compound of embodiment 358, wherein J₁₁ is F.
Embodiment 362. The oligomeric compound of embodiment 358, wherein J₁₁ is OCH₃.
Embodiment 363. The oligomeric compound of embodiment 358, wherein J₁₁ is OCH₂CH₂OCH₃.
Embodiment 364. The oligomeric compound of embodiment 358, wherein J₁₁ is O—C₁-C₆ alkoxy.
Embodiment 365. The oligomeric compound of embodiment 358, wherein J₁₁ is SCH₃.
Embodiment 366. The oligomeric compound of any of embodiments 358-365, wherein J₁₂ is H.
Embodiment 367. The oligomeric compound of any of embodiments 358-365, wherein J₁₂ is OH.
Embodiment 368. The oligomeric compound of any of embodiments 358-365, wherein J₁₂ is F.
Embodiment 369. The oligomeric compound of any of embodiments 358-365, wherein J₁₂ is OCH₃.
Embodiment 370. The oligomeric compound of any of embodiments 358-365, wherein J₁₂ is OCH₂CH₂OCH₃.
Embodiment 371. The oligomeric compound of any of embodiments 358-365, wherein J₁₂ is O—C₁-C₆ alkoxy.
Embodiment 372. The oligomeric compound of any of embodiments 358-365, wherein J₁₂ is SCH₃.
Embodiment 373. The oligomeric compound of any of embodiments 281-372 comprising at least one stereo-non-standard nucleoside having a structure of Formula VII.
Embodiment 374. The oligomeric compound of embodiment 373, wherein J₁₃ is H.
Embodiment 375. The oligomeric compound of embodiment 373, wherein J₁₃ is OH.
Embodiment 376. The oligomeric compound of embodiment 373, wherein J₁₃ is F.
Embodiment 377. The oligomeric compound of embodiment 373, wherein J₁₃ is OCH₃.
Embodiment 378. The oligomeric compound of embodiment 373, wherein J₁₃ is OCH₂CH₂OCH₃.
Embodiment 379. The oligomeric compound of embodiment 373, wherein J₁₃ is O—C₁-C₆ alkoxy.
Embodiment 380. The oligomeric compound of embodiment 373, wherein J₁₃ is SCH₃.
Embodiment 381. The oligomeric compound of any of embodiments 373-380, wherein J₁₄ is H.
Embodiment 382. The oligomeric compound of any of embodiments 373-380, wherein J₁₄ is OH.
Embodiment 383. The oligomeric compound of any of embodiments 373-380, wherein J₁₄ is F.
Embodiment 384. The oligomeric compound of any of embodiments 373-380, wherein J₁₄ is OCH₃.
Embodiment 385. The oligomeric compound of any of embodiments 373-380, wherein J₁₄ is OCH₂CH₂OCH₃.
Embodiment 386. The oligomeric compound of any of embodiments 373-380, wherein J₁₄ is O—C₁-C₆ alkoxy.
Embodiment 387. The oligomeric compound of any of embodiments 373-380, wherein J₁₄ is SCH₃.
Embodiment 388. The oligomeric compound of any of embodiments 276-387, wherein Bx is selected from uracil, thymine, cytosine, 5-methyl cytosine, adenine and guanine.
Embodiment 389. The oligomeric compound any of embodiments 276-388, wherein exactly one nucleoside of the modified oligonucleotide is a stereo-non-standard nucleoside.
Embodiment 390. The oligomeric compound any of embodiments 276-388, wherein exactly two nucleosides of the modified oligonucleotide are stereo-non-standard nucleosides.
Embodiment 391. The oligomeric compound any of embodiments 276-388, wherein exactly three nucleosides of the modified oligonucleotide are stereo-non-standard nucleosides.
Embodiment 392. The oligomeric compound any of embodiments 276-388, wherein exactly four nucleosides of the modified oligonucleotide are stereo-non-standard nucleosides.
Embodiment 393. The oligomeric compound any of embodiments 276-388, wherein exactly five nucleosides of the modified oligonucleotide are stereo-non-standard nucleosides.
Embodiment 394. The oligomeric compound any of embodiments 276-388, wherein at least six nucleosides of the modified oligonucleotide are stereo-non-standard nucleosides.
Embodiment 395. The oligomeric compound of any of embodiments 276-388, wherein each nucleoside of the modified oligonucleotide is a stereo-non-standard nucleoside.
Embodiment 396. The oligomeric compound of any of embodiments 276-388 or 390-395, wherein the modified oligonucleotide has at least two stereo-non-standard nucleosides that are the same type of stereo-non-standard nucleoside as one another.
Embodiment 397. The oligomeric compound of any of embodiments 276-121, wherein the oligomeric compound is an RNAi compound.
Embodiment 398. The RNAi compound of embodiment 397, wherein the RNAi compound is a single-stranded RNAi compound comprising an antisense siRNA oligonucleotide.
Embodiment 399. The RNAi compound of embodiment 398, wherein the RNAi compound is a double-stranded RNAi compound comprising an antisense siRNA oligomeric compound an antisense siRNA oligonucleotide and a sense siRNA oligomeric compound comprising a sense siRNA oligonucleotide, wherein at least one of the antisense siRNA oligomeric compound and the sense siRNA oligomeric compound is an oligomeric compound according to any of embodiments 276-396.
Embodiment 400. The RNAi compound of embodiment 398 or 399, wherein the antisense siRNA oligonucleotide consists of 17-30 linked nucleosides.
Embodiment 401. The RNAi compound of any of embodiments 398-400, wherein the antisense siRNA oligomeric compound is an oligomeric compound of any of embodiments 276-396.
Embodiment 402. The RNAi compound of any of embodiments 398-401 wherein at least one of the first 5 nucleosides from the 5′-end of the antisense siRNA oligonucleotide is a stereo-non-standard nucleoside.
Embodiment 403. The RNAi compound of any of embodiments 398-402, wherein at least one of the last 5 nucleosides counting back from the 3′-end of the antisense siRNA modified oligonucleotide is a stereo-non-standard nucleoside.
Embodiment 404. The RNAi compound of any of embodiments 398-403, wherein at least one nucleoside within the seed region of the antisense siRNA modified oligonucleotide is a stereo-non-standard nucleoside.
Embodiment 405. The RNAi compound of any of embodiments 398-404, wherein the nucleoside at position 1 (from 5′ to 3′) of the antisense siRNA modified oligonucleotide is a stereo-non-standard nucleoside.
Embodiment 406. The RNAi compound of embodiment 405, wherein the stereo-non-standard nucleoside at position 1 (from 5′ to 3′) is linked to the nucleoside at position 2 with a mesyl phosphoramidate internucleoside linkage.
Embodiment 407. The RNAi compound of any of embodiments 398-406, wherein the nucleoside at position 2 (from 5′ to 3′) of the antisense siRNA modified oligonucleotide is a stereo-non-standard nucleoside.
Embodiment 408. The RNAi compound of any of embodiments 398-407, wherein the nucleoside at position 9 (from 5′ to 3′) of the antisense siRNA modified oligonucleotide is a stereo-non-standard nucleoside.
Embodiment 409. The RNAi compound of any of embodiments 398-408, wherein the nucleoside at position 14 (from 5′ to 3′) of the antisense siRNA modified oligonucleotide is a stereo-non-standard nucleoside.
Embodiment 410. The RNAi compound of any of embodiments 398-409, wherein the two 3′-most nucleosides of the antisense siRNA modified oligonucleotide are stereo-non-standard nucleosides.
Embodiment 411. The RNAi compound of any of embodiments 398-410, wherein the stereo-non-standard nucleoside comprises a sugar moiety selected from: a 2′-β-L-deoxyribosyl sugar moiety, a 2′-α-D-deoxyribosyl sugar moiety, a 2′-α-L-deoxyribosyl sugar moiety, a 2′-β-D-deoxyxylosyl sugar moiety, a 2′-β-L-deoxyxylosyl sugar moiety, 2′-α-D-deoxyxylosyl sugar moiety, a 2′-α-L-deoxyxylosyl sugar moiety, a 2′-fluoro-β-D-arabinosyl sugar moiety, a 2′-fluoro-β-D-xylosyl sugar moiety, a 2′-fluoro-α-D-ribosyl sugar moiety, a 2′-fluoro-α-D-arabinosyl sugar moiety, a 2′-fluoro-α-D-xylosyl sugar moiety, 2′-fluoro-α-L-ribosyl sugar moiety, a 2′-fluoro-β-L-xylosyl sugar moiety, a 2′-fluoro-α-L-arabinosyl sugar moiety, 2′-fluoro-α-L-xylosyl sugar moiety, 2′-fluoro-β-L-ribosyl sugar moiety, or a 2′-fluoro-β-L-arabinosyl sugar moiety.
Embodiment 412. The RNAi compound of any of embodiments 398-411, wherein the stereo-non-standard nucleoside comprises a sugar moiety selected from: a 2′-β-L-deoxyribosyl sugar moiety, a 2′-α-D-deoxyribosyl sugar moiety, a 2′-α-L-deoxyribosyl sugar moiety, 2′-α-D-deoxyxylosyl sugar moiety, a 2′-α-L-deoxyxylosyl sugar moiety, a 2′-fluoro-α-D-ribosyl sugar moiety, a 2′-fluoro-α-D-xylosyl sugar moiety, 2′-fluoro-α-L-xylosyl sugar moiety, or a 2′-fluoro-β-D-xylosyl sugar moiety.
Embodiment 413. The RNAi compound of any of embodiments 398-412, wherein at least one nucleoside of the antisense siRNA oligonucleotide is a stereo-standard nucleoside or a bicyclic nucleoside.
Embodiment 414. The RNAi compound of embodiment 413, wherein at least one nucleoside of the antisense siRNA oligonucleotide is a substituted stereo-standard nucleoside or a bicyclic nucleoside.
Embodiment 415. The RNAi compound of embodiment 413 or 414, wherein at least one stereo-standard or bicyclic nucleoside of the antisense siRNA oligonucleotide is selected from: an LNA nucleoside, a cEt nucleoside, an ENA nucleoside, a 2′-MOE nucleoside, a 2′-OMe nucleoside, a 2′-F nucleoside, a 2′-NMA nucleoside, a 5′-Me nucleoside, a DNA nucleoside, and a RNA nucleoside.
Embodiment 416. The RNAi compound of any of embodiments 413-415, wherein each stereo-standard or bicyclic nucleoside of the antisense siRNA oligonucleotide is selected from: an LNA nucleoside, a cEt nucleoside, an ENA nucleoside, a 2′-MOE nucleoside, a 2′-OMe nucleoside, a 2′-F nucleoside, a 2′-NMA nucleoside, a 5′-Me nucleoside, a DNA nucleoside, and a RNA nucleoside.
Embodiment 417. The RNAi compound of any of embodiments 413-416, wherein at least one nucleoside of the antisense siRNA oligonucleotide is selected from: a 2′-OMe nucleoside, a 2′-F nucleoside, and a stereo-standard RNA nucleoside.
Embodiment 418. The RNAi compound of any of embodiments 413-417, wherein at least one nucleoside of the antisense siRNA oligonucleotide is a 2′-OMe nucleoside, and at least one nucleoside of the modified oligonucleotide is a stereo-standard RNA nucleoside.
Embodiment 419. The RNAi compound of any of embodiments 413-415 or 417-418, wherein at least one nucleoside of the antisense siRNA oligonucleotide comprises the sugar surrogate (S)-GNA.
Embodiment 420. The RNAi compound of embodiment 419, wherein the (S)-GNA is at position 7 of the antisense strand as counted from the 5′ end.
Embodiment 421. The RNAi compound of any of embodiments 398-421, wherein the antisense siRNA oligonucleotide has at least one region of alternating nucleoside types having the motif ABABA wherein each A is a nucleoside having a sugar moiety of a first type and each B is a nucleoside having a sugar moiety of a second type, wherein the first type and the second type are different from one another.
Embodiment 422. The RNAi compound of embodiment 421, wherein A and B are selected from 2′-F nucleosides, 2′-OMe nucleosides, and RNA nucleosides.
Embodiment 423. The RNAi compound of embodiment 421, wherein A each A is a stereo-standard 2′-OMe nucleoside and each B is an independently selected stereo standard or stereo-non-standard 2′-F nucleoside.
Embodiment 424. The RNAi compound of any of embodiments 398-423, wherein the 5′-end of the antisense siRNA oligonucleotide comprises a stabilized phosphate group.
Embodiment 425. The RNAi compound of embodiment 424, wherein the stabilized phosphate group is 5′-vinyl phosphonate.
Embodiment 426. The RNAi compound of embodiment 424, wherein the stabilized phosphate group is 5′-cyclopropyl phosphonate.
Embodiment 427. The RNAi compound of embodiment 424, wherein the stabilized phosphate group is a 5′-mesyl phosphoramidate.
Embodiment 428. The RNAi compound of any of embodiments 425-427, wherein the stabilized phosphate group is linked to the remainder of the antisense siRNA oligonucleotide through a 2′-5′ internucleoside linkage.
Embodiment 429. The RNAi compound of any of embodiments 399-428, wherein the sense siRNA oligonucleotide consists of 17-30 linked nucleosides.
Embodiment 430. The RNAi compound of any of embodiments 399-429, wherein the sense siRNA oligomeric compound is an oligomeric compound of any of embodiments 276-396.
Embodiment 431. The RNAi compound of any of embodiments 399-430, wherein at least one of the first 5 nucleosides from the 5′-end of the sense siRNA oligonucleotide is a stereo-non-standard nucleoside.
Embodiment 432. The RNAi compound of any of embodiments 399-431, wherein at least one of the last 5 nucleosides counting back from the 3′-end of the sense siRNA modified oligonucleotide is a stereo-non-standard nucleoside.
Embodiment 433. The RNAi compound of any of embodiments 399-432, wherein at least one nucleoside within the seed region of the sense siRNA modified oligonucleotide is a stereo-non-standard nucleoside.
Embodiment 434. The RNAi compound of any of embodiments 399-433, wherein at least one nucleoside within the seed region of the sense siRNA modified oligonucleotide is a stereo-non-standard nucleoside.
Embodiment 435. The RNAi compound of any of embodiments 399-434, wherein the 10^{th} nucleoside of the sense siRNA modified oligonucleotide, counting from the 5′ end, is a stereo-non-standard nucleoside.
Embodiment 436. The RNAi compound of any of embodiments 399-435, wherein the stereo-non-standard nucleoside comprises a sugar moiety selected from: a 2′-fluoro-β-D-arabinosyl sugar moiety, a 2′-fluoro-β-D-xylosyl sugar moiety, a 2′-fluoro-α-D-ribosyl sugar moiety, a 2′-fluoro-α-D-arabinosyl sugar moiety, a 2′-fluoro-α-D-xylosyl sugar moiety, 2′-fluoro-α-L-ribosyl sugar moiety, a 2′-fluoro-β-L-xylosyl sugar moiety, a 2′-fluoro-α-L-arabinosyl sugar moiety, 2′-fluoro-α-L-xylosyl sugar moiety, 2′-fluoro-β-L-ribosyl sugar moiety, or a 2′-fluoro-β-L-arabinosyl sugar moiety.
Embodiment 437. The RNAi compound of embodiment 436, wherein the stereo-non-standard nucleoside comprises a sugar moiety selected from: a 2′-fluoro-β-D-xylosyl sugar moiety, 2′-fluoro-α-D-ribosyl sugar moiety, or a 2′-fluoro-α-D-xylosyl sugar moiety.
Embodiment 438. The RNAi compound of any of embodiments 399-437, wherein at least one nucleoside of the sense siRNA oligonucleotide is a stereo-standard nucleoside or a bicyclic nucleoside.
Embodiment 439. The RNAi compound of embodiment 438, wherein at least one nucleoside of the sense siRNA oligonucleotide is a substituted stereo-standard nucleoside or a bicyclic nucleoside.
Embodiment 440. The RNAi compound of embodiment 438 or 439 wherein at least one stereo-standard or bicyclic nucleoside of the sense siRNA oligonucleotide is selected from: an LNA nucleoside, a cEt nucleoside, an ENA nucleoside, a 2′-MOE nucleoside, a 2′-OMe nucleoside, a 2′-F nucleoside, a 2′-NMA nucleoside, a 5′-Me nucleoside, a DNA nucleoside, and a RNA nucleoside.
Embodiment 441. The RNAi compound of any of embodiments 399-440 wherein each stereo-standard or bicyclic nucleoside of the sense siRNA oligonucleotide is selected from: an LNA nucleoside, a cEt nucleoside, an ENA nucleoside, a 2′-MOE nucleoside, a 2′-OMe nucleoside, a 2′-F nucleoside, a 2′-NMA nucleoside, a 5′-Me nucleoside, a DNA nucleoside, and a RNA nucleoside.
Embodiment 442. The RNAi compound of any of embodiments 399-441, wherein at least one nucleoside of the sense siRNA oligonucleotide is selected from: a 2′-OMe nucleoside, a 2′-F nucleoside, and a stereo-standard RNA nucleoside.
Embodiment 443. The RNAi compound of any of embodiments 399-442 wherein at least one nucleoside of the sense siRNA oligonucleotide is a 2′-OMe nucleoside, and at least one nucleoside of the sense siRNA oligonucleotide is a stereo-standard 2′-F nucleoside.
Embodiment 444. The RNAi compound of any of embodiments 399-443, wherein at least one nucleoside of the sense siRNA oligonucleotide is an unlocked nucleic acid.
Embodiment 445. The RNAi compound of any of embodiments 399-444 wherein the sense siRNA oligonucleotide has at least one region of alternating nucleoside types having the motif ABABA wherein each A is a stereo-standard nucleoside having a sugar moiety of a first type and each B is a stereo-standard nucleoside having a sugar moiety of a second type, wherein the first type and the second type are different from one another.
Embodiment 446. The RNAi compound of embodiment 445 wherein A and B are selected from 2′-F nucleosides and 2′-OMe nucleosides.
Embodiment 447. The RNAi compound of any of embodiments 399-446, wherein the 5′-end of the sense siRNA oligonucleotide comprises a stabilized phosphate group.
Embodiment 448. The RNAi compound of any of embodiments 399-447, wherein at least one nucleoside of the antisense siRNA oligonucleotide and at least one nucleoside of the sense siRNA oligonucleotide is a stereo-non-standard nucleoside.
Embodiment 449. The RNAi compound of any of embodiments 399-448, wherein the antisense siRNA oligonucleotide has a nucleobase sequence comprising a targeting region comprising at least 15 contiguous nucleobases, wherein the nucleobase sequence of targeting region is at least 85% complementary to an equal length portion of the nucleobase sequence of a target RNA.
Embodiment 450. The RNAi compound of embodiment 449, wherein the nucleobase sequence of the targeting region is at least 90% complementary to the target RNA.
Embodiment 451. The RNAi compound of embodiment 450, wherein the nucleobase sequence of the targeting region is at least 95% complementary to the target RNA.
Embodiment 452. The RNAi compound of embodiment 450, wherein the nucleobase sequence of the targeting region is 100% complementary to the target RNA.
Embodiment 453. The RNAi compound of any of embodiments 449-452, wherein the targeting region comprises at least 18 contiguous nucleobases.
Embodiment 454. The RNAi compound of any of embodiments 449-453, wherein no more than 6 nucleobases of the antisense siRNA oligonucleotide are outside the targeting region.
Embodiment 455. The RNAi compound of any of embodiments 449-454, wherein the target RNA is a target mRNA, a target pre-mRNA, or a target microRNA.
Embodiment 456. The RNAi compound of embodiment 455, wherein the target RNA is a target mRNA.
Embodiment 457. The RNAi compound of any of embodiments 399-456, wherein the nucleobase sequence of the sense siRNA oligonucleotide comprises a duplexing region comprising at least 15 contiguous nucleobases, wherein the nucleobase sequence of the duplexing region of the sense siRNA oligonucleotide is at least 85% complementary to an equal length region portion of the nucleobase sequence of the antisense siRNA oligonucleotide.
Embodiment 458. The RNAi compound of embodiment 457, wherein the nucleobase sequence of the duplexing region is at least 90% complementary to the antisense siRNA oligonucleotide.
Embodiment 459. The RNAi compound of embodiment 457, wherein the nucleobase sequence of the duplexing region is at least 95% complementary to the antisense siRNA oligonucleotide.
Embodiment 460. The RNAi compound of embodiment 457, wherein the nucleobase sequence of the duplexing region is 100% complementary to the antisense siRNA oligonucleotide.
Embodiment 461. The RNAi compound of any of embodiments 457-460, wherein the duplexing region comprises at least 18 contiguous nucleobases.
Embodiment 462. The RNAi compound of any of embodiments 457-461, wherein no more than 6 nucleobases of the sense siRNA oligonucleotide are outside the duplexing region.
Embodiment 463. The RNAi compound of any of embodiments 398-462 comprising a conjugate.
Embodiment 464. The RNAi compound of embodiment 463, wherein a conjugate is attached to the antisense siRNA oligonucleotide.
Embodiment 465. The RNAi compound of embodiment 463 or 464, wherein a conjugate is attached to the sense siRNA oligonucleotide.
Embodiment 466. The RNAi compound of any of embodiments 463-465, wherein the conjugate comprises a GalNAc moiety.
Embodiment 467. The RNAi compound of any of embodiments 463-466, wherein the conjugate group comprises 1-5 linker-nucleosides.
Embodiment 468. The RNAi compound of any of embodiments 398-467, wherein at least one stereo non-standard nucleoside is a independently a stereo-non-standard nucleoside of Formula I-VII.
Embodiment 469. The RNAi compound of any of embodiments 398-467, wherein each stereo non-standard nucleoside is a independently a stereo-non-standard nucleoside of Formula I-VII.
Embodiment 470. A pharmaceutical composition comprising the oligomeric compound, the RNAi compound, or the RNAi compound of any of embodiments 276-469.
Embodiment 471. The pharmaceutical composition of embodiment 470 comprising a pharmaceutically acceptable diluent.
Embodiment 472. A method comprising contacting a cell with the oligomeric compound, the RNAi compound, or the siRNA compound of any of embodiments 276-469 or the pharmaceutical composition of embodiment 470 or 471.
Embodiment 473. A method of administering to an animal the oligomeric compound, the RNAi compound, or the siRNA compound of any of embodiments 276-469 or the pharmaceutical composition of embodiment 470 or 471.
Embodiment 474. The oligomeric compound of any of embodiments 276-396, wherein the oligomeric compound is a CRISPR compound.
Embodiment 475. The CRISPR compound of embodiment 474 comprising a CRISPR oligonucleotide consisting of 20-120 linked nucleosides, wherein the CRISPR oligonucleotide is a modified oligonucleotide according to any of embodiments 1-121.
Embodiment 476. The CRISPR compound of embodiment 475, wherein the CRISPR oligonucleotide consists of 50-120 linked nucleosides.
Embodiment 477. The CRISPR compound of embodiment 475, wherein the CRISPR oligonucleotide consists of 20-50 linked nucleosides
Embodiment 478. The CRISPR compound of embodiment 475, wherein the CRISPR oligonucleotide consists of 29-32 linked nucleosides.
Embodiment 479. The CRISPR compound of embodiment 475, wherein the CRISPR oligonucleotide consists of 20-28 linked nucleosides.
Embodiment 480. The CRISPR compound of embodiment 475, wherein the CRISPR oligonucleotide consists of 29 linked nucleosides.
Embodiment 481. The CRISPR compound of embodiment 475, wherein the CRISPR oligonucleotide consists of 32 linked nucleosides.
Embodiment 482. The CRISPR compound of any of embodiments 475-481, wherein at least one of the first 5 nucleosides from the 5′-end of the CRISPR oligonucleotide is a stereo-non-standard nucleoside.
Embodiment 483. The CRISPR compound of any of embodiments 475-482, wherein at least one of the last 5 nucleosides counting back from the 3′-end of the CRISPR oligonucleotide is a stereo-non-standard nucleoside.
Embodiment 484. The CRISPR compound of any of embodiments 475-483, wherein at least one nucleoside of the CRISPR oligonucleotide is a stereo-standard nucleoside or a bicyclic nucleoside.
Embodiment 485. The CRISPR compound of embodiment 484, wherein at least one nucleoside of the CRISPR oligonucleotide is a substituted stereo-standard nucleoside.
Embodiment 486. The CRISPR compound of embodiment 484 or 485, wherein at least one nucleoside of the CRISPR oligonucleotide is a bicyclic nucleoside.
Embodiment 487. The CRISPR compound of any of embodiments 484-486, wherein at least one stereo-standard or bicyclic nucleoside of the CRISPR oligonucleotide is selected from: an LNA nucleoside, a cEt nucleoside, an ENA nucleoside, a 2′-MOE nucleoside, a 2′-OMe nucleoside, a 2′-F nucleoside, a 2′-NMA nucleoside, a 5′-Me nucleoside, a DNA nucleoside, and a RNA nucleoside.
Embodiment 488. The CRISPR compound of any of embodiments 484-487, wherein each stereo-standard or bicyclic nucleoside of the modified CRISPR oligonucleotide is selected from: an LNA nucleoside, a cEt nucleoside, an ENA nucleoside, a 2′-MOE nucleoside, a 2′-OMe nucleoside, a 2′-F nucleoside, a 2′-NMA nucleoside, a 5′-Me nucleoside, a DNA nucleoside, and a RNA nucleoside.
Embodiment 489. The CRISPR compound of any of embodiments 484-488, wherein at least one nucleoside of the modified CRISPR oligonucleotide is selected from: a 2′-OMe nucleoside, a 2′-F nucleoside, and a stereo-standard RNA nucleoside.
Embodiment 490. The CRISPR compound of any of embodiments 484-489 wherein at least one nucleoside of the modified CRISPR oligonucleotide is a 2′-OMe nucleoside, and at least one nucleoside of the modified oligonucleotide is a stereo-standard RNA nucleoside.
Embodiment 491. The CRISPR compound of embodiment any of embodiments 475-490, wherein the CRISPR oligonucleotide is a modified crRNA oligonucleotide.
Embodiment 492. The CRISPR compound of embodiment 496, wherein the tracrRNA recognition portion of the crRNA consists of 12 or fewer linked nucleosides.
Embodiment 493. The CRISPR compound of any of embodiments 475-492, wherein the DNA recognition portion of the CRISPR oligonucleotide consists of 17 or fewer linked nucleosides.
Embodiment 494. The CRISPR compound of any of embodiments 475-493, wherein at least one stereo non-standard nucleoside is a independently a stereo-non-standard nucleoside of Formula I-VII.
Embodiment 495. The CRISPR compound of any of embodiments 475-494, wherein each stereo non-standard nucleoside is a independently a stereo-non-standard nucleoside of Formula I-VII.
Embodiment 496. A method comprising contacting a cell with the CRISPR oligonucleotide of any of embodiments 475-495.
Embodiment 497. The method of embodiment 496 comprising contacting the cell with a plasmid that encodes Cas9 or Cpf1.
Embodiment 498. The method of embodiment 497, wherein the plasmid encodes a tracrRNA.
Embodiment 499. The method of embodiment 496, comprising contacting the cell with an mRNA that encodes Cas9 or Cpf1.
Embodiment 500. The method of embodiment 496, comprising contacting the cell with a plasmid that encodes a tracrRNA.
Embodiment 501. The method of any of embodiments 496-500 wherein a target gene is edited.
Embodiment 502. A pharmaceutical composition comprising the CRISPR compound or the CRISPR oligonucleotide of any of embodiments 475-501.
Embodiment 503. The pharmaceutical composition of embodiment 502 comprising a pharmaceutically acceptable diluent.
Embodiment 504. A method comprising contacting a cell with the CRISPR compound or the CRISPR oligonucleotide of any of embodiments 475-495 or the pharmaceutical composition of embodiment 502 or 503.
Embodiment 505. A method of administering to an animal the CRISPR compound or the CRISPR oligonucleotide of any of embodiments 475-495 or the pharmaceutical composition of embodiment 502 or 503.
Embodiment 506. The oligomeric compound of any of embodiments 276-396, wherein the oligomeric compound is an artificial mRNA compound.
Embodiment 507. The artificial mRNA compound of embodiment 506, wherein the artificial mRNA compound is an artificial mRNA oligonucleotide consisting of 17-3000 linked nucleosides.
Embodiment 508. The artificial mRNA compound of any of embodiments 506-507, wherein at least one of the first 5 nucleosides from the 5′-end of the artificial mRNA oligonucleotide is a stereo-non-standard nucleoside.
Embodiment 509. The artificial mRNA oligonucleotide of any of embodiments 506-508, wherein at least one of the last 5 nucleosides counting back from the 3′-end of the artificial mRNA oligonucleotide is a stereo-non-standard nucleoside.
Embodiment 510. The artificial mRNA compound of any of embodiments 506-509, wherein at least one nucleoside of the artificial mRNA oligonucleotide is a stereo-standard nucleoside.
Embodiment 511. The artificial mRNA compound of any of embodiments 506-510, wherein at least one nucleoside of the 5′-UTR of the artificial mRNA oligonucleotide is a stereo-non-standard nucleoside.
Embodiment 512. The artificial mRNA compound of any of embodiments 506-511, wherein at least one nucleoside of the 3′-UTR of the artificial mRNA oligonucleotide is a stereo-non-standard nucleoside.
Embodiment 513. The artificial mRNA compound of any of embodiments 506-512, wherein at least one nucleoside of the poly-A tail region of the artificial mRNA oligonucleotide is a stereo-non-standard nucleoside.
Embodiment 514. The artificial mRNA compound of any of embodiments 506-513, wherein at least one nucleoside of the coding region of the artificial mRNA oligonucleotide is a stereo-non-standard nucleoside.
Embodiment 515. The artificial mRNA compound of any of embodiments 506-514, wherein at least one stereo non-standard nucleoside of the artificial mRNA oligonucleotide is a independently a stereo-non-standard nucleoside of Formula I-VII.
Embodiment 516. The artificial mRNA compound of any of embodiments 506-515, wherein each stereo non-standard nucleoside of the artificial mRNA oligonucleotide is a independently a stereo-non-standard nucleoside of Formula I-VII.
Embodiment 517. A pharmaceutical composition comprising the artificial mRNA compound of any of embodiments 506-516, and a pharmaceutically acceptable carrier or diluent.
Embodiment 518. A pharmaceutical composition comprising the artificial mRNA compound of any of embodiments 506-516 and a lipid nanoparticle.
Embodiment 519. A method comprising contacting a cell with the artificial mRNA compound of any of embodiments 506-516 or the pharmaceutical composition of embodiment 517 or 518.
Embodiment 520. A method of administering to an animal the artificial mRNA compound any of embodiments 506-516 or the pharmaceutical composition of embodiment 518 or 519.

### Certain Compounds

In certain embodiments, compounds described herein are oligomeric compounds comprising or consisting of oligonucleotides consisting of linked nucleosides and having at least one stereo-non-standard nucleoside. Oligonucleotides may be unmodified oligonucleotides or may be modified oligonucleotides. Modified oligonucleotides comprise at least one modification relative to an unmodified oligonucleotide (i.e., comprise at least one modified nucleoside (comprising a modified sugar moiety, a stereo-non-standard nucleoside, and/or a modified nucleobase) and/or at least one modified internucleoside linkage).

I. Modifications

A. Modified Nucleosides

Modified nucleosides comprise a stereo-non-standard nucleoside, or a modified sugar moiety, or a modified nucleobase, or any combination thereof.

1. Certain Modified Sugar Moieties

In certain embodiments, modified sugar moieties are stereo-non-standard sugar moieties. In certain embodiments, sugar moieties are substituted furanosyl stereo-standard sugar moieties. In certain embodiments, modified sugar moieties are bicyclic or tricyclic furanosyl sugar moieties. In certain embodiments, modified sugar moieties are sugar surrogates. Such sugar surrogates may comprise one or more substitutions corresponding to those of other types of modified sugar moieties.

a. Stereo-Non-Standard Sugar Moieties

In certain embodiments, modified sugar moieties are stereo-non-standard sugar moieties shown in one of Formula I, Formula II, Formula III, Formula IV, Formula V, Formula VI, and Formula VII:

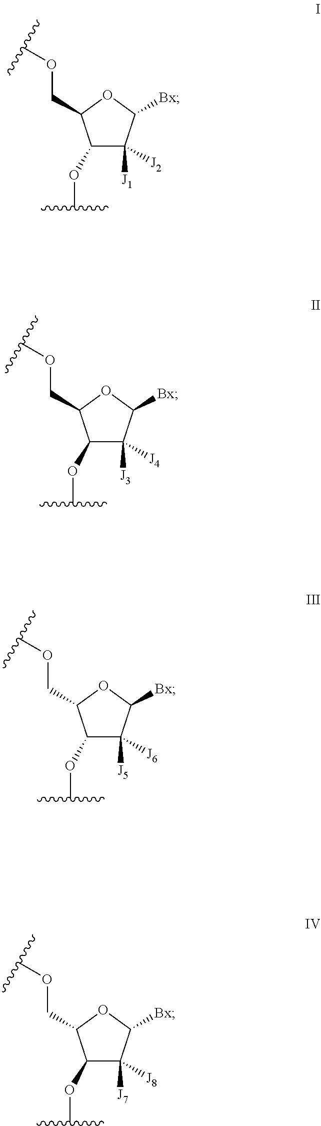

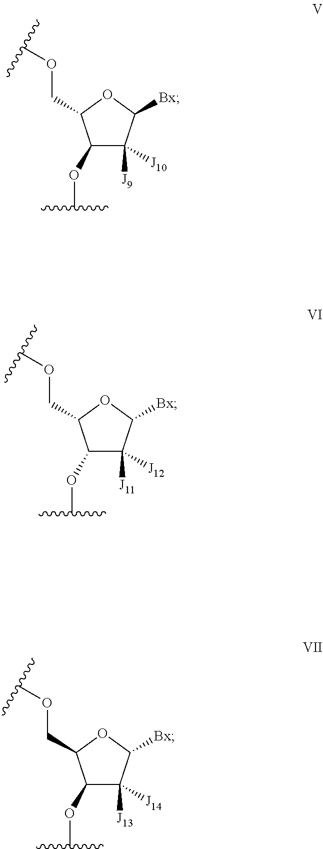

wherein

one of J₁ and J₂ is H and the other of J₁ and J₂ is selected from H, OH, F, OCH₃, OCH₂CH₂OCH₃, O—C₁-C₆ alkoxy, and SCH₃;

one of J₃ and J₄ is H and the other of J₃ and J₄ is selected from H, OH, F, OCH₃, OCH₂CH₂OCH₃, O—C₁-C₆ alkoxy, and SCH₃; and wherein

one of J₅ and J₆ is H and the other of J₅ and J₆ is selected from H, OH, F, OCH₃, OCH₂CH₂OCH₃, O—C₁-C₆ alkoxy, and SCH₃; and wherein

one of J₇ and J₈ is H and the other of J₇ and J₈ is selected from H, OH, F, OCH₃, OCH₂CH₂OCH₃, O—C₁-C₆ alkoxy, and SCH₃; and wherein

one of J₉ and J₁₀ is H and the other of J₉ and J₁₀ is selected from H, OH, F, OCH₃, OCH₂CH₂OCH₃, O—C₁-C₆ alkoxy, and SCH₃; and wherein

one of J₁₁ and J₁₂ is H and the other of J₁₁ and J₁₂ is selected from H, OH, F, OCH₃, OCH₂CH₂OCH₃, O—C₁-C₆ alkoxy, and SCH₃; and wherein

one of J₁₃ and J₁₄ is H and the other of J₁₃ and J₁₄ is selected from H, OH, F, OCH₃, OCH₂CH₂OCH₃, O—C₁-C₆ alkoxy, and SCH₃; and

Bx is a is a heterocyclic base moiety.

Certain stereo-non-standard sugar moieties have been previously described in, e.g., Seth et al., WO2020/072991 and Seth et al., WO2019/157531, both of which are incorporated by reference herein in their entirety.

b. Substituted Stereo-Standard Sugar Moieties

In certain embodiments, modified sugar moieties are substituted furanosyl sugar moieties comprising one or more acyclic substituent, including but not limited to substituents at the 2′, 3′, 4′, and/or 5′ positions. In certain embodiments, the furanosyl sugar moiety is a ribosyl sugar moiety. In certain embodiments one or more acyclic substituent of substituted stereo-standard sugar moieties is branched. Examples of 2′-substituent groups suitable for substituted stereo-standard sugar moieties include but are not limited to: 2′-F, 2′-OCH₃ (“2′-OMe” or “2′-O-methyl”), and 2′-O(CH₂)₂OCH₃ (“2′-MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 3′-substituent groups include 3′-methyl (see Frier, et al., The ups and downs of nucleic acid duplex stability: structure-stability studies on chemically-modified DNA:RNA duplexes. Nucleic Acids Res., 25, 4429-4443, 1997.) Examples of 4′-substituent groups suitable for substituted stereo-standard sugar moieties include but are not limited to alkoxy (e.g., methoxy), alkyl, and those described in Manoharan et al., WO 2015/106128. Examples of 5′-substituent groups suitable for substituted stereo-standard sugar moieties include but are not limited to: 5′-methyl (R or S), 5′-allyl, 5′-ethyl, 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836. 2′,4′-difluoro modified sugar moieties have been described in Martinez-Montero, et al., Rigid 2′,4′-difluororibonucleosides: synthesis, conformational analysis, and incorporation into nascent RNA by HCV polymerase. J. Org. Chem., 2014, 79:5627-5635. Modified sugar moieties comprising a 2′-modification (OMe or F) and a 4′-modification (OMe or F) have also been described in Malek-Adamian, et al., J. Org. Chem, 2018, 83: 9839-9849.

In certain embodiments, a 2′-substituted stereo-standard nucleoside comprises a sugar moiety comprising a non-bridging 2′-substituent group selected from: F, NH₂, N₃, OCF₃, OCH₃, SCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted stereo-standard nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted stereo-standard comprises a sugar moiety comprising a non-bridging 2′-substituent group selected from: F, OCH₃, and OCH₂CH₂OCH₃.

In certain embodiments, the 4′ 0 of 2′-deoxyribose can be substituted with a S to generate 4′-thio DNA (see Takahashi, et al., Nucleic Acids Research 2009, 37: 1353-1362). This modification can be combined with other modifications detailed herein. In certain such embodiments, the sugar moiety is further modified at the 2′ position. In certain embodiments the sugar moiety comprises a 2′-fluoro. A thymidine with this sugar moiety has been described in Watts, et al., J. Org. Chem. 2006, 71(3): 921-925 (4′-S-fluoro5-methylarauridine or FAMU).

c. Bicyclic Nucleosides

Certain nucleosides comprise modified sugar moieties that comprise a bridging sugar substituent that forms a second ring resulting in a bicyclic sugar moiety. In certain such embodiments, the bicyclic sugar moiety comprises a bridge between the 4′ and the 2′ furanose ring atoms. In certain such embodiments, the furanose ring is a ribose ring. Examples of sugar moieties comprising such 4′ to 2′ bridging sugar substituents include but are not limited to bicyclic sugars comprising: 4′-CH₂-2′, 4′-(CH₂)₂-2′, 4′-(CH₂)₃-2′, 4′-CH₂—O-2′ (“LNA”), 4′-CH₂—S-2′, 4′- (CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672), 4′-C(═O)—N(CH₃)₂-2′, 4′-C(═O)—N(R)₂-2′, 4′-C(═S)—N(R)₂-2′ and analogs thereof (see, e.g., Obika et al., WO2011052436A1, Yusuke, WO2017018360A1).

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2017, 129, 8362-8379; Elayadi et al.; Christiansen, et al., J. Am. Chem. Soc. 1998, 120, 5458-5463; Wengel et al., U.S. Pat. No. 7,053,207; Imanishi et al., U.S. Pat. No. 6,268,490; Imanishi et al. U.S. Pat. No. 6,770,748; Imanishi et al., U.S. Pat. No. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

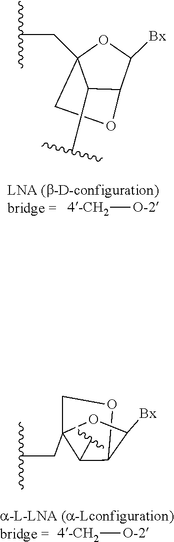

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

The term “substituted” following a position of the furanosyl ring, such as “2′-substituted” or “2′-4′-substituted”, indicates that is the only position(s) having a substituent other than those found in unmodified sugar moieties in oligonucleotides.

d. Sugar Surrogates

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), altritol nucleic acid (“ANA”), mannitol nucleic acid (“MNA”) (see, e.g., Leumann, C J. Bioorg. & Med. Chem. 2002, 10, 841-854), fluoro HNA (“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran).

In certain embodiments, sugar surrogates comprise rings having no heteroatoms. For example, nucleosides comprising bicyclo [3.1.0]-hexane have been described (see, e.g., Marquez, et al., J. Med. Chem. 1996, 39:3739-3749).

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate comprising the following structure:

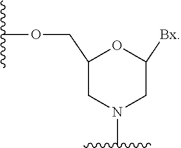

In certain embodiments, morpholinos may be modified, for example by adding or altering various substituent groups from the above morpholino structure. Such sugar surrogates are referred to herein as “modified morpholinos.” In certain embodiments, morpholino residues replace a full nucleotide, including the internucleoside linkage, and have the structures shown below, wherein Bx is a heterocyclic base moiety.

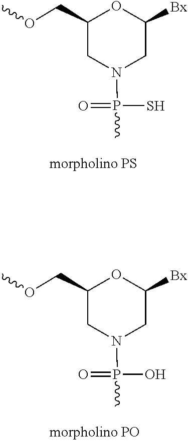

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), glycol nucleic acid (“GNA”, see Schlegel, et al., J. Am. Chem. Soc. 2017, 139:8537-8546) and nucleosides and oligonucleotides described in Manoharan et al., WO2011/133876.

Many other bicyclic and tricyclic sugar and sugar surrogate ring systems are known in the art that can be used in modified nucleosides. Certain such ring systems are described in Hanessian, et al., J. Org. Chem., 2013, 78: 9051-9063 and include bcDNA and tcDNA. Modifications to bcDNA and tcDNA, such as 6′-fluoro, have also been described (Dogovic and Leumann, J. Org. Chem., 2014, 79: 1271-1279).

2. Modified Nucleobases

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deazaadenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443. In certain embodiments, modified nucleosides comprise double-headed nucleosides having two nucleobases. Such compounds are described in detail in Sorinas et al., J. Org. Chem, 2014 79: 8020-8030.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, compounds comprise or consist of a modified oligonucleotide complementary to an target nucleic acid comprising one or more modified nucleobases. In certain embodiments, the modified nucleobase is 5-methylcytosine. In certain embodiments, each cytosine is a 5-methylcytosine.

B. Modified Internucleoside Linkages

In certain embodiments, compounds described herein having one or more modified internucleoside linkages are selected over compounds having only phosphodiester internucleoside linkages because of desirable properties such as, for example, enhanced cellular uptake, enhanced affinity for target nucleic acids, and increased stability in the presence of nucleases.

In certain embodiments, compounds comprise or consist of a modified oligonucleotide complementary to a target nucleic acid comprising one or more modified internucleoside linkages. In certain embodiments, the modified internucleoside linkages are phosphorothioate linkages. In certain embodiments, each internucleoside linkage of an antisense compound is a phosphorothioate internucleoside linkage.

In certain embodiments, nucleosides of modified oligonucleotides may be linked together using any internucleoside linkage. The two main classes of internucleoside linkages are defined by the presence or absence of a phosphorus atom. Representative phosphorus-containing internucleoside linkages include unmodified phosphodiester internucleoside linkages, modified phosphotriesters such as THP phosphotriester and isopropyl phosphotriester, phosphonates such as methylphosphonate, isopropyl phosphonate, isobutyl phosphonate, and phosphonoacetate, phosphoramidates, including mesyl phosphoramidates, phosphorothioate, and phosphorodithioate (“HS—P═S”). Representative non-phosphorus containing internucleoside linkages include but are not limited to methylenemethylimino (—CH₂—N(CH₃)—O—CH₂—), thiodiester, thionocarbamate (—O—C(═O)(NH)—S—); siloxane (—O—SiH₂—O—); formacetal, thioacetamido (TANA), alt-thioformacetal, glycine amide, and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. All phosphorothioate linkages described herein are stereorandom unless otherwise specified. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

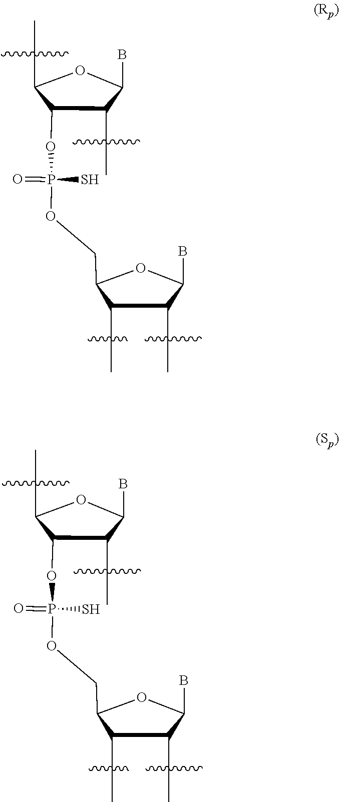

Unless otherwise indicated, chiral internucleoside linkages of modified oligonucleotides described herein can be stereorandom or in a particular stereochemical configuration.

Neutral internucleoside linkages include, without limitation, phosphotriesters, phosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, nucleic acids can be linked 2′ to 5′ rather than the standard 3′ to 5′ linkage. Such a linkage is illustrated below.

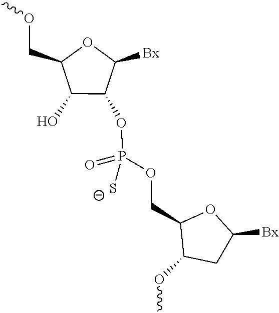

In certain embodiments, nucleosides can be linked by vicinal 2′,3′-phosphodiester bonds. In certain such embodiments, the nucleosides are threofuranosyl nucleosides (TNA; see Bala, et al., J Org. Chem. 2017, 82:5910-5916). A TNA linkage is shown below.

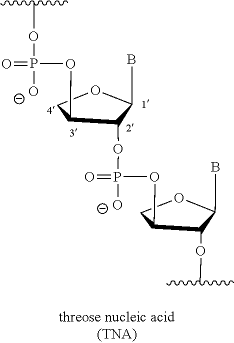

Additional modified linkages include α,β-D-CNA type linkages and related conformationally-constrained linkages, shown below. Synthesis of such molecules has been described previously (see Dupouy, et al., Angew. Chem. Int. Ed. Engl., 2014, 45: 3623-3627; Borsting, et al. Tetahedron, 2004, 60:10955-10966; Ostergaard, et al., ACS Chem. Biol. 2014, 9: 1975-1979; Dupouy, et al., Eur. J Org. Chem., 2008, 1285-1294; Martinez, et al., PLoS One, 2011, 6:e25510; Dupouy, et al., Eur. J. Org. Chem., 2007, 5256-5264; Boissonnet, et al., New J. Chem., 2011, 35: 1528-1533)

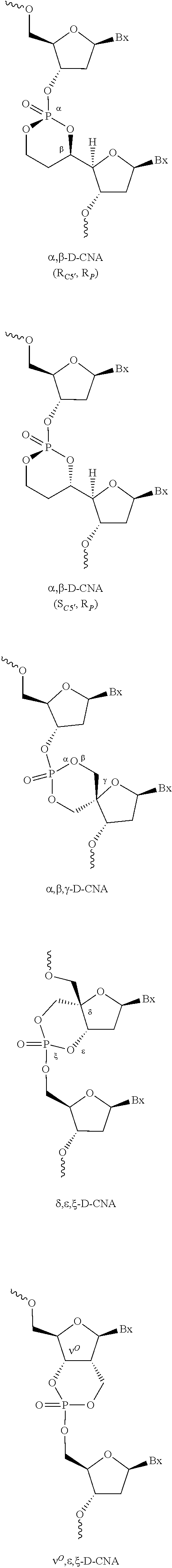

II. Certain Motifs

In certain embodiments, oligomeric compounds described herein comprise or consist of oligonucleotides. Modified oligonucleotides can be described by their motif, e.g. a pattern of unmodified and/or modified sugar moieties, nucleobases, and/or internucleoside linkages. In certain embodiments, modified oligonucleotides comprise one or more stereo-non-standard nucleosides. In certain embodiments, modified oligonucleotides comprise one or more stereo-standard nucleosides. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns or motifs of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

A. Certain Sugar Motifs

In certain embodiments, oligomeric compounds described herein comprise or consist of oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include without limitation any of the sugar modifications discussed herein.

In certain embodiments, an oligomeric compound is an siRNA compound comprising an antisense siRNA oligonucleotide and a sense siRNA oligonucleotide. In certain embodiments, the antisense siRNA oligonucleotide comprises at least one stereo-non-standard nucleoside having any of Formula I-VII. In certain embodiments, the antisense siRNA oligonucleotide comprises at least two, at least three, at least four, at least five, or at least six stereo-non-standard nucleosides selected from Formula I-VII. In certain embodiments, the antisense siRNA oligonucleotide comprises exactly one stereo-non-standard nucleoside. In certain embodiments, the antisense siRNA oligonucleotide comprises exactly two stereo-non-standard nucleosides. In certain embodiments, the antisense siRNA oligonucleotide comprises exactly three stereo-non-standard nucleosides. In certain embodiments, the antisense siRNA oligonucleotide comprises exactly four stereo-non-standard nucleosides. In certain embodiments, the antisense siRNA oligonucleotide comprises exactly five stereo-non-standard nucleosides. In certain embodiments, the antisense siRNA oligonucleotide comprises exactly 6, 7, 8, 9, 10, 11, 12, 13, 14, 15, 16, 17, 18, 19, 20, 21, or 22 stereo-non-standard nucleosides. In certain embodiments, at least one of the first 5 nucleosides from the 5′ end of the antisense siRNA oligonucleotide is a stereo-non-standard nucleoside of Formula I-VII. In certain embodiments, at least one of the last 5 nucleosides counting back from the 3′ end of the antisense siRNA oligonucleotide is a stereo-non-standard nucleoside of Formula I-VII. In certain embodiments, at least one nucleoside of the seed region of the antisense siRNA oligonucleotide is a stereo-non-standard nucleoside of Formula I-VII. In certain embodiments, at least one nucleoside within nucleosides 2 to 8 of the antisense siRNA oligonucleotide, counting from the 5′ end, is a stereo-non-standard nucleoside of Formula I-VII. In certain embodiments, each remaining nucleoside of the antisense siRNA oligonucleotide is selected from stereo-standard nucleosides and bicyclic nucleosides. In certain embodiments, each remaining nucleoside of the antisense siRNA oligonucleotide is selected from 2′-OMe, 2′-F, and stereostandard RNA nucleosides. In certain embodiments, the antisense siRNA oligonucleotide comprises at least one stereo-non-standard nucleoside selected from Formula I-VII and at least one (S)-GNA. In certain embodiments, the (S)-GNA is at position 7 of the antisense strand as counted from the 5′ end. In certain embodiments, the antisense siRNA oligonucleotide comprises at least one stereo-non-standard nucleoside selected from Formula I-VII and at least one region of alternating nucleoside types having the motif ABABA, wherein each A is a stereo-standard or bicyclic nucleoside having a sugar moiety of a first type and each B is a stereo-standard or bicyclic nucleoside having a sugar moiety of a second type, wherein the first type and second type are different from each other. In certain embodiments, A and B are selected from 2′-OMe, 2′-F, and stereo-standard RNA nucleosides.

In certain embodiments, the sense siRNA oligonucleotide comprises at least one stereo-non-standard nucleoside selected from Formula I-VII. In certain embodiments, the sense siRNA oligonucleotide comprises at least two, at least three, at least four, at least five, or at least six stereo-non-standard nucleosides selected from Formula I-VII. In certain embodiments, the sense siRNA oligonucleotide comprises exactly one stereo-non-standard nucleoside. In certain embodiments, the sense siRNA oligonucleotide comprises exactly two stereo-non-standard nucleosides. In certain embodiments, the sense siRNA oligonucleotide comprises exactly three stereo-non-standard nucleosides. In certain embodiments, the sense siRNA oligonucleotide comprises exactly four stereo-non-standard nucleosides. In certain embodiments, the sense siRNA oligonucleotide comprises exactly five stereo-non-standard nucleosides. In certain embodiments, the sense siRNA oligonucleotide comprises exactly 6, 7, 8, 9, 10, 11, 12, 13, 14, 15, 16, 17, 18, 19, 20, 21, or 22 stereo-non-standard nucleosides. In certain embodiments, at least one of the first 5 nucleosides from the 5 end of the sense siRNA oligonucleotide is a stereo-non-standard nucleoside of Formula I-VII. In certain embodiments, at least one of the last 5 nucleosides counting back from the 3′ end of the sense siRNA oligonucleotide is a stereo-non-standard nucleoside of Formula I-VII. In certain embodiments, each remaining nucleoside of the sense siRNA oligonucleotide is selected from stereo-standard nucleosides and bicyclic nucleosides. In certain embodiments, each remaining nucleoside of the sense siRNA oligonucleotide is selected from 2′-OMe, 2′-F, and stereostandard RNA nucleosides. In certain embodiments, the sense siRNA oligonucleotide comprises at least one stereo-non-standard nucleoside selected from Formula I-VII and at least one unlocked nucleic acid. In certain embodiments, the sense siRNA oligonucleotide comprises at least one stereo-non-standard nucleoside selected from Formula I-VII and at least one region of alternating nucleoside types having the motif ABABA, wherein each A is a stereo-standard or bicyclic nucleoside having a sugar moiety of a first type and each B is a stereo-standard or bicyclic nucleoside having a sugar moiety of a second type, wherein the first type and second type are different from each other. In certain embodiments, A and B are selected from 2′-OMe, 2′-F, and stereo-standard RNA nucleosides. In certain embodiments, an oligomeric compound is an siRNA compound comprising a single-stranded RNAi oligonucleotide. In certain embodiments, the single-stranded RNAi oligonucleotide comprises at least one stereo-non-standard nucleoside selected from Formula I-VII. In certain embodiments, the single-stranded RNAi oligonucleotide comprises at least two, at least three, at least four, at least five, or at least six stereo-non-standard nucleosides selected from Formula I-VII. In certain embodiments, the single-stranded RNAi oligonucleotide comprises exactly one stereo-non-standard nucleoside. In certain embodiments, the single-stranded RNAi oligonucleotide comprises exactly two stereo-non-standard nucleosides. In certain embodiments, the single-stranded RNAi oligonucleotide comprises exactly three stereo-non-standard nucleosides. In certain embodiments, the single-stranded RNAi oligonucleotide comprises exactly four stereo-non-standard nucleosides. In certain embodiments, the single-stranded RNAi oligonucleotide comprises exactly five stereo-non-standard nucleosides. In certain embodiments, the single-stranded RNAi oligonucleotide comprises exactly 6, 7, 8, 9, 10, 11, 12, 13, 14, 15, 16, 17, 18, 19, 20, 21, or 22 stereo-non-standard nucleosides. In certain embodiments, at least one of the first 5 nucleosides from the 5′ end of the single-stranded RNAi oligonucleotide is a stereo-non-standard nucleoside of Formula I-VII. In certain embodiments, at least one of the last 5 nucleosides counting back from the 3′ end of the single-stranded RNAi oligonucleotide is a stereo-non-standard nucleoside of Formula I-VII. In certain embodiments, each remaining nucleoside of the single-stranded RNAi oligonucleotide is selected from stereo-standard nucleosides and bicyclic nucleosides. In certain embodiments, each remaining nucleoside of the single-stranded RNAi oligonucleotide is selected from 2′-OMe, 2′-F, and stereostandard RNA nucleosides. In certain embodiments, the single-stranded RNAi oligonucleotide comprises at least one stereo-non-standard nucleoside selected from Formula I-VII and at least one region of alternating nucleoside types having the motif ABABA, wherein each A is a stereo-standard or bicyclic nucleoside having a sugar moiety of a first type and each B is a stereo-standard or bicyclic nucleoside having a sugar moiety of a second type, wherein the first type and second type are different from each other. In certain embodiments, A and B are selected from 2′-OMe, 2′-F, and stereo-standard RNA nucleosides.

In certain embodiments, CRISPR compounds are modified oligonucleotides. In certain embodiments, CRISPR modified oligonucleotides have a DNA recognition region and a tracrRNA recognition region. In certain embodiments, the DNA recognition region includes a seed region. In certain embodiments, the CRISPR modified oligonucleotide comprises at least one stereo-non-standard nucleoside selected from Formula I-VII. In certain embodiments, the CRISPR modified oligonucleotide comprises at least two, at least three, at least four, at least five, or at least six stereo-non-standard nucleosides selected from Formula I-VII. In certain embodiments, the CRISPR modified oligonucleotide comprises exactly one stereo-non-standard nucleoside. In certain embodiments, the CRISPR modified oligonucleotide comprises exactly two stereo-non-standard nucleosides. In certain embodiments, the CRISPR modified oligonucleotide comprises exactly three stereo-non-standard nucleosides. In certain embodiments, the CRISPR modified oligonucleotide comprises exactly four stereo-non-standard nucleosides. In certain embodiments, the CRISPR modified oligonucleotide comprises exactly five stereo-non-standard nucleosides. In certain embodiments, the CRISPR modified oligonucleotide comprises exactly 6, 7, 8, 9, 10, 11, 12, 13, 14, 15, 16, 17, 18, 19, 20, 21, or 22 stereo-non-standard nucleosides. In certain embodiments, at least one of the first 5 nucleosides from the 5′-end of the CRISPR modified oligonucleotide is a stereo-non-standard nucleoside of formula I-VII. In certain embodiments, at least one of the last 5 nucleosides counting back from the 3′ end of the CRISPR modified oligonucleotide is a stereo-non-standard nucleoside of formula I-VII. In certain embodiments, at least one nucleoside of the DNA recognition region of the CRISPR modified oligonucleotide is a stereo-non-standard nucleoside of formula I-VII. In certain embodiments, at least one nucleoside of the seed region of the CRISPR modified oligonucleotide is a stereo-non-standard nucleoside of Formula I-VII. In certain embodiments, at least one nucleoside of the tracrRNA recognition region of the CRISPR modified oligonucleotide is a stereo-non-standard nucleoside of Formula I-VII. In certain embodiments, each remaining nucleoside of the CRISPR modified oligonucleotide is selected from stereo-standard nucleosides and bicyclic nucleosides. In certain embodiments, the CRISPR modified oligonucleotide has at least one region of alternating nucleoside types having the motif ABABA wherein each A is a stereo-standard nucleoside having a sugar moiety of a first type and each B is a stereo-standard nucleoside having a sugar moiety of a second type, wherein the first type and the second type are different from one another. In certain embodiments, A and B are selected from 2′-OMe, 2′-F, and stereo-standard RNA nucleosides.

In certain embodiments, modified oligonucleotides are artificial mRNA oligonucleotides. In certain embodiments, the artificial mRNA oligonucleotide comprises at least one stereo-non-standard nucleoside selected from Formula I-VII. In certain embodiments, the artificial mRNA oligonucleotide comprises at least two, at least three, at least four, at least five, or at least six stereo-non-standard nucleosides selected from Formula I-VII. In certain embodiments, the artificial mRNA oligonucleotide comprises exactly one stereo-non-standard nucleoside. In certain embodiments, the artificial mRNA oligonucleotide comprises exactly two stereo-non-standard nucleosides. In certain embodiments, the artificial mRNA oligonucleotide comprises exactly three stereo-non-standard nucleosides. In certain embodiments, the artificial mRNA oligonucleotide comprises exactly four stereo-non-standard nucleosides. In certain embodiments, the artificial mRNA oligonucleotide comprises exactly five stereo-non-standard nucleosides. In certain embodiments, the artificial mRNA oligonucleotide comprises exactly 6, 7, 8, 9, 10, 11, 12, 13, 14, 15, 16, 17, 18, 19, 20, 21, or 22 stereo-non-standard nucleosides. In certain embodiments, the artificial mRNA oligonucleotide comprises more than 10, more than 20, more than 30, more than 40, more than 50, or more than 100 stereo-non-standard nucleosides. In certain embodiments, at least one of the first 5 nucleosides from the 5 end of the artificial mRNA oligonucleotide is a stereo-non-standard nucleoside of Formula I-VII. In certain embodiments, at least one of the last 5 nucleosides counting back from the 3′ end of the artificial mRNA oligonucleotide is a stereo-non-standard nucleoside of Formula I-VII. In certain embodiments, at least one nucleoside of the 5′-UTR of the artificial mRNA oligonucleotide is a stereo-non-standard nucleoside of Formula I-VII. In certain embodiments, at least one nucleoside of the 3′-UTR of the artificial mRNA oligonucleotide is a stereo-non-standard nucleoside of Formula I-VII. In certain embodiments, at least one nucleoside of the coding region of the artificial mRNA oligonucleotide is a stereo-non-standard nucleoside of Formula I-VII. In certain embodiments, each remaining nucleoside of the artificial mRNA oligonucleotide is selected from stereo-standard nucleosides and bicyclic nucleosides.

B. Certain Nucleobase Motifs

In certain embodiments, oligomeric compounds described herein comprise or consist of oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, one nucleoside comprising a modified nucleobase is in the central region of a modified oligonucleotide. In certain such embodiments, the sugar moiety of said nucleoside is a 2′-β-D-deoxyribosyl moiety. In certain such embodiments, the modified nucleobase is selected from: 5-methyl cytosine, 2-thiopyrimidine, 2-thiothymine, 6-methyladenine, inosine, pseudouracil, or 5-propynepyrimidine.

C. Certain Internucleoside Linkage Motifs

In certain embodiments, oligomeric compounds described herein comprise or consist of oligonucleotides. In certain embodiments, oligonucleotides comprise modified and/or unmodified internucleoside linkages arranged along the oligonucleotide or region thereof in a defined pattern or motif. In certain embodiments, each internucleoside linkage is a phosphodiester internucleoside linkage (P═O). In certain embodiments, each internucleoside linkage of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the internucleoside linkages within the central region of a modified oligonucleotide are all modified. In certain such embodiments, some or all of the internucleoside linkages in the 5′-region and 3′-region are unmodified phosphate linkages. In certain embodiments, the terminal internucleoside linkages are modified. In certain embodiments, the internucleoside linkage motif comprises at least one phosphodiester internucleoside linkage in at least one of the 5′-region and the 3′-region, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the 5′-region and 3′-region are (Sp) phosphorothioates, and the central region comprises at least one Sp, Sp, Rp motif. In certain embodiments, populations of modified oligonucleotides are enriched for modified oligonucleotides comprising such internucleoside linkage motifs.

In certain embodiments, oligonucleotides comprise a region having an alternating internucleoside linkage motif. In certain embodiments, oligonucleotides comprise a region of uniformly modified internucleoside linkages. In certain such embodiments, the internucleoside linkages are phosphorothioate internucleoside linkages. In certain embodiments, all of the internucleoside linkages of the oligonucleotide are phosphorothioate internucleoside linkages. In certain embodiments, each internucleoside linkage of the oligonucleotide is selected from phosphodiester or phosphate and phosphorothioate. In certain embodiments, each internucleoside linkage of the oligonucleotide is selected from phosphodiester or phosphate and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise one or more methylphosphonate linkages. In certain embodiments, modified oligonucleotides comprise a linkage motif comprising all phosphorothioate linkages except for one or two methylphosphonate linkages. In certain embodiments, one methylphosphonate linkage is in the central region of an oligonucleotide.

In certain embodiments, it is desirable to arrange the number of phosphorothioate internucleoside linkages and phosphodiester internucleoside linkages to maintain nuclease resistance. In certain embodiments, it is desirable to arrange the number and position of phosphorothioate internucleoside linkages and the number and position of phosphodiester internucleoside linkages to maintain nuclease resistance. In certain embodiments, the number of phosphorothioate internucleoside linkages may be decreased and the number of phosphodiester internucleoside linkages may be increased. In certain embodiments, the number of phosphorothioate internucleoside linkages may be decreased and the number of phosphodiester internucleoside linkages may be increased while still maintaining nuclease resistance. In certain embodiments it is desirable to decrease the number of phosphorothioate internucleoside linkages while retaining nuclease resistance. In certain embodiments it is desirable to increase the number of phosphodiester internucleoside linkages while retaining nuclease resistance.

III. Certain Modified Oligonucleotides

In certain embodiments, oligomeric compounds described herein comprise or consist of modified oligonucleotides. In certain embodiments, the above modifications (sugar, nucleobase, internucleoside linkage) are incorporated into a modified oligonucleotide. In certain embodiments, modified oligonucleotides are characterized by their modifications, motifs, and overall lengths. In certain embodiments, such parameters are each independent of one another. Thus, unless otherwise indicated, each internucleoside linkage of a modified oligonucleotide may be modified or unmodified and may or may not follow the modification pattern of the sugar moieties. Likewise, such modified oligonucleotides may comprise one or more modified nucleobase independent of the pattern of the sugar modifications. Furthermore, in certain instances, a modified oligonucleotide is described by an overall length or range and by lengths or length ranges of two or more regions (e.g., a region of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions or segments, A, B, and C, wherein region or segment A consists of 2-6 linked nucleosides having a specified sugar moiety, region or segment B consists of 6-10 linked nucleosides having a specified sugar moiety, and region or segment C consists of 2-6 linked nucleosides having a specified sugar moiety. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of 20 for the overall length of the modified oligonucleotide. Unless otherwise indicated, all modifications are independent of nucleobase sequence except that the modified nucleobase 5-methylcytosine is necessarily a “C” in an oligonucleotide sequence. In certain embodiments, when a DNA nucleoside or DNA-like nucleoside that comprises a T in a DNA sequence is replaced with an RNA-like nucleoside, the nucleobase T is replaced with the nucleobase U. Each of these compounds has an identical target RNA.

In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments oligonucleotides have a nucleobase sequence that is complementary to a second oligonucleotide or an identified reference nucleic acid, such as a target nucleic acid. In certain embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 70%, at least 80%, at least 90%, at least 95%, or 100% complementary to the second oligonucleotide or nucleic acid, such as a target nucleic acid.

IV. Certain Conjugated Compounds

In certain embodiments, the oligomeric compounds described herein comprise or consist of an oligonucleotide (modified or unmodified) and optionally one or more conjugate groups and/or terminal groups. Conjugate groups consist of one or more conjugate moiety and a conjugate linker that links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide.

Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J Pharmacol. Exp. Ther., 1996, i, 923-937),_a tocopherol group (Nishina et al., Molecular Therapy Nucleic Acids, 2015, 4, e220; doi:10.1038/mtna.2014.72 and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain oligomeric compounds, a conjugate linker is a single chemical bond (i.e. conjugate moiety is attached to an oligonucleotide via a conjugate linker through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to oligomeric compounds, such as the oligonucleotides provided herein. In general, a bifunctional linking moiety comprises at least two functional groups. One of the functional groups is selected to bind to a particular site on an oligomeric compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such a compound is more than 30. Alternatively, an oligomeric compound may comprise a modified oligonucleotide consisting of 8-30 nucleosides and no conjugate group. The total number of contiguous linked nucleosides in such a compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the compound has been taken up, it is desirable that the conjugate group be cleaved to release the unconjugated oligonucleotide. Thus, certain conjugate may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate or phosphodiester linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is a nucleoside comprising a 2′-deoxyfuranosyl that is attached to either the 3′ or 5-terminal nucleoside of an oligonucleotide by a phosphodiester internucleoside linkage and covalently attached to the remainder of the conjugate linker or conjugate moiety by a phosphodiester or phosphorothioate linkage. In certain such embodiments, the cleavable moiety is a nucleoside comprising a 2′-β-D-deoxyribosyl sugar moiety. In certain such embodiments, the cleavable moiety is 2′-deoxyadenosine.

3. Certain Cell-Targeting Conjugate Moieties

In certain embodiments, a conjugate group comprises a cell-targeting conjugate moiety. In certain embodiments, a conjugate group has the general formula:

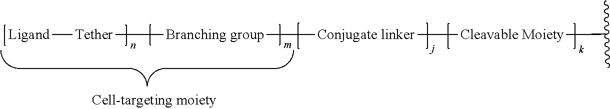

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian lung cell.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808, which are incorporated herein by reference in their entirety). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, oligomeric compounds described herein comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

### Compositions and Methods for Formulating Pharmaceutical Compositions

Oligomeric compounds described herein may be admixed with pharmaceutically acceptable active or inert substances for the preparation of pharmaceutical compositions. Compositions and methods for the formulation of pharmaceutical compositions are dependent upon a number of criteria, including, but not limited to, route of administration, extent of disease, or dose to be administered.

Certain embodiments provide pharmaceutical compositions comprising one or more oligomeric compounds or a salt thereof. In certain embodiments, the oligomeric compounds comprise or consist of a modified oligonucleotide having at least one stereo-non-standard nucleoside. In certain such embodiments, the pharmaceutical composition comprises a suitable pharmaceutically acceptable diluent or carrier. In certain embodiments, a pharmaceutical composition comprises a sterile saline solution and one or more oligomeric compound. In certain embodiments, such pharmaceutical composition consists of a sterile saline solution and one or more oligomeric compound. In certain embodiments, the sterile saline is pharmaceutical grade saline. In certain embodiments, a pharmaceutical composition comprises one or more oligomeric compound and sterile water. In certain embodiments, a pharmaceutical composition consists of one oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, a pharmaceutical composition consists of one or more oligomeric compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

An oligomeric compound described herein complementary to a target nucleic acid can be utilized in pharmaceutical compositions by combining the oligomeric compound with a suitable pharmaceutically acceptable diluent or carrier and/or additional components such that the pharmaceutical composition is suitable for injection. In certain embodiments, a pharmaceutically acceptable diluent is phosphate buffered saline. Accordingly, in one embodiment, employed in the methods described herein is a pharmaceutical composition comprising an oligomeric compound complementary to a target nucleic acid and a pharmaceutically acceptable diluent. In certain embodiments, the pharmaceutically acceptable diluent is phosphate buffered saline. In certain embodiments, the oligomeric compound comprises or consists of a modified oligonucleotide provided herein.

Pharmaceutical compositions comprising oligomeric compounds provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the oligomeric compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

### Certain Mechanisms

In certain embodiments, oligomeric compounds described herein comprise or consist of modified oligonucleotides. In certain such embodiments, the oligomeric compounds described herein are capable of hybridizing to a target nucleic acid, resulting in at least one antisense activity. In certain embodiments, compounds described herein selectively affect one or more target nucleic acid. Such compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Further, in certain embodiments, one or more non-DNA-like nucleoside in in the RNA:DNA duplex is tolerated.

In certain antisense activities, hybridization of a compound described herein to a target nucleic acid results in modulation of the splicing of a target pre-mRNA. For example, in certain embodiments, hybridization of a compound described herein will increase exclusion of an exon. For example, in certain embodiments, hybridization of a compound described herein will increase inclusion of an exon.

In certain antisense activities, compounds described herein or a portion of the compound is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi compounds. RNAi compounds may be double-stranded (siRNA) or single-stranded (ssRNA).

In certain antisense activities, compounds described herein result in a CRISPR system cleaving a target DNA.

In certain embodiments, compounds described herein are artificial mRNA compounds, the nucleobase sequence of which encodes for a protein.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

### Certain Oligomeric Compounds

In certain embodiments, oligomeric compounds described herein having one or more stereo-non-standard nucleosides are selected over compounds lacking such stereo-non-standard nucleosides because of one or more desirable properties. In certain embodiments, oligomeric compounds described herein having one or more stereo-non-standard nucleosides have enhanced cellular uptake. In certain embodiments, oligomeric compounds described herein having one or more stereo-non-standard nucleosides have enhanced bioavailability. In certain embodiments, oligomeric compounds described herein having one or more stereo-non-standard nucleosides have enhanced affinity for target nucleic acids. In certain embodiments, oligomeric compounds described herein having one or more stereo-non-standard nucleosides have increased stability in the presence of nucleases. In certain embodiments, oligomeric compounds described herein having one or more stereo-non-standard nucleosides have increased interactions with certain proteins. In certain embodiments, oligomeric compounds described herein having one or more stereo-non-standard nucleosides have decreased interactions with certain proteins. In certain embodiments, oligomeric compounds described herein having one or more stereo-non-standard nucleosides have increased RNAi activity. In certain embodiments, oligomeric compounds described herein having one or more stereo-non-standard nucleosides have increased CRISPR activity. In certain such embodiments, the stereo-non-standard nucleoside is a stereo-non-standard nucleoside of Formula I, Formula II, Formula III, Formula IV, Formula V, Formula VI, or Formula VII.

In certain embodiments, oligomeric compounds described herein having one or more stereo-non-standard nucleosides are RNAi compounds. In certain embodiments, stereo-non-standard nucleosides can replace one or more stereo-standard nucleoside in any RNAi motif. Certain RNAi motifs are described in, e.g., Freier, et al., WO2020/160163, incorporated by reference herein in its entirety; as well as, e.g., Rajeev, et al., WO2013/075035; Maier, et al., WO2016/028649; Theile, et al., WO2018/098328; Nair, et al., WO2019/217459; each of which is incorporated by reference herein.

### Target Nucleic Acids, Target Regions and Nucleotide Sequences

In certain embodiments, compounds described herein comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain embodiments, a pre-mRNA and corresponding mRNA are both target nucleic acids of a single compound. In certain such embodiments, the target region is entirely within an intron of a target pre-mRNA. In certain embodiments, the target region spans an intron/exon junction. In certain embodiments, the target region is at least 50% within an intron. In certain embodiments, the target nucleic acid is a microRNA.

### Certain Compounds

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or $ such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms. All tautomeric forms of the compounds provided herein are included unless otherwise indicated.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples are intended to illustrate certain aspects of the invention and are not intended to limit the invention in any way.

### Example 1: Design and Activity of Modified Oligonucleotides with 2′-Substituted Stereo-Standard Nucleosides and 2′-Substituted Stereo-Non-Standard Nucleosides

As described in Table 1, below modified oligonucleotides having either 2′-substituted stereo standard nucleosides or 2′-substituted stereo non-standard nucleosides in the gap were synthesized using standard techniques. The modified oligonucleotides were compared to compound 558807, which is a 3-10-3 cEt gapmer, having uniform phosphorothioate (P═S) internucleoside linkages throughout the compound.

TABLE 1
Design and activity of modified oligonucleotides containing
2′-substituted stereo-standard nucleosides and
2′-substituted stereo-non-standard nucleosides

| Compound | | IC50 | |
|---|---|---|---|
| Number | Chemistry Notation (5′-3′) | (nM) | SEQ ID NO. |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 67 | 5 |
| 1385844 | Gₖₛ^{m}CₖₛAₖₛTₘ₂ₛG_{ds}T_{ds}T_{ds}^{m}C_{ds}T_{ds}^{m}C_{ds}A_{ds}^{m}C_{ds}A_{ds}TₖₛTₖₛAₖ | 101 | 5 |
| 1385840 | Gₖₛ^{m}CₖₛAₖₛT_{ds}G_{ds}Tₘ₂ₛT_{ds}^{m}C_{ds}T_{ds}^{m}C_{ds}A_{ds}^{m}C_{ds}A_{ds}TₖₛTₖₛAₖ | 135 | 5 |
| 1385841 | Gₖₛ^{m}CₖₛAₖₛT_{ds}G_{ds}T_{ds}Tₘ₂ₛ^{m}C_{ds}T_{ds}^{m}C_{ds}A_{ds}^{m}C_{ds}A_{ds}TₖₛTₖₛAₖ | 162 | 5 |
| 1385845 | Gₖₛ^{m}CₖₛAₖₛT_{ds}G_{ds}T_{ds}T_{ds}^{m}C_{ds}Tₘ₂ₛ^{m}C_{ds}A_{ds}^{m}C_{ds}A_{ds}TₖₛTₖₛAₖ | 93 | 5 |
| 1385842 | Gₖₛ^{m}CₖₛAₖₛT_{mLs}G_{ds}T_{ds}T_{ds}^{m}C_{ds}T_{ds}^{m}C_{ds}A_{ds}^{m}C_{ds}A_{ds}TₖₛTₖₛAₖ | 114 | 5 |
| 1385838 | Gₖₛ^{m}CₖₛAₖₛT_{ds}G_{ds}T_{mLs}T_{ds}^{m}C_{ds}T_{ds}^{m}C_{ds}A_{ds}^{m}C_{ds}A_{ds}TₖₛTₖₛAₖ | 111 | 5 |
| 1385839 | Gₖₛ^{m}CₖₛAₖₛT_{ds}G_{ds}T_{ds}T_{mLs}^{m}C_{ds}T_{ds}^{m}C_{ds}A_{ds}^{m}C_{ds}A_{ds}TₖₛTₖₛAₖ | 262 | 5 |
| 1385843 | Gₖₛ^{m}CₖₛAₖₛT_{ds}G_{ds}T_{ds}T_{ds}^{m}C_{ds}T_{mLs}^{m}C_{ds}A_{ds}^{m}C_{ds}A_{ds}TₖₛTₖₛAₖ | 101 | 5 |

In Table 1 above, a subscript “s” indicates a phosphorothioate internucleoside linkage, a subscript “k” represents a cEt modified sugar moiety, a subscript “d” represents a stereo-standard DNA nucleoside, and a superscript “m” indicates 5-methyl Cytosine. A subscript “m2” indicates a substituted stereo-standard nucleoside having a 2′-methylthio modification, which is shown below and wherein Bx is a nucleobase:

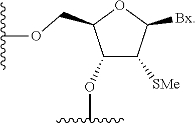

A subscript “mL” indicates a 2′-substituted stereo-non-standard nucleoside having the alpha-L-ribose configuration and a 2′-OCH₃ modification, which is shown below and wherein Bx is a nucleobase:

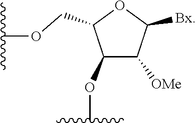

A “mL” nucleoside is a nucleoside of Formula V, wherein J₉ is H and J₁₀ is OCH₃.

The compounds in Table 1 above are 100% complementary to mouse CXCL12, GENBANK NT_039353.7 truncated from 69430515 to 69445350 (SEQ ID NO: 1), at position 6877 to 6892.

Cultured mouse 3T3-L1 cells at a density of 20,000 cells per well were transfected using electroporation with modified oligonucleotides diluted to 20 μM, 7 μM, 2 μM, 0.7 μM, 0.3 μM, 0.1 μM, and 0.03 μM. After a treatment period of approximately 16 hours, CXCL12 RNA levels were measured using mouse primer-probe set RTS2605 (forward sequence CCAGAGCCAACGTCAAGCAT, SEQ ID NO: 2; reverse sequence: CAGCCGTGCAACAATCTGAA, SEQ ID NO: 3; probe sequence: TGAAAATCCTCAACACTCCAAACTGTGCC, SEQ ID NO: 4). CXCL12 RNA levels were normalized to total RNA content, as measured by RIBOGREEN®. Activity of modified oligonucleotides was calculated using the log (inhibitor) vs response (three parameter) function in GraphPad Prism 7 and is presented in Table 1 above as the half maximal inhibitory concentration (IC₅₀).

### Example 2: Caspase Activity of Modified Oligonucleotides Containing 2′-Substituted Stereo-Standard Nucleosides and 2′-Substituted Stereo-Non-Standard Nucleosides In Vitro

Caspase Activity mediated by the modified oligonucleotides was tested in a series of experiments that had similar culture conditions. The results for each experiment are presented in separate tables shown below. Cultured mouse HEPA1-6 cells at a density of 20,000 cells per well were transfected using electroporation with modified oligonucleotides diluted to 20 μM. After a treatment period of approximately 16 hours, caspase-3 and caspase-7 activation was measured using the Caspase-Glo® 3/7 Assay System (G8090, Promega). Increased levels of caspase activation correlate with apoptotic cell death. As seen in the table below, there is a significant reduction in caspase activation and cytotoxicity of the newly designed modified oligonucleotides containing 2′-substituted stereo-standard nucleosides and 2′-substituted stereo-non-standard nucleosides compared to compound 558807.

TABLE 2
In vitro Caspase activation by modified oligonucleotides
containing 2′-substituted stereo-standard nucleosides and
2′-substituted stereo-non-standard nucleosides

| | Caspase |
|---|---|
| Compound | Activation |
| No. | (% Mock) |
| 558807 | 1153 |
| 1385844 | 174 |
| 1385840 | 118 |
| 1385841 | 171 |
| 1385845 | 331 |
| 1385842 | 120 |
| 1385838 | 124 |
| 1385839 | 109 |
| 1385843 | 223 |

### Example 3: Stability of Modified Oligonucleotides Containing 2′-Substituted Stereo-Standard Nucleosides and 2′-Substituted Stereo-Non-Standard Nucleosides

The thermal stability (Tm) of duplexes of each of modified oligonucleotides described in the examples above with a complementary RNA 20-mer having the sequence GAUAAUGUGAGAACAUGCCU (SEQ ID NO: 6) was tested. Each modified oligonucleotide was separately hybridized with the complementary RNA strand to form a duplex. Once the duplex was formed, it was slowly heated and the melting temperature was measured using a spectrophotometer and the hyperchromicity method. Results are provided in Table 3, below. This example demonstrates that 2′-substituted stereo-standard nucleosides and 2′-substituted stereo-non-standard nucleosides can be incorporated into modified oligonucleotides without significantly destabilizing the interaction between the modified oligonucleotide and its complement.

TABLE 3
Tm of modified oligonucleotides complementary to CXCL12

| Compound | |
|---|---|
| No. | Tm (° C.) |
| 558807 | 64.22 |
| 1385844 | 64.37 |
| 1385840 | 61.39 |
| 1385841 | 62.32 |
| 1385845 | 61.27 |
| 1385842 | 60.55 |
| 1385838 | 62.17 |
| 1385839 | 64.49 |
| 1385843 | 64.46 |

### Example 4: In Vivo Activity and Tolerability of Modified Oligonucleotides Containing 2′-Substituted Stereo-Standard Nucleosides and 2′-Substituted Stereo-Non-Standard Nucleosides

Groups of 3 Balb/c mice were injected subcutaneously with 1.9, 5.6, 16.7, 50 and 150 mg/kg of compound 1385838, 1385839, 1385840, or 1385841. One group of three Balb/c mice was injected subcutaneously with 1.8, 5.5, 16.7 and 50 mg/kg of compound 558807. One group of four Balb/c mice was injected with PBS. Mice were euthanized 72 hours following the administration of compound and plasma chemistries and RNA was analyzed.

### Plasma Chemistry Markers

In vivo tolerability of the modified oligonucleotides was determined by measuring plasma levels of aspartate aminotransferase (AST) and alanine aminotransferase (ALT) using an automated clinical chemistry analyzer. All the newly designed modified oligonucleotides show improvement in tolerability markers compared to compound 558807.

TABLE 4
Plasma chemistry markers in vivo

| Compound | Concentration | AST | ALT |
|---|---|---|---|
| No. | (mg/kg) | (IU/L) | (IU/L) |
| PBS | N/A | 30 | 54 |
| 558807 | 50 | 4767 | 6391 |
| | 16.7 | 228 | 270 |
| | 5.5 | 30 | 68 |
| | 1.8 | 41 | 60 |
| 1385838 | 150 | 41 | 70 |
| | 50 | 50 | 104 |
| | 16.7 | 27 | 50 |
| | 5.5 | 34 | 104 |
| | 1.8 | 39 | 76 |
| 1385839 | 150 | 53 | 116 |
| | 50 | 32 | 64 |
| | 16.7 | 39 | 105 |
| | 5.5 | 33 | 50 |
| | 1.8 | 28 | 49 |
| 1385840 | 150 | 47 | 74 |
| | 50 | 47 | 81 |
| | 16.7 | 55 | 86 |
| | 5.5 | 31 | 46 |
| | 1.8 | 48 | 86 |
| 1385841 | 150 | 64 | 115 |
| | 50 | 31 | 49 |
| | 16.7 | 36 | 57 |
| | 5.5 | 37 | 81 |
| | 1.8 | 31 | 55 |

### RNA Analysis

To evaluate the effect of the modified oligonucleotides on CXCL12 levels, CXCL12 RNA levels in liver were measured using mouse primer-probe set RTS2605, which is described in Example 1. CXCL12 RNA levels were normalized to total RNA content, as measured by RIBOGREENR Reduction of CXCL12 RNA is presented in the tables below as percent CXCL12 RNA levels relative to saline control.

TABLE 5
Activity of modified oligonucleotides in vivo

| Concentration | CXCL12 mRNA (% PBS control) | | | | |
|---|---|---|---|---|---|
| (mg/kg) | 558807 | 1385838 | 1385839 | 1385840 | 1385841 |
| 150 | N/A | 3 | 15 | 6 | 9 |
| 50 | 2 | 6 | 18 | 10 | 12 |
| 16.7 | 4 | 14 | 29 | 24 | 27 |
| 5.6 | 12 | 35 | 55 | 46 | 50 |
| 1.9 | 46 | 68 | 82 | 64 | 83 |

### Example 5: Effect of Stereo-Non-Standard DNA Nucleosides on In Vitro Activity of Modified Oligonucleotides Complementary to Mouse CXCL12

The newly designed modified oligonucleotides described in Table 6 below have either a 2′-β-Xylo-deoxyribosyl stereo-non-standard DNA nucleoside in the gap (a nucleoside of Formula II, wherein J₃ and J₄ are each H), a 2′-α-L-deoxyribosyl stereo-non-standard DNA nucleoside in the gap (a nucleoside of Formula V, wherein J₉ and J₁₀ are each H), or a 2′-substituted stereo-standard modified nucleoside with a 2′-OCH₃ modification in the gap. The precise chemical notation of compound 558807 as well as the newly designed modified oligonucleotides are listed in the table below. A subscript “s” indicates a phosphorothioate internucleoside linkage, a subscript “m” represents a 2′-substituted stereo-standard modified nucleoside with a 2′OCH₃ modification, a subscript “k” represents a cEt modified sugar moiety, a subscript “d” represents a stereo-standard DNA nucleoside, and a superscript “m” indicates 5-methyl Cytosine. A subscript “[dx]” represents a 2′-β-Xylo-deoxyribosyl moiety, which is shown below, wherein Bx is a nucleobase:

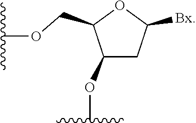

A subscript “[aLd]” represents a 2′-α-L-deoxyribosyl sugar moiety, which is shown below, wherein Bx is a nucleobase:

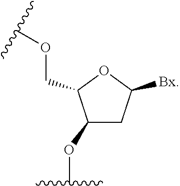

The compounds in Table 6 below are 100% complementary to mouse CXCL12, GENBANK NT_039353.7 truncated from 69430515 to 69445350 (SEQ ID NO: 1), at position 6877 to 6892. The modified oligonucleotides were tested in a series of experiments. The results for each experiment are presented in separate tables shown below. Cultured mouse 3T3-L1 cells at a density of 20,000 cells per well were transfected using electroporation with the modified oligonucleotides diluted to 20 μM, 7 μM, 2 μM, 0.7 μM, 0.3 μM, 0.1 μM, and 0.03 μM. After a treatment period of approximately 16 hours, CXCL12 RNA levels were measured using mouse primer-probe set RTS2605 (forward sequence CCAGAGCCAACGTCAAGCAT, SEQ ID NO: 2; reverse sequence: CAGCCGTGCAACAATCTGAA, SEQ ID NO: 3; probe sequence: TGAAAATCCTCAACACTCCAAACTGTGCC, SEQ ID NO: 4). CXCL12 RNA levels were normalized to total RNA content, as measured by RIBOGREEN®. Activity of the modified oligonucleotides is presented below using the half maximal inhibitory concentration (IC₅₀) values, calculated using the log (inhibitor) vs response (three parameter) function in GraphPad Prism 7. This example demonstrates that modified oligonucleotides having stereo-non-standard DNA nucleosides at certain positions in the gap have similar potency compared to an otherwise identical modified oligonucleotide without any stereo-non-standard DNA nucleosides in the gap.

TABLE 6
Design and activity of modified oligonucleotides having
having sterio-non-standard DNA nucleosides

| | position of | | | | |
|---|---|---|---|---|---|
| | altered | sugar | | | |
| | nucleotide | modification | | | |
| Compound | in central | of altered | | IC₅₀ | SEQ |
| Number | region | nucleotide | Chemistry Notation (5′-3′) | (nM) | ID NO |
| 558807 | n/a | n/a | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 62 | 5 |
| 1382781 | 1 | β-Xylo-DNA | Gₖₛ^{m}CₖₛAₖₛT_{[dx]s}G_{ds}T_{ds}T_{ds}^{m}C_{ds}T_{ds}^{m}C_{ds}A_{ds}^{m}C_{ds}A_{ds}TₖₛTₖₛAₖ | 77 | 5 |
| 1382782 | 2 | β-Xylo-DNA | Gₖₛ^{m}CₖₛAₖₛT_{ds}G_{[dx]s}T_{ds}T_{ds}^{m}C_{ds}T_{ds}^{m}C_{ds}A_{ds}^{m}C_{ds}A_{ds}TₖₛTₖₛAₖ | 120 | 5 |
| 1263776 | 3 | β-Xylo-DNA | Gₖₛ^{m}CₖₛAₖₛT_{ds}G_{ds}T_{[dx]s}T_{ds}^{m}C_{ds}T_{ds}^{m}C_{ds}A_{ds}^{m}C_{ds}sA_{ds}TₖₛTₖₛAₖ | 110 | 5 |
| 1263777 | 4 | β-Xylo-DNA | Gₖₛ^{m}CₖₛAₖₛT_{ds}G_{ds}T_{ds}T_{[dx]s}^{m}C_{ds}T_{ds}^{m}C_{ds}A_{ds}^{m}C_{ds}A_{ds}TₖₛTₖₛAₖ | 66 | 5 |
| 1382783 | 5 | β-Xylo-DNA | Gₖₛ^{m}CₖₛAₖₛT_{ds}G_{ds}T_{ds}T_{ds}^{m}C_{[dx]s}T_{ds}^{m}C_{ds}A_{ds}^{m}C_{ds}A_{ds}TₖₛTₖₛAₖ | 46 | 5 |
| 1382784 | 6 | β-Xylo-DNA | Gₖₛ^{m}CₖₛAₖₛT_{ds}G_{ds}T_{ds}T_{ds}^{m}C_{ds}T_{[dx]s}^{m}C_{ds}A_{ds}^{m}C_{ds}A_{ds}TₖₛTₖₛAₖ | 66 | 5 |
| 1382785 | 7 | β-Xylo-DNA | Gₖₛ^{m}CₖₛAₖₛT_{ds}G_{ds}T_{ds}T_{ds}^{m}C_{ds}T_{ds}^{m}C_{[dx]s}A_{ds}^{m}C_{ds}A_{ds}TₖₛTₖₛAₖ | 52 | 5 |
| 1382786 | 8 | β-Xylo-DNA | Gₖₛ^{m}CₖₛAₖₛT_{ds}G_{ds}T_{ds}T_{ds}^{m}C_{ds}T_{ds}^{m}C_{ds}A_{[dx]s}^{m}C_{ds}A_{ds}TₖₛTₖₛAₖ | 54 | 5 |
| 1382787 | 9 | β-Xylo-DNA | Gₖₛ^{m}CₖₛAₖₛT_{ds}G_{ds}T_{ds}T_{ds}^{m}C_{ds}T_{ds}^{m}C_{ds}A_{ds}^{m}C_{[dx]s}A_{ds}TₖₛTₖₛAₖ | 44 | 5 |
| 1382788 | 10 | β-Xylo-DNA | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{[dx]s}TₖₛTₖₛAₖ | 66 | 5 |
| 936053 | 2 | 2′-OMe | Gₖₛ^{m}CₖₛAₖₛT_{ds}GₘₛT_{ds}T_{ds}^{m}C_{ds}T_{ds}^{m}C_{ds}A_{ds}^{m}C_{ds}A_{ds}TₖₛTₖₛAₖ | ND | 5 |
| 1368053 | 2 | α-L DNA | Gₖₛ^{m}CₖₛAₖₛT_{ds}G_{[aLd]s}T_{ds}T_{ds}^{m}C_{ds}T_{ds}^{m}C_{ds}A_{ds}^{m}C_{ds}A_{ds}TₖₛTₖₛAₖ | ND | 5 |

### Example 6: Caspase Activity of Modified Oligonucleotides Having Stereo-Non-Standard DNA Nucleosides In Vitro

Caspase activity of modified oligonucleotides having stereo-non-standard DNA nucleosides was tested in a series of experiments that had similar culture conditions. The results are presented in Table 7 below. Cultured mouse HEPA1-6 cells at a density of 20,000 cells per well were transfected using electroporation with modified oligonucleotides diluted to 20 μM. After a treatment period of approximately 16 hours, caspase-3 and caspase-7 activation was measured using the Caspase-Glo® 3/7 Assay System (G8090, Promega). Levels of caspase activation correlate with apoptotic cell death. This example demonstrates that placement of stereo-non-standard DNA nucleosides at certain positions in the gap of a modified oligonucleotide reduces cytotoxicity compared to an otherwise identical modified oligonucleotide without any stereo-non-standard DNA nucleosides in the gap.

TABLE 7
In vitro Caspase activation by modified oligonucleotides
having stereo-non-standard DNA nucleosides

| Compound | Caspase |
|---|---|
| Number | % mock |
| 558807 | 1402 |
| 1382781 | 203 |
| 1382782 | 140 |
| 1263776 | 543 |
| 1263777 | 1146 |
| 1382783 | 492 |
| 1382784 | 646 |
| 1382785 | 949 |
| 1382786 | 965 |
| 1382787 | 1352 |
| 1382788 | 1043 |

### Example 7: Stability of Modified Oligonucleotides Having Stereo-Non-Standard DNA Nucleosides

The thermal stability (Tm) of duplexes of each of modified oligonucleotides described in the examples above with a complementary RNA 20-mer having the sequence GAUAAUGUGAGAACAUGCCU (SEQ ID NO: 6) was tested. Each modified oligonucleotide was separately hybridized with the complementary RNA strand to form a duplex. Once the duplex was formed, it was slowly heated and the melting temperature was measured using a spectrophotometer and the hyperchromicity method. Results are provided in Table 8, below. This example demonstrates that stereo-non-standard DNA nucleosides can be incorporated into modified oligonucleotides without destabilizing the interaction between the modified oligonucleotide and its complement.

TABLE 8
Tm of modified oligonucleotides complementary to
CXCL12 and having non-standard DNA nucleosides

| Compound | |
|---|---|
| Number | Tm (° C.) |
| 558807 | 64.4 |
| 1382781 | 63.8 |
| 1382782 | 63.4 |
| 1263776 | 63.1 |
| 1263777 | 64.8 |
| 1382783 | 63.1 |
| 1382784 | 63.2 |
| 1382785 | 64.8 |
| 1382786 | 63.2 |
| 1382787 | 65.2 |
| 1382788 | 63.2 |

### Example 8: In Vivo Activity and Tolerability of Modified Oligonucleotides Having Stereo-Non-Standard DNA Nucleosides

Groups of 3 Balb/c mice were injected subcutaneously with 1.8, 5.5, 16.7, 50 and 150 mg/kg of compound 1368053, 1382781, 1382782, or 936053. One group of four Balb/c mice was injected with PBS. Mice were euthanized 72 hours following the subcutaneous injection, and plasma chemistry and RNA was analyzed.

### Plasma Chemistry Markers

In vivo tolerability of the modified oligonucleotides was determined by measuring plasma levels of aspartate aminotransferase (AST) and alanine aminotransferase (ALT) using an automated clinical chemistry analyzer. The newly designed modified oligonucleotides having stereo-non-standard DNA nucleosides show good tolerability over a range of doses, including comparable tolerability to a modified oligonucleotide having a 2′-substituted stereo-standard nucleoside with a 2′-OCH₃ modification at the 2 position of the gap (compound 936053). For mice injected with PBS, ALT is observed to be 28 IU/L, and AST is 37 IU/L.

TABLE 9
Plasma chemistry markers in vivo

| | position | | | | | | |
|---|---|---|---|---|---|---|---|
| | of altered | sugar | | | | | |
| | nucleotide | modification | ALT (IU/L) | | | | |
| Compound | in central | of altered | 150 | 50 | 16.7 | 5.5 | 1.8 |
| Number | region | nucleotide | mg/kg | mg/kg | mg/kg | mg/kg | mg/kg |
| 936053 | 2 | 2′-OMe | 56 | 36 | 31 | 26 | 33 |
| 1368053 | 2 | α-L DNA | 125 | 35 | 33 | 23 | 33 |
| 1382781 | 1 | β-Xylo-DNA | 2389 | 92 | 28 | 24 | 31 |
| 1382782 | 2 | β-Xylo-DNA | 34 | 28 | 36 | 32 | 35 |

TABLE 10
Plasma chemistry markers in vivo

| | position | | | | | | |
|---|---|---|---|---|---|---|---|
| | of altered | sugar | | | | | |
| | nucleotide | modification | AST (IU/L) | | | | |
| Compound | in central | of altered | 150 | 50 | 16.7 | 5.5 | 1.8 |
| Number | region | nucleotide | mg/kg | mg/kg | mg/kg | mg/kg | mg/kg |
| 936053 | 2 | 2′-OMe | 61 | 46 | 46 | 40 | 43 |
| 1368053 | 2 | α-L DNA | 109 | 58 | 44 | 48 | 43 |
| 1382781 | 1 | β-Xylo-DNA | 1692 | 124 | 44 | 38 | 47 |
| 1382782 | 2 | β-Xylo-DNA | 44 | 40 | 46 | 61 | 51 |

### RNA Analysis

To evaluate the effect of the modified oligonucleotides on CXCL12 levels, CXCL12 RNA levels in liver were measured using mouse primer-probe set RTS2605, which is described in Example 1. CXCL12 RNA levels were normalized to total RNA content, as measured by RIBOGREENR Reduction of CXCL12 RNA is presented in the tables below as percent CXCL12 RNA levels relative to saline control.

TABLE 11
Activity of sugar-modified oligonucleotides in vivo

| Concentration | CXCL12 mRNA (% PBS) | | | |
|---|---|---|---|---|
| (mg/kg) | 936053 | 1368053 | 1382781 | 1382782 |
| 150 | 10 | 3 | 4 | 6 |
| 50 | 13 | 5 | 8 | 9 |
| 16.7 | 19 | 10 | 13 | 12 |
| 5.5 | 38 | 22 | 20 | 22 |
| 1.8 | 55 | 40 | 39 | 45 |

This example demonstrates that modified oligonucleotides having stereo-non-standard DNA nucleosides in the gap have similar tolerability over a range of doses as compared to a modified oligonucleotide having a 2′-substituted stereo-standard nucleoside with a 2′-OCH₃ modification at the 2 position of the gap. Additionally, modified oligonucleotides having stereo-non-standard DNA nucleosides in the gap have better potency as compared to a modified oligonucleotide having a 2′-substituted stereo-standard nucleoside with a 2′-OCH₃ modification at the 2 position of the gap.

### Example 9: In Vivo Activity and Tolerability of Modified Oligonucleotides Having Stereo-Non-Standard DNA Nucleosides

Groups of 3 Balb/c mice were injected subcutaneously with 10 and 150 mg/kg of newly synthesized compounds 1263776, 1263777, or 936053. One group of four Balb/c mice was injected with PBS. Mice were euthanized 72 hours following the administration of compound. Plasma chemistry and RNA was then analyzed.

### Plasma Chemistry Markers

In vivo tolerability of the modified oligonucleotides was determined by measuring plasma levels of aspartate aminotransferase (AST), alanine aminotransferase (ALT) using an automated clinical chemistry analyzer. For mice injected with PBS, ALT is observed to be 26 IU/L, and AST is 53 IU/L.

TABLE 12
Plasma chemistry markers in vivo

| | position | | | |
|---|---|---|---|---|
| | of altered | sugar | | |
| | nucleotide | modification | ALT (IU/L) | |
| Compound | in central | of altered | 150 | 10 |
| Number | region | nucleotide | mg/kg | mg/kg |
| 936053 | 2 | 2′-OMe | 83 | 23 |
| 1263776 | 3 | β-Xylo-DNA | 9234 | 27 |
| 1263777 | 4 | β-Xylo-DNA | ND | 58 |

TABLE 13
Plasma chemistry markers in vivo

| | position | | | |
|---|---|---|---|---|
| | of altered | sugar | | |
| | nucleotide | modification | AST (IU/L) | |
| Compound | in central | of altered | 150 | 10 |
| Number | region | nucleotide | mg/kg | mg/kg |
| 936053 | 2 | 2′-OMe | 88 | 45 |
| 1263776 | 3 | β-Xylo-DNA | 10075 | 54 |
| 1263777 | 4 | β-Xylo-DNA | ND | 102 |

### RNA Analysis

To evaluate the effect of the modified oligonucleotides on CXCL12 levels, CXCL12 RNA levels in liver were measured using mouse primer-probe set RTS2605, which is described in Example 1. CXCL12 RNA levels were normalized to total RNA content, as measured by RIBOGREEN®. Reduction of CXCL12 RNA is presented in the tables below as percent CXCL12 RNA levels relative to saline control.

TABLE 14
Activity of sugar-modified oligonucleotides in vivo

| Concentration | CXCL12 mRNA (% PBS) | | |
|---|---|---|---|
| (mg/kg) | 936053 | 1263776 | 1263777 |
| 150 | 13 | 11 | ND |
| 10 | 37 | 19 | 13 |

### Example 10: In Vivo Activity and Tolerability of Modified Oligonucleotides Having Stereo-Non-Standard DNA Nucleosides

Modified oligonucleotides having a stereo-non-standard DNA nucleoside at positions 1-5 of the gap were synthesized and are described in Table 15 below. The compounds in Table 15 below are 100% complementary to mouse CXCL12, GENBANK NT_039353.7 truncated from 69430515 to 69445350 (SEQ ID NO: 1), at position 6877 to 6892.

In Table 15 below, a subscript “s” indicates a phosphorothioate internucleoside linkage, a subscript “k” represents a cEt modified sugar moiety, a subscript “d” represents a stereo-standard DNA nucleoside, and a superscript “m” indicates 5-methyl Cytosine.

A subscript “[aLd]” represents a 2′-α-L-deoxyribosyl sugar moiety, which is shown below, wherein Bx is a nucleobase:

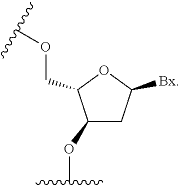

A “aLd” nucleoside is a nucleoside of Formula V, wherein J₉ and J₁₀ are each H.

TABLE 15
Modified oligonucleotides complementary to CXCL12

| | position of | | | |
|---|---|---|---|---|
| | altered | sugar | | |
| | nucleotide | modification | | |
| Compound | in central | of altered | | SEQ |
| Number | region | nucleotide | Chemistry Notation (5′-3′) | ID No. |
| 558807 | n/a | n/a | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 5 |
| 1368034 | 1 | α-L DNA | Gₖₛ^{m}CₖₛAₖₛT_{[aLd]s}G_{ds}T_{ds}T_{ds}^{m}C_{ds}T_{ds}^{m}C_{ds}A_{ds}^{m}C_{ds}A_{ds}TₖₛTₖₛAₖ | 5 |
| 1368053 | 2 | α-L DNA | Gₖₛ^{m}CₖₛAₖₛT_{ds}G_{[aLd]s}T_{ds}T_{ds}^{m}C_{ds}T_{ds}^{m}C_{ds}A_{ds}^{m}C_{ds}A_{ds}TₖₛTₖₛAₖ | 5 |
| 1215461 | 3 | α-L DNA | Gₖₛ^{m}CₖₛAₖₛT_{ds}G_{ds}T_{[aLd]s}T_{ds}^{m}C_{ds}T_{ds}^{m}C_{ds}A_{ds}^{m}C_{ds}A_{ds}TₖₛTₖₛAₖ | 5 |
| 1215462 | 4 | α-L DNA | Gₖₛ^{m}CₖₛAₖₛT_{ds}G_{ds}T_{ds}T_{[aLd]s}C_{ds}T_{ds}^{m}C_{ds}A_{ds}^{m}C_{ds}A_{ds}TₖₛTₖₛAₖ | 5 |
| 1368054 | 5 | α-L DNA | Gₖₛ^{m}CₖₛAₖₛT_{ds}G_{ds}T_{ds}T_{ds}^{m}C_{[aLd]s}T_{ds}^{m}C_{ds}A_{ds}^{m}C_{ds}A_{ds}TₖₛTₖₛAₖ | 5 |

Groups of 3 Balb/c mice were injected subcutaneously with 1.8, 5.5, 16.7, 50 and 150 mg/kg of newly synthesized modified oligonucleotides 1368034, 1368053, 1215461, 1215462, or 1368054. One group of three Balb/c mice was injected subcutaneously with 1.8, 5.5, 16.7 and 50 mg/kg of compound 558807. One group of four Balb/c mice was injected with PBS. Mice were euthanized 72 hours following the administration of compound. Plasma chemistry and RNA was then analyzed.

### Plasma Chemistry Markers

In vivo tolerability of the modified oligonucleotides was determined by measuring plasma levels of aspartate aminotransferase (AST), alanine aminotransferase (ALT) using an automated clinical chemistry analyzer. All the newly designed modified oligonucleotides having a stereo-non-standard DNA nucleoside show improvement in tolerability markers compared to compound 558807. For mice injected with PBS, ALT is observed to be 23 IU/L, and AST is 43 IU/L.

TABLE 16
Plasma chemistry markers in vivo

| | position of | | | | | | |
|---|---|---|---|---|---|---|---|
| | altered | sugar | | | | | |
| | nucleotide | modification | ALT (IU/L) | | | | |
| Compound | in central | of altered | 150 | 50 | 16.7 | 5.5 | 1.8 |
| Number | region | nucleotide | mg/kg | mg/kg | mg/kg | mg/kg | mg/kg |
| 558807 | n/a | n/a | n/a | 4035 | 273 | 26 | 40 |
| 1368034 | 1 | α-L DNA | 47 | 50 | 30 | 29 | 27 |
| 1368053 | 2 | α-L DNA | 50 | 39 | 23 | 23 | 29 |
| 1215461 | 3 | α-L DNA | 4561 | 667 | 28 | 27 | 27 |
| 1215462 | 4 | α-L DNA | 933 | 45 | 22 | 35 | 26 |
| 1368054 | 5 | α-L DNA | 1190 | 100 | 30 | 40 | 28 |

TABLE 17
Plasma chemistry markers in vivo

| | position of | | | | | | |
|---|---|---|---|---|---|---|---|
| | altered | sugar | | | | | |
| | nucleotide | modification | AST (IU/L) | | | | |
| Compound | in central | of altered | 150 | 50 | 16.7 | 5.5 | 1.8 |
| Number | region | nucleotide | mg/kg | mg/kg | mg/kg | mg/kg | mg/kg |
| 558807 | n/a | n/a | n/a | 4870 | 328 | 69 | 80 |
| 1368034 | 1 | α-L DNA | 73 | 86 | 53 | 63 | 57 |
| 1368053 | 2 | α-L DNA | 74 | 122 | 68 | 48 | 111 |
| 1215461 | 3 | α-L DNA | 4815 | 636 | 58 | 53 | 47 |
| 1215462 | 4 | α-L DNA | 1135 | 107 | 47 | 64 | 47 |
| 1368054 | 5 | α-L DNA | 914 | 104 | 49 | 72 | 89 |

### RNA Analysis

To evaluate the effect of the modified oligonucleotides on CXCL12 levels, CXCL12 RNA levels in liver were measured using mouse primer-probe set RTS2605, which is described in Example 1. CXCL12 RNA levels were normalized to total RNA content, as measured by RIBOGREEN®. Reduction of CXCL12 RNA is presented in the tables below as percent CXCL12 RNA levels relative to saline control.

TABLE 18
Activity of sugar-modified oligonucleotides in vivo

| Concentration | CXCL12 mRNA (% PBS) | | | | | |
|---|---|---|---|---|---|---|
| (mg/kg) | 558807 | 1368034 | 1368053 | 1215461 | 1215462 | 1368054 |
| 150 | n/a | 9 | 5 | 3 | 4 | 2 |
| 50 | 4 | 10 | 8 | 3 | 5 | 4 |
| 16.7 | 4 | 18 | 12 | 5 | 11 | 7 |
| 5.5 | 12 | 35 | 31 | 20 | 26 | 21 |
| 1.8 | 43 | 73 | 62 | 51 | 53 | 53 |

### Example 11: Design and Synthesis of Stereo-Non-Standard Nucleosides and 2′-Substituted Stereo-Non-Standard Nucleosides

2′-substituted stereo-non-standard nucleosides and stereo-non-standard nucleosides described herein were prepared as amidites as described below. The stereo-non-standard nucleoside amidites may then be incorporated into a modified oligonucleotide during modified oligonucleotide synthesis. Further details of these syntheses are provided in Examples 29 and 33-40.

Compound 1a, an amidite of a stereo-non-standard nucleoside, was prepared according to the scheme below:

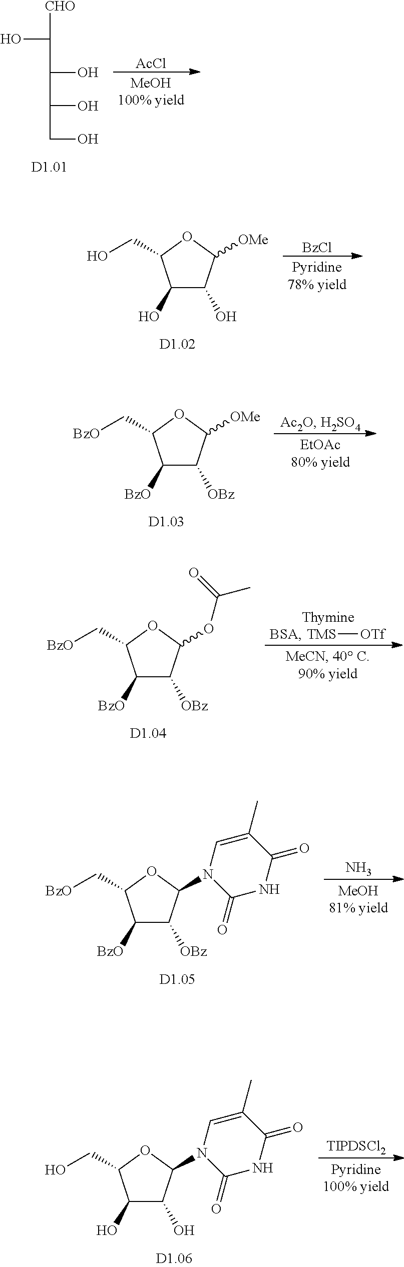

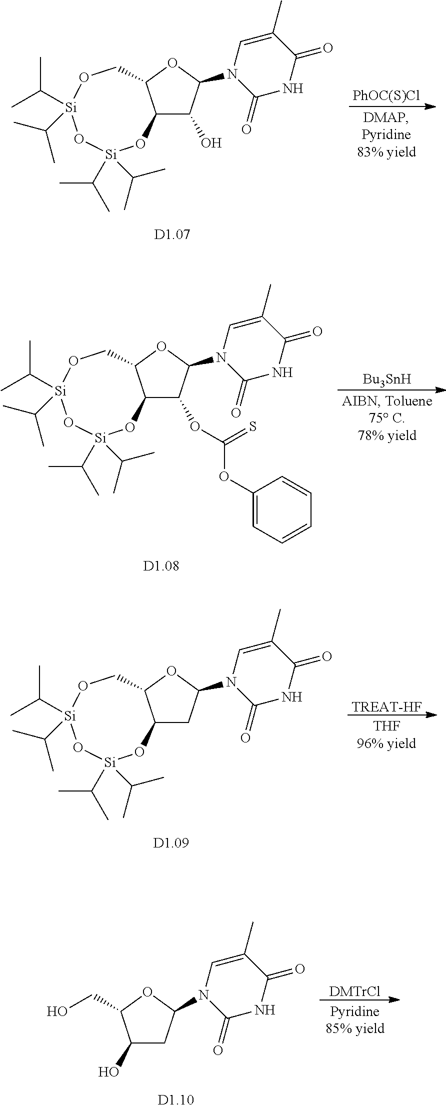

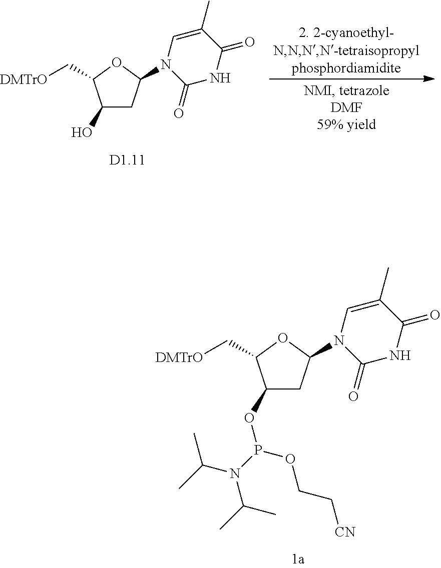

Compound 2a, an amidite of a stereo-non-standard nucleoside, was prepared according to the scheme below:

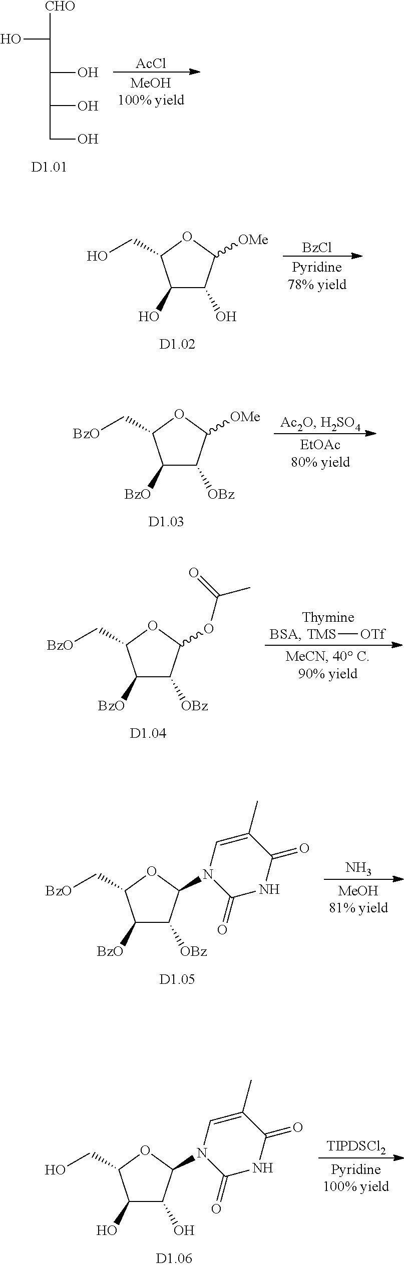

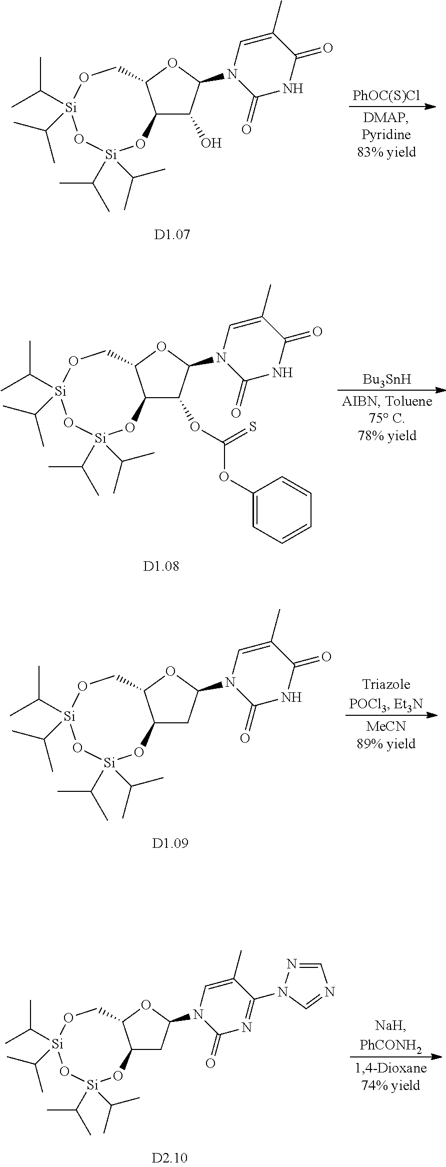

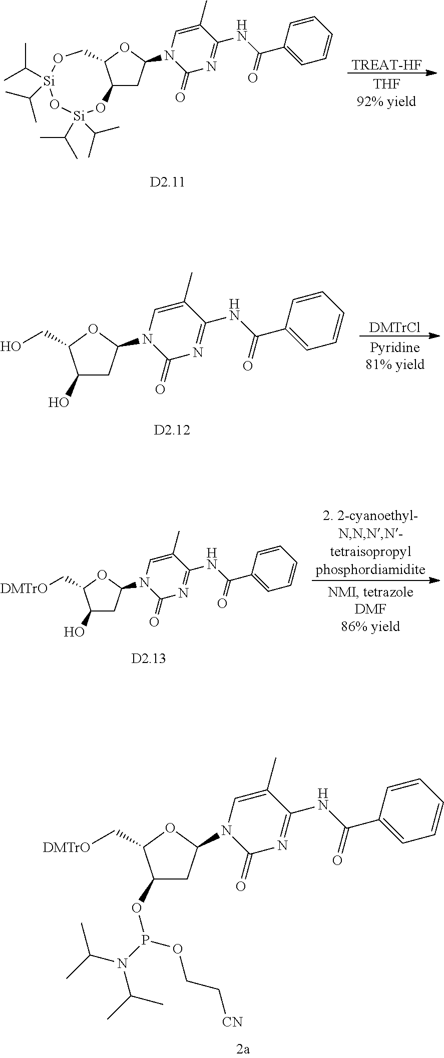

Compound 3a, an amidite of a stereo-non-standard nucleoside, was prepared according to the scheme below:

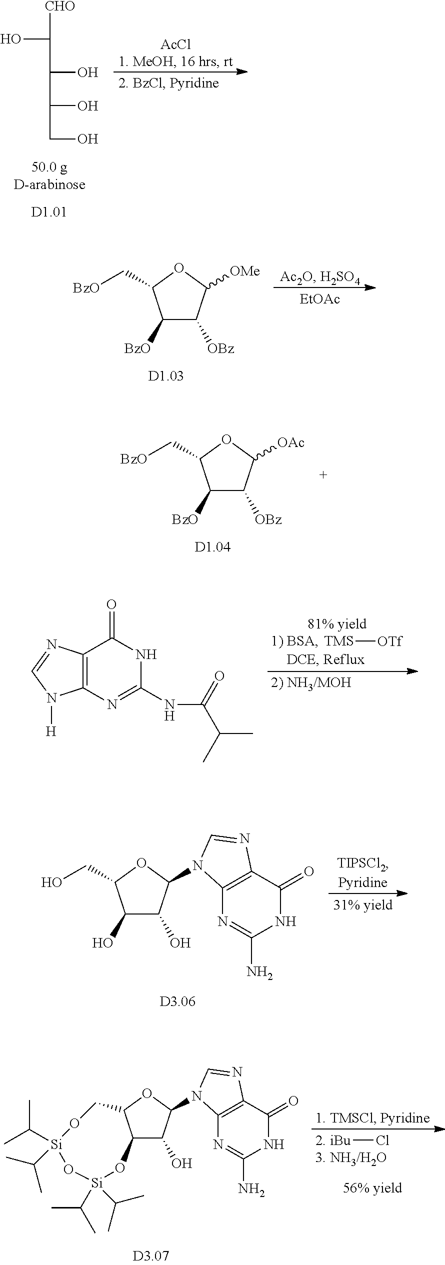

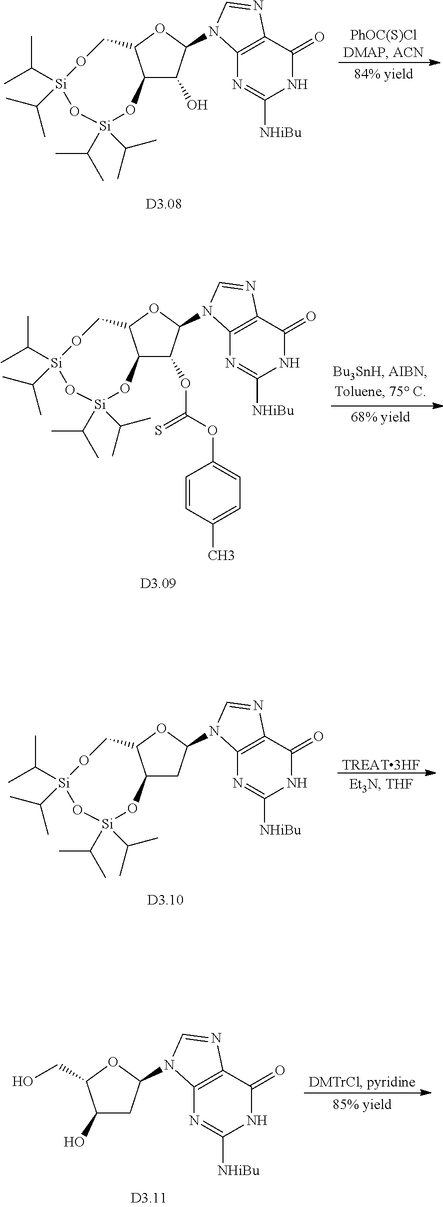

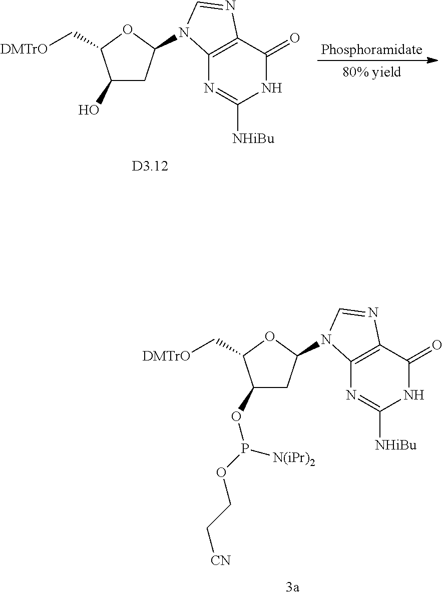

Compound 4a, an amidite of a stereo-non-standard nucleoside, was prepared according to the scheme below:

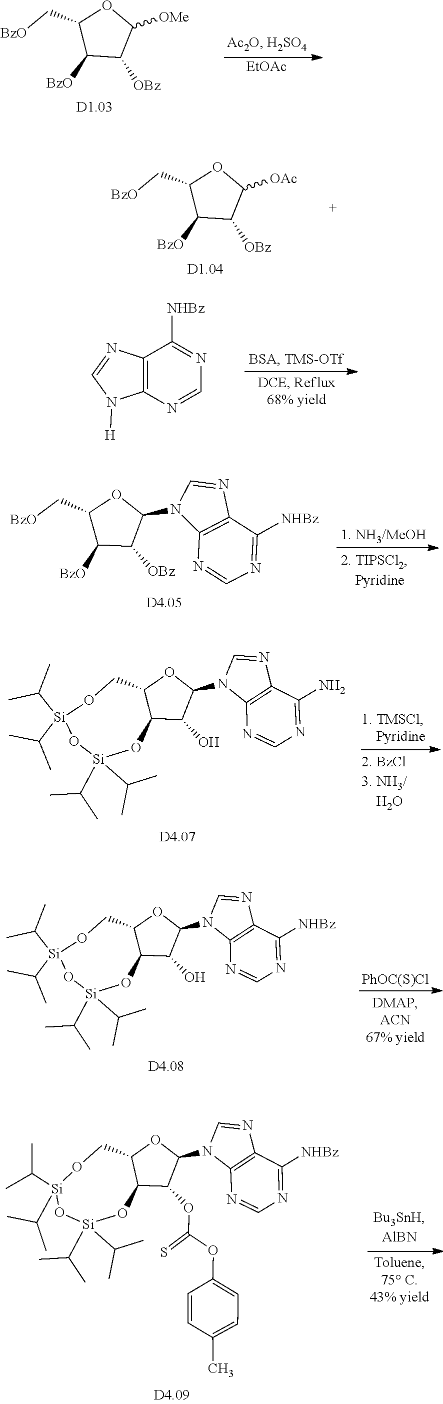

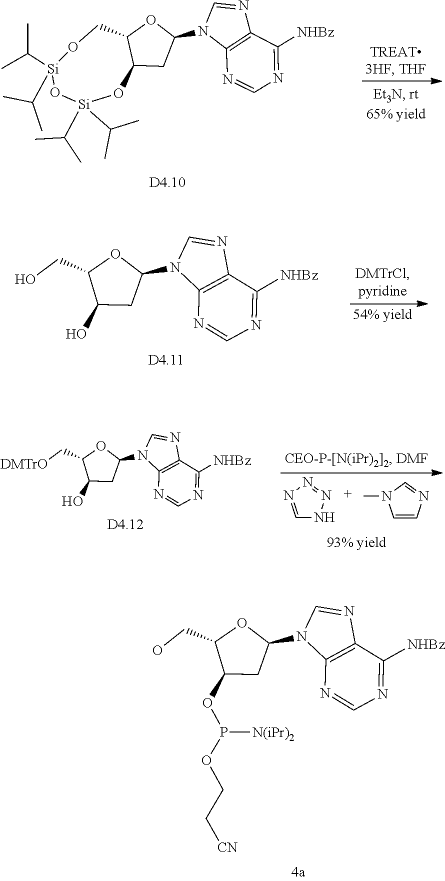

Compounds 5a and 6a, amidites of stereo-non-standard nucleosides, were prepared according to the scheme below:

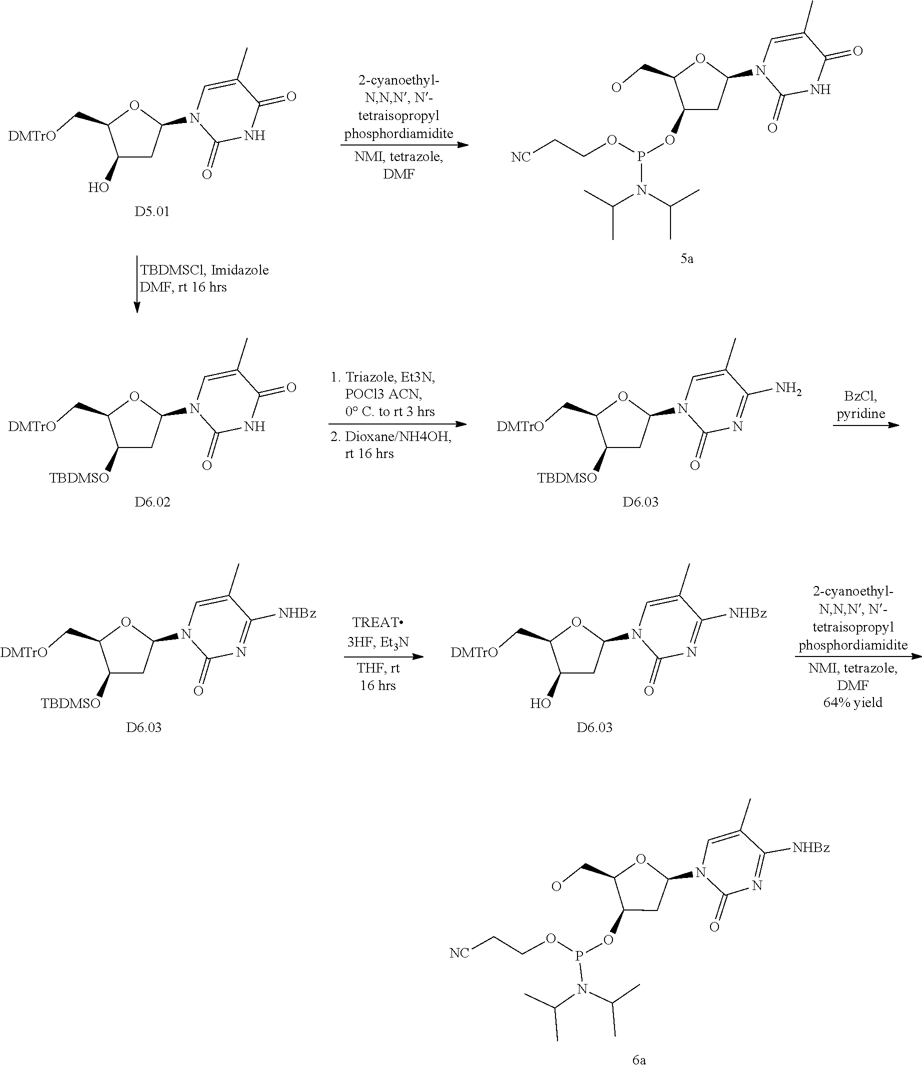

Compound 7a, an amidite of a stereo-non-standard nucleoside, was prepared according to the scheme below:

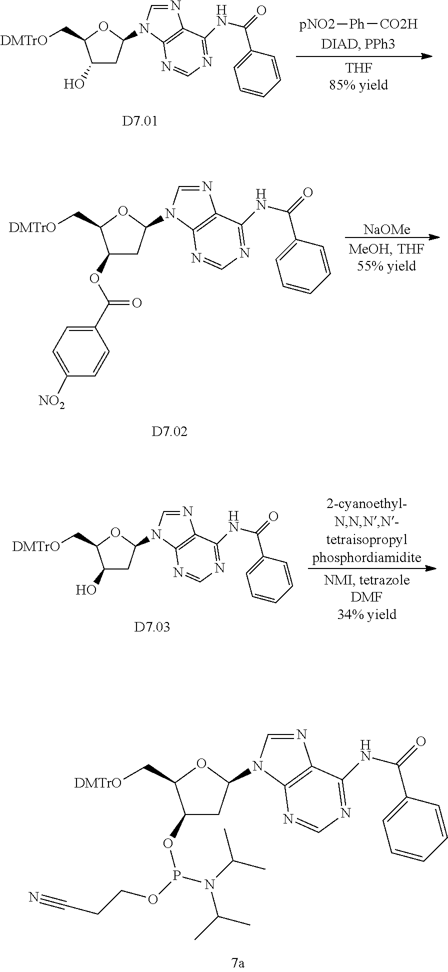

Compound 8a, an amidite of a stereo-non-standard nucleoside, was prepared according to the scheme below:

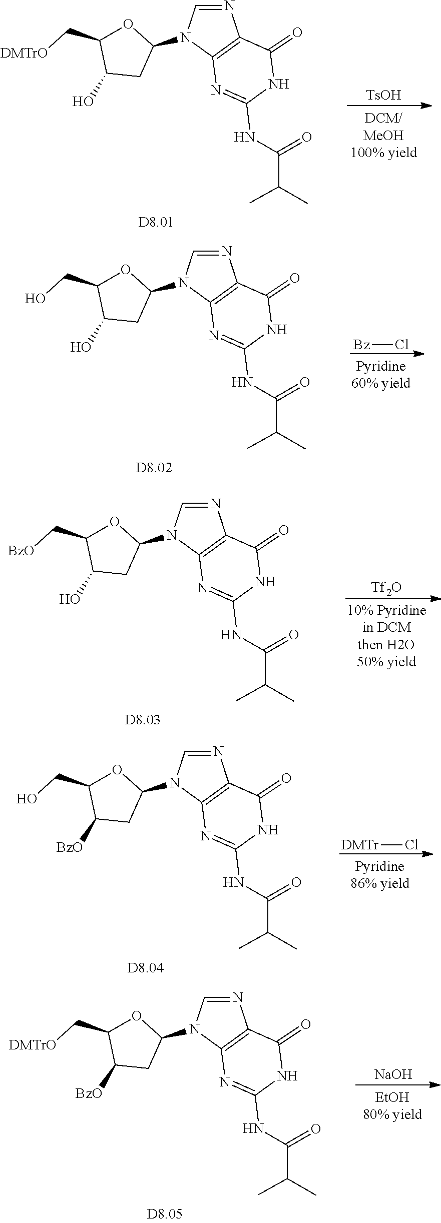

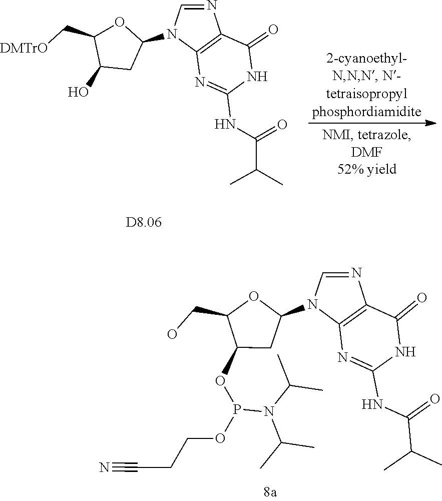

Compound 9a, an amidite of a 2′ substituted stereo-non-standard nucleoside, was prepared according to the scheme below. Further details of this synthesis are provided in Example 29.

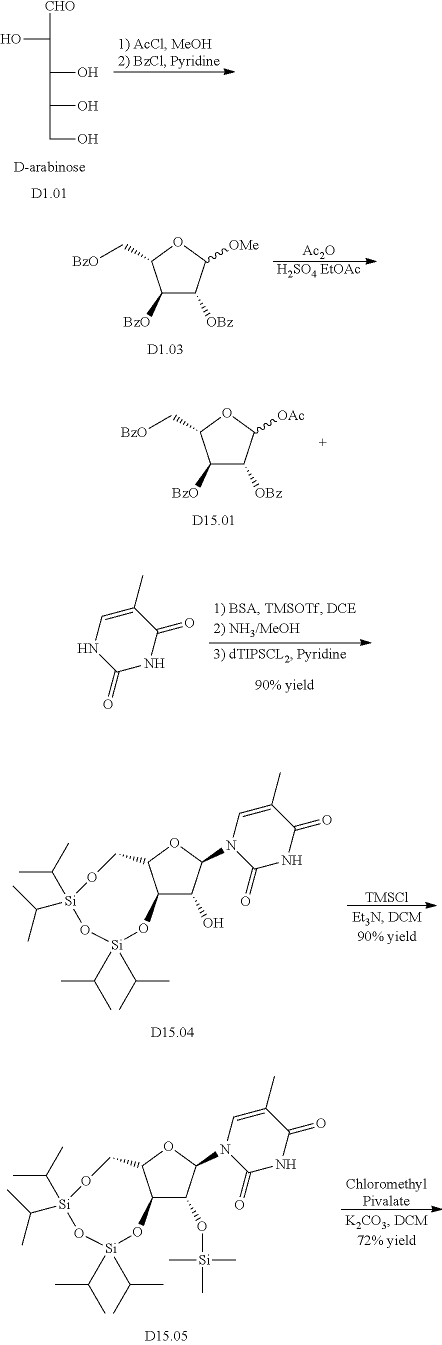

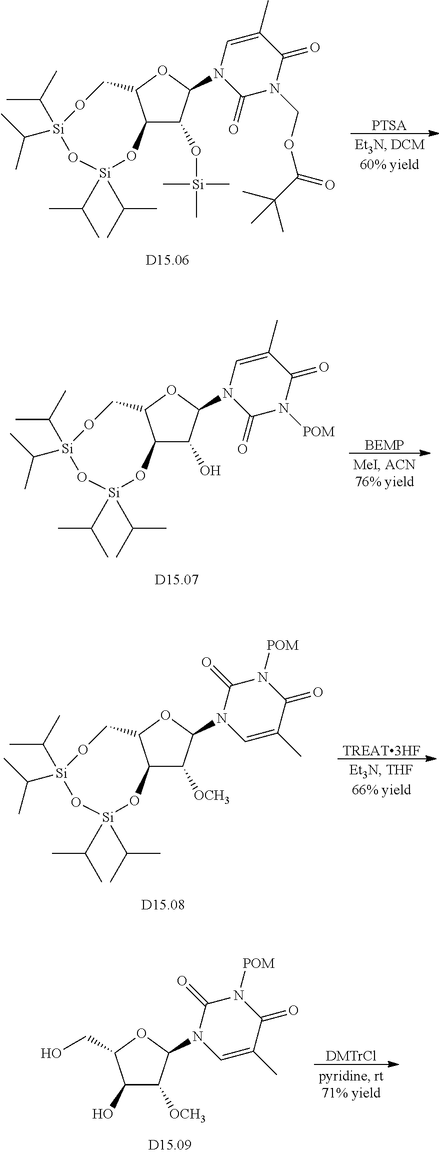

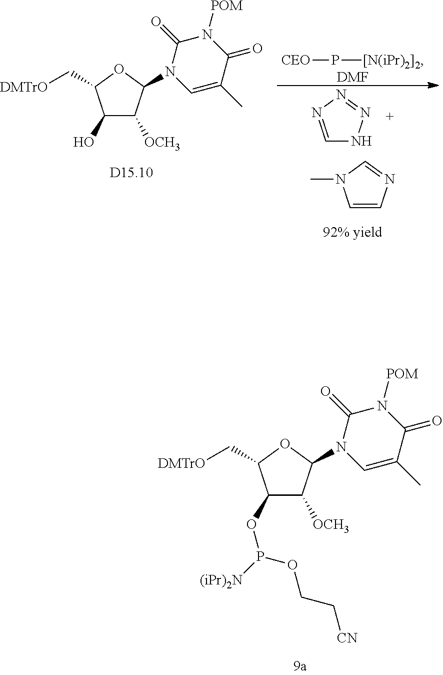

### Example 12: Design and Synthesis of 2′-Substituted Stereo-Standard Nucleosides, Stereo-Non-Standard Nucleosides, and 2′-Substituted Stereo-Non-Standard Nucleosides

2′-substituted stereo-non-standard nucleosides and stereo-non-standard nucleosides described herein may be prepared as amidites as described below. The 2′-substituted stereo-non-standard nucleoside amidites and stereo-non-standard nucleoside amidites may then be incorporated into a modified oligonucleotide during modified oligonucleotide synthesis. Further synthetic details are provided in Example 31 and 41-43.

A scheme for the synthesis of an amidite of the stereo-non-standard nucleoside 10a is shown below:

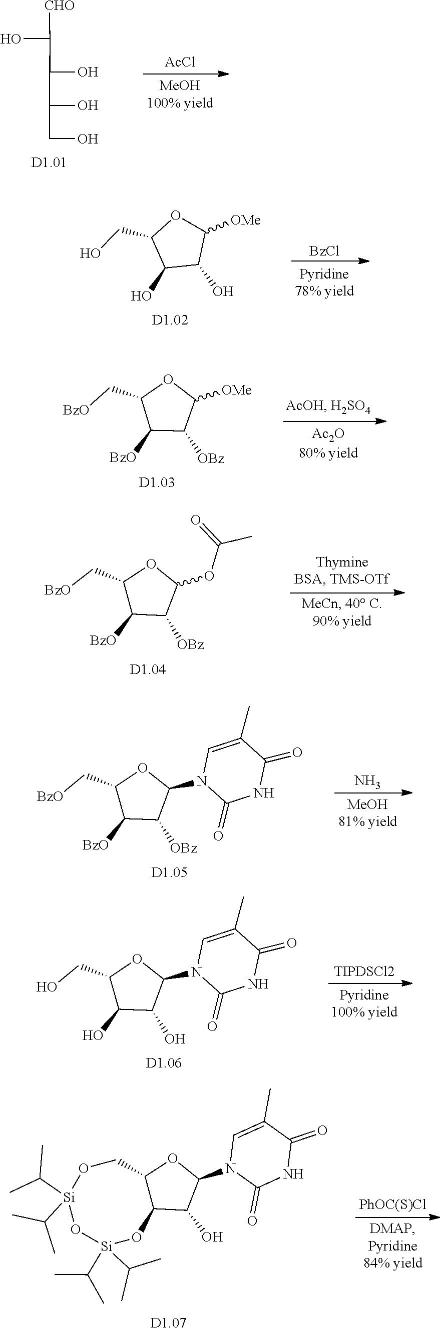

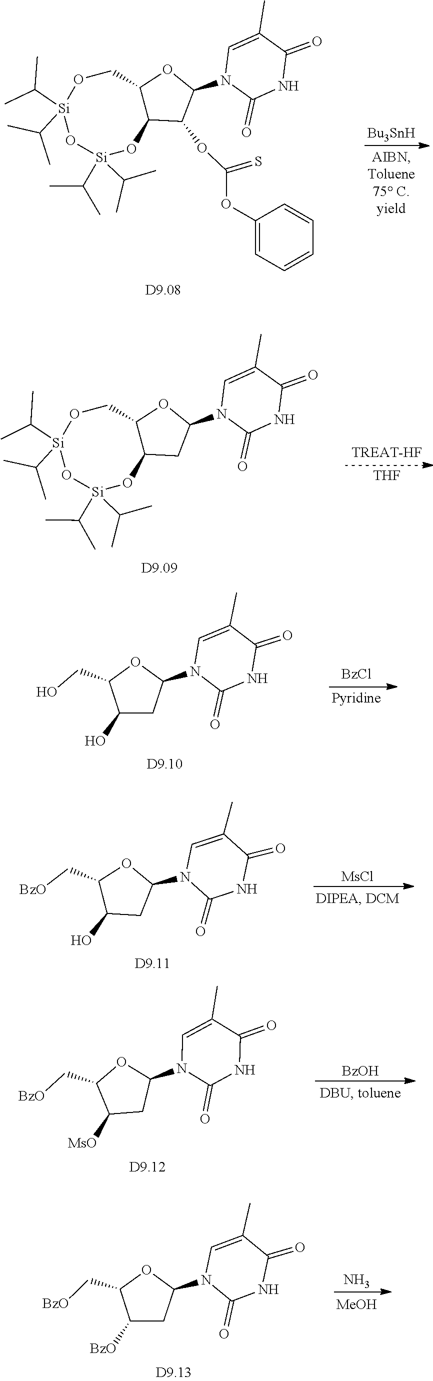

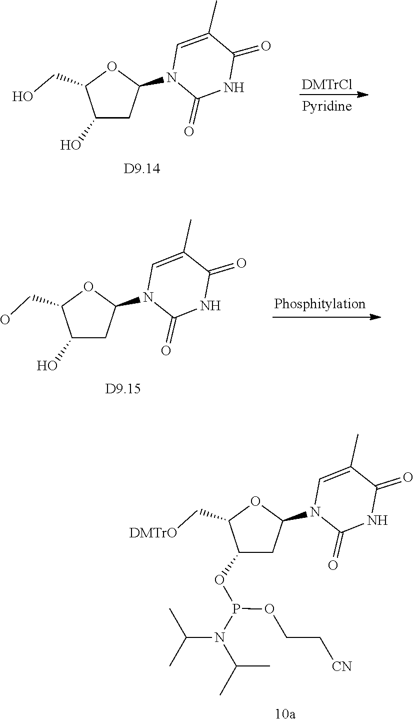

A scheme for the synthesis of an amidite of the stereo-non-standard nucleoside 11a is shown below:

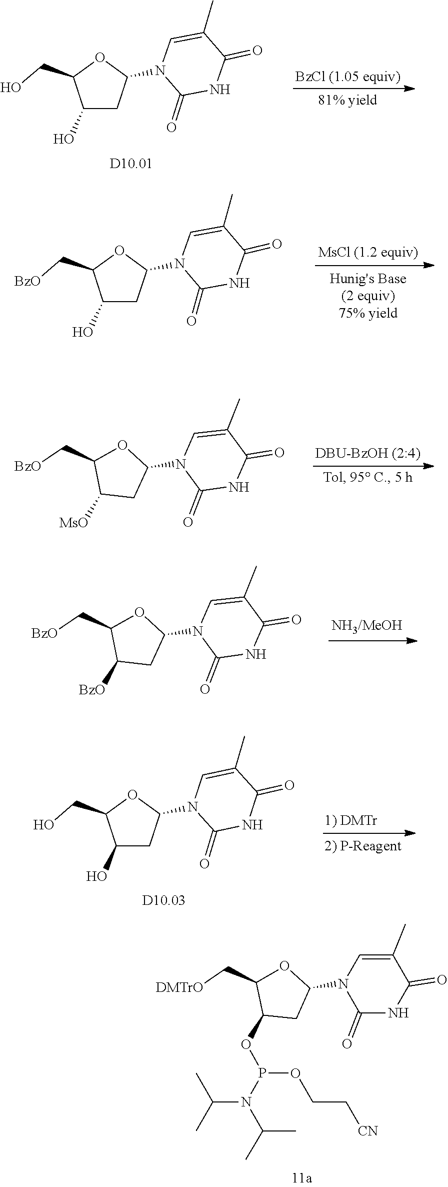

A scheme for the synthesis of an amidite of the stereo-non-standard nucleoside 12a is shown below:

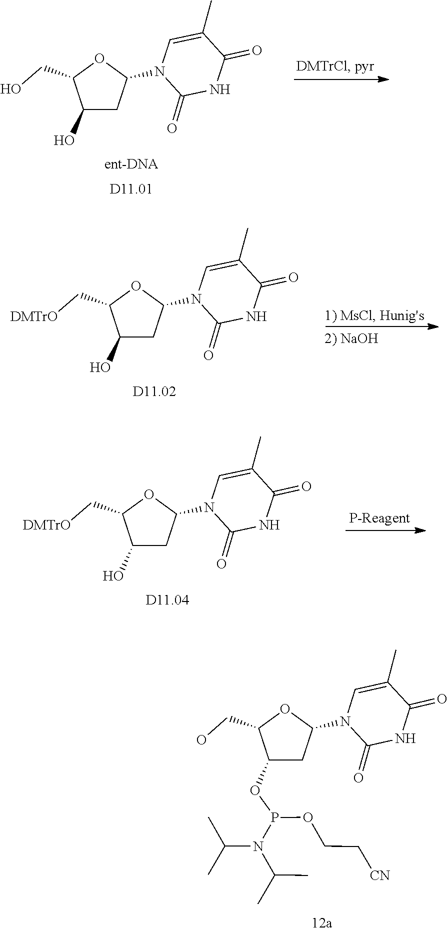

A scheme for the synthesis of an amidite of the 2′ substituted stereo-standard nucleoside 13a is shown below; however, an alternative synthesis is described in Example 30:

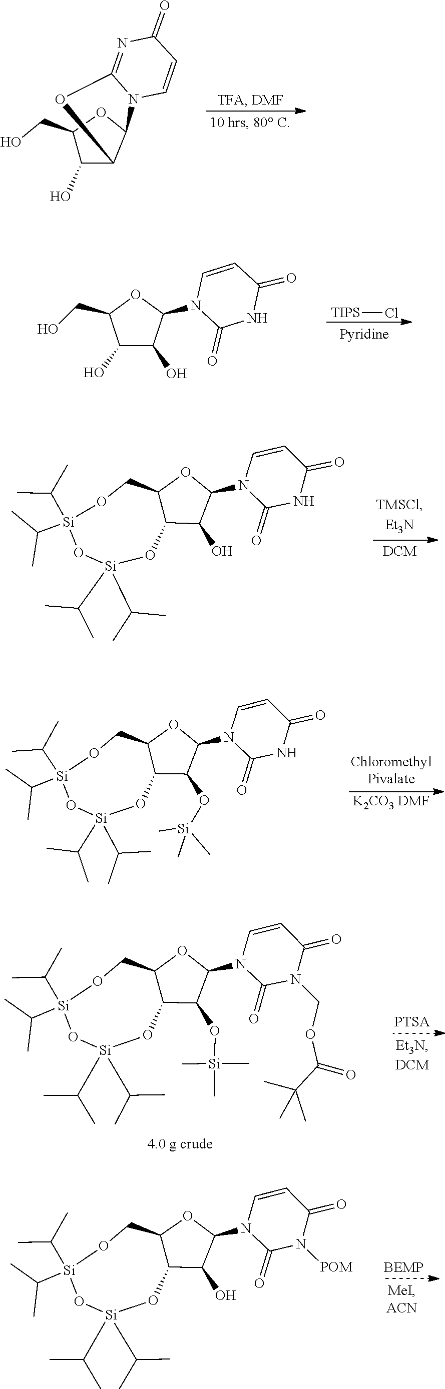

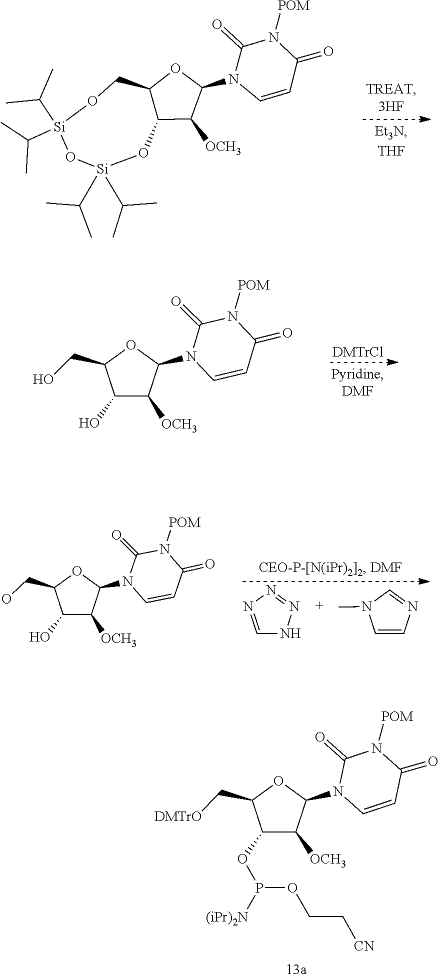

Schemes for the synthesis of amidites of the 2′ substituted stereo-non-standard nucleosides 14a, 15a, and 16a are shown below:

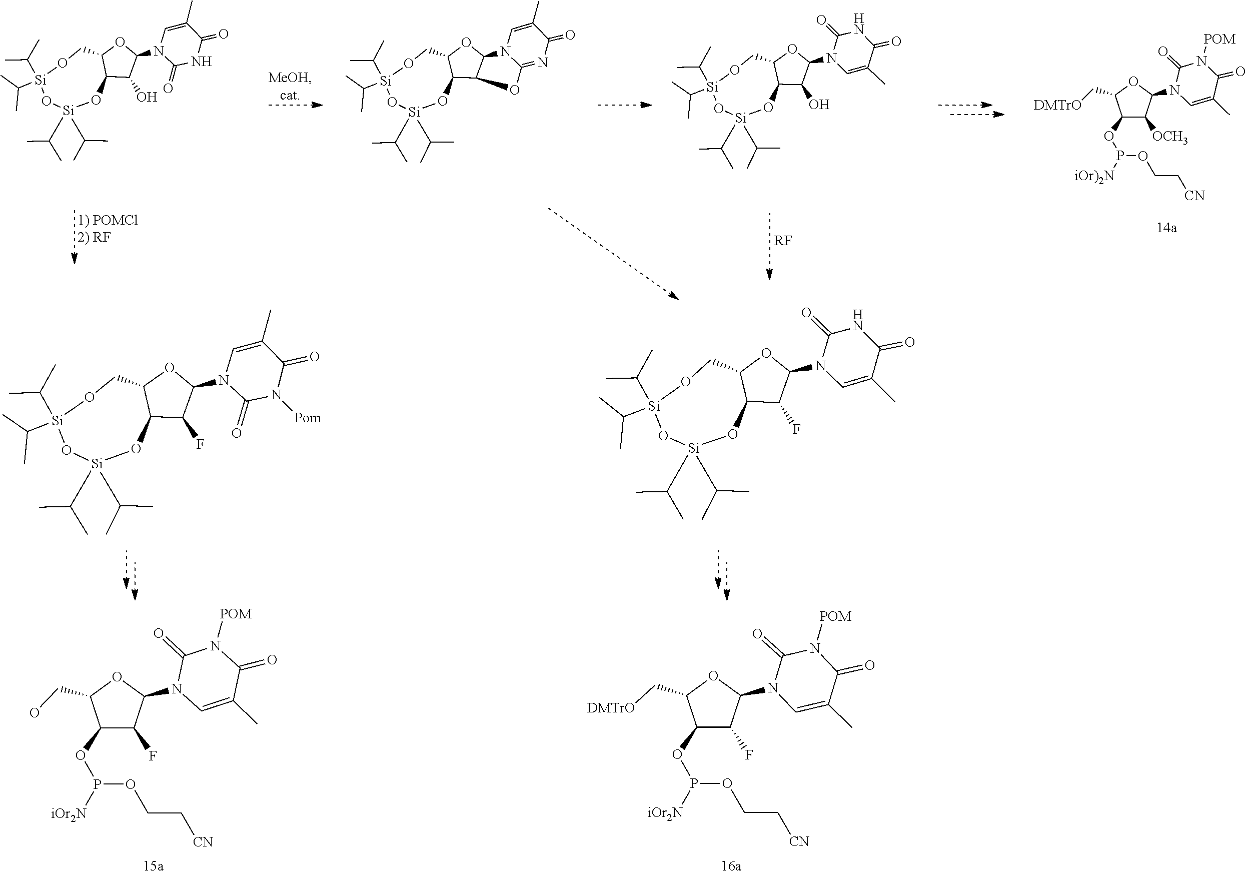

A scheme for the synthesis of an amidite of the 2′ substituted stereo-non-standard nucleoside 17a is shown below:

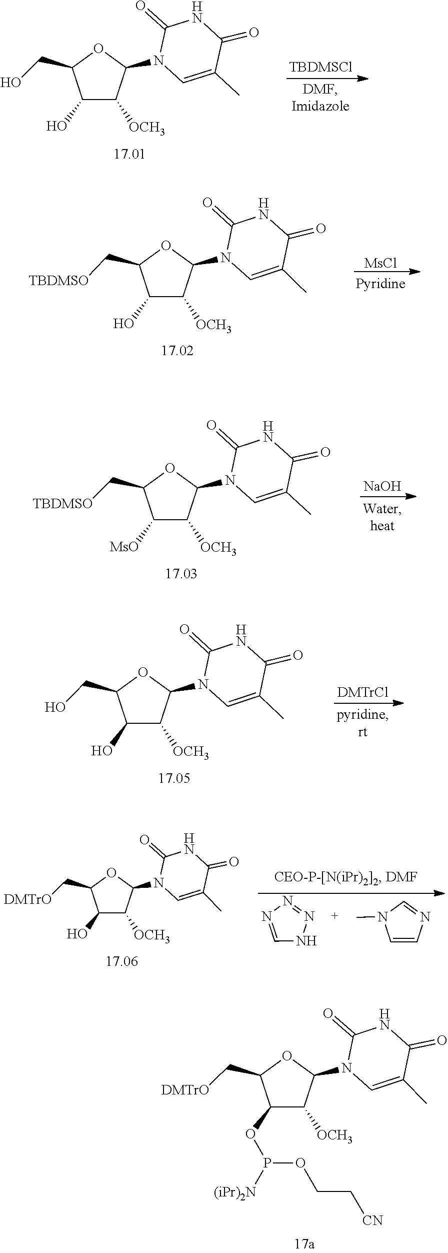

A scheme for the synthesis of an amidite of the 2′ substituted stereo-non-standard nucleoside 18a is shown below:

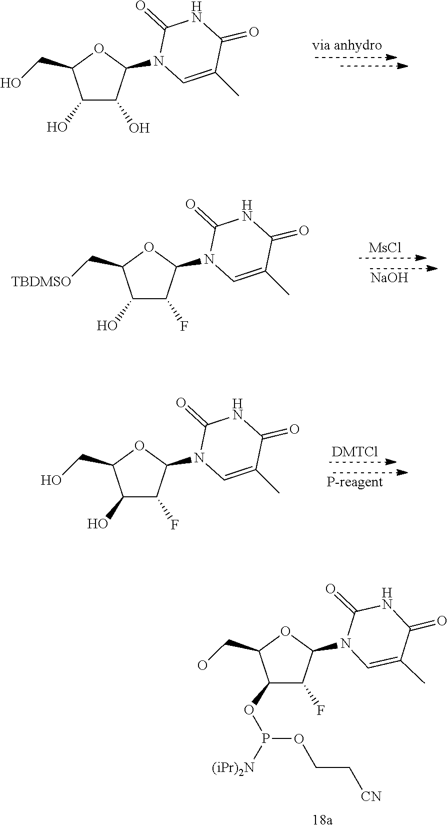

### Example 13: Endonuclease Stability of Modified Oligonucleotides Having Stereo-Standard Nucleosides and Stereo-Non-Standard Nucleosides

Modified oligonucleotides containing stereo-non-standard nucleotides were synthesized using standard techniques or those described herein. The compounds in the table below each have a 5′ wing and a 3′ wing each consisting of three linked cEt nucleosides and a central region comprising nucleosides each comprising 2′-β-D-deoxyribosyl sugar moieties aside from a single stereo-non-standard nucleoside, as indicated in the table below. Each oligonucleotide in the table below has the sequence GCATGTTCTCACATTA (SEQ ID NO: 5). Phosphodiester internucleoside linkages are incorporated on each side of the stereo-non-standard nucleoside, as indicated in the table below, while the remaining internucleoside linkages are phosphorothioate internucleoside linkages. With the exception of compound 1244451, all compounds in the table below contain 5-methyl cytosine for all cytosine nucleosides. Compound 1244451 contains unmethylated cytosine nucleosides in the central region of the compound.

The modified oligonucleotides were incubated at 1 μM concentration in RIPA buffer (50 mM Tris-HCl, pH 7.4, 20 mM MgCl₂, 150 mM NaCl, 0.5% NP-40) with 20% rat tritosomes (Xenotech). Tritosomes are purified lysosomes frequently utilized for determination of in vitro metabolic stability. Aliquots were removed at 0 and 24 hours, enzyme activity quenched (20% ACN, 3 M Urea, 25 mM Tris, pH 8, 1 mM EDTA), and the amount of full length modified oligonucleotide was determined by SAX-HPLC with complementary fluorescent labeled PNA probe (Roehl, I., et al., “Nucleic Acid Polymers with Accelerated Plasma and Tissue Clearance for Chronic Hepatitis B Therapy.” Molecular Therapy—Nucleic Acids 8: 1-12, 2017)]. Rat tritosome stability was determined by calculating the % peak area ratio for the 24 hour compared to the 0 hour time point, which is presented as % full-length (% FL) in the table below. Multiple values separated by commas represent replicates.

TABLE 19
Exonuclease resistance of modified oligonucleotides
having stereo-non-standard nucleosides

| Compound | | % FL at | |
|---|---|---|---|
| ID | Chemistry Notation (5′ to 3′) | 24 hours | SEQ ID NO. |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 93, 91, 86 | 5 |
| 1061316 | Gₖₛ^{m}CₖₛAₖₛT_{ds}G_{ds}T_{ds}Uᵣₛ^{m}C_{ds}T_{ds}^{m}C_{ds}A_{ds}^{m}C_{ds}A_{ds}TₖₛTₖₛAₖ | 12, 0, 0 | 5 |
| 813303 | Gₖₛ^{m}CₖₛAₖₛT_{ds}G_{ds}T_{ds}T_{ds}^{m}C_{do}T_{do}^{m}C_{ds}A_{ds}^{m}C_{ds}A_{ds}TₖₛTₖₛAₖ | 12 | 5 |
| 813312 | Gₖₛ^{m}CₖₛAₖₛT_{ds}G_{ds}T_{ds}T_{ds}^{m}C_{do}T_{ds}^{m}C_{ds}A_{ds}^{m}C_{ds}A_{ds}TₖₛTₖₛAₖ | 10 | 5 |
| 1215460 | Gₖₛ^{m}CₖₛAₖₛT_{ds}G_{ds}T_{ds}T_{[bLd]s}^{m}C_{ds}T_{ds}^{m}C_{ds}A_{ds}^{m}C_{ds}A_{ds}TₖₛTₖₛAₖ | 99 | 5 |
| 1427908 | Gₖₛ^{m}CₖₛAₖₛT_{ds}G_{ds}T_{ds}T_{[bLd]o}^{m}C_{ds}T_{ds}^{m}C_{ds}A_{ds}^{m}C_{ds}A_{ds}TₖₛTₖₛAₖ | 20 | 5 |
| 1427909 | Gₖₛ^{m}CₖₛAₖₛT_{ds}G_{ds}T_{do}T_{[bLd]s}^{m}C_{ds}T_{ds}^{m}C_{ds}A_{ds}^{m}C_{ds}A_{ds}TₖₛTₖₛAₖ | 104 | 5 |
| 1427910 | Gₖₛ^{m}CₖₛAₖₛT_{ds}G_{ds}T_{do}T_{[bLd]o}^{m}C_{ds}T_{ds}^{m}C_{ds}A_{ds}^{m}C_{ds}A_{ds}TₖₛTₖₛAₖ | 32 | 5 |
| 1244451 | Gₖₛ^{m}CₖₛAₖₛT_{ds}G_{ds}T_{ds}T_{[aDd]s}C_{ds}T_{ds}C_{ds}A_{ds}C_{ds}A_{ds}TₖₛTₖₛAₖ | 86 | 5 |
| 1427911 | Gₖₛ^{m}CₖₛAₖₛT_{ds}G_{ds}T_{ds}T_{[aDd]o}^{m}C_{ds}T_{ds}^{m}C_{ds}A_{ds}^{m}C_{ds}A_{ds}TₖₛTₖₛAₖ | 44 | 5 |
| 1427912 | Gₖₛ^{m}CₖₛAₖₛT_{ds}G_{ds}T_{do}T_{[aDd]s}^{m}C_{ds}T_{ds}^{m}C_{ds}A_{ds}^{m}C_{ds}A_{ds}TₖₛTₖₛAₖ | 52 | 5 |
| 1427913 | Gₖₛ^{m}CₖₛAₖₛT_{ds}G_{ds}T_{do}T_{[aDd]o}^{m}C_{ds}T_{ds}^{m}C_{ds}A_{ds}^{m}C_{ds}A_{ds}TₖₛTₖₛAₖ | 58 | 5 |
| 1215462 | Gₖₛ^{m}CₖₛAₖₛT_{ds}G_{ds}T_{ds}T_{[aLd]s}^{m}C_{ds}T_{ds}^{m}C_{ds}A_{ds}^{m}C_{ds}dA_{ds}TₖₛTₖₛAₖ | 92 | 5 |
| 1471967 | Gₖₛ^{m}CₖₛAₖₛT_{ds}G_{ds}T_{ds}T_{[aLd]o}^{m}C_{ds}T_{ds}^{m}C_{ds}A_{ds}^{m}C_{ds}A_{ds}TₖₛTₖₛAₖ | 61 | 5 |
| 1471968 | Gₖₛ^{m}CₖₛAₖₛT_{ds}G_{ds}T_{do}T_{[aLd]s}^{m}C_{ds}T_{ds}^{m}C_{ds}A_{ds}^{m}C_{ds}A_{ds}TₖₛTₖₛAₖ | 66 | 5 |
| 1471969 | Gₖₛ^{m}CₖₛAₖₛT_{ds}G_{ds}T_{do}T_{[aLd]o}^{m}C_{ds}T_{ds}^{m}C_{ds}A_{ds}^{m}C_{ds}A_{ds}TₖₛTₖₛAₖ | 56 | 5 |
| 1263777 | Gₖₛ^{m}CₖₛAₖₛT_{ds}G_{ds}T_{ds}T_{[dx]s}^{m}C_{ds}T_{ds}^{m}C_{ds}A_{ds}^{m}C_{ds}A_{ds}TₖₛTₖₛAₖ | 88 | 5 |
| 1471970 | Gₖₛ^{m}CₖₛAₖₛT_{ds}G_{ds}T_{ds}T_{[dx]o}^{m}C_{ds}T_{ds}^{m}C_{ds}A_{ds}^{m}C_{ds}A_{ds}TₖₛTₖₛAₖ | 79 | 5 |
| 1471971 | Gₖₛ^{m}CₖₛAₖₛT_{ds}G_{ds}T_{do}T_{[dx]s}^{m}C_{ds}T_{ds}^{m}C_{ds}A_{ds}^{m}C_{ds}A_{ds}TₖₛTₖₛAₖ | 89 | 5 |
| 1471972 | Gₖₛ^{m}CₖₛAₖₛT_{ds}G_{ds}T_{do}T_{[dx]o}^{m}C_{ds}T_{ds}^{m}C_{ds}A_{ds}^{m}C_{ds}A_{ds}TₖₛTₖₛAₖ | 80 | 5 |
| 1436557 | Gₖₛ^{m}CₖₛAₖₛT_{ds}G_{ds}T_{ds}T_{[bLdx]s}^{m}C_{ds}T_{ds}^{m}C_{ds}A_{ds}^{m}C_{ds}A_{ds}TₖₛTₖₛAₖ | 81 | 5 |
| 1471973 | Gₖₛ^{m}CₖₛAₖₛT_{ds}G_{ds}T_{ds}T_{[bLdx]o}^{m}C_{ds}T_{ds}^{m}C_{ds}A_{ds}^{m}C_{ds}A_{ds}TₖₛTₖₛAₖ | 49 | 5 |
| 1471974 | Gₖₛ^{m}CₖₛAₖₛT_{ds}G_{ds}T_{do}T_{[bLdx]s}^{m}C_{ds}T_{ds}^{m}C_{ds}A_{ds}^{m}C_{ds}A_{ds}TₖₛTₖₛAₖ | 67 | 5 |
| 1471975 | Gₖₛ^{m}CₖₛAₖₛT_{ds}G_{ds}T_{do}T_{[bLdx]o}^{m}C_{ds}T_{ds}^{m}C_{ds}A_{ds}^{m}C_{ds}A_{ds}TₖₛTₖₛAₖ | 64 | 5 |
| 1436549 | Gₖₛ^{m}CₖₛAₖₛT_{ds}G_{ds}T_{ds}T_{[aDdx]s}^{m}C_{ds}T_{ds}^{m}C_{ds}A_{ds}^{m}C_{ds}A_{ds}TₖₛTₖₛAₖ | 84 | 5 |
| 1490288 | Gₖₛ^{m}CₖₛAₖₛT_{ds}G_{ds}T_{ds}T_{[aDdx]o}^{m}C_{ds}T_{ds}^{m}C_{ds}A_{ds}^{m}C_{ds}A_{ds}TₖₛTₖₛAₖ | 38 | 5 |
| 1490289 | Gₖₛ^{m}CₖₛAₖₛT_{ds}G_{ds}T_{do}T_{[aDdx]s}^{m}C_{ds}T_{ds}^{m}C_{ds}A_{ds}^{m}C_{ds}A_{ds}TₖₛTₖₛAₖ | 62 | 5 |
| 1490290 | Gₖₛ^{m}CₖₛAₖₛT_{ds}G_{ds}T_{do}T_{[aDdx]o}^{m}C_{ds}T_{ds}^{m}C_{ds}A_{ds}^{m}C_{ds}A_{ds}TₖₛTₖₛAₖ | 40 | 5 |
| 1436553 | Gₖₛ^{m}CₖₛAₖₛT_{ds}G_{ds}T_{ds}T_{[aLdx]s}^{m}C_{ds}T_{ds}^{m}C_{ds}A_{ds}^{m}C_{ds}A_{ds}TₖₛTₖₛAₖ | 85 | 5 |
| 1490291 | Gₖₛ^{m}CₖₛAₖₛT_{ds}G_{ds}T_{ds}T_{[aLdx]o}^{m}C_{ds}T_{ds}^{m}C_{ds}A_{ds}^{m}C_{ds}A_{ds}TₖₛTₖₛAₖ | 30 | 5 |
| 1490292 | Gₖₛ^{m}CₖₛAₖₛT_{ds}G_{ds}T_{do}T_{[aLdx]s}^{m}C_{ds}T_{ds}^{m}C_{ds}A_{ds}^{m}C_{ds}A_{ds}TₖₛTₖₛAₖ | 56 | 5 |
| 1490293 | Gₖₛ^{m}CₖₛAₖₛT_{ds}G_{ds}T_{do}T_{[aLdx]o}^{m}C_{ds}T_{ds}^{m}C_{ds}A_{ds}^{m}C_{ds}A_{ds}TₖₛTₖₛAₖ | 40 | 5 |

In the table above, a superscript “in” indicates a 5-methylcytosine, a subscript “k” represents a cEt modified sugar moiety, a subscript “s” indicates a phosphorothioate internucleoside linkage, a subscript “o” represents a phosphodiester internucleoside linkage, and a subscript “d” represents a stereo-standard DNA sugar moiety.

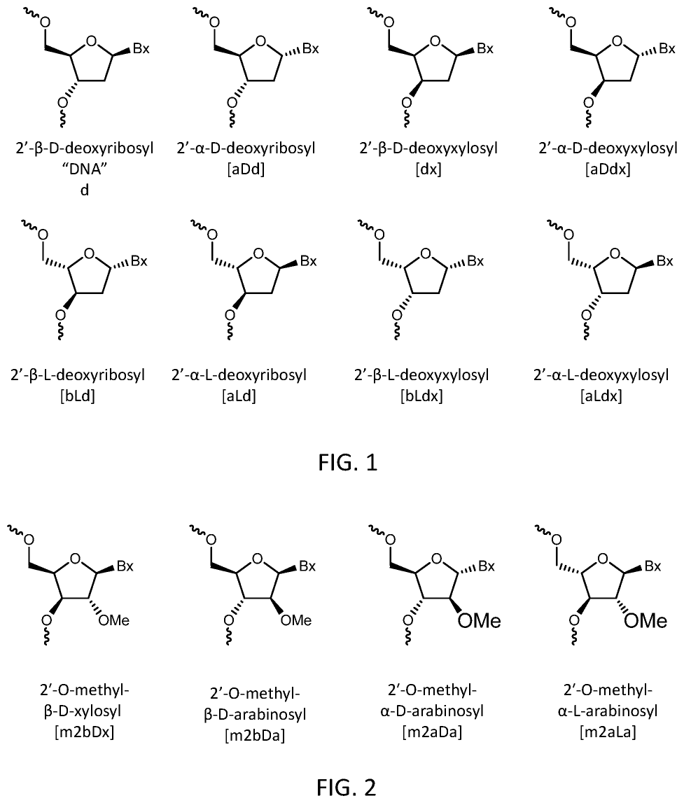

A subscript “[bLd]” represents a 2′-β-L-deoxyribosyl sugar moiety, a subscript “[aDd]” represents a 2′-α-D-deoxyribosyl sugar moiety, a subscript “[aLd]l” represents a 2′-α-L-deoxyribosyl sugar moiety, a subscript “[dx]” represents a 2′-β-D-deoxyxylosyl sugar moiety, a subscript “[bLdx]I” represents a 2′-β-L-deoxyxylosyl sugar moiety, a subscript “[aDdx]” represents a 2′-α-D-deoxyxylosyl sugar moiety, and a subscript “[aLdx]” represents a 2′-α-L-deoxyxylosyl sugar moiety. (See FIG. 1).

### Example 14: Design and Activity of siRNA with Stereo-Standard Nucleosides and Stereo-Non-Standard Nucleosides to HPRT1 In Vitro

siRNA

Modified oligonucleotides in the table below having either stereo-standard nucleosides or stereo-non-standard nucleosides were synthesized using standard techniques. Each antisense strand has the sequence AUAAAAUCUACAGUCAUAGGATT (SEQ ID NO: 7). The first 21 nucleosides of each antisense strand are 100% complementary to GenBank NM_000194.2 (SEQ ID NO: 8) from 446 to 466.

The sense strand (Compound ID: 1505889) has the chemical notation (5′ to 3′): U_{ys}C_{ys}C_{yo}U_{yo}A_{yo}U_{yo}G_{fo}A_{yo}C_{fo}U_{fo}G_{fo}U_{yo}A_{yo}G_{yo}A_{yo}U_{yo}U_{yo}U_{yo}U_{ys}A_{ys}U_{y} (SEQ ID NO: 9), wherein a subscript “f” represents a 2′-F modified nucleoside, a subscript “y” represents a 2′-OMe modified nucleoside, a subscript “s” indicates a phosphorothioate internucleoside linkage, and a subscript “o” represents a phosphodiester internucleoside linkage. The sense strand is 100% complementary to the complement of GenBank NM_000194.2 (SEQ ID NO: 8) from 446 to 466. The sense oligonucleotide is complementary to the first of the 21 nucleosides of the antisense oligonucleotide (from 5′ to 3′) wherein the last two 3′-nucleosides of the antisense oligonucleotides are not paired with the sense oligonucleotide (are overhanging nucleosides).

TABLE 20
Design of antisense strand modified oligonucleotides targeted to HPRT1
containing stereo-standard nucleosides and stereo-non-standard nucleosides

| Compound | | SEQ |
|---|---|---|
| ID | Chemical Notation (5′ to 3′) | ID NO. |
| 1512927 | A_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1512928 | A_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{[bLd]s}T_{[bLd]} | 7 |
| 1512929 | A_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{[aDd]s}T_{[aDd]} | 7 |
| 1512930 | A_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{[aLd]s}T_{[aLd]} | 7 |
| 1512931 | A_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{[dx]s}T_{[dx]} | 7 |
| 1512932 | A_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{[bLdx]s}T_{[bLdx]} | 7 |
| 1512933 | A_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{[aDdx]s}T_{[aDdx]} | 7 |
| 1512934 | A_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{[aLdx]s}T_{[aLdx]} | 7 |

In the table above, a subscript “f” represents a 2′-F modified nucleoside, a subscript “y” represents a 2′-OMe modified nucleoside, a subscript “s” indicates a phosphorothioate internucleoside linkage, a subscript “o” represents a phosphodiester internucleoside linkage, and a subscript “d” represents a stereo-standard DNA nucleoside. A subscript “[bLd]” represents a 2′-β-L-deoxyribosyl sugar moiety, a subscript “[aDd]” represents a 2′-α-D-deoxyribosyl sugar moiety, a subscript “[aLd]” represents a 2′-α-L-deoxyribosyl sugar moiety, a subscript “[dx]” represents a 2′-β-D-deoxyxylosyl sugar moiety, a subscript “[bLdx]” represents a 2′-β-L-deoxyxylosyl sugar moiety, a subscript “[aDdx]” represents a 2′-α-D-deoxyxylosyl sugar moiety, and a subscript “[aLdx]” represents a 2′-α-L-deoxyxylosyl sugar moiety. (See FIG. 1).

### Activity Assay

Activity of various siRNA formed by annealing one antisense strand and one sense strand described above was tested in HeLa cells. HeLa cells were transfected with RNAiMAX formulated siRNA. Each siRNA compound was transfected at a starting concentration of 10 nM with 5-fold serial dilutions for a total of 8 dilutions. After a treatment period of approximately 6 hours, RNA was isolated and RNA expression was analyzed via RT-qPCR using primer probe set RTS35336 (forward sequence TTGTTGTAGGATATGCCCTTGA, SEQ ID NO: 10; reverse sequence: GCGATGTCAATAGGACTCCAG, SEQ ID NO: 11; probe sequence: AGCCTAAGATGAGAGTTCAAGTTGAGTTTGG, SEQ ID NO: 12). HPRT1 RNA levels were normalized to total RNA content, as measured by RIBOGREEN®.

The results show that the incorporation of stereo-non-standard nucleosides at the 3′ end of the antisense strand of siRNA does not adversely affect activity.

TABLE 21
Activity of siRNAs targeted to HPRT1 containing
stereo-standard nucleosides and stereo-non-standard
nucleosides

| Antisense Strand | Sense Strand | IC₅₀ (nM) |
|---|---|---|
| 1512927 | 1505889 | 0.184 |
| 1512928 | 1505889 | 0.255 |
| 1512929 | 1505889 | 0.232 |
| 1512930 | 1505889 | 0.309 |
| 1512931 | 1505889 | 0.255 |
| 1512932 | 1505889 | 0.205 |
| 1512933 | 1505889 | 0.164 |
| 1512934 | 1505889 | 0.552 |

### Example 15: Design and Activity of siRNA with Stereo-Standard Nucleosides and Stereo-Non-Standard Nucleosides to HPRT1 In Vitro

siRNA

Modified oligonucleotides in the tables below having either stereo-standard nucleosides or stereo-non-standard nucleosides were synthesized using standard techniques. Each antisense strand has the sequence AUAAAAUCUACAGUCAUAGGATT (SEQ ID NO: 7). The first 21 nucleosides of each antisense strand are 100% complementary to GenBank NM_000194.2 (SEQ ID NO: 8) from 446 to 466, and each antisense strand has a 5′-phosphate.

The sense strand (Compound ID: 1505889) has the chemical notation (5′ to 3′): U_{ys}C_{ys}C_{yo}U_{yo}A_{yo}U_{yo}G_{fo}A_{yo}C_{fo}U_{fo}G_{fo}U_{yo}A_{yo}G_{yo}A_{yo}U_{yo}U_{yo}U_{yo}U_{ys}A_{ys}U_{y} (SEQ ID NO: 9), wherein a subscript “f” represents a 2′-F modified nucleoside, a subscript “y” represents a 2′-OMe modified nucleoside, a subscript “s” indicates a phosphorothioate internucleoside linkage, and a subscript “o” represents a phosphodiester internucleoside linkage. The sense strand is 100% complementary to the complement of GenBank NM_000194.2 (SEQ ID NO: 8) from 446 to 466. The sense oligonucleotide is complementary to the first of the 21 nucleosides of the antisense oligonucleotide (from 5′ to 3′) wherein the last two 3′-nucleosides of the antisense oligonucleotides are not paired with the sense oligonucleotide (are overhanging nucleosides).

TABLE 22
Design of antisense strand modified oligonucleotides targeted to HPRT1
containing stereo-standard nucleosides and stereo-non-standard nucleosides

| Compound | | SEQ |
|---|---|---|
| ID | Chemical Notation (5′ to 3′) | ID NO. |
| 1455005 | p.A_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1512935 | p.T_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 13 |
| 1512936 | p.T_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{[bLd]s}T_{[bLd]} | 13 |
| 1512937 | p.T_{ys}U_{fs}A_{yo}A_{yo}A_{yo}A_{fo}U_{yo}C_{fo}UfA_{yo}C_{yo}A_{yo}G_{yo}U_{fo}C_{yo}A_{fo}U_{yo}A_{yo}G_{yo}G_{yo}A_{ys}T_{[aDd]s}T_{[aDd]} | 13 |
| 1512938 | p.T_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{[aLd]s}T_{[aLd]} | 13 |
| 1512939 | p.T_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{[dx]s}T_{[dx]} | 13 |
| 1512940 | p.T_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{[bLdx]s}T_{[bLdx]} | 13 |
| 1512941 | p.T_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{[aDdx]s}T_{[aDdx]} | 13 |
| 1512942 | p.T_{ys}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{[aLdx]s}T_{[aLdx]} | 13 |
| 1512943 | p.T_{[m2bDx]s}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 13 |
| 1512944 | p.T_{[m2bDa]s}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 13 |
| 1512945 | p.T_{[m2aDa]s}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 13 |
| 1512946 | p.T_{[m2aLa]s}U_{fs}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 13 |

In the table above, a “p.” represents a 5′-phosphate, a subscript “f” represents a 2′-F modified nucleoside, a subscript “y” represents a 2′-OMe modified nucleoside, a subscript “s” indicates a phosphorothioate internucleoside linkage, a subscript “o” represents a phosphodiester internucleoside linkage, and a subscript “d” represents a stereo-standard DNA nucleoside. A subscript “[bLd]” represents a 2′-β-L-deoxyribosyl sugar moiety, a subscript “[aDd]” represents a 2′-α-D-deoxyribosyl sugar moiety, a subscript “[aLd]” represents a 2′-α-L-deoxyribosyl sugar moiety, a subscript “[dx]” represents a 2′-β-D-deoxyxylosyl sugar moiety, a subscript “[bLdx]” represents a 2′-β-L-deoxyxylosyl sugar moiety, a subscript “[aDdx]” represents a 2′-α-D-deoxyxylosyl sugar moiety, and a subscript “[aLdx]” represents a 2′-α-L-deoxyxylosyl sugar moiety. (See FIG. 1). A subscript “[m2bDx]” represents a 2′-O-methyl-β-D-xylosyl sugar moiety, a subscript “[m2bDa]” represents a 2′-O-methyl-β-D-arabinosyl sugar moiety, a subscript “[m2aDa]” represents a 2′-O-methyl-α-D-arabinosyl sugar moiety, a subscript “[m2aLa]” represents a 2′-O-methyl-α-L-arabinosyl sugar moiety. (See FIG. 2).

### Activity Assay

Activity of various siRNA formed by annealing one antisense strand and one sense strand described above was tested in HeLa cells. HeLa cells were transfected with RNAiMAX formulated siRNA. Each siRNA compound was transfected at a starting concentration of 10 nM with 5-fold serial dilutions for a total of 8 dilutions. After a treatment period of approximately 6 hours, RNA was isolated and RNA expression was analyzed via RT-qPCR using primer probe set RTS35336 (forward sequence TTGTTGTAGGATATGCCCTTGA, SEQ ID NO: 10; reverse sequence: GCGATGTCAATAGGACTCCAG, SEQ ID NO: 11; probe sequence: AGCCTAAGATGAGAGTTCAAGTTGAGTTTGG, SEQ ID NO: 12). HPRT1 RNA levels were normalized to total RNA content, as measured by RIBOGREEN®.

TABLE 23
In vitro activity of siRNAs targeted to HPRT1 containing
stereo-standard nucleosides and stereo-non-standard
nucleosides

| Antisense Strand | Sense Strand | IC₅₀ (nM) |
|---|---|---|
| 1455005 | 1505889 | 0.045 |
| 1512935 | 1505889 | 0.061 |
| 1512936 | 1505889 | 0.066 |
| 1512937 | 1505889 | 0.073 |
| 1512938 | 1505889 | 0.089 |
| 1512939 | 1505889 | 0.328 |
| 1512940 | 1505889 | 0.179 |
| 1512941 | 1505889 | 0.063 |
| 1512942 | 1505889 | 0.034 |
| 1512943 | 1505889 | 0.115 |
| 1512944 | 1505889 | 0.117 |
| 1512945 | 1505889 | 0.120 |
| 1512946 | 1505889 | 0.200 |

### Example 16: Design and Activity of siRNA with Stereo-Standard Nucleosides and Stereo-Non-Standard Nucleosides to HPRT1 In Vitro

Design of siRNA

Double-stranded siRNA comprising modified oligonucleotides having either stereo-standard nucleosides or stereo-non-standard nucleosides were synthesized and tested. Each antisense strand has the sequence AUAAAAUCUACAGUCAUAGGATT (SEQ ID NO: 7). The first 21 nucleosides of each antisense strand are 100% complementary to GenBank NM_000194.2 (SEQ ID NO: 8) from 446 to 466, and each antisense strand has a 5′-phosphate. Each sense strand has the sequence UCCUAUGACUGUAGAUUUUAU (SEQ ID NO: 9). Each sense strand is 100% complementary to the complement of GenBank NM_000194.2 (SEQ ID NO: 8) from 446 to 466. The sense oligonucleotide is complementary to the first 21 nucleosides of the antisense oligonucleotide (from 5′ to 3′).

TABLE 24
Design of antisense strand modified oligonucleotides targeted to HPRT1
containing stereo-standard nucleosides and stereo-non-standard nucleosides

| Compound | | SEQ |
|---|---|---|
| ID | Chemistry Notation (5′ to 3′) | ID NO. |
| 1455005 | p.AysUfsA_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537066 | p.A_{ys}U_{[2bDa]s}A_{yo}A_{yo}A_{yo}A_{fo}U_{yo}C_{fo}U_{fo}A_{yo}C_{yo}A_{yo}G_{yo}U_{[f2bLa]o}C_{yo}A_{fo}U_{yo}A_{yo}G_{yo}G_{yo}A_{ys}T_{ds}T_{d} | 7 |
| 1537045 | p.A_{ys}U_{[f2bDx]s}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537048 | p.A_{ys}U_{[f2aDr]s}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537069 | p.A_{ys}U_{[f2aDa]s}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537051 | p.A_{ys}U_{[2aDx]s}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537072 | p.A_{ys}U_{[2aLr]s}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537054 | p.A_{ys}U_{[f2bLx]s}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537075 | p.A_{ys}U_{[f2aLa]s}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537057 | p.A_{ys}U_{[f2aLx]s}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537060 | p.A_{ys}U_{[f2bLr]s}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537063 | p.A_{ys}U_{[f2bLa]s}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537067 | p.A_{ys}U_{fs}A_{yo}A_{yo}A_{yo}A_{fo}U_{yo}C_{fo}U_{[f2bDa]o}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537046 | p.A_{ys}U_{fs}A_{yo}A_{yo}A_{yo}A_{fo}U_{yo}C_{fo}U_{[f2bDx]o}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537050 | p.A_{ys}U_{fs}A_{yo}A_{yo}A_{yo}A_{fo}U_{yo}C_{fo}U_{[f2aDr]o}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537070 | p.A_{ys}U_{fs}A_{yo}A_{yo}A_{yo}A_{fo}U_{yo}C_{fo}U_{[f2aDa]o}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537052 | p.A_{ys}U_{fs}A_{yo}A_{yo}A_{yo}A_{fo}U_{yo}C_{fo}U_{[f2aDx]o}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537073 | p.A_{ys}U_{fs}A_{yo}A_{yo}A_{yo}A_{fo}U_{yo}C_{fo}U_{[f2aLr]o}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537055 | p.A_{ys}U_{fs}A_{yo}A_{yo}A_{yo}A_{fo}U_{yo}C_{fo}U_{[f2bLx]o}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537076 | p.A_{ys}U_{fs}A_{yo}A_{yo}A_{yo}A_{fo}U_{yo}C_{fo}U_{[f2aLa]o}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537058 | p.A_{ys}U_{fs}A_{yo}A_{yo}A_{yo}A_{fo}U_{yo}C_{fo}U_{[f2aLx]o}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537061 | p.A_{ys}U_{fs}A_{yo}A_{yo}A_{yo}A_{fo}U_{yo}C_{fo}U_{[f2bLr]o}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537064 | p.A_{ys}U_{fs}A_{yo}A_{yo}A_{yo}A_{fo}U_{yo}C_{fo}U_{[f2bLa]o}A_{yo}C_{yo}A_{yo}G_{yo}U_{fo}C_{yo}A_{fo}U_{yo}A_{yo}G_{yo}G_{yo}A_{ys}T_{ds}T_{d} | 7 |
| 1537068 | p.A_{ys}U_{fs}A_{yo}A_{yo}A_{yo}A_{fo}U_{yo}C_{fo}U_{fo}A_{yo}C_{yo}A_{yo}G_{yo}U_{[f2bDa]o}C_{yo}A_{fo}U_{yo}A_{yo}G_{yo}G_{yo}A_{ys}T_{ds}T_{d} | 7 |
| 1537047 | p.A_{ys}U_{fs}A_{yo}A_{yo}A_{yo}A_{fo}U_{yo}C_{fo}U_{fo}A_{yo}C_{yo}A_{yo}G_{yo}U_{[f2bDx]o}C_{yo}A_{fo}U_{yo}A_{yo}G_{yo}G_{yo}A_{ys}T_{ds}T_{d} | 7 |
| 1537049 | p.A_{ys}U_{fs}A_{yo}A_{yo}A_{yo}A_{fo}U_{yo}C_{fo}U_{fo}A_{yo}C_{yo}A_{yo}G_{yo}U_{[f2aDr]o}C_{yo}A_{fo}U_{yo}A_{yo}G_{yo}G_{yo}A_{ys}T_{ds}T_{d} | 7 |
| 1537071 | p.A_{ys}U_{fs}A_{yo}A_{yo}A_{yo}A_{fo}U_{yo}C_{fo}U_{fo}A_{yo}C_{yo}A_{yo}G_{yo}U_{[f2aDa]}oC_{yo}A_{fo}U_{yo}A_{yo}G_{yo}G_{yo}A_{ys}T_{ds}T_{d} | 7 |
| 1537053 | p.A_{ys}U_{fs}A_{yo}A_{yo}A_{yo}A_{fo}U_{yo}C_{fo}U_{fo}A_{yo}C_{yo}A_{yo}GyU_{[f2aDx]o}C_{yo}A_{fo}U_{yo}A_{yo}G_{yo}G_{yo}A_{ys}T_{ds}T_{d} | 7 |
| 1537074 | p.A_{ys}U_{fs}A_{yo}A_{yo}A_{yo}A_{fo}U_{yo}C_{fo}U_{fo}A_{yo}C_{yo}A_{yo}G_{yo}U_{[f2aLr]o}C_{yo}A_{fo}U_{yo}A_{yo}G_{yo}G_{yo}A_{ys}T_{ds}T_{d} | 7 |
| 1537056 | P-A_{ys}U_{fs}A_{yo}A_{yo}A_{yo}A_{fo}U_{yo}C_{fo}U_{fo}A_{yo}C_{yo}A_{yo}G_{yo}U_{[f2bLx]o}C_{yo}A_{fo}U_{yo}A_{yo}G_{yo}G_{yo}A_{ys}T_{ds}T_{d} | 7 |
| 1537077 | p.A_{ys}U_{fs}A_{yo}A_{yo}A_{yo}A_{fo}U_{yo}C_{fo}U_{fo}A_{yo}C_{yo}A_{yo}G_{yo}U_{[f2aLa]o}C_{yo}A_{fo}U_{yo}A_{yo}G_{yo}G_{yo}A_{ys}T_{ds}T_{d} | 7 |
| 1537059 | p.A_{ys}U_{fs}A_{yo}A_{yo}A_{yo}A_{fo}U_{yo}C_{fo}U_{fo}A_{yo}C_{yo}A_{yo}G_{yo}U_{[f2aLx]o}C_{yo}A_{fo}U_{yo}A_{yo}G_{yo}G_{yo}A_{ys}T_{ds}T_{d} | 7 |
| 1537062 | p.A_{ys}U_{fs}A_{yo}A_{yo}A_{yo}A_{fo}U_{yo}C_{fo}U_{fo}A_{yo}C_{yo}A_{yo}G_{yo}U_{[f2bLr]o}C_{yo}A_{fo}U_{yo}A_{yo}G_{yo}G_{yo}A_{ys}T_{ds}T_{d} | 7 |
| 1537065 | p.A_{ys}U_{fs}A_{yo}A_{yo}A_{yo}A_{fo}U_{yo}C_{fo}U_{fo}A_{yo}C_{yo}A_{yo}G_{yo}U_{[f2bLa]o}C_{yo}A_{fo}U_{yo}A_{yo}G_{yo}G_{yo}A_{ys}T_{ds}T_{d} | 7 |

TABLE 25
Design of sense strand modified oligonucleotides containing
stereo-standard nucleosides and stereo-non-standard nucleosides

| Compound | | SEQ |
|---|---|---|
| ID | Chemistry Notation (5′ to 3′) | ID NO. |
| 1505889 | U_{ys}C_{ys}C_{yo}U_{yo}A_{yo}U_{yo}G_{fo}A_{yo}C_{fo}U_{fo}G_{fo}U_{yo}A_{yo}G_{yo}A_{yo}U_{yo}U_{yo}U_{yo}U_{ys}A_{ys}U_{y} | 9 |
| 1537088 | U_{ys}C_{ys}C_{yo}U_{yo}A_{yo}U_{yo}G_{fo}A_{yo}C_{fo}U_{[f2bda]o}G_{fo}U_{yo}A_{yo}G_{yo}A_{yo}U_{yo}U_{yo}U_{yo}U_{ys}A_{ys}U_{y} | 9 |
| 1537078 | U_{ys}C_{ys}C_{yo}U_{yo}A_{yo}U_{yo}G_{fo}A_{yo}C_{fo}U_{[f2bDx]o}G_{fo}U_{yo}A_{yo}G_{yo}A_{yo}U_{yo}U_{yo}U_{yo}U_{ys}A_{ys}U_{y} | 9 |
| 1537079 | U_{ys}C_{ys}C_{yo}U_{yo}A_{yo}U_{yo}G_{fo}A_{yo}C_{fo}U_{[f2aDr]o}G_{fo}U_{yo}A_{yo}G_{yo}A_{yo}U_{yo}U_{yo}U_{yo}U_{ys}A_{ys}U_{y} | 9 |
| 1537080 | U_{ys}C_{ys}C_{yo}U_{yo}A_{yo}U_{yo}G_{fo}A_{yo}C_{fo}U_{[f2aDa]o}G_{fo}U_{yo}A_{yo}G_{yo}A_{yo}U_{yo}U_{yo}U_{yo}U_{ys}A_{ys}U_{y} | 9 |
| 1537081 | U_{ys}C_{ys}C_{yo}U_{yo}A_{yo}U_{yo}G_{fo}A_{yo}C_{fo}U_{[f2aDx]o}G_{fo}U_{yo}A_{yo}G_{yo}A_{yo}U_{yo}U_{yo}U_{yo}U_{ys}A_{ys}U_{y} | 9 |
| 1537082 | U_{ys}C_{ys}C_{yo}U_{yo}A_{yo}U_{yo}G_{fo}A_{yo}C_{fo}U_{[f2aLr]o}G_{fo}U_{yo}A_{yo}G_{yo}A_{yo}U_{yo}U_{yo}U_{yo}U_{ys}A_{ys}U_{y} | 9 |
| 1537083 | U_{ys}C_{ys}C_{yo}U_{yo}A_{yo}U_{yo}G_{fo}A_{yo}C_{fo}U_{[f2bLx]o}G_{fo}U_{yo}A_{yo}G_{yo}A_{yo}U_{yo}U_{yo}U_{yo}U_{ys}A_{ys}U_{y} | 9 |
| 1537084 | U_{ys}C_{ys}C_{yo}U_{yo}A_{yo}U_{yo}G_{fo}A_{yo}C_{fo}U_{[f2aLa]o}G_{fo}U_{yo}A_{yo}G_{yo}A_{yo}U_{yo}U_{yo}U_{yo}U_{ys}A_{ys}U_{y} | 9 |
| 1537085 | U_{ys}C_{ys}C_{yo}U_{yo}A_{yo}U_{yo}G_{fo}A_{yo}C_{fo}U_{[f2aLx]o}G_{fo}U_{yo}A_{yo}G_{yo}A_{yo}U_{yo}U_{yo}U_{yo}U_{ys}A_{ys}gU_{y} | 9 |
| 1537086 | U_{ys}C_{ys}C_{yo}U_{yo}A_{yo}U_{yo}G_{fo}A_{yo}C_{fo}U_{[f2bLr]o}G_{fo}U_{yo}A_{yo}G_{yo}A_{yo}U_{yo}U_{yo}U_{yo}U_{ys}A_{ys}U_{y} | 9 |
| 1537087 | U_{ys}C_{ys}C_{yo}U_{yo}A_{yo}U_{yo}G_{fo}A_{yo}C_{fo}U_{[f2bLa]o}G_{fo}U_{yo}A_{yo}G_{yo}A_{yo}U_{yo}U_{yo}U_{yo}U_{ys}A_{ys}U_{y} | 9 |

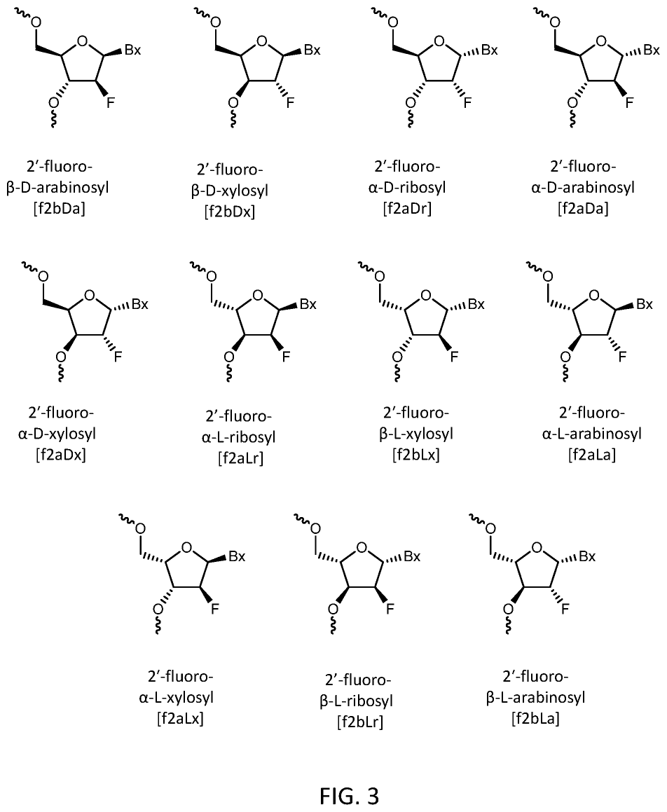

In the tables above, a subscript “f” represents a 2′-F modified nucleoside, a subscript “y” represents a 2′-OMe modified nucleoside, a subscript “s” indicates a phosphorothioate internucleoside linkage, and a subscript “o” represents a phosphodiester internucleoside linkage. Additionally, the following subscripts are used in the tables below: A subscript “[f2bDa]” represents a 2′-fluoro-β-D-arabinosyl sugar moiety, a subscript “[f2bDx]” represents a 2′-fluoro-$-D-xylosyl sugar moiety, a subscript “[f2aDr]” represents a 2′-fluoro-α-D-ribosyl sugar moiety, a subscript “[f2aDa]” represents a 2′-fluoro-α-D-arabinosyl sugar moiety, a subscript “[f2aDx]” represents a 2′-fluoro-α-D-xylosyl sugar moiety, a subscript “[f2aLr]” represents a 2′-fluoro-α-L-ribosyl sugar moiety, a subscript “[f2bLx]” represents a 2′-fluoro-β-L-xylosyl sugar moiety, a subscript “[f2aLa]” represents a 2′-fluoro-α-L-arabinosyl sugar moiety, a subscript “[f2aLx]” represents a 2′-fluoro-α-L-xylosyl sugar moiety, a subscript “[f2bLr]” represents a 2′-fluoro-β-L-ribosyl sugar moiety, and a subscript “[f2bLa]” represents a 2′-fluoro-β-L-arabinosyl sugar moiety. (See FIG. 3)

### Activity Assay

Activity of various siRNA formed by annealing one antisense strand and one sense strand described above was tested in HeLa cells. HeLa cells were transfected with RNAiMAX formulated siRNA. Each siRNA compound was transfected at a starting concentration of 10 nM with 5-fold serial dilutions for a total of 8 dilutions. After a treatment period of approximately 6 hours, RNA was isolated and RNA expression was analyzed via RT-qPCR using primer probe set RTS35336 (forward sequence TTGTTGTAGGATATGCCCTTGA, SEQ ID NO: 10; reverse sequence: GCGATGTCAATAGGACTCCAG, SEQ ID NO: 11; probe sequence: AGCCTAAGATGAGAGTTCAAGTTGAGTTTGG, SEQ ID NO: 12). HPRT1 RNA levels were normalized to total RNA content, as measured by RIBOGREEN®.

TABLE 26
Activity of siRNAs targeted to HPRT1 containing stereo-standard
nucleosides and stereo-non-standard nucleosides at position 2 (5′ to 3′)
of the antisense strand

| Duplex | | Position | Sense | Position | IC₅₀ |
|---|---|---|---|---|---|
| ID | Antisense Strand | Antisense* | Strand | Sense* | (nM) |
| 1542867 | 1455005 | N/A | 1505889 | N/A | 0.01 |
| 1542868 | 1537066 | 2 | 1505889 | N/A | 0.37 |
| 1542869 | 1537045 | 2 | 1505889 | N/A | 0.03 |
| 1542870 | 1537048 | 2 | 1505889 | N/A | 1.23 |
| 1542871 | 1537069 | 2 | 1505889 | N/A | 1.19 |
| 1542872 | 1537051 | 2 | 1505889 | N/A | 0.70 |
| 1542873 | 1537072 | 2 | 1505889 | N/A | 2.39 |
| 1542874 | 1537054 | 2 | 1505889 | N/A | 0.43 |
| 1542875 | 1537075 | 2 | 1505889 | N/A | 1.84 |
| 1542876 | 1537057 | 2 | 1505889 | N/A | 1.17 |
| 1542877 | 1537060 | 2 | 1505889 | N/A | 17.13 |
| 1542878 | 1537063 | 2 | 1505889 | N/A | 2.95 |
| 1542879 | 1537067 | 9 | 1505889 | N/A | 0.11 |
| 1542880 | 1537046 | 9 | 1505889 | N/A | 0.01 |
| 1542881 | 1537050 | 9 | 1505889 | N/A | 0.08 |
| 1542883 | 1537052 | 9 | 1505889 | N/A | 0.04 |
| 1542884 | 1537073 | 9 | 1505889 | N/A | 0.27 |
| 1542885 | 1537055 | 9 | 1505889 | N/A | 0.05 |
| 1542886 | 1537076 | 9 | 1505889 | N/A | 0.36 |
| 1542887 | 1537058 | 9 | 1505889 | N/A | 0.05 |
| 1542888 | 1537061 | 9 | 1505889 | N/A | 0.04 |
| 1542889 | 1537064 | 9 | 1505889 | N/A | 0.04 |
| 1542890 | 1537068 | 14 | 1505889 | N/A | 0.1 |
| 1542891 | 1537047 | 14 | 1505889 | N/A | 0.04 |
| 1542892 | 1537049 | 14 | 1505889 | N/A | 0.47 |
| 1542894 | 1537053 | 14 | 1505889 | N/A | 0.87 |
| 1542895 | 1537074 | 14 | 1505889 | N/A | 0.12 |
| 1542896 | 1537056 | 14 | 1505889 | N/A | 4.57 |
| 1542897 | 1537077 | 14 | 1505889 | N/A | 0.1 |
| 1542898 | 1537059 | 14 | 1505889 | N/A | 0.36 |
| 1542899 | 1537062 | 14 | 1505889 | N/A | 0.06 |
| 1542900 | 1537065 | 14 | 1505889 | N/A | 0.06 |
| 1542901 | 1455005 | N/A | 1537088 | 10 | 0.03 |
| 1542902 | 1455005 | N/A | 1537078 | 10 | 0.01 |
| 1542903 | 1455005 | N/A | 1537082 | 10 | 0.03 |
| 1542904 | 1455005 | N/A | 1537079 | 10 | 0.01 |
| 1542905 | 1455005 | N/A | 1537080 | 10 | 0.02 |
| 1542906 | 1455005 | N/A | 1537081 | 10 | 0.01 |
| 1542907 | 1455005 | N/A | 1537083 | 10 | 0.05 |
| 1542908 | 1455005 | N/A | 1537084 | 10 | 0.04 |
| 1542909 | 1455005 | N/A | 1537085 | 10 | 0.03 |
| 1542910 | 1455005 | N/A | 1537086 | 10 | 0.03 |
| 1542911 | 1455005 | N/A | 1537087 | 10 | 0.04 |

*Position of stereo-non-standard nucleoside(s) in the antisense or sense strand, from 5′ to 3′

### Example 17: Design and Activity of siRNA with Mesyl Phosphoramidate Internucleoside Linkages and Stereo-Non-Standard Nucleosides to HPRT1 In Vitro

Design of siRNAs

Double-stranded siRNAs comprising modified oligonucleotides having mesyl phosphoramidate internucleoside linkages (z. below) and having either stereo-standard nucleosides or stereo-non-standard nucleosides were synthesized and tested. Each internucleoside linkage is either a phosphorothioate internucleoside linkage (“s”), a phosphodiester internucleoside linkage (“o”), or a mesyl phosphoramidate internucleoside linkage (“z”).

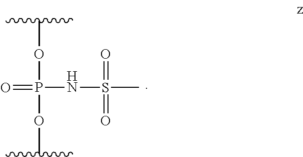 [SMILES: *OP(=O)(NS(C)(=O)=O)O*]

Each antisense strand has either the sequence (from 5′ to 3′): TUAAAAUCUACAGUCAUAGGATT (SEQ ID NO: 13) or UUAAAAUCUACAGUCAUAGGATT (SEQ ID NO: 14), wherein the sequence (from 5′ to 3′) UAAAAUCUACAGUCAUAGGA (SEQ TD NO: 15) is 100% complementary to GenBank Accession No. NM_000194.2 (SEQ TD NO: 8) from 446 to 465, and each antisense strand has a 5′-phosphate.

The sense strand (Compound ID: 1505889) has the chemical notation (5′ to 3′): U_{ys}C_{ys}C_{yo}U_{yo}A_{yo}U_{yo}G_{fo}A_{yo}C_{fo}U_{yo}A_{yo}G_{yo}A_{yo}U_{yo}U_{yo}U_{yo}U_{ys}A_{ys}U_{y} (SEQGID NO: 9), wherein a subscript “f” represents a 2′-F modified nucleoside, a subscript “y” represents a 2′-OMe modified nucleoside, a subscript “s” indicates a phosphorothioate internucleoside linkage, and a subscript “o” represents a phosphodiester internucleoside linkage.

TABLE 27
Design of antisense strand modified oligonucleotides targeted to HPRT1
containing mesyl phosphoramidate internucleoside linkages

| Compound | | SEQ |
|---|---|---|
| ID | Chemistry Notation (5′ to 3′) | ID NO. |
| 1512935 | p.T_{ys}UfsA_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 13 |
| 1534483 | p.U_{ys}UfsA_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 14 |
| 1534484 | p. [symbol] U_{fo}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 13 |
| 1534485 | p. [symbol] U_{fo}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 14 |
| 1534486 | p. [symbol] UfA_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 14 |
| 1534487 | p. [symbol] U_{fo}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 14 |
| 1534488 | p.U_{[f2bDx]o} [symbol] A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 14 |
| 1534489 | p.U_{[2aDr]o} [symbol] A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 14 |
| 1534490 | p.U_{[f2aDa]o} [symbol] A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 14 |
| 1534491 | p.U_{[2aDx]o} [symbol] A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 14 |
| 1534493 | p.U_{[f2aLr]o} [symbol] A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 14 |
| 1534494 | p.U_{[f2bLx]o} [symbol] A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 14 |
| 1534495 | p.U_{[f2aLa]o} [symbol] A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 14 |
| 1534496 | p.U_{[f2aLx]o} [symbol] A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 14 |
| 1534497 | p.U_{[f2bLr]o} [symbol] A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 14 |
| 1534492 | p.U_{[f2bLa]o} [symbol] A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 14 |
| 1537089 | p. [symbol] U_{fo}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 13 |
| 1537090 | p. [symbol] U_{fo}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 13 |
| 1537091 | p. [symbol] U_{fo}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 13 |
| 1537092 | p. [symbol] U_{fo}A_{yo}A_{yo}A_{yo}A_{fo}U_{yo}C_{fo}U_{fo}A_{yo}C_{yo}A_{yo}G_{yo}U_{fo}C_{yo}A_{fo}U_{yo}A_{yo}G_{yo}G_{yo}A_{ys}T_{ds}T_{d} | 13 |

In the table above, a “p.” represents a 5′-phosphate, a subscript “d” represents a stereo-standard DNA nucleoside, a subscript “y” represents a 2′-OMe modified nucleoside, a subscript “f” represents a 2′-F modified nucleoside, a subscript “s” indicates a phosphorothioate internucleoside linkage, a subscript “o” indicates a phosphodiester internucleoside linkage, and a subscript “z” represents an internucleoside linkage of formula IX, which is a mesyl phosphoramidate linkage. Subscripts of nucleotides having an internucleoside linkage of formula IX are bold and underlined. A subscript “[f2bDa]” represents a 2′-fluoro-β-D-arabinosyl sugar moiety, a subscript “[f2bDx]” represents a 2′-fluoro-β-D-xylosyl sugar moiety, a subscript “[f2aDr]” represents a 2′-fluoro-α-D-ribosyl sugar moiety, a subscript “[f2aDa]” represents a 2′-fluoro-α-D-arabinosyl sugar moiety, a subscript “[f2aDx]” represents a 2′-fluoro-α-D-xylosyl sugar moiety, a subscript “[f2aLr]” represents a 2′-fluoro-α-L-ribosyl sugar moiety, a subscript “[f2bLx]” represents a 2′-fluoro-β-L-xylosyl sugar moiety, a subscript “[f2aLa]” represents a 2′-fluoro-α-L-arabinosyl sugar moiety, a subscript “[f2aLx]” represents a 2′-fluoro-α-L-xylosyl sugar moiety, a subscript “[f2bLr]” represents a 2′-fluoro-β-L-ribosyl sugar moiety, a subscript “[f2bLa]” represents a 2′-fluoro-β-L-arabinosyl sugar moiety, a subscript “[m2bDx]” represents a 2′-O-methyl-β-D-xylosyl sugar moiety, a subscript “[m2bDa]” represents a 2′-O-methyl-β-D-arabinosyl sugar moiety, a subscript “[m2aDa]” represents a 2′-O-methyl-α-D-arabinosyl sugar moiety, a subscript “[m2aLa]” represents a 2′-O-methyl-α-L-arabinosyl sugar moiety. (See FIG. 2 and FIG. 3)

### Activity Assay

Activity of various siRNA formed by annealing one antisense strand and one sense strand described above was tested in HeLa cells. HeLa cells were transfected with RNAiMAX formulated siRNA. Each siRNA compound was transfected at a starting concentration of 10 nM with 5-fold serial dilutions for a total of 8 dilutions. After a treatment period of approximately 6 hours, RNA was isolated and RNA expression was analyzed via quantitative RTPCR using primer probe set RTS35336 (forward sequence TTGTTGTAGGATATGCCCTTGA, SEQ ID NO: 10; reverse sequence: GCGATGTCAATAGGACTCCAG, SEQ ID NO: 11; probe sequence: AGCCTAAGATGAGAGTTCAAGTTGAGTTTGG, SEQ ID NO: 12). HPRT1 RNA levels were normalized to total RNA content, as measured by RIBOGREEN®. IC₅₀ values were calculated and are presented in the table below.

TABLE 28
Activity of siRNAs targeted to HPRT1 containing
mesyl phosphoramidate internucleoside linkages
and/or stereo-non-standard nucleosides

| Antisense | Sense | IC₅₀ |
|---|---|---|
| Strand | Strand | (nM) |
| 1455005 | 1505889 | 0.01 |
| 1512935 | 1505889 | 0.03 |
| 1534483 | 1505889 | 0.02 |
| 1534484 | 1505889 | 0.06 |
| 1534485 | 1505889 | 0.03 |
| 1534486 | 1505889 | 0.04 |
| 1534487 | 1505889 | 0.04 |
| 1534488 | 1505889 | 0.10 |
| 1534489 | 1505889 | 0.04 |
| 1534491 | 1505889 | 0.04 |
| 1534494 | 1505889 | 0.18 |
| 1534496 | 1505889 | 0.04 |
| 1534497 | 1505889 | 0.08 |
| 1534492 | 1505889 | 0.07 |
| 1537090 | 1505889 | 0.05 |
| 1537091 | 1505889 | 0.06 |
| 1537092 | 1505889 | 0.13 |

### Example 18: Exonuclease Stability of Modified Oligonucleotides with Stereo-Standard Nucleosides and Stereo-Non-Standard Nucleosides

Oligonucleotides comprising stereo-standard and stereo-non-standard nucleosides were synthesized using standard techniques or those described herein. Each oligonucleotide in the table below has the sequence TTTTTTTTTTTT (SEQ ID NO: 16).

The oligonucleotides described below were incubated at 5 μM concentration in buffer with snake venom phosphodiesterase (SVPD, Sigma P4506, Lot #SLBV4179), a strong 3′-exonuclease, at the standard concentration of 0.5 mU/mL and at a higher concentration of 2 mU/mL. SVPD is commonly used to measure the stability of modified nucleosides (see, e.g., Antisense Drug Technology, Crooke S. T., Ed., CRC Press, 2008). Aliquots were removed at various time points and analyzed by MS-HPLC with an internal standard. Relative peak areas were plotted versus time and half-life was determined using GraphPad Prism. A longer half-life means the 3′-terminal nucleosides have increased resistance to the SVPD exonuclease.

The results in the table below show that stereo-non-standard DNA isomers are significantly more stable to exonuclease degradation than unmodified DNA, and several stereo-non-standard DNA isomers are significantly more stable than 2′-MOE or 2′-4′-LNA modified DNA.

TABLE 29
Exonuclease resistance of stereo-non-standard nucleosides

| Compound | | SVPD | T½ | SEQ |
|---|---|---|---|---|
| ID | Chemistry Notation (5′ to 3′) | (mU/mL) | (min) | ID NO. |
| 7157 | T_{do}T_{do}T_{do}T_{do}T_{do}T_{do}T_{do}T_{do}T_{do}T_{do}T_{do}T_{d} | 0.5 | 0.4 | 16 |
| 395421 | T_{do}l_{do}T_{do}T_{do}T_{do}T_{do}T_{do}T_{do}T_{do}T_{do}TₑₒTₑ | 0.5 | 7.1, 4.8 | 16 |
| 395422 | T_{do}T_{do}T_{do}T_{do}T_{do}T_{do}T_{do}T_{do}T_{do}T_{do}TₗₒTₗ | 0.5 | 27.8 | 16 |
| 1506055 | T_{do}T_{do}T_{do}T_{do}T_{do}T_{do}T_{do}T_{do}T_{do}T_{do}T_{ds}T_{d} | 0.5 | 46.8 | 16 |
| 1506055 | T_{do}T_{do}T_{do}T_{do}T_{do}T_{do}T_{do}T_{do}T_{do}T_{do}T_{ds}T_{d} | 2 | 8.9 | 16 |
| 1427914 | T_{do}T_{do}T_{do}T_{do}T_{do}T_{do}T_{do}T_{do}T_{do}T_{do}T_{[m2aLa]o}T_{[m2aLa]} | 2 | >200 | 16 |
| 1427915 | T_{do}T_{do}T_{do}T_{do}T_{do}T_{do}T_{do}T_{do}T_{do}T_{do}T_{[bLd]o}T_{[bLd]} | 2 | 28.7 | 16 |
| 1427916 | T_{do}T_{do}T_{do}T_{do}T_{do}T_{do}T_{do}T_{do}T_{do}T_{do}T_{[dx]o}T_{[dx]} | 2 | 7.1 | 16 |
| 1427917 | T_{do}T_{do}T_{do}T_{do}T_{do}T_{do}T_{do}T_{do}T_{do}T_{do}T_{[aLd]o}T_{[aLd]} | 2 | 70.1 | 16 |
| 1427918 | T_{do}T_{do}T_{do}T_{do}T_{do}T_{do}T_{do}T_{do}T_{do}T_{do}T_{[aDd]o}T_{[aDd]} | 0.5 | 4 | 16 |

In the table above, a subscript “d” indicates a nucleoside comprising an unmodified, 2′-β-D-deoxyribosyl sugar moiety. A subscript “l” indicates a LNA. A subscript “o” indicates a phosphodiester internucleoside linkage. Additionally, the following subscripts are used in the table above:

A subscript “[m2aLa]” represents a 2′-O-methyl-α-L-arabinosyl sugar moiety (see FIG. 2), a subscript “[bLd]” represents a 2′-β-L-deoxyribosyl sugar moiety, a subscript “[dx]” represents a 2′-β-D-deoxyxylosyl sugar moiety, a subscript “[aLd]” represents a 2′-α-L-deoxyribosyl sugar moiety, a subscript “[aDd]” represents a 2′-α-D-deoxyribosyl sugar moiety (See FIG. 1).

### Example 19: Synthesis of an Amidite of a 2′-Substituted Stereo-Non-Standard Nucleoside Comprising a 2′-fluoro-α-D-ribosyl Sugar Moiety

Compound 1.11, an amidite of a stereo-non-standard nucleoside comprising a 2′-fluoro-α-D-ribosyl sugar moiety, was prepared according to the scheme below:

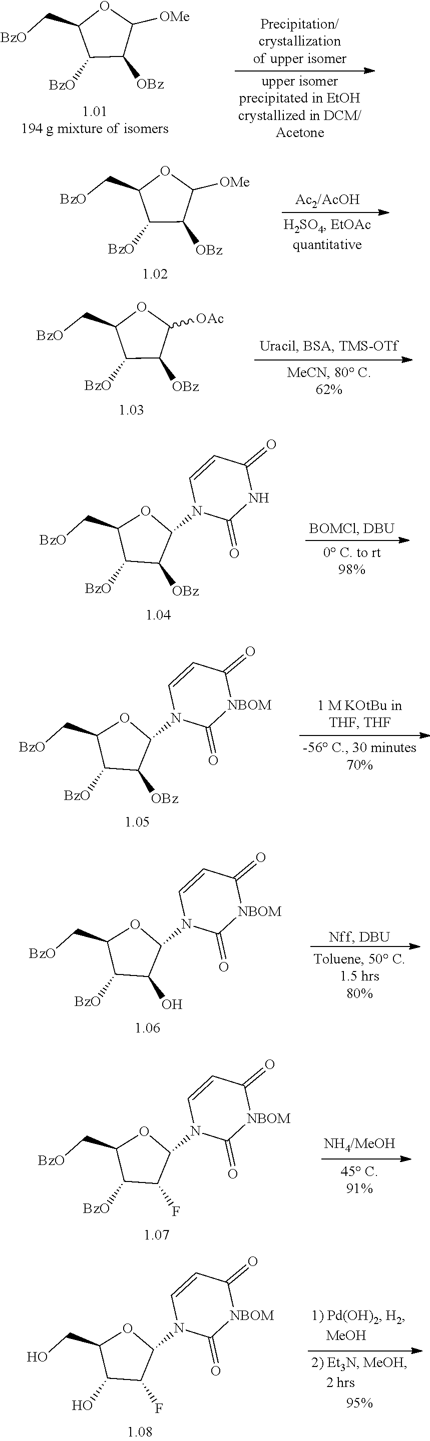

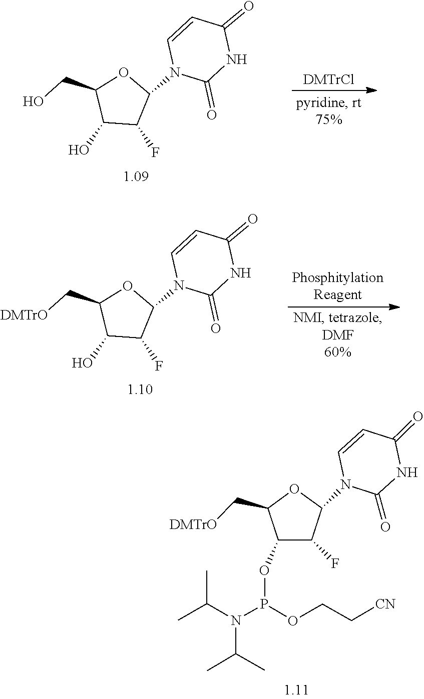

### Preparation of Compound 1.02

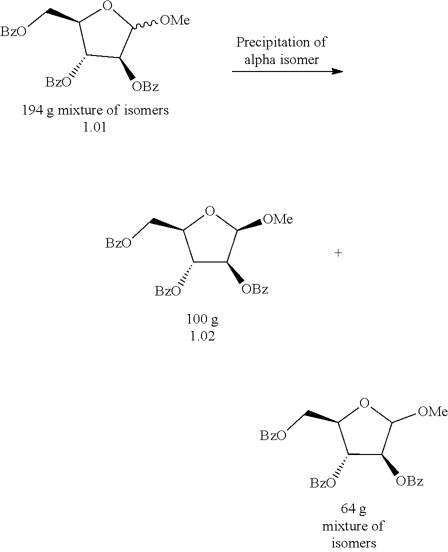

A mixture of isomers (1.01) (2R,3R,4S)-2-((benzoyloxy)methyl)-5-methoxytetrahydrofuran-3,4-diyl dibenzoate (194 g) was suspended in ethanol (80 mL) and vigorously stirred using a mechanical stirrer. The alpha isomer precipitated out as white solid. The solid was filtered and dry under high vacuum to obtain 100 g of the upper isomer (1.02). 50 g of this material is used for synthesis. The filtrate containing alpha and beta isomer was evaporated under reduced pressure to obtain a mixture of isomers (64 g).

### Preparation of Compound 1.03

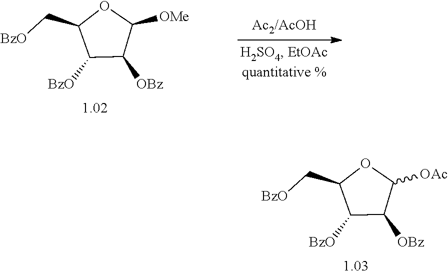

(3S,4R,5R)-2-acetoxy-5-((benzoyloxy)methyl)tetrahydrofuran-3,4-diyl dibenzoate (1.02) (50 g, 109.13 mmol) was dissolved in ethyl acetate (250.0 mL). Acetic anhydride (30.94 mL, 327.40 mmol, 3 eq) was added followed by sulfuric acid (1.17 mL, 21.83 mmol, 0.20 eq). After 3 hours stirring at room temperature, TLC in EtOAc/hexane (8/2) indicated that the reaction was complete. The reaction was diluted with saturated aqueous sodium bicarbonate solution (200 mL) and ethyl acetate (200 mL). Note: added some NaHCO₃ salt to pH 7. The aqueous layer was extracted with ethyl acetate. The combined organics were washed with saturated sodium bicarbonate solution (aq), water and brine, followed by concentration under reduced pressure to give a crude oil. Without any further purification, the crude oil was co-evaporated with toluene (3×50 mL) at 60° C. and used for the next step.

### Preparation of Compound 1.04

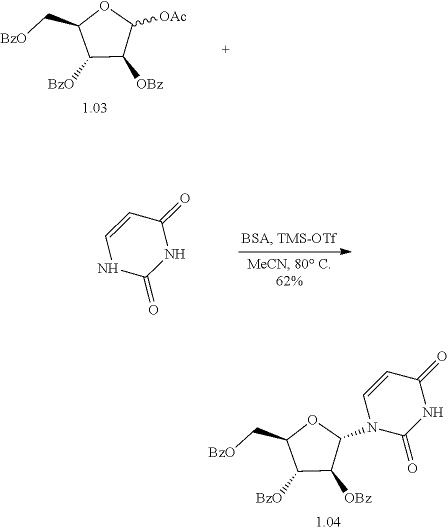

(3S,4R,5R)-2-acetoxy-5-((benzoyloxy)methyl)tetrahydrofuran-3,4-diyl dibenzoate (1.03) (55.0 g, 39.64 mmol) and pyrimidine-2,4(1H,3H)-dione (24.44 g, 218 mmol, 2 eq) was co-evaporated at 60° C. with anhydrous acetonitrile (3×50 mL). The mixture was suspended in anhydrous acetonitrile (800 mL), and N,O-Bis(trimethylsilyl)acetamide (106.63 mL, 436 mmol, 4.0 eq) was added. The reaction was heated at 80° C. for 30 minutes to obtain a clear solution and then cooled down reaction with an ice bath to 0° C. Trimethylsilyl trifluoromethanesulfonate (14.10 g, 63.43 mmol, 1.6 eq) was added and the mixture was stirred for 3 hours at 80° C. TLC in hexane/EtOAc (1/1) indicated that the reaction was complete. The reaction was cooled down to room temperature and evaporated solvent under reduced pressure to obtain crude oil. The crude material was dissolved in ethyl acetate (500 mL) and washed with plain DI water, followed with saturated sodium bicarbonate solution to pH 7. The aqueous layer was removed and continue washed the organic layer with saturated brine. The organic layer was dried over Na₂SO₄ for 15 minutes, filtered and concentrated under reduced pressure to obtain a crude oil. Purification by Biotage (Si, 350 g col, 40-60% EtOAc/hexane) afforded the desired product (1.04) as a white solid (37.70 g, 62% yield).

### Preparation of Compound 1.05

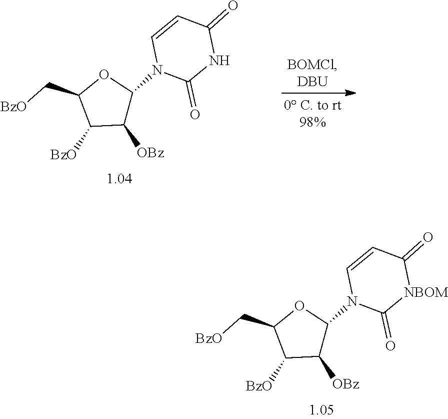

(2R,3R,4S,5S)-2-((benzoyloxy)methyl)-5-(2,4-dioxo-3,4-dihydropyrimidin-1(2H)-yl)tetrahydrofuran-3,4-diyl dibenzoate (1.04) (20 g, 35.94 mmol) dissolved in anhydrous dimethylformamide (100 mL) was stirred under nitrogen at room temperature. 2,3,4,6,7,8,9,10-octahydropyrimido[1,2-a]azepine (10.94 g, 71.87 mmol, 2.0 eq) was added and the reaction was cooled down in an ice bath to 0° C. ((Chloromethoxy)methyl)benzene (6.70 mL, 53.91 mmol, 1.5 eq.) was added dropwise, and the reaction was stirred at room temperature for 4 hours. TLC in EtOAc/hexane (4/6) indicated that the reaction was complete. The reaction was quenched by adding sat. NaHCO₃ solution (50 mL), transferred solution to a separatory funnel, and the product was extracted with ethyl acetate (2×50 mL). The organic layer was washed with sat. NaHCO₃ solution, sat. brine solution. The organic layer was dried over Na₂SO₄ for 15 minutes, filtered, and concentrated under reduced pressure to obtain a crude oil. The crude material was dissolved in ethyl acetate/hexane (1/1) and load to silica gel chromatography (Si, 50 g col, 5-30% ethyl acetate/hexane) which afforded the desired product (1.05) as a white solid (24.00 g, 98.69% yield).

### Preparation of Compound 1.06

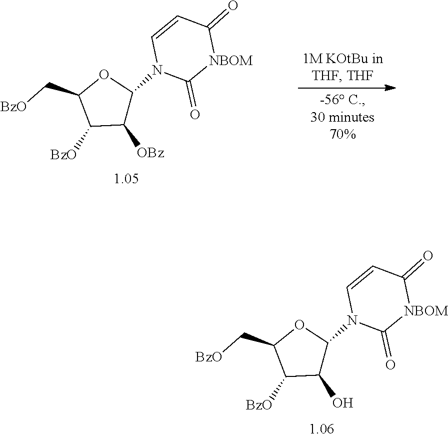

((2R,3S,4S,5S)-3-(benzoyloxy)-5-(3-((benzyloxy)methyl)-2,4-dioxo-3,4-dihydropyrimidin-1(2H)-yl)-4-hydroxytetrahydrofuran-2-yl)methyl benzoate (1.05) (8.88 g, 13.0 mmol) was dissolved in THF (400 mL) and cooled down with acetone/dry ice to −55° C. 1 N Potassium ter-butoxide in THF (19.51 mL, 19.51 mmol, 1.5 eq) was added dropwise over a period of 10 minutes to obtain a light yellow solution. The reaction was stirred at −55° C. for 15 minutes, monitored by LC/MS. The reaction was quenched by adding 1 N HCl dropwise, removing the cooling system, and stirring the reaction for 30 minutes. The solvent was evaporated under reduced pressure to obtain crude oil. The crude oil was suspended in ethyl acetate (100 mL) and washed with DI water (100 mL), sat. NaHCO₃ solution, sat. brine. The organic [?] was dried over Na₂SO₄ for 10 minutes, filtered, and the solvent was evaporated under reduced pressure. The crude material was dissolved in DCM and loaded onto a column on Biotage (Si, 100 g col, 40-60% acetone/DCM) to afford the desired product (1.06) as a white solid (5.15 g, 70% yield).

### Preparation of Compound 1.07

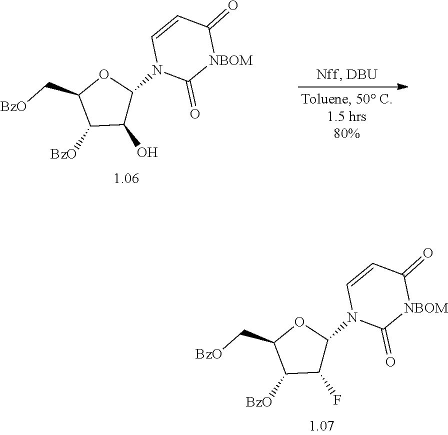

To a solution of ((2R,3S,4S,5S)-3-(benzoyloxy)-5-(3-((benzyloxy)methyl)-2,4-dioxo-3,4-dihydropyrimidin-1(2H)-yl)-4-hydroxytetrahydrofuran-2-yl)methyl benzoate (1.06) (5.10 g, 8.91 mmol) in anhydrous toluene (35 mL) was added 1,8-Diazabicyclo[5.4.0]undec-7-ene (2.66 mL, 17.81 mmol, 2.0 eq) followed with dropwise addition of 1,1,2,2,3,3,4,4,4-nonafluorobutane-1-sulfonyl fluoride (3.20 mL, 17.81 mmol, 2 eq). The reaction was heated at 50° C. for 1.5 hours. TLC in EtOAc/hexane (1/1) indicated that the reaction was complete. The reaction was cooled to room temperature, diluted with ethyl acetate (50 mL) and the organic [layer?] was washed with DI water (50 mL), sat. NaHCO₃ solution, sat. brine. The organic was dried over Na₂SO₄ for 10 minutes, filtered the solvent was evaporated under reduced pressure. The crude material was dissolved in DCM and loaded to column Biotage (Si, 100 g col, 0-40% EtOAc/hexane) to afford the desired product (1.07) as a light yellow solid (4.10 g, 80% yield).

### Preparation of Compound 1.08

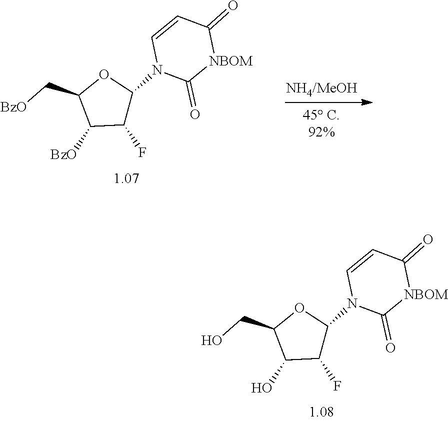

A solution of ((2R,3R,4R,5S)-3-(benzoyloxy)-5-(3-((benzyloxy)methyl)-2,4-dioxo-3,4-dihydropyrimidin-1(2H)-yl)-4-fluorotetrahydrofuran-2-yl)methyl benzoate (1.07) (4.10 g, 7.14 mmol) in ammonia solution 7 N in methanol (40 mL) was prepared. The reaction was heated at 45° C. overnight. The next day, TLC in DCM/MeOH (95/5) indicated that the reaction was complete. The solvent was evaporated under reduced pressure. The material was dissolved in DCM/MeOH (95/5) and loaded onto column Biotage (Si, 50 g col, 0-5% DCM/MeOH) to afford the desired product (1.08) as a white solid (2.40 g, 92% yield).

### Preparation of Compound 1.09

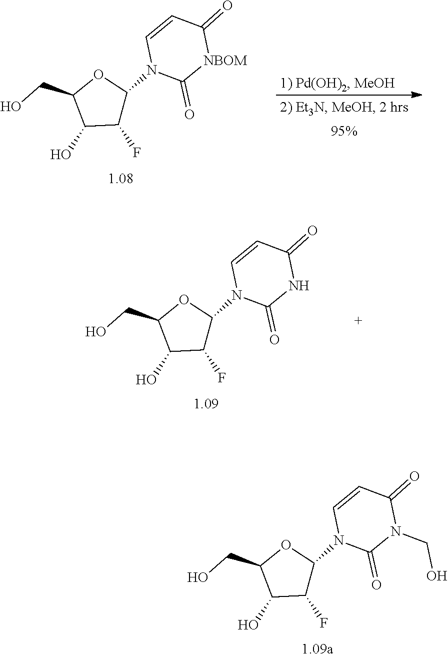

Pd(OH)₂ and H₂ was added to a solution of 3-((benzyloxy)methyl)-1-((2S,3R,4R,5R)-3-fluoro-4-hydroxy-5-(hydroxymethyl)tetrahydrofuran-2-yl)pyrimidine-2,4(1H,3H)-dione (1.08) (2.70 g, 7.37 mmol) in methanol (16 mL) under nitrogen and the reaction was stirred overnight. The next day, LC/MS indicated that the reaction was complete with a minor side product of (1.09a). Upon completion, the reaction solution was filtered through a plug of Celite and rinsed with methanol. The filtrate was evaporated under reduced pressure to obtain white solid of the crude mixture. The crude material was dissolved in methanol (10 mL) and triethylamine (2 mL) and stirred for 2 hours at room temperature. LC/MS indicated full conversion of compound (1.08). The solvent was evaporated under reduced pressure to obtain pure compound (1.09) (1.72 g, 95% yield).

### Preparation of Compound 1.10

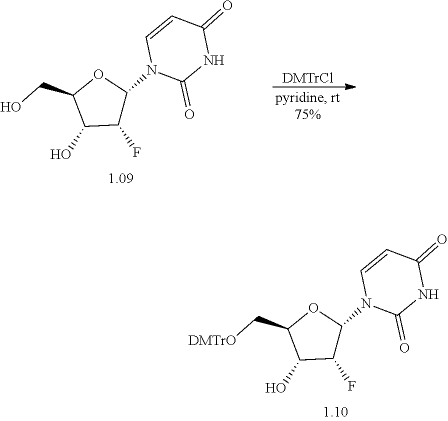

DMTrCl (2.81 g, 8.29 mmol, 1.20 eq) was added to a solution of 1-((2S,3R,4R,5R)-3-fluoro-4-hydroxy-5-(hydroxymethyl)tetrahydrofuran-2-yl)pyrimidine-2,4(1H,3H)-dione (1.09) (1.70 g, 6.91 mmol), in pyridine (15 mL) at room temperature and stirred for 3 hours. TLC in EtOAc/hexane (7/3) indicated that the reaction was complete. The solution was transferred into a separatory funnel and diluted with ethyl acetate (50 mL) and washed with DI water (2×50 mL). The aqueous layer was removed and organic continued washed with sat. NaHCO₃, sat. brine and dried over Na₂SO₄ filtered and evaporated solvent under reduced pressure to obtain crude material. The crude material was dissolved in DCM and loaded to silica gel chromatography. Purification by Biotage (Si, 50 g col, 5-60% ethyl acetate/hexane+1% Et₃N) afforded the desired product (1.10) as a white solid (2.84 g, 75% yield).

### Preparation of Compound 1.11

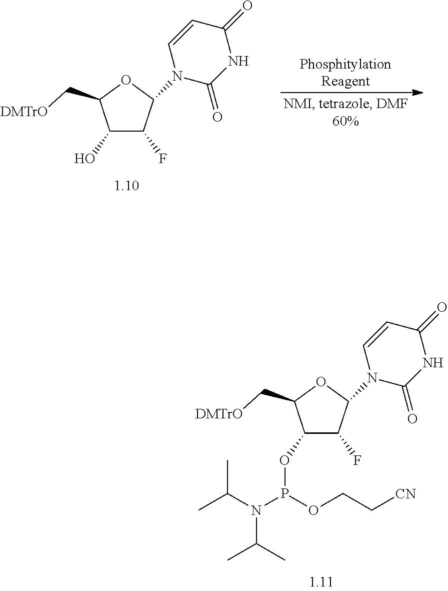

1H-Tetrazole (261.81 mg, 3.79 mmol, 0.8 eq) and 1-methylimidazole (94.45 mL, 1.18 mmol, 0.25 eq) were added to a solution of 1-((2S,3R,4R,5R)-5-((bis(4-methoxyphenyl)(phenyl)methoxy)methyl)-3-fluoro-4-hydroxytetrahydrofuran-2-yl)pyrimidine-2,4(1H,3H)-dione (1.10) (2.60 g, 4.74 mmol) in anhydrous DMF (20 mL) at room temperature under an atmosphere of nitrogen. 3-bis(diisopropylamino)phosphanyloxypropanenitrile (2.26 mL, 7.11 mmol, 1.5 eq) was then added dropwise and the reaction was stirred at room temperature for 10 hours. The reaction solution was transferred to a separatory funnel, diluted by adding a 3:1 mixture of toluene/hexanes (30 mL), and the organic layer was washed 4×(30 mL) with a 3:2 mixture of DMF/H₂O. The organic layer was washed with saturated sodium bicarbonate solution, brine, dried over solid sodium sulfate and concentrated under reduced pressure to a white foam. Purification by Biotage (Si, 50 g col, 20-50% ethyl acetate/hexanes+1% triethylamine) afforded the desired product (1.11) as a white solid (2.14 g, 60% yield).

### Example 20: Synthesis of an Amidite of a 2′-Substituted Stereo-Non-Standard Nucleoside Comprising a 2′-fluoro-α-D-arabinosyl Sugar Moiety

Compound 2.08 an amidite of a stereo-non-standard nucleoside comprising a 2′-fluoro-α-D-arabinosyl sugar moiety, was prepared according to the scheme below:

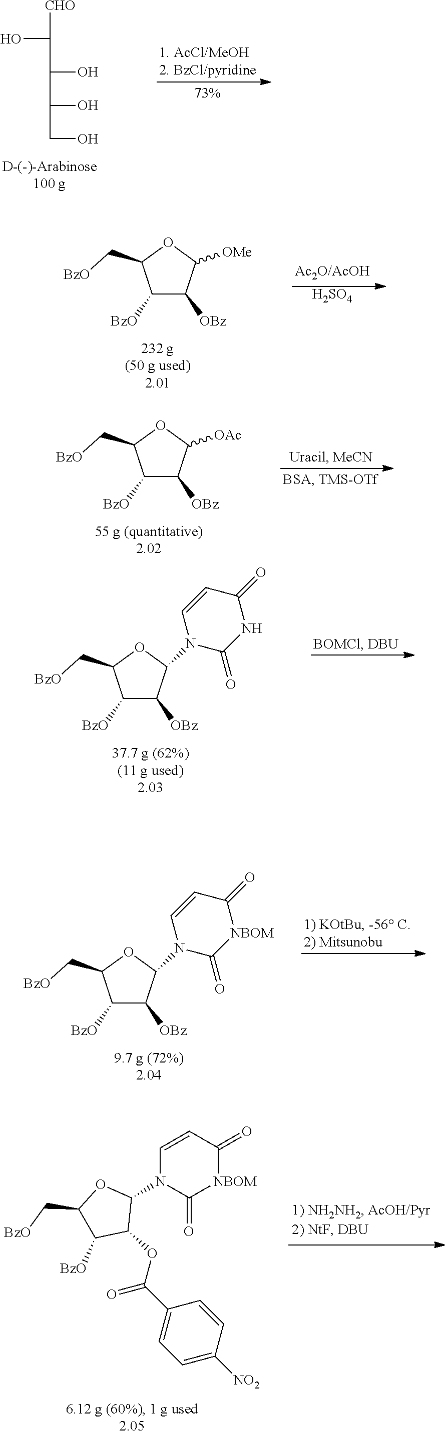

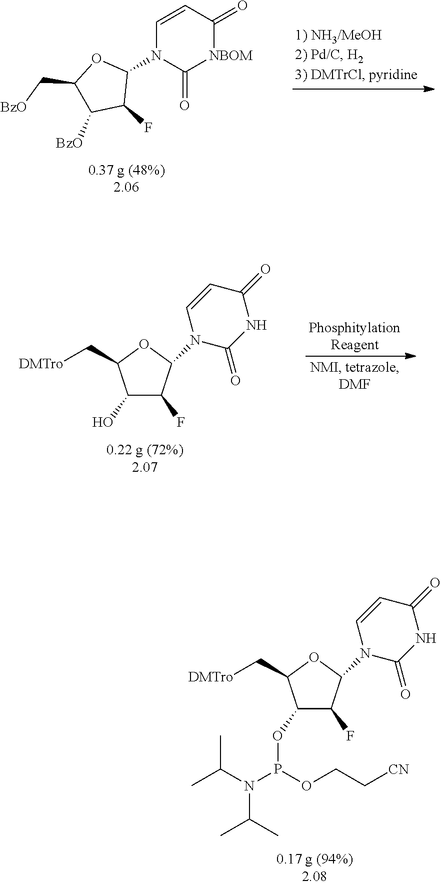

### Preparation of Compound 2.01

Acetyl chloride (50 mL, 700 mmol) was added to MeOH (600 mL) in a three-neck flask in an ice bath under nitrogen atmosphere dropwise. After the addition was completed, the reaction was removed from ice bath and stirred at room temperature for another 30 min. The methanolic hydrogen chloride solution thus generated was added via cannula slowly to a solution of D-(−)-arabinose (100 g, 666 mmol) in methanol (2 L) at room temperature and the reaction mixture was stirred at room temperature for 12 h. The solution became clear after 2 h. After 12 h, pyridine (60 mL) was added and the reaction mixture concentrated. The residue was co-evaporated with toluene (3×60 mL) and dried under high vacuum for 12 h. The colorless oil was used for next step without any further purification.

To a solution of D-(−)-arabinose 1′ methyl ether (109 g, 666 mmol) in pyridine (750 mL), benzoyl chloride (309 mL, 2662 mmol) was added at 0° C. The reaction mixture was warmed to room temperature and stirred overnight. The mixture was diluted with water (2000 mL) and extracted with dichloromethane (1400 mL). The organic phase was washed with water (1000 mL), 10% aqueous HCl solution (2×100 mL) and saturated sodium bicarbonate aqueous solution. The organic phase was dried over Na₂SO₄ and concentrated to dryness under reduced pressure. The crude product was purified by silica gel column chromatography and eluted with 10-30% ethyl acetate in hexanes gradient to yield compound 2.01 (232 g, 73%). Spectral data are consistent with the structure of compound 2.01. Mass Calcd 476.5, Found 499.1.

### Preparation of Compound 2.02

(3S,4R,5R)-2-acetoxy-5-((benzoyloxy)methyl)tetrahydrofuran-3,4-diyl dibenzoate (2.01) (50 g, 109.13 mmol) was dissolved in ethyl acetate (250.0 mL). Acetic anhydride (30.94 mL, 327.40 mmol, 3 eq) was added followed by sulfuric acid (1.17 mL, 21.83 mmol, 0.20 eq). After 3 hours stirring at room temperature, TLC in EtOAc/hexane (8/2) indicated that the reaction was complete. The reaction was diluted with saturated aqueous sodium bicarbonate solution (200 mL) and ethyl acetate (200 mL). NaHCO₃ salt was used to adjust to pH 7. The aqueous layer was extracted with ethyl acetate. The combined organics were washed with saturated sodium bicarbonate solution (aq), water and brine, followed by concentration under reduced pressure to give a crude oil. Without any further purification, the crude oil was co-evaporated with toluene (3×50 mL) at 60° C. and used for the next step.

### Preparation of Compound 2.03

(3S,4R,5R)-2-acetoxy-5-((benzoyloxy)methyl)tetrahydrofuran-3,4-diyl dibenzoate (2.02) (55.0 g, 39.64 mmol) and pyrimidine-2,4(1H,3H)-dione (24.44 g, 218 mmol, 2 eq) was co-evaporated at 60° C. with anhydrous acetonitrile (3×50 mL). The mixture was suspended in anhydrous acetonitrile (800 mL) and N,O-Bis(trimethylsilyl)acetamide (106.63 mL, 436 mmol, 4.0 eq) was added. The reaction was heated at 80° C. for 30 minutes to obtain a clear solution, and then cooled down reaction with an ice bath to 0° C. Trimethylsilyl trifluoromethanesulfonate (14.10 g, 63.43 mmol, 1.6 eq) was added and the reaction was stirred for 3 hours at 80° C. TLC in hexane/EtOAc (1/1) indicated that the reaction was complete. The reaction was cooled to room temperature and evaporated solvent under reduced pressure to obtain crude oil. The crude material was dissolved in ethyl acetate (500 mL) and washed with plain DI water, followed with saturated sodium bicarbonate solution to pH 7. The aqueous layer was removed and the organic layer was washed with saturate brine. The organic layer was dried over Na₂SO₄ for 15 minutes, filtered and concentrated under reduced pressure to obtain a crude oil. Purification by Biotage (Si, 350 g col, 40-60% EtOAc/hexane) afforded the desired product (2.03) as a white solid (37.70 g, 62% yield).

### Preparation of Compound 2.04

To a solution of compound 2.03 (11 g, 20 mmol) in DMF (60 mL) at 0° C., DBU (6 mL, 40 mmol) and BOMCl (4.2 mL, 30 mmol) were added sequentially. The reaction was stirred at 0° C. for 4 hours and quenched with saturated NaHCO₃ aqueous solution (150 mL). The mixture was extracted with ethyl acetate (200 mL) and washed with brine (3×200 mL). The combined ethyl acetate solution was washed with water (300 mL) and concentrated to dryness. The crude product was purified by silica gel column chromatography and eluted with 20-50% ethyl acetate in hexanes gradient to yield compound 2.04 (9.68 g, white foam, 72%). Spectral data are consistent with the structure of compound 2.04. Mass calculated: 676.7, mass found: 677.2.

### Preparation of Compound 2.05

To a solution of Compound 2.04 (9.63 g, 14.23 mmol) in THF (380 mL), chilled at −56° C. in dry-ice-acetonitrile bath, was added potassium tert-butoxide 1M THF solution (21.35 mL, 21.35 mmol) with vigorous stirring. After 25 minutes, 2 N HCl aqueous solution (22 mL, 44 mmol) was added and the mixture was stirred for another 5 minutes. The reaction was concentrated with reduced pressure and the residue was extracted with dichloromethane (150 mL). The dichloromethane solution was washed with water (200 mL) and the organic solution was concentrated to dryness. The crude product was purified by silica gel column chromatography and eluted 0-50% ethyl acetate in dichloromethane gradient to yield the Selective de-benzoyl nucleoside (6.21 g, 76.2%).

To a THF solution (110 mL) of the selective de-benzoyl nucleoside (6.15 g, 10.74 mmol), p-nitrobenzoic acid (3.59 g, 21.48 mmol) and Ph₃P (5.63 g, 21.48 mmol), DIAD (4.16 mL, 21.48 mmol) was dropwise added at room temperature. The reaction was stirred at room temperature for 12 hours. Then the reaction was concentrated to dryness and it was re-dissolved in dichloromethane. The DCM solution was washed with brine and concentrated. The crude product was purified by silica gel column chromatography and eluted with 0-50% ethyl acetate in dichloromethane gradient to yield compound 2.05 (6.12 g, 79%). Spectral data are consistent with the structure of compound 2.05. Mass calculated: 721.7, mass found: 722.2.

### Preparation of Compound 2.06

To a solution of Compound 2.05 (1.04 g, 1.44 mmol) in 15 mL of acetic acid/pyridine (1:4) was added N₂H₄.xH₂O (0.18 mL, 5.76 mmol). After 15 hours, acetone (1 mL) was added and the reaction was continued to stir for another 2 hours. Then the reaction was concentrated, and the residue was partitioned between water and ethyl acetate. The combined organic solution was washed with saturated sodium bicarbonate aqueous solution, water and concentrated. The residue was co-evaporated with toluene (2×2 mL) and concentrated to dryness. The crude product was purified by silica gel column chromatography and eluted with 0-50% ethyl acetate in dichloromethane gradient to yield mono hydroxyl nucleoside (0.65 g, 79%). Spectral data are consistent with the structure of mono hydroxyl nucleoside. Mass calculated: 572.6, mass found: 573.2.

To a toluene (10 mL) solution of mono hydroxyl nucleoside (0.61 g, 1.07 mmol), DBU (0.32 mL, 2.13 mmol) and NfF (0.38 mL, 2.13 mmol) were added. The reaction was stirred at 50° C. for one hour. The reaction was extracted with ethyl acetate (100 mL), washed with brine (250). The ethyl acetate solution was concentrated to dryness and the residue was purified by silica gel column chromatography and eluted with 0-30% ethyl acetate in dichloromethane gradient to yield compound 2.06 (0.37 g, 61%). Spectral data are consistent with the structure of mono hydroxyl nucleoside. Mass calculated: 574.5, mass found: 575.3.

### Preparation of Compound 2.07

To a vial charged with compound 2.06 (0.35 g, 0.61 mmol), 7 N NH₃ methanol solution (2 mL, 14 mmol) was added. The flask was sealed and stirred at 55° C. for 12 hours. The reaction was concentrated to dryness and the residue was purified by silica gel column chromatography and eluted with 9-10% MeOH in dichloromethane gradient to yield the de-benzoylated product (0.18 g, 82%). Spectral data are consistent with the structure of the de-benzoylated product. Mass calculated: 366.5, Mass found: 367.1.

To a MeOH solution (5 mL) of de-benzoylated product (0.18 g, 0.49 mmol), Pd(OH)₂ (0.036 g) was added. The reaction was stirred at rt under H₂ for 12 hours, filtered through a Celite padding and rinsed with MeOH. The MeOH solution was concentrated to dryness. The mixture was treated with TEA (0.1 mL) for 30 minutes and concentrated to dryness with reduced pressure. The De-BOM nucleoside was obtained as white foam (0.12 g, quantitative).

To a pyridine solution (4.7 mL) of the De-BOM nucleoside (0.115 g, 0.47 mmol) at 0° C., DMTrCl (0.24 g, 0.71 mmol) was added. The reaction mixture was warmed to room temperature and stirred for 2 hours. The reaction was quenched with MeOH (0.2 mL) and stirred at rt for 30 minutes. The reaction was treated with water (40 mL), extracted with ethyl acetate (20 mL). The ethyl acetate solution was washed with water (30 mL×2) and concentrated to dryness. The crude product was purified by silica gel column chromatography and eluted with 0-30% ethyl acetate in dichloromethane gradient to yield compound 2.07 (0.22 g, 88%). Spectral data are consistent with the structure of compound 2.07.

### Preparation of Compound 2.08

To a DMF (2.3 mL) solution of compound 2.07 (0.2 g, 0.36 mmol) and tetrazole (0.020 g, 0.29 mmol) at 0° C., 1-methylimidazole (0.007 mL, 0.09 mmol) and 2-Cyanoethyl N,N,N′,N′-tetraisopropylphosphorodiamidite (0.23 mL, 0.72 mmol) were added. The reaction was warmed to room temperature and stirred at this temperature for 2 hours. The reaction mixture was extracted with ethyl acetate (100 mL), washed with sat. NaHCO₃ (150 mL), brine and dried over Na₂SO₄. The ethyl acetate solution was concentrated to dryness. The crude product was purified by silica gel column chromatography and eluted with 50% ethyl acetate in hexanes to yield compound 2.08 (0.25 g, 93%). Spectral data are consistent with the structure of compound 2.08.

### Example 21: Synthesis of an Amidite of a 2′-Substituted Stereo-Non-Standard Nucleoside Comprising a 2′-fluoro-α-L-ribosyl Sugar Moiety

Compound 3.09, an amidite of a stereo-non-standard nucleoside comprising a 2′-fluoro-α-L-ribosyl sugar moiety, was prepared according to the scheme below:

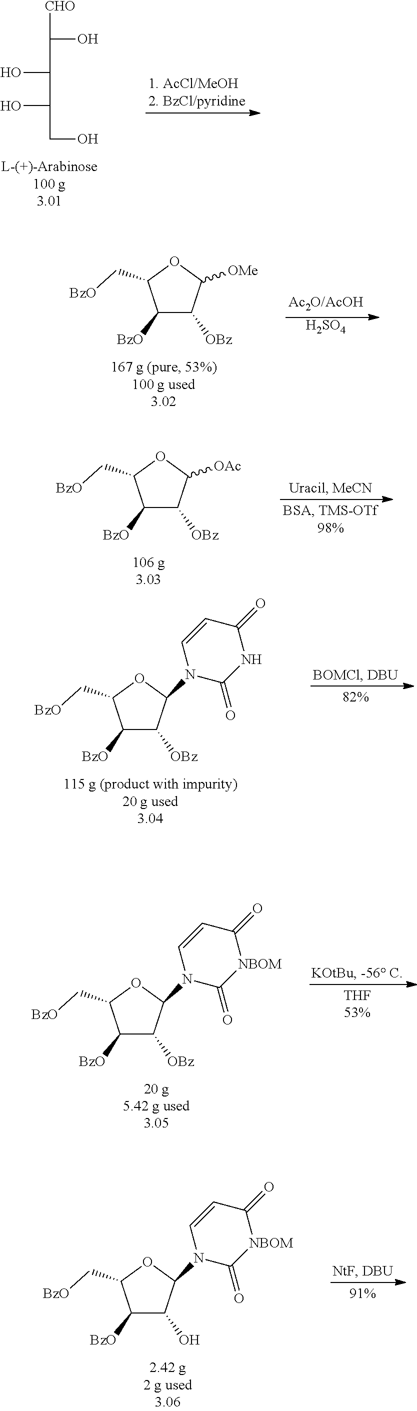

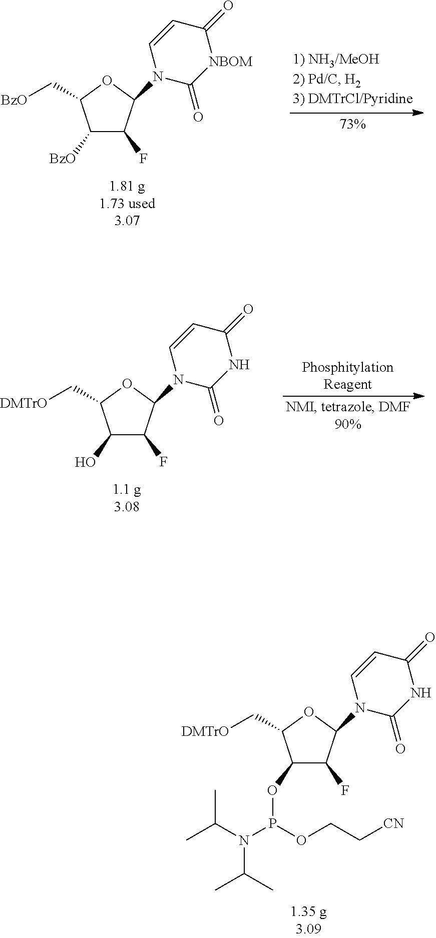

### Preparation of Compound 3.02

In a three-neck flask charged with MeOH (600 mL) in an NaCl ice bath under nitrogen flow, acetyl chloride (50 mL, 700 mmol) was added dropwise over 14 min. The reaction was stirred at room temperature for another 30 min. The methanolic hydrogen chloride solution was added via cannula slowly to a solution of L-(+)-arabinose (3.01) (100 g, 666 mmol) in methanol (2 L) suspension at room temperature over 20 min and the reaction was stirred at room temperature for 12 h. The reaction was neutralized with adding 60 mL of pyridine. The solution was concentrated, and crude oil was co-evaporated with toluene three times (60 mL×3). The remaining oil was dried under high vacuum for 12 hours. The colorless oil was used for next step without any further purification.

L-(+)-arabinose 1′ methyl ether (109 g, 666 mmol) was dissolved in pyridine (750 mL) and cooled to 0° C. Benzoyl chloride (309 mL, 2662 mmol) was added to the pyridine solution slowly. The reaction was warmed to room temperature and stirred overnight. To the reaction, water (2 L) was added, and the mixture was extracted with DCM (2×750 mL). The combined DCM solution was concentrated and co-evaporated with toluene (3×100 mL). The residue was dried over reduced pressure for 12 h. The crude product was purified by silica gel column chromatography and eluted with ethyl acetate hexanes solution to yield compound 3.02 (167 g, 52%).

### Preparation of Compound 3.03

A solution of compound 3.02 (100 g, 210 mmol) in glacial acetic acid (600 mL) and acetic anhydride (65 mL, 693 mmol) was cooled to 0° C. in an ice bath. Sulfuric acid (2.24 mL, 42 mmol) was added dropwise. The reaction was warmed to room temperature and stirred for 12 h. The reaction mixture was quenched with water/brine (1.6 L/1.6 L) with an ice bath and extracted with dichloromethane (3×500 mL). The resulting dichloromethane solution was washed with saturated sodium bicarbonate solution aqueous (2×600 mL) and brine (2×600 mL) and dried over sodium sulfate. Concentration under reduced pressure gave an oil as desired product 3.03 (109.51 g, quantitative).

### Preparation of Compound 3.04

Compound 3.03 (106 g, 210 mmol) and uracil (30.6 g, 273 mmol) was dried under reduced pressure for 12 hours. To this flask, acetonitrile (1 L) and BSA (208 mL, 849 mmol) were added. The mixture was heated with a heat gun to be a clear solution, then cooled to 0° C. To this solution in an ice bath, TMSOTf (61 mL, 336 mmol) was slowly added, and then the reaction was warmed to room temperature. The mixture was stirred at 85° C. for 3 hours and cooled to room temperature. The reaction was quenched with saturated sodium bicarbonate aqueous solution and extracted with ethyl acetate (1 L). The ethyl acetate solution was concentrated to dryness. The crude product was purified by silica gel column chromatography and eluted with ethyl acetate dichloromethane solution to yield compound 3.04 (115 g, 98%).

### Preparation of Compound 3.05

To a solution of compound 3.04 (20 g, 36 mmol) in DMF (110 mL) at 0° C., DBU (10.77 mL, 71 mmol) was added and then BOMCl (7.5 mL, 54 mmol) was added. The reaction was stirred at 0° C. and monitored with LCMS. After 4 h, the reaction was quenched with saturated NaHCO₃ aqueous solution (200 mL). The mixture was extracted with ethyl acetate (300 mL) and washed with brine 3×(200 mL). The combined ethyl acetate solution was washed with water (300 mL) and dried over Na₂SO₄. The resulting ethyl acetate solution was concentrated to dryness. The crude product was purified by silica gel column chromatography and eluted with ethyl acetate hexanes solution to yield compound 3.05 (20 g, 82%).

### Preparation of Compound 3.06

To a solution of compound 3.05 (5.42 g, 8.01 mmol) in THF (220 mL), chilled at −56° C. in dry-ice-acetonitrile bath, was added potassium tert-butoxide 1M THF solution (12 mL, 12.01 mmol) with vigorous stirring. After 13 minutes, 2 N HCl aqueous solution (12 mL, 24.02 mmol) was added and the mixture was stirred for 5 minutes. The reaction was concentrated with reduced pressure and the residue was extracted with ethyl acetate (200 mL). The ethyl acetate solution was washed with water (250 mL) and the organic solution was concentrated to dryness. The crude product was purified by silica gel column chromatography and eluted with ethyl acetate dichloromethane solution to yield compound 3.06 (2.43 g, 53%).

### Preparation of Compound 3.07

To a toluene (30 mL) solution of compound 3.06 (2 g, 3.49 mmol), DBU (1 mL, 6.99 mmol) and NfF (1.25 mL, 6.99 mmol) were added. The reaction was stirred at 50° C. for 12 h. The reaction was extracted with ethyl acetate (100 mL), washed with brine (300 mL). The ethyl acetate solution was concentrated to dryness. The residue was purified by silica gel column chromatography and eluted with ethyl acetate hexanes solution to yield compound 3.07 (1.81 g, 91%).

### Preparation of Compound 3.08

To a pressure flask charged with compound 3.07 (1.73 g, 3.01 mmol) and MeOH (4 mL), 7 N NH₃ methanol solution (6 mL, 42 mmol) was added. The flask was sealed and stirred at 55° C. for 12 h. The reaction was concentrated to dryness. The residue was purified by silica gel column chromatography and eluted with an MeOH/dichloromethane solution to yield de-benzoylated product (0.88 g, 80%).

To a MeOH solution of de-benzoylated product (0.84 g, 2.29 mmol), Pd(OH)₂ (0.42 g) was added. The reaction was stirred at room temperature under H₂ for 12 h. The reaction was filtered through a Celite padding and rinsed with MeOH. The MeOH solution was concentrated to dryness with reduced pressure and co-evaporated with ACN. De-BOM product was obtained as white foam (0.61 g, quantitative).

To a pyridine solution (25 mL) of de-BOM nucleoside (0.57 g, 2.32 mmol) at 0° C., DMTrCl (0.98 g, 2.89 mmol) was added. The reaction mixture was warmed to room temperature and stirred for 2 h. The reaction was quenched with MeOH (0.2 mL) and stirred at room temperature for 30 minutes. The reaction was treated with water (100 mL) and extracted with ethyl acetate (100 mL). The ethyl acetate solution was washed with water (200 mL) and concentrated to dryness. The crude product was purified by silica gel column chromatography and eluted with ethyl acetate dichloromethane solution to yield compound 3.08 (1.16 g, 91.34%).

### Preparation of Compound 3.09

To a DMF (10 mL) solution of compound 3.08 (1.1 g, 2.01 mmol) and tetrazole (0.11 g, 1.61 mmol) at 0° C., 1-methylimidazole (0.040 mL, 0.5 mmol) and 2-Cyanoethyl N,N,N′,N′-tetraisopropylphosphorodiamidite (1.3 mL, 4.02 mmol) were added. The reaction was warmed to room temperature and stirred at this temperature for 2 h. The reaction mixture was extracted with ethyl acetate (100 mL), washed with sat. NaHCO₃ (150 mL), brine and dried over Na₂SO₄. The ethyl acetate solution was concentrated to dryness. The crude product was purified by silica gel column chromatography and eluted with ethyl acetate hexanes solution to yield compound 3.09 (1.35 g, 90%).

### Example 22: Synthesis of an Amidite of a 2′-Substituted Stereo-Non-Standard Nucleoside Comprising a 2′-fluoro-β-L-ribosyl Sugar Moiety

Compound 4.06, an amidite of a stereo-non-standard nucleoside comprising a 2′-fluoro-β-L-ribosyl sugar moiety, was prepared according to the scheme below:

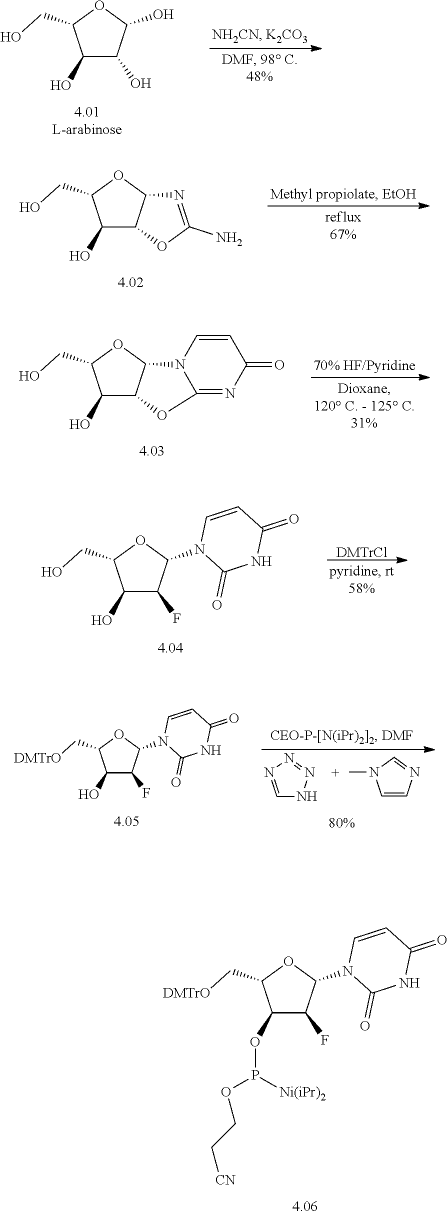

Steps of this synthesis have been previously described, see, e.g., Ross, U.S. Pat. No. 6,642,367; Gaubert, et al., Tetrahedron, 2006.

### Preparation of Compound 4.02

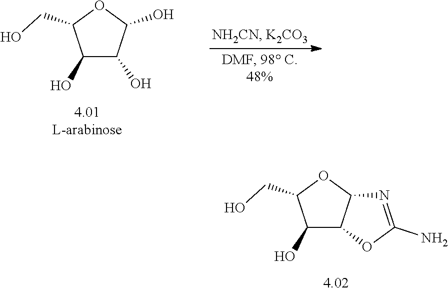 [SMILES: OC[C@@H]1O[C@H](O)[C@H](O)[C@H]1O ; NC1=N[C@H]2O[C@@H](CO)[C@H](O)[C@H]2O1]

A mixture of L-arabinose (4.01) (30.0 g, 199.83 mmol), cyanamide (10.10 g, 239.79 mmol, 1.20 eq) and potassium bicarbonate (280 mg, 2.0 mmol, 0.10 eq) was stirred at 90° C. for 1.5 hours in anhydrous DMF (225 mL). After cooling at room temperature, the solvent was evaporated under reduced pressure to about 50 mL, and the solution was set at 0° C. overnight. The next day, product precipitated out from solution as a white solid. The solid was filtered and rinsed with diethyl ether (40 mL) followed with ethanol (100 mL). The solid was collected and dried under high vacuum over P₂O₅ at 35° C. to obtain compound 4.02 (16.73 g, 48% yield).

### Preparation of Compound 4.03

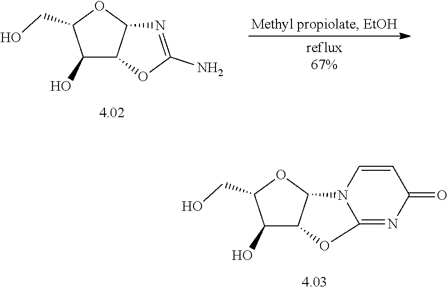 [SMILES: NC1=N[C@H]2O[C@@H](CO)[C@H](O)[C@H]2O1 ; O=c1ccn2c(n1)O[C@@H]1[C@@H](O)[C@H](CO)O[C@@H]12]

A mixture of (3aS,5S,6S,6aR)-2-amino-5-(hydroxymethyl)-3a,5,6,6a-tetrahydrofuro[2,3-d]oxazol-6-ol (4.02) (16.70 g, 95.89 mmol) and methyl propionate (17.10 mL, 191.78 mmol, 2 eq.) was dissolved in (1/1) aqueous ethanol solution (250 mL) and refluxed at 100° C. for two hours to obtain a light brown solution. TLC in DCM/acetone/MeOH (20 mL/5 mL/3 mL) indicated that the reaction was complete.

The solvent was evaporated under reduced pressure to obtain a crude oil that was dissolved in acetone (220 mL) and let set at 0° C. overnight. The next day, the product was precipitated out as white solid. The solid was filtered and rinsed with fresh acetone. The solid was then collected and dried under high vacuum over P₂O₅ at 35° C. to obtain compound 4.03 (14.66 g, 68% yield).

### Preparation of Compound 4.04

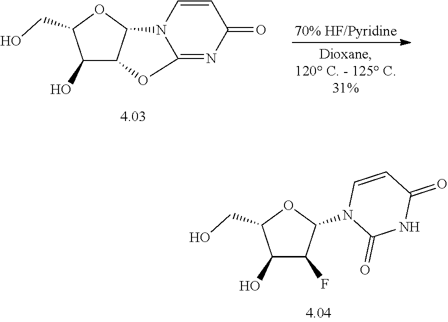 [SMILES: O=c1ccn2c(n1)O[C@@H]1[C@@H](O)[C@H](CO)O[C@@H]12 ; O=c1ccn([C@H]2O[C@@H](CO)[C@H](O)[C@@H]2F)c(=O)[nH]1]

(2S,3S,3aR,9aS)-3-hydroxy-2-(hydroxymethyl)-2,3,3a,9a-tetrahydro-6H-furo[2′,3′:4,5]oxazolo[3,2-a]pyrimidin-6-one (4.03) (5.0 g, 22.11 mmol) was suspended in dioxane (50 mL) in a 500 mL stainless steel bomb. 70% HF/Pyridine (11.95 mL, 132.63 mmol, 6 eq) was added. The stainless steel bomb was closed and heated for 16 hours to 120° C.-125° C. The next day, the bomb was cooled down to room temperature, and the mixture was poured in 100 mL of ice. Saturated NaHCO₃ (50 mL) was added while stirring for 10 minutes. NaHCO₃ was added to adjust the solution to pH 7.

The solvent was evaporated to obtain crude oil, which was suspended in DCM/acetone/methanol (200 mL) (20 mL/5 mL/5 mL), and stirred mixture vigorously to obtain a large amount of precipitate salt. The solid was filtered and solvent was evaporated under reduced pressure to obtain crude oil. The crude material was dissolved in DCM/MeOH (95/5) and loaded to silica gel chromatography (Si, 50 g col, 0-10% MeOH/DCM) to afford the desired product (4.04) as a white solid (1.70 g, 31% yield).

### Preparation of Compound 4.05

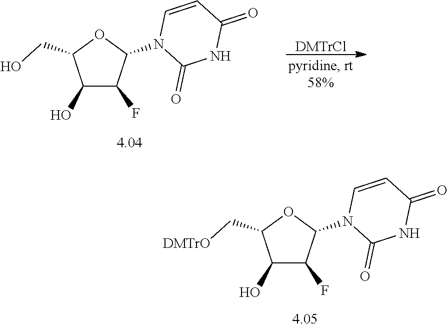

DMTrCl (2.81 g, 8.29 mmol) was added to a solution of 1-((2S,3S,4S,5S)-3-fluoro-4-hydroxy-5-(hydroxymethyl)tetrahydrofuran-2-yl)pyrimidine-2,4(1H,3H)-dione (4.04) (1.70 g, 6.94 mmol) in pyridine (30 mL) at room temperature and stirred for 3 hours. TLC in EtOAc/hexane (7/3) indicated that the reaction was complete. The solution was transferred into a separatory funnel and diluted with ethyl acetate (50 mL) and washed with DI water (2×50 mL). The aqueous layer was removed, and the organic layer was washed with sat. NaHCO₃, sat. brine and dried over Na₂SO₄. The resulting solution was filtered, and solvent was evaporated under reduced pressure to obtain crude material. The crude material was dissolved in DCM and purified by silica gel chromatography. Biotage (Si, 50 g col, 5-60% ethyl acetate/hexane+1% Et₃N) afforded the desired product (4.05) as a white solid (2.20 g, 58% yield).

### Preparation of Compound 4.06

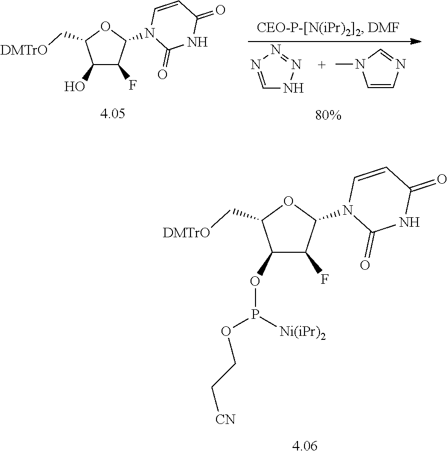

1H-Tetrazole (211 mg, 3.0 mmol, 0.8 eq) and 1-methylimidazole (76.30 μL, 957.0 μmol, 0.25 eq) were added to a solution of 1-((2S,3S,4S,5S)-5-((bis(4-methoxyphenyl)(phenyl)methoxy)methyl)-3-fluoro-4-hydroxytetrahydrofuran-2-yl)pyrimidine-2,4(1H,3H)-dione (4.05) (2.10 g, 3.83 mmol) in anhydrous DMF (15 mL) at room temperature under an atmosphere of nitrogen. 3-bis(diisopropylamino)phosphanyloxypropanenitrile (1.82 mL, 5.74 mmol, 1.5 eq) was then added dropwise and the reaction was stirred at room temperature for 10 hours. The reaction solution was transferred to a separatory funnel, diluted with a 3:1 mixture of toluene/hexanes (30 mL), and the organic layer was washed with 4×(30 mL) with a 3:2 mixture of DMF/H₂O. The organic layer was washed with saturated sodium bicarbonate solution and brine, dried over solid sodium sulfate, and concentrated under reduced pressure to a white foam. Purification by Biotage (Si, 50 g col, 20-50% ethyl acetate/hexanes+1% triethylamine) afforded the desired product (4.06) as a white solid (2.30 g, 80% yield).

### Example 23: Synthesis of an Amidite of a 2′-Substituted Stereo-Non-Standard Nucleoside Comprising a 2′-fluoro-α-L-arabinosyl Sugar Moiety

Compound 5.06, an amidite of a stereo-non-standard nucleoside comprising a 2′-fluoro-ax-L-arabinosyl was prepared according to the scheme below:

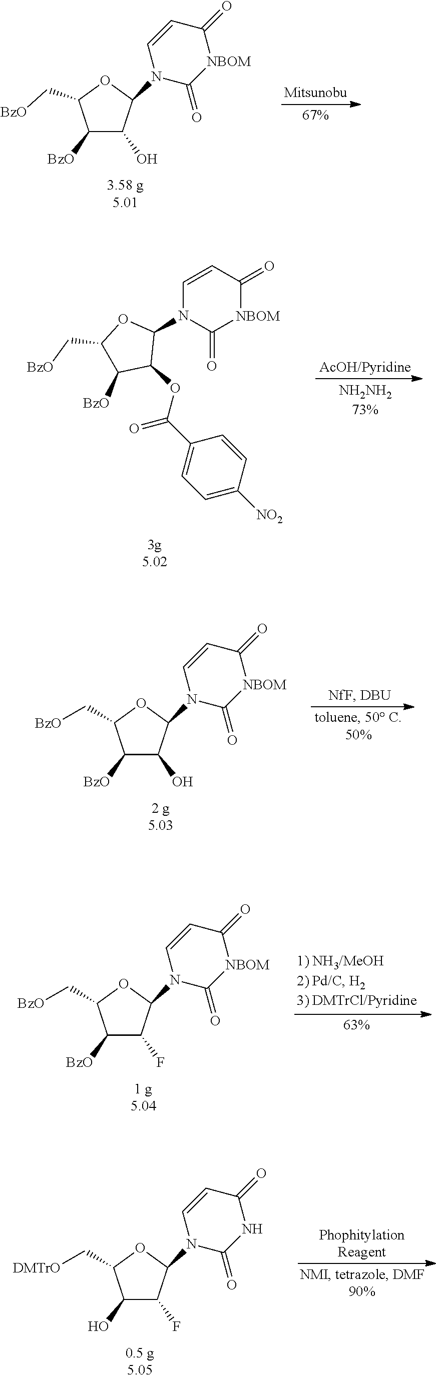

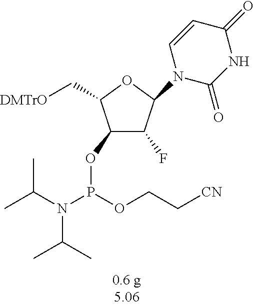

### Preparation of Compound 5.02

To a THF solution (46 mL) of compound 5.01 (2.63 g, 4.59 mmol), p-nitrobenzoic acid (1.54 g, 9.19 mmol) and Ph₃P (2.4 g, 9.19 mmol), DIAD (1.8 mL, 9.19 mmol) was dropwise added at room temperature. The reaction was stirred at room temperature for 12 h and concentrated. The residue was re-dissolved in dichloromethane and washed with brine. The concentrated crude product was purified by silica gel column chromatography and eluted with ethyl acetate dichloromethane solution to yield compound 5.02 (2.24 g, 68%).

### Preparation of Compound 5.03

To a 30 mL of acetic acid/pyridine (1:4) solution of compound 5.02 (2.24 g, 3.10 mmol), H₂H₄.xH₂O (0.39 mL, 12.40 mmol) was added. The reaction mixture was stirred for 14 h and quenched with acetone (10 mL). The mixture was stirred for another 2 h. The reaction was concentrated, and the residue was partitioned between water and ethyl acetate. The combined organic phase was washed with saturated sodium bicarbonate aqueous solution, water and concentrated. The crude product was purified by silica gel column chromatography and eluted with ethyl acetate dichloromethane solution to yield compound 5.03 (1.45 g, 81%).

### Preparation of Compound 5.04

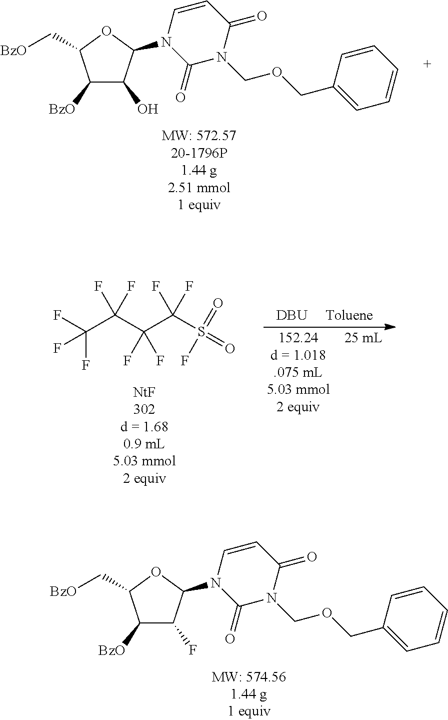

To a toluene (25 mL) solution of compound 5.03 (1.44 g, 2.51 mmol), DBU (0.75 mL, 5.03 mmol) and NfF (0.9 mL, 5.03 mmol) were added. The reaction was stirred at 50° C. for 12 h. The reaction was extracted with ethyl acetate (100 mL) and washed with brine (250). The ethyl acetate solution was concentrated. The crude product was purified by silica gel column chromatography and eluted with ethyl acetate hexanes solution to yield compound 5.04 (0.91 g, 63%).

### Preparation of Compound 5.05

To a pressure flask charged with compound 5.04 (0.88 g, 1.53 mmol) and MeOH (3 mL), 7 N NH₃ methanol solution (4 mL) was added. The flask was sealed and stirred at 55° C. for 12 hours. The reaction was concentrated. The residue was purified by silica gel column chromatography and eluted with an MeOH/dichloromethane solution to yield de-benzoylated product (0.56 g, quantitative).

To a MeOH solution (18 mL) of the de-benzoylated product (0.56 g, 1.53 mmol), Pd(OH)₂ (0.35 g) was added. The reaction was stirred at room temperature under H₂ for 12 h. The reaction was filtered through a Celite padding and rinsed with MeOH. The MeOH solution was concentrated to dryness. The de-BOM product was obtained as white foam (0.38 g, quantitative).

To a pyridine solution (16 mL) of de-BOM product (0.38 g, 1.54 mmol) at 0° C., DMTrCl (0.78 g, 2.32 mmol) was added. The reaction mixture was warmed to room temperature and stirred for 2 h. The reaction was quenched with MeOH (0.4 mL) and stirred at rt for 30 minutes. The reaction was extracted with ethyl acetate (100 mL) and washed with water (200 mL). The resulting ethyl acetate solution was concentrated to dryness. The crude product was purified by silica gel column chromatography and eluted with ethyl acetate dichloromethane solution to yield compound 5.05 (0.65 g, 77%).

### Preparation of Compound 5.06

To a DMF (6 mL) solution of compound 5.05 (0.6 g, 1.09 mmol) and tetrazole (0.061 g, 0.88 mmol) at 0° C., 1-methylimidazole (0.022 mL, 0.23 mmol) and 2-cyanoethyl N,N,N′,N′-tetraisopropylphosphorodiamidite (0.69 mL, 2.18 mmol) were added. The reaction was warmed to room temperature and stirred at this temperature for 2 h. The reaction mixture was extracted with ethyl acetate (100 mL), washed with sat. NaHCO₃ (150 mL), brine and dried over Na₂SO₄. The ethyl acetate solution was concentrated to dryness. The crude product was purified by silica gel column chromatography and eluted with ethyl acetate hexanes solution to yield compound 5.06 (0.65 g, 79%).

### Example 24: Synthesis of an Amidite of a 2′-Substituted Stereo-Non-Standard Nucleoside Comprising a 2′-Fluoro-β-L-arabinosyl Sugar Moiety

Compound 6.09, an amidite of a stereo-non-standard nucleoside comprising a 2′-fluoro-β-L-arabinosyl sugar moiety, was prepared according to the scheme below:

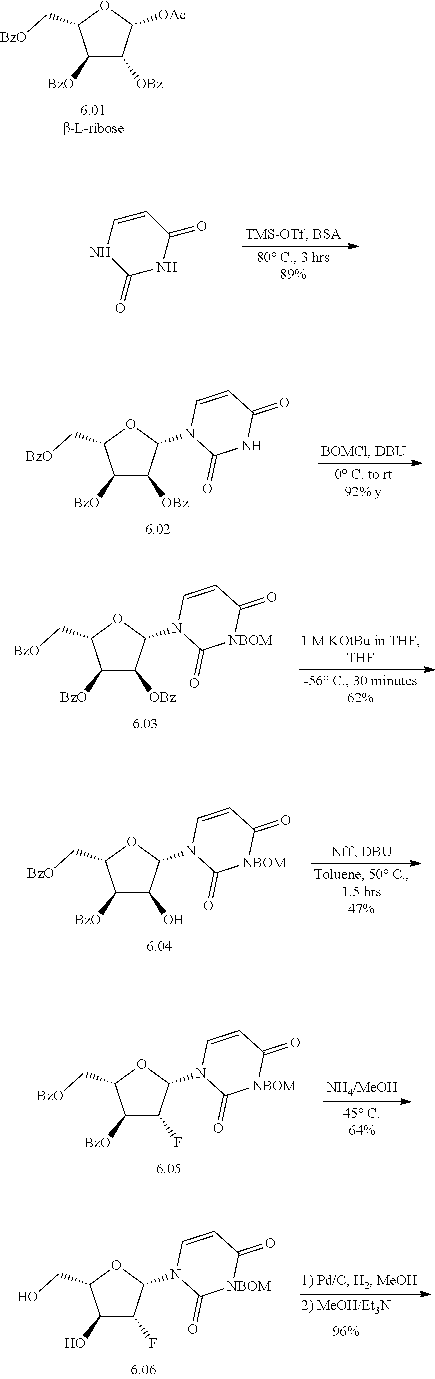

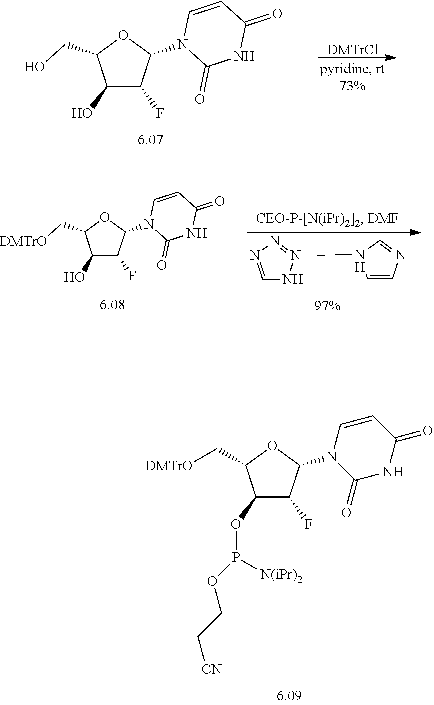

Steps of this synthesis have been previously described, see, e.g., Takamatsu, et al., 2002; Pankiewicz, et al., 1993; Seth, 2012; Nishino, Tetrahedron, 1986.

### Preparation of Compound 6.02

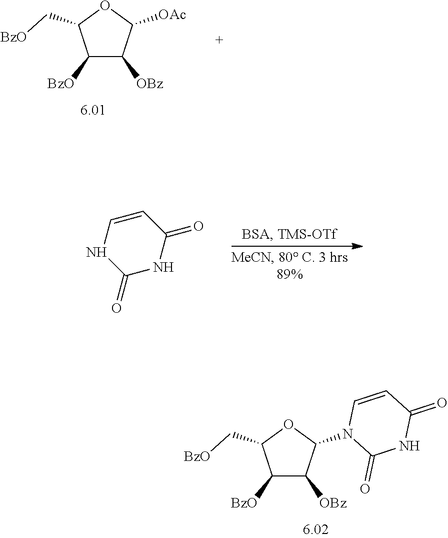

(2R,3S,4S,5S)-2-acetoxy-5-((benzoyloxy)methyl)tetrahydrofuran-3,4-diyl dibenzoate (6.01) (20.0 g, 39.64 mmol) and pyrimidine-2,4(1H,3H)-dione (8.90 g, 79.29 mmol, 2 eq) were co-evaporated at 60° C. with anhydrous acetonitrile (3×50 mL). The mixture was suspended in anhydrous acetonitrile (100 mL), and N,O-Bis(trimethylsilyl)acetamide (38.77 mL, 158.58 mmol, 4.0 eq) was added. The reaction was heated at 80° C. for 30 minutes to obtain a clear solution. The reaction was cooled down with an ice bath to 0° C. Trimethylsilyl trifluoromethanesulfonate (14.10 g, 63.43 mmol, 1.6 eq) was added, and the reaction was stirred for 3 hours at 80° C. TLC in hexane/EtOAc (1/1) indicated that the reaction was complete. The solvent was evaporated under reduced pressure to obtain crude oil. The crude material was dissolved in ethyl acetate (500 mL) and washed with plain DI water, followed with saturated sodium bicarbonate solution to pH 7. The aqueous layer was removed. The organic layer was washed with saturated brine, dried over Na₂SO₄ for 15 minutes, filtered, and concentrated under reduced pressure to obtain a crude oil. The product was precipitated from dichloromethane (50 mL) to obtain product 6.02 (19.65 g, 89% yield).

### Preparation of Compound 6.03

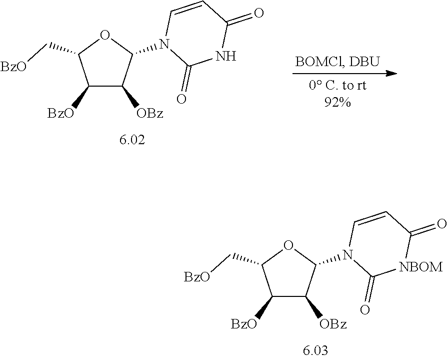

(2S,3S,4S,5S)-2-((benzoyloxy)methyl)-5-(2,4-dioxo-3,4-dihydropyrimidin-1(2H)-yl)tetrahydrofuran-3,4-diyl dibenzoate (6.02) (9.0 g, 16.17 mmol) dissolved in anhydrous dimethylformamide (60 mL) was stirred under nitrogen at room temperature. 2,3,4,6,7,8,9,10-octahydropyrimido[1,2-a]azepine (4.92 g, 32.34 mmol, 2.0 eq) was added, and the reaction was cooled down using an ice bath to 0° C. ((Chloromethoxy)methyl)benzene (3.80 g, 24.26 mmol, 1.5 3q.) was added dropwise to the reaction which was stirred at room temperature for 4 hours. TLC in EtOAc/hexane (4/6) indicated that the reaction was complete. The reaction was quenched by adding to the reaction 50 mL sat. NaHCO₃ solution. The solution was transferred to a separatory funnel, and the product was extracted with ethyl acetate (2×50 mL). The organic layer was washed with sat. NaHCO₃ solution and sat. brine solution, then dried over Na₂SO₄ for 15 minutes, filtered, and concentrated under reduced pressure to obtain a crude oil. The crude material was dissolved in ethyl acetate/hexane (1/1) and purified by silica gel chromatography (Si, 50 g col, 5-30% ethyl acetate/hexane) to afford the desired product (6.03) as a white solid (24.00 g, 98.69% yield).

### Preparation of Compound 6.04

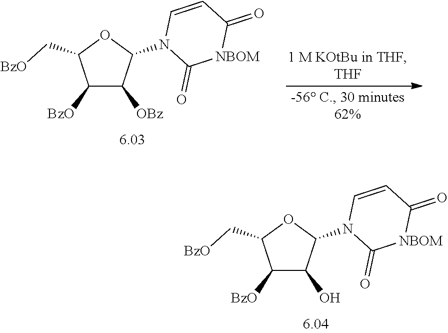

(2S,3S,4S,5S)-2-((benzoyloxy)methyl)-5-(3-((benzyloxy)methyl)-2,4-dioxo-3,4-dihydropyrimidin-1(2H)-yl)tetrahydrofuran-3,4-diyl dibenzoate (6.03) (10.0 g, 14.78 mmol) was dissolved in THF (400 mL) then cooled down with acetone/dry ice to −55° C. To this was added dropwise 1 N Potassium ter-butoxide in THF (22 mL) over a period of 10 minutes to obtain a light yellow solution. The reaction was stirred at −55° C. for 15 minutes and monitored by LC/MS. The reaction was then quenched by adding dropwise 1 N HCl. The cooling system was removed, and the reaction was stirred for 30 minutes. The solvent was removed under reduced pressure to obtain crude oil. The crude oil was suspended in ethyl acetate (100 mL) and washed with DI water (100 mL), sat. NaHCO₃ solution, and sat. brine. The organic layer was dried over Na₂SO₄ for 10 minutes and filtered. Solvent was evaporated under reduced pressure. Crude material was dissolved in DCM and load to column on Biotage (Si, 100 g col, 0-8% acetone/DCM) to afford the desired product (6.04) as a white solid (5.22 g, 62% yield).

### Preparation of Compound 6.05

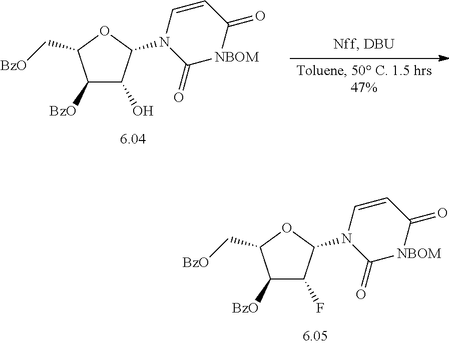

To a solution of ((2S,3R,4S,5S)-3-(benzoyloxy)-5-(3-((benzyloxy)methyl)-2,4-dioxo-3,4-dihydropyrimidin-1(2H)-yl)-4-hydroxytetrahydrofuran-2-yl)methyl benzoate (6.04) (4.97 g, 8.68 mmol) in anhydrous toluene (50 mL) was added 1,8-Diazabicyclo[5.4.0]undec-7-ene (2.60 mL, 17.36 mmol, 2.0 eq) followed with dropwise addition of 1,1,2,2,3,3,4,4,4-nonafluorobutane-1-sulfonyl fluoride (3.12 mL, 17.36 mmol, 2 eq). The reaction was heated at 50° C. overnight. The next day, TLC in EtOAc/hexane (1/1) indicated that the reaction was complete. The reaction was cooled to room temperature, and the solution was diluted with ethyl acetate (50 mL). The organic layer was washed with DI water (50 mL), sat. NaHCO₃ solution, and sat. brine. The organic layer was dried over Na₂SO₄ for 10 minutes, then filtered. Solvent was evaporated under reduced pressure. Crude material was dissolved in DCM and load to column Biotage (Si, 100 g col, 1% acetone/DCM) to afford the desired product (6.05) as a light yellow solid (2.35 g, 47% yield).

### Preparation of Compound 6.06

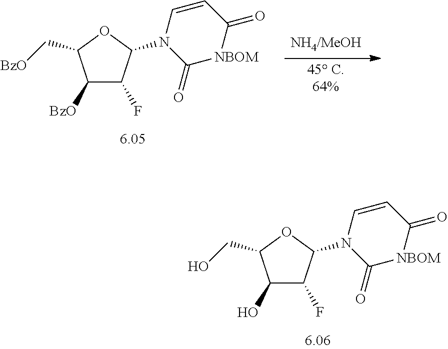

((2S,3S,4R,5S)-3-(benzoyloxy)-5-(3-((benzyloxy)methyl)-2,4-dioxo-3,4-dihydropyrimidin-1(2H)-yl)-4-fluorotetrahydrofuran-2-yl)methyl benzoate (6.05) (2.15 g, 3.15 mmol) in a 7 N NH₃ methanol solution (30 mL) was heated at 45° C. overnight. The next day, TLC in DCM/MeOH (95/5) indicated that the reaction was complete. Solvent was evaporated under reduced pressure. The material was dissolved in DCM/MeOH (95/5) and load to column Biotage (Si, 50 g col, 0-5% DCM/MeOH) afford the desired product (6.06) as a white solid (950 mg, 64% yield).

### Preparation of Compound 6.07

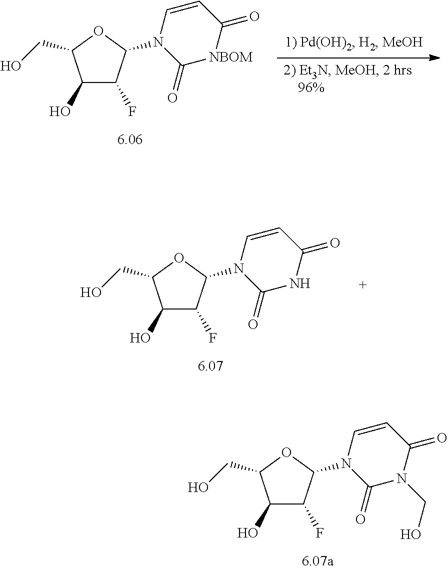

To a solution of 3-((benzyloxy)methyl)-1-((2S,3R,4S,5S)-3-fluoro-4-hydroxy-5-(hydroxymethyl)tetrahydrofuran-2-yl)pyrimidine-2,4(1H,3H)-dione (6.06) (950 mg, 2.59 mmol) in methanol (8 mL) under nitrogen was added Pd(OH)₂. H₂ was introduced to the reaction using a double-folded balloon, and the reaction was stirred overnight. The next day, LC/MS indicated that the reaction was complete with a minor side product of 6.07a. Upon completion, the reaction solution was filtered through a plug of Celite and rinsed with methanol. The filtrate was evaporated under reduced pressure to obtain white solid of the crude mixture. The material was dissolved in methanol (5 mL) and triethylamine (1 mL), the solution was stirred for 2 hours at room temperature. LC/MS indicated full conversion to compound 6.07. The solvent was evaporated under reduced pressure to obtain pure compound 6.07 (610 mg, 96% yield).

### Preparation of Compound 6.08

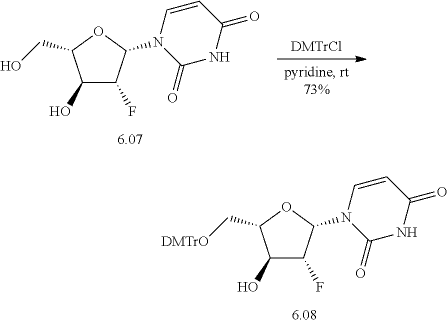

DMTrCl (1.20 g, 2.97 mmol, 1.20 eq) was added to a solution of 1-((2S,3R,4S,5S)-3-fluoro-4-hydroxy-5-(hydroxymethyl)tetrahydrofuran-2-yl)pyrimidine-2,4(1H,3H)-dione (6.07) (610 mg, 2.48 mmol), in pyridine (15 mL) at room temperature and stirred for 3 hours. TLC in EtoAc/hexane (7/3) indicated that the reaction was complete. Solution was transferred into a separatory funnel and diluted with ethyl acetate (50 mL) and washed with DI water (2×50 mL). The aqueous layer was removed. The organic layer was washed with sat. NaHCO₃ and sat. brine, then dried over Na₂SO₄ and filtered. The solvent was evaporated under reduced pressure to obtain crude material, which was dissolved in DCM and loaded to silica gel chromatography. Biotage (Si, 50 g col, 5-60% ethyl acetate/hexane+1% Et₃N) afforded the desired product (6.08) as a white solid (1.0 g, 73% yield).

### Preparation of Compound 6.09

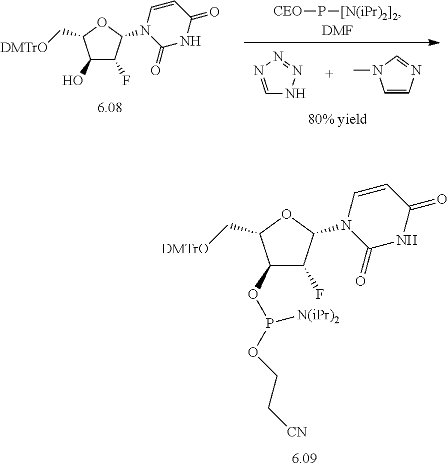

1H-Tetrazole (88.68 mg, 1.28 mmol, 0.8 eq) and 1-methylimidazole (42 μL, 32 μmol, 0.25 eq) were added to a solution of 1-((2S,3R,4S,5S)-5-((bis(4-methoxyphenyl)(phenyl)methoxy)methyl)-3-fluoro-4-hydroxytetrahydrofuran-2-yl)pyrimidine-2,4(1H,3H)-dione (6.08) (900 mg, 1.61 mmol) in anhydrous DMF (15 mL) at room temperature under an atmosphere of nitrogen. 3-bis(diisopropylamino)phosphanyloxypropanenitrile (0.74 mL, 2.41 mmol, 1.5 eq) was then added dropwise and the reaction was stirred at room temperature for 10 hours. The reaction solution was transferred to a separatory funnel and diluted by adding a 3:1 mixture of toluene/hexanes (30 mL). The organic layer was washed (4×30 mL) with a 3:2 mixture of DMF/H₂O. The organic layer was washed with saturated sodium bicarbonate solution and brine, then dried over solid sodium sulfate and concentrated under reduced pressure to a white foam. Purification by Biotage (Si, 50 g col, 20-50% ethyl acetate/hexanes+1% triethylamine) afforded the desired product (6.09) as a white solid (980 mg, 80% yield)

### Example 25: Synthesis of 2′-Substituted Stereo-Non-Standard Nucleosides Comprising 2′-fluoro-α-D-xylosyl or 2′-fluoro-β-D-xylosyl Sugar Moieties

Compound 7.13-A, a stereo-non-standard nucleoside comprising a 2′-fluoro-α-D-xylosyl sugar moiety and compound 7.13-B, a stereo-non-standard nucleoside comprising a 2′-fluoro-β-D-xylosyl sugar moiety, were prepared according to the scheme below:

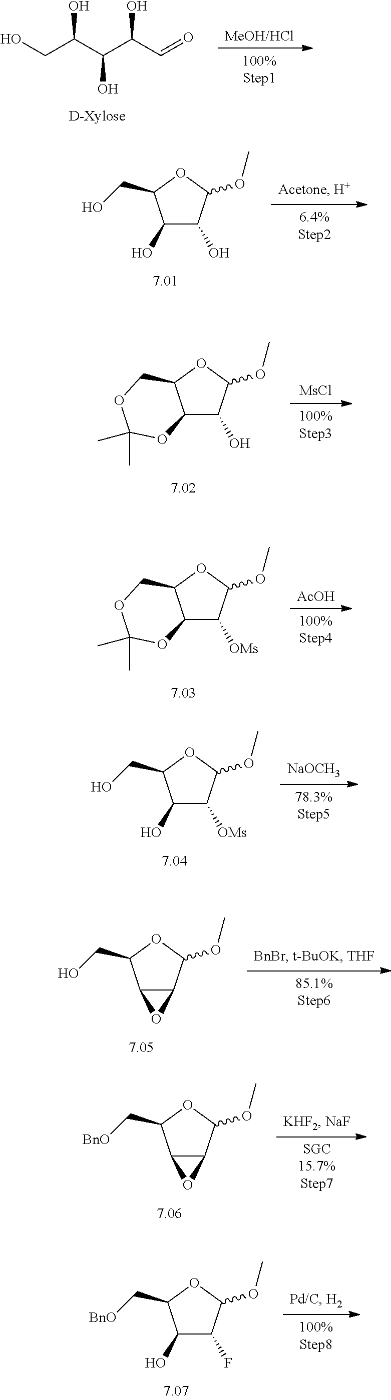

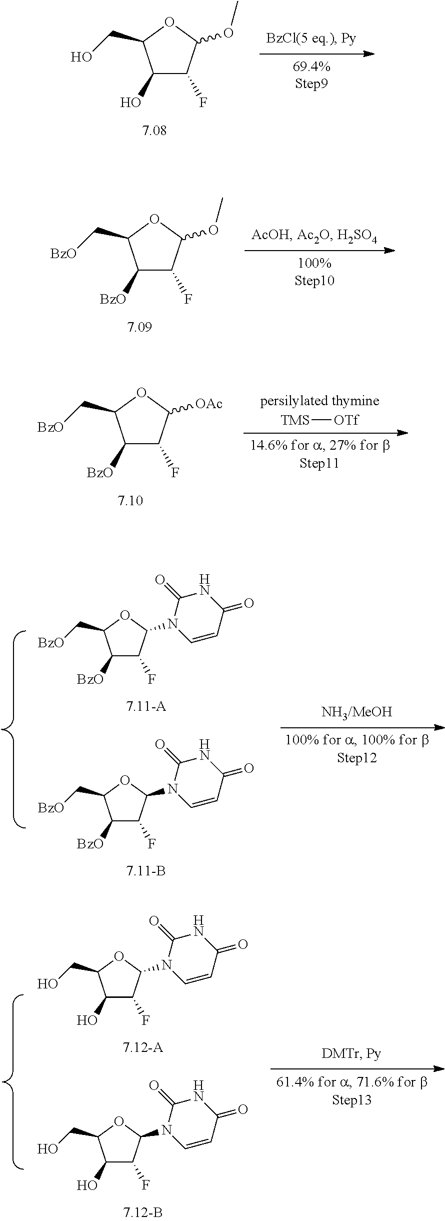

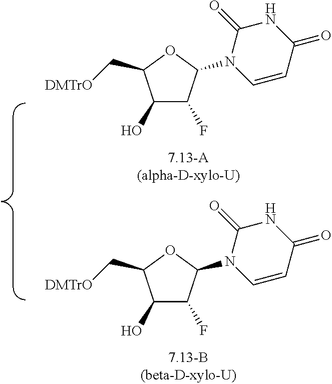

### Preparation of Compound 7.01

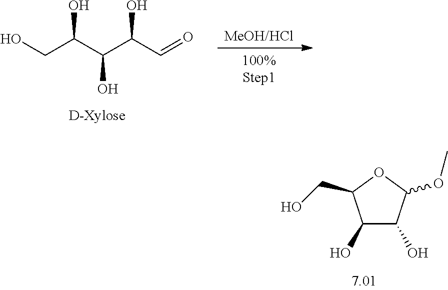

MeOH (23 v) was charged to a 4-necked round bottom flask under stirring conditions. Concentrated HCl (0.25 wt.) was charged to the flask, then D-xylose (700 g) was charged to the mixed solution. The reaction was heated to 55±5° C. and stirred for 21 h. TLC (DCM:MeOH=3:1) showed that D-xylose almost disappeared. The reaction was cooled to 25±5° C., then Ag₂CO₃ (0.55 wt.) was charged to the reaction which was then stirred for 0.5 h. The reaction mixture was filtered through Celite (1 wt.) and rinsed with MeOH (2 v). The organic solution was combined and concentrated to almost no fraction under vacuum at 40±5° C. to get a residue. The residue was washed with DCM (5 v*3) and concentrated under vacuum at 35±5° C. Acetone (8 v) was charged to the residue. The mixture 7.01 (crude; in theory yield) was used in the next step directly.

### Preparation of Compound 7.02

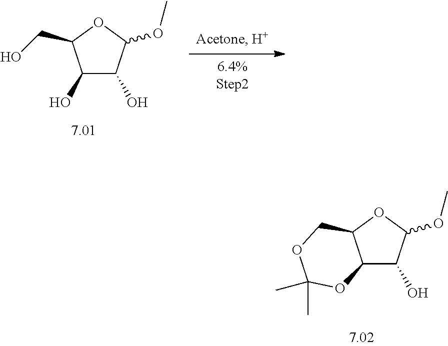

7.01 crude in acetone (˜765 g of 7.01) was charged to a 4-necked round bottom flask in an N₂ atmosphere under stirring conditions. To the flask was charged CuSO₄ (2.1 wt.) and H₂SO₄ (2N, 16 mL). The reaction was heated to 35±5° C. and stirred for 24 h. TLC (DCM:MeOH=5:1) showed 7.01 material. The reaction time was prolonged by 48 h, but there was no obvious increase in 7.02 product and no obvious decrease in 7.01 starting material. The reaction mixture was filtered and rinsed with acetone (3 v). The filtrate was combined and concentrated NH₃ (aq., 26 mL) was added. The solution was concentrated to no obvious fraction under vacuum at 35±5° C. DCM (2 v) and H₂O (1 v) was charged to the residue, and the mixture was stirred for 10 min and left to sit for 10 min before separation. The aqueous phase was extracted with DCM (1 v*2). The organic phase was combined and washed with H₂O (0.15 v). The organic phase was temporarily stored.

The aqueous phase was combined and concentrated to no obvious fraction under vacuum at 55±5° C. The residue was washed with acetone (5 v*3) and concentrated under vacuum at 35±5° C. Acetone (8 v) was charged to the residue which was then transferred to a 4-necked round bottom flask.

The above reaction was repeated three more times, each time starting with the residue from the aqueous phase concentrated in acetone.

All of the organic phase temporarily stored from each reaction were then combined and concentrated to no obvious fraction under vacuum at 35±5° C. The residue was purified through silica gel (100-200 mesh, 10 wt.) and eluted with a solution of (Acetone:DCM=0 to 1:5). The fraction of 7.02 was collected and concentrated to dry under vacuum at 35±5° C. (61 g, 6.4%)

### Preparation of Compound 7.03

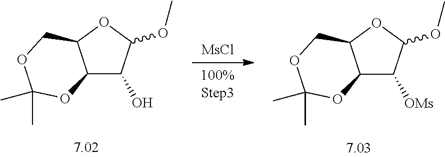

Py (2.7 v) was charged to a 3-necked round bottom flask in an N₂ atmosphere under stirring conditions. 7.02 was then charged to the flask, and CH₃SO₂Cl (0.6 v) was added dropwise to the mixture controlling the temperature at 25±5° C. The reaction was stirred for 3 h. TLC (DCM:Acetone=8:1) showed no 7.02 present. H₂O (14 v) was added to the reaction mixture dropwise at 5±5° C. The mixture was extracted with DCM (7 v*2). The organic phase was combined. The organic phase was washed with toluene (3.5 v*4) and concentrated under vacuum at 55±5° C. The residue was collected as 7.03 crude (in theory yield) and used in the next step.

### Preparation of Compound 7.04

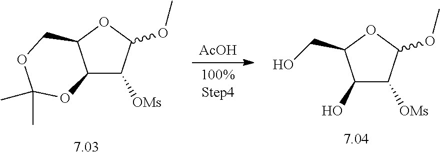

Crude 7.03 in AcOH (1.8 v for 7.02) was charged to a 3-necked round bottom flask under stirring conditions. H₂O (0.75 v for 7.02) was charged to the flask, which was then heated to 55±5° C. The reaction was stirred for 3 h at least at 55±5° C. TLC (DCM:Acetone=8:1) showed no 7.03. Toluene (2 v for 7.02) was charged to the reaction mixture. EtOH (0.4 v for 7.02) was charged to the same mixture to get a clear solution. The clear solution was concentrated to no fraction under vacuum at 55±5° C. Addition of toluene and EtOH followed by concentration was repeated three more times. The residue was collected as 7.04 crude (in theory yield).

### Preparation of Compound 7.05

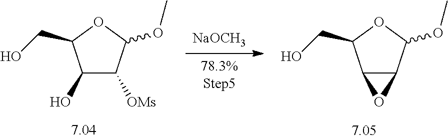

Crude 7.04 in MeOH (3.4 v for 7.02) was charged to a 3-necked round bottom flask in an N₂ atmosphere under stirring conditions. The reaction was heated to 30±5° C. while stirring to get a clear brown solution. The reaction was then cooled to 5±5° C. NaOCH₃ was charged to neutralize the solution and keep the pH>11. The mixture was then heated to 25±5° C. and stirred for at least 16 h keeping the pH>11 and temperature at 25±5° C. TLC (DCM:EA=1:1) showed no 7.04 remaining. The reaction mixture was neutralized with AcOH to pH˜ 7. H₂O was charged to dissolve the precipitated solid. The mixture was extracted with EA (5 v each time) until no 7.05 was found in aqueous phase. All of the organic phase was combined and concentrated to no obvious fraction under vacuum at 40±5° C. to get a residue. The residue was purified through silica gel (100-200 mesh, 10 wt. for 7.02), then eluted with a solution of (EA:DCM=1:50 to 1:1). The pure 7.05 fraction was collected and concentrated under vacuum at 40±5° C. to dry. (32 g, 78.3%)

### Preparation of Compound 7.06

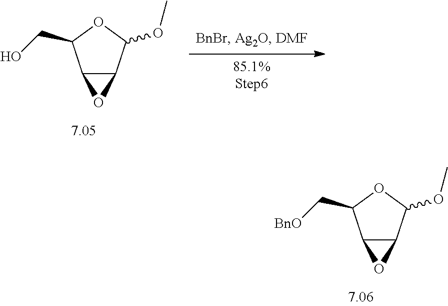

7.05 in THF (10 v) was dissolved in an eggplant-shaped flask. The solution was concentrated to 2 v under vacuum at 40±5° C. THF (8 v) was charged to the residue. KF was sampled and determined to be less than 0.5%. The solution was transferred to a 4-necked round bottom flask in an N₂ atmosphere under stirring conditions. The reaction was cooled to 5±5° C., and t-BuOK (1.1 eq.) was charged to the mixture at 5±5° C. BnBr (1.3 eq) was added dropwise to the reaction mixture, and the temperature was kept at not more than 30° C. The reaction was stirred for 16 h and at a temperature of at least 25±5° C. TLC (EA:DCM=1:1) showed no 7.05 material left. The reaction mixture was concentrated to no obvious fraction under vacuum at 40±5° C. EA (5 v) was charged to the residue, and the mixture was neutralized with AcOH to pH˜7. H₂O (5 v) was charged to the mixture. The layers were separated, and the aqueous phase was extracted with EA (5 v). All of the EA phase was combined and concentrated to no obvious fraction under vacuum at 40±5° C. The residue was purified through silica gel (100-200 mesh, 10 wt.) and eluted with (EA:DCM=0 to 1:10). The pure 7.06 fraction was collected and concentrated under vacuum at 40±5° C. to dry. (44 g, 85.1%) Preparation of Compound 7.07

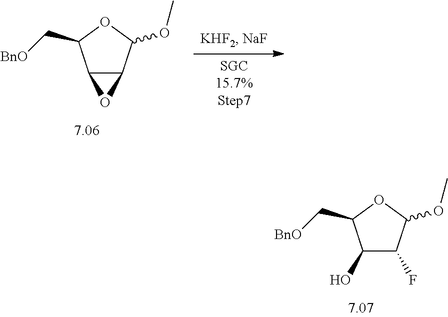

7.06 in DCM (10 v) was dissolved in an eggplant-shaped flask. The solution was concentrated under vacuum at 35±5° C. to no obvious fraction. DCM (10 v) was charged to the residue, and the solution was concentrated under vacuum at 35±5° C. to no obvious fraction. Ethane-1,2-diol (10 v) was charged to the residue. KF was sampled and determined to be less than 0.5%. The mixture was transferred to a 4-necked round bottom flask in an N₂ atmosphere. KHF₂ (3.0 eq.) and NaF (5.6 eq.) were charged to the mixture. The reaction was heated to 165±5° C. and stirred for 2 h. TLC (EA:DCM=1:5) showed no remaining 7.06. The reaction was cooled to 25±5° C. Saturated NaHCO₃ solution (50 v) was added dropwise to the reaction mixture. The organic material was extracted with DCM (20 v*4). All of the DCM solution was combined and concentrated to no fraction under vacuum at 35±5° C. The residue was purified through silica gel (100-200 mesh, 10 wt.) and eluted with a solution of (EA:PE=1:20 to 1:1). The pure 7.07 fraction was collected and concentrated to dry under vacuum at 40±5° C. (7.5 g, 15.7%)

### Preparation of Compound 7.08

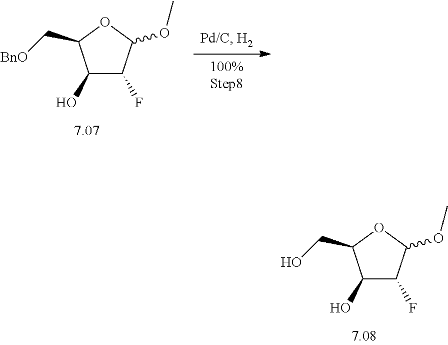 [SMILES: COC1O[C@H](COCc2ccccc2)[C@H](O)[C@H]1F ; "100%Step8" ; "Pd/C, H2" ; COC1O[C@H](CO)[C@H](O)[C@H]1F]

7.07 in EtOH (20 v) was charged into a 4-necked round bottom flask under stirring conditions. The flask was placed under vacuum to ≤−0.08 MPa and then placed under nitrogen atmosphere (repeated three times). Then 10% anhydrous Pd/C (0.2 wt.) was charged to the reaction solution under N₂ protection. Once again, the system was placed under vacuum to ≤−0.08 MPa and then placed under nitrogen atmosphere (repeated three times). The flask was then inflated to an H₂ atmosphere, and the reaction was stirred for 16 h at least at 25±5° C., keeping the H₂ atmosphere. TLC (EA:PE=1:1) showed no remaining 7.07. The reaction mixture was filtered through Celite (1 wt.) under an N₂ atmosphere. The filter cake was rinsed with EtOH (5 v). The filtrate was combined and concentrated to no fraction under vacuum at 45±5° C. EtOH contained in the residue was swapped with DCM (5 v*5) and was dried under vacuum at 45±5° C. The dry residue was collected as 7.08 (in theory yield).

### Preparation of Compound 7.09

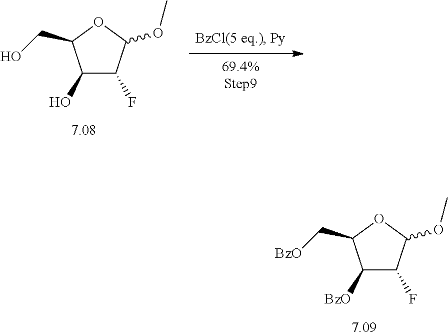

7.08 in Py (26 v) was charged to a 4-necked round bottom flask in an N₂ atmosphere under stirring conditions. The solution was cooled to at 5±5° C., and Bz-Cl (2.5 eq.) was added dropwise to the mixture at 5±5° C. The reaction was stirred for 2 h at least at 5±5° C. TLC (EA:PE=1:3) showed no remaining 7.08. The reaction mixture was diluted with DCM (100 v), then washed with H₂O (30 v*2). The organic phase was washed with 10% citric acid solution (30 v*2) and washed again with H₂O (30 v*2). Silica gel (100-200 mesh, 1.5 wt.) was charged to the organic phase. The organic phase was concentrated to dry under vacuum at 35±5° C. The residue was purified through silica gel (100-200 mesh, 10 wt.) and eluted with the solution of (EA:PE=1:100 to 1:25). The organic fraction was collected and concentrated to dry under vacuum at 40±5° C., leaving pure 7.09 as a viscous residue. (7.6 g, 69.4%)

### Preparation of Compound 7.10

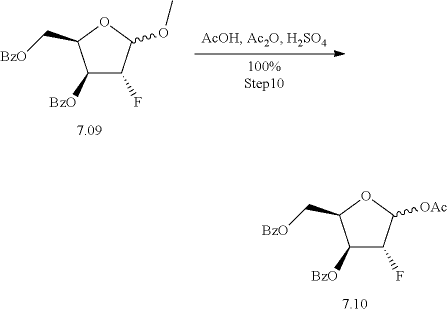

ACOH (9 wt.) was charged to a 3-necked round bottom flask in an N₂ atmosphere under stirring conditions. Ac₂O (9 eq.) was charged to the flask under N₂ protection. The solution was stirred for 5 min. The solution was then charged to dissolve 7.09 resulting in a clear solution. The clear solution was transferred to a 3-necked round bottom flask under N₂ protection under stirring conditions. The reaction was cooled to 5±5° C., then 98% H₂SO₄ (3 eq.) was added dropwise to the mixture at 5±5° C. The reaction was heated to 30±5° C. and stirred for 3 h. TLC (EA:PE=1:5) showed no remaining 7.09. The reaction mixture was added dropwise to saturated NaHCO₃ (500 v) at 5±5° C. The mixture was extracted with DCM (150 v*3). The DCM solution was combined and washed with H₂O (150 v*2). The organic phase was concentrated to no obvious fraction under vacuum at 35±5° C. The water contained in the residue was swapped with DCM (10 v*5) until KF<0.1%. The dry residue was collected as crude 7.10 (in theory yield).

### Preparation of Compounds 7.11-A and 7.11-B

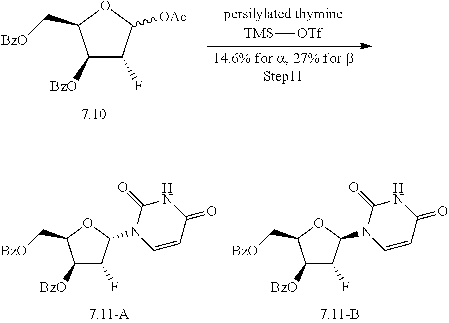

7.10 crude (1 eq.) was dissolved in DCE (20 v) in an N₂ atmosphere. KF was sampled and determined to be less than 0.1%. The solution was transferred to a 4-necked round bottom flask in an N₂ atmosphere under stirring conditions. Uracil (1.1 eq.), HMDS (3.0 eq.), DCE (10 v), and (NH₄)₂SO₄ (0.022 eq.) were charged in turn to another 4-necked round bottom flask under N₂ protection under stirring conditions and heated to 85±5° C. (suspension solution). The solution was stirred for 3 h at 85±5° C., resulting in a clear solution. The clear solution was cooled to 30±5° C. and charged to the 7.10-in-DCE solution under an N₂ atmosphere. TMSOTf (3.3 eq.) was added dropwise to the mixed solution, which was then heated to 85±5° C. and stirred for 3 h. The solution was sampled for LCMS, and no remaining 7.10 was observed. The reaction was cooled to 25±5° C., diluted with DCM (100 v), washed with saturated NaHCO₃ solution (100 v*2), and washed with H₂O (100 v*2). The collected organic phase was concentrated to no fraction under vacuum at 30±5° C. to get a residue. The residue was purified by reverse phase chromatography (5% 7.11 sample in DMSO, C18, Agela-1(HP-Flash-53), ACN-0.05% TFA in water, 30% for 30 min, then 35% to 55% for 40 min). The pure 7.11 fraction (˜500 v) and 7.11 fraction (˜1000 v) were collected.

The 7.11-A fraction was extracted with DCM (500 v*2), and this solution was washed with H₂O (200 v*1). The solution was concentrated to no fraction under vacuum at 35±5° C., leaving residue 7.11-A. (1.35 g, 14.6%)

The 7.11-B fraction was extracted with DCM (1000 v*2), and this solution was washed with H₂O (400 v*1). The solution was concentrated to no fraction under vacuum at 35±5° C., leaving residue 7.11-B. (2.5 g, 27%)

### Preparation of Compound 7.12-A

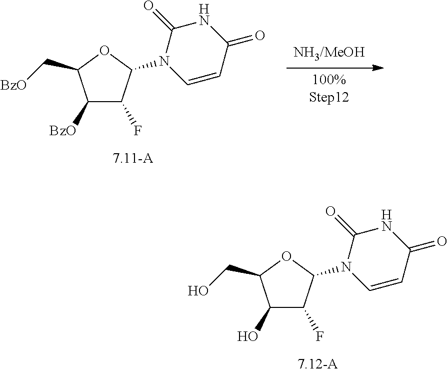

7.11-A (1 eq.) was dissolved in MeOH (58 v) under stirring conditions. NH₃ (aq., 46 wt.) was charged to the solution. The reaction was stirred for 12 h at a temperature of at least 25±5° C. The solution was sampled for LCMS, and no remaining 7.11-A was observed. The reaction solution was concentrated to no obvious fraction under vacuum at 55±5° C. H₂O was removed by azeotropic distillation with MeOH (10 v*5). Residual MeOH was swapped with DCM (20 v*5) under vacuum at 35±5° C. Py (20 v) and DCM (10 v) were charged to the residue. The mixture was concentrated to 20 v under vacuum at 35±5° C. Twice more, DCM (10 v) was charged to the residue, and the mixture was concentrated to 20 v under vacuum at 35±5° C. KF was sampled and determined to be less than 0.05%. The solution was collected as 7.12-A (in theory yield).

### Preparation of Compound 7.12-B

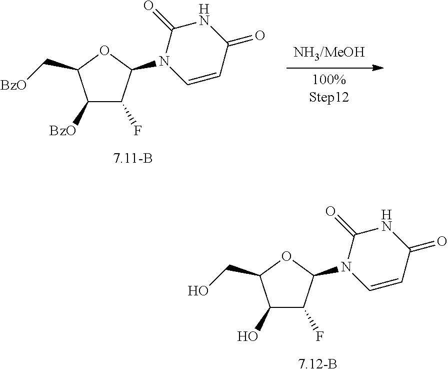

7.11-B (1 eq.) was dissolved in MeOH (58 v) under stirring conditions. NH₃ (aq., 46 wt.) was charged to the solution. The reaction was stirred for 12 h at a temperature of at least 25±5° C. The solution was sampled for LCMS, and no remaining 7.11-B was observed. The reaction solution was concentrated to no obvious fraction under vacuum at 55±5° C. H₂O was removed by azeotropic distillation with MeOH (10 v*5). Residual MeOH was swapped with DCM (20 v*5) under vacuum at 35±5° C. Py (20 v) and DCM (10 v) were charged to the residue. The mixture was concentrated to 20 v under vacuum at 35±5° C. Twice more, DCM (10 v) was charged to the residue, and the mixture was concentrated to 20 v under vacuum at 35±5° C. KF was sampled and determined to be less than 0.05%. The solution was collected as 7.12-B (in theory yield).

### Preparation of Compound 7.13-A

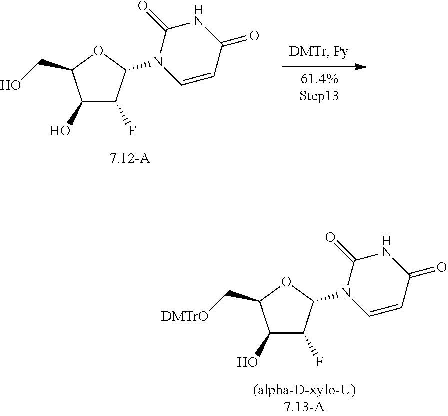

7.12-A in a Py (33 v) solution was transferred to a 4-necked round bottom flask under an N₂ atmosphere under stirring conditions. The reaction was cooled to 5±5° C., and DMTr (0.35 eq. each time, 3 times, total 1.05 eq.) was charged batch-wise at 5±5° C. The reaction was heated to 25±5° C. and stirred for 2 h. TLC (DCM:MeOH=10:1) showed no remaining 7.12-A. The reaction solution was diluted with DCM (100 v), washed with saturated NaHCO₃ (100 v*1), washed with H₂O (100 v*3), and washed with saturated NaCl solution (100 v*1). Silica gel (1.5 wt.) was charged to the organic phase. The mixture was concentrated to dry under vacuum at 35±5° C. leaving a residue. The residue was purified through silica gel (100 mesh, 50 wt.), eluted with PE containing 0.5% TEA, then eluted with a solution of (PE:DCM=1:1) containing 0.5% TEA to remove DMTr and Py, and then eluted with a solution of (DCM:EA=5) containing 0.5% TEA. The target fraction was concentrated to dry under vacuum at 40±5° C., leaving pure residue 7.13-A. (1.0 g, 61.4%)

### Preparation of Compound 7.13-B

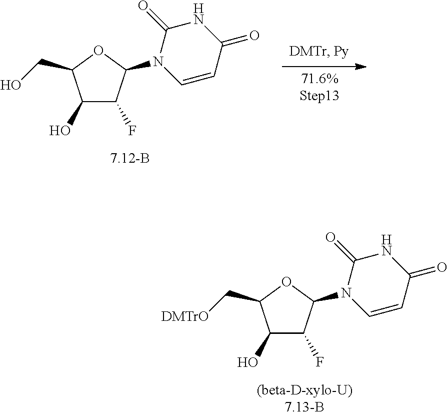

7.12-B in a Py (33 v) solution was transferred to a 4-necked round bottom flask under an N₂ atmosphere under stirring conditions. The reaction was cooled to 5±5° C., and DMTr (0.35 eq. each time, 3 times, total 1.05 eq.) was charged batch-wise at 5±5° C. The reaction was heated to 25±5° C. and stirred for 2 h. TLC (DCM:MeOH=10:1) showed no remaining 7.12-B. The reaction solution was diluted with DCM (100 v), washed with saturated NaHCO₃ (100 v*1), washed with H₂O (100 v*3), and washed with saturated NaCl solution (100 v*1). Silica gel (1.5 wt.) was charged to the organic phase. The mixture was concentrated to dry under vacuum at 35±5° C. leaving a residue. The residue was purified through silica gel (100 mesh, 50 wt.), eluted with PE containing 0.5% TEA, then eluted with a solution of (PE:DCM=1:1) containing 0.5% TEA to remove DMTr and Py, and then eluted with a solution of (DCM:EA=5) containing 0.5% TEA. The target fraction was concentrated to dry under vacuum at 40±5° C., leaving pure residue 7.13-B. (2.16 g, 71.6%)

### Example 26: Synthesis of 2′-Substituted Stereo-Non-Standard Nucleosides Comprising 2′-fluoro-α-L-xylosyl or 2′-fluoro-β-L-xylosyl Sugar Moieties

Compound 8.13-A, a stereo-non-standard nucleoside comprising a 2′-fluoro-α-L-xylosyl sugar moiety and compound 8.13-B, a stereo-non-standard nucleoside comprising a 2′-fluoro-β-L-xylosyl sugar moiety, were prepared according to the scheme below:

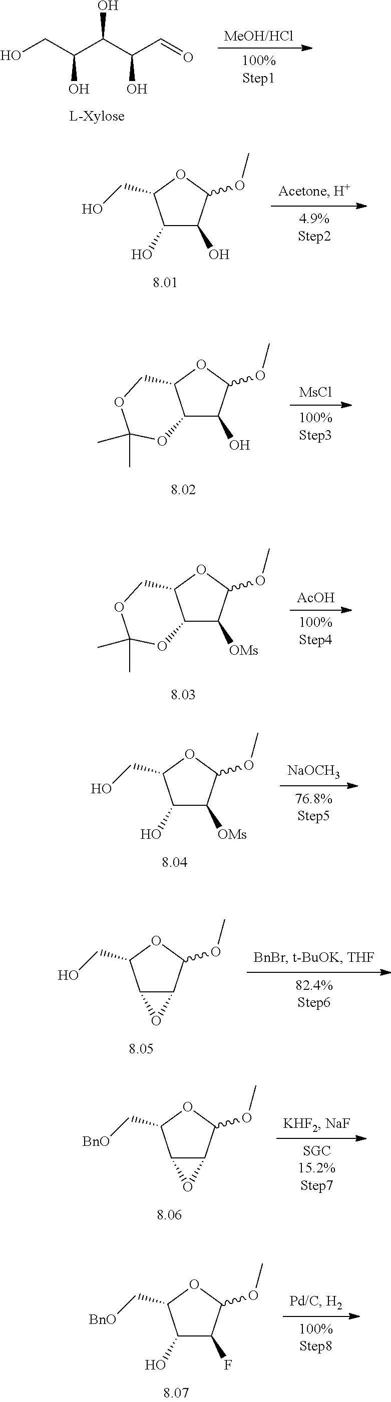

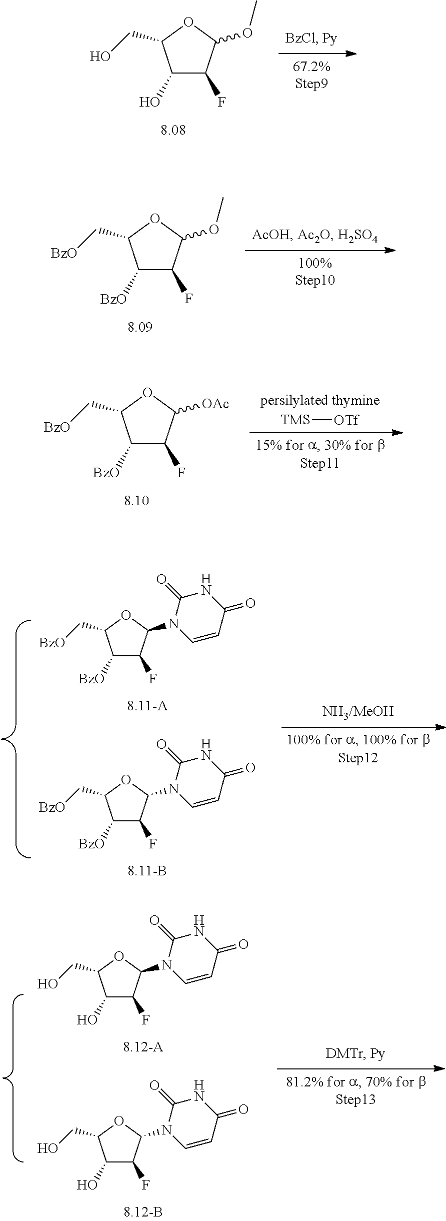

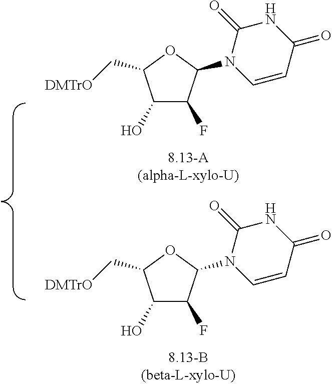

### Preparation of Compound 8.01

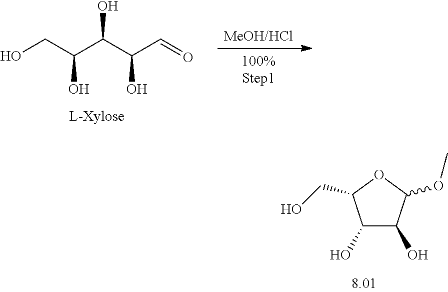

MeOH (23 v) was charged to a 4-necked round flask under stirring conditions. Concentrated HCl (0.25 wt.) and L-xylose (700 g) were charged to the flask. The reaction was heated to 55±5° C. and stirred for 21 h. TLC (DCM:MeOH=3:1) showed that L-xylose almost disappeared. The reaction was cooled to 25±5° C., and Ag₂CO₃ (0.55 wt.) was charged to the reaction mixture after which the reaction was stirred for 0.5 h at least at 25±5° C. The reaction mixture was filtered through Celite (1 wt.), and the filter cake was rinsed with MeOH (2 v). All the organic solution was combined and concentrated to almost no fraction under vacuum at 40±5° C. to get a residue. The residue was washed with DCM (5 v*3) and concentrated under vacuum at 35±5° C. Acetone (8 v) was charged to the residue, which was used directly in the next step as crude 8.01 (in theory yield).

### Preparation of Compound 8.02

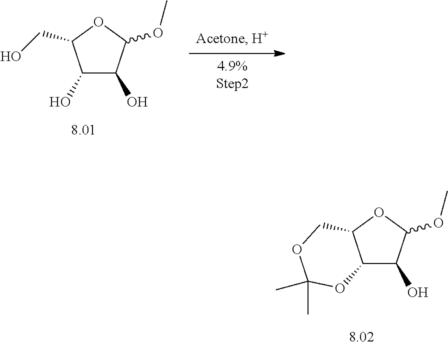

Crude 8.01 in acetone (˜765 g of 8.01) was charged to a 4-necked round bottom flask under an N₂ atmosphere under stirring conditions. CuSO₄ (2.1 wt.) and H₂SO₄ (2N, 16 mL) were charged to the flask. The reaction was heated to 35±5° C. and stirred for 24 h at least at 35±5° C. TLC (DCM:MeOH=5:1) showed 8.01 material. The reaction time was prolonged by 48 h, but there was no obvious increase in 8.02 product and no obvious decrease in 8.01 starting material. The reaction mixture was filtered and rinsed with acetone (3 v). The filtrate was combined and concentrated NH₃ (aq., 26 mL) was added. The solution was concentrated to no obvious fraction under vacuum at 35±5° C. DCM (2 v) and H₂O (1 v) was charged to the residue, and the mixture was stirred for 10 min and left to sit for 10 min before separation. The aqueous phase was extracted with DCM (1 v*2). The organic phase was combined and washed with H₂O (0.15 v). The organic phase was temporarily stored.

The aqueous phase was combined and concentrated to no obvious fraction under vacuum at 55±5° C. The residue was washed with acetone (5 v*3) and concentrated under vacuum at 35±5° C. Acetone (8 v) was charged to the residue which was then transferred to a 4-necked round bottom flask.

The above reaction was repeated three more times, each time starting with the residue from the aqueous phase concentrated in acetone.

All of the organic phase temporarily stored from each reaction were then combined and concentrated to no obvious fraction under vacuum at 35±5° C. The residue was purified through silica gel (100-200 mesh, 10 wt.) and eluted with a solution of (Acetone:DCM=0 to 1:5). The fraction of 8.02 was collected and concentrated to dry under vacuum at 35±5° C. (46.4 g, 4.9%)

### Preparation of Compound 8.03

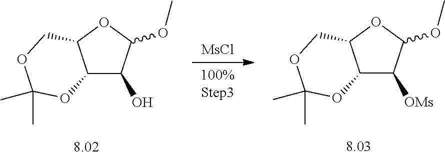

Py (2.7 v) was charged to a 3-necked round bottom flask in an N₂ atmosphere under stirring conditions. 8.02 was then charged to the flask, and CH₃SO₂Cl (0.6 v) was added dropwise to the mixture controlling the temperature at 25±5° C. The reaction was stirred for 3 h. TLC (DCM:Acetone=8:1) showed no 8.02 present. H₂O (14 v) was added to the reaction mixture dropwise at 5±5° C. The mixture was extracted with DCM (7 v*2). The organic phase was combined. The organic phase was swapped with toluene (3.5 v*4) until no fraction under vacuum at 55±5° C. The residue was collected as 8.03 crude (in theory yield) and used in the next step.

### Preparation of Compound 8.04

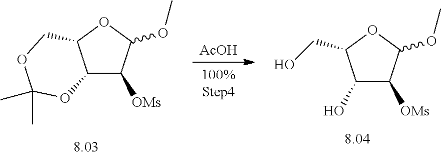

Crude 8.03 in AcOH (1.8 v for 8.02) was charged to a 3-necked round bottom flask under stirring conditions. H₂O (0.75 v for 8.02) was charged to the flask, which was then heated to 55±5° C. The reaction was stirred for 3 h at least at 55±5° C. TLC (DCM:Acetone=8:1) showed no 8.03. Toluene (2 v for 8.02) was charged to the reaction mixture. EtOH (0.4 v for 8.02) was charged to the same mixture to get a clear solution. The clear solution was concentrated to no fraction under vacuum at 55±5° C. Addition of toluene and EtOH followed by concentration was repeated three more times. The residue was collected as 8.04 crude (in theory yield).

### Preparation of Compound 8.05

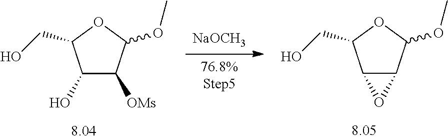

Crude 8.04 in MeOH (3.4 v for 8.02) was charged to a 3-necked round bottom flask in an N₂ atmosphere under stirring conditions. The reaction was heated to 30±5° C. while stirring to get a clear brown solution. The reaction was then cooled to 5±5° C. NaOCH₃ was charged to neutralize the solution and keep the pH>11. The mixture was then heated to 25±5° C. and stirred for at least 16 h keeping the pH>11 and temperature at 25±5° C. TLC (DCM:EA=1:1) showed no 8.04 remaining. The reaction mixture was neutralized with AcOH to pH˜ 7. H₂O was charged to dissolve the precipitated solid. The mixture was extracted with EA (5 v each time) until no 8.05 was found in aqueous phase. All of the organic phase was combined and concentrated to no obvious fraction under vacuum at 40±5° C. to get a residue. The residue was purified through silica gel (100-200 mesh, 10 wt. for 8.02), then eluted with a solution of (EA:DCM=1:50 to 1:1). The pure 8.05 fraction was collected and concentrated under vacuum at 40±5° C. to dry. (25.5 g, 76.8%)

### Preparation of Compound 8.06

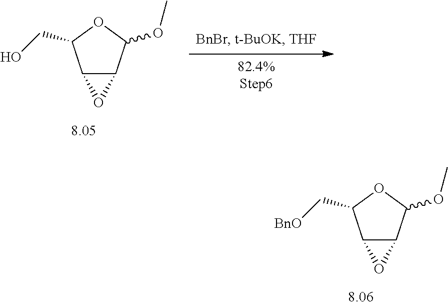

8.05 in THF (10 v) was dissolved in an eggplant-shaped flask. The solution was concentrated to 2 v under vacuum at 40±5° C. THF (8 v) was charged to the residue. KF was sampled and determined to be less than 0.5%. The solution was transferred to a 4-necked round bottom flask in an N₂ atmosphere under stirring conditions. The reaction was cooled to 5±5° C., and t-BuOK (1.1 eq.) was charged to the mixture at 5±5° C. BnBr (1.3 eq) was added dropwise to the reaction mixture, and the temperature was kept at not more than 30° C. The reaction was stirred for 16 h and at a temperature of at least 25±5° C. TLC (EA:DCM=1:1) showed no 8.05 material left. The reaction mixture was concentrated to no obvious fraction under vacuum at 40±5° C. EA (5 v) was charged to the residue, and the mixture was neutralized with AcOH to pH˜ 7. H₂O (5 v) was charged to the mixture. The layers were separated, and the aqueous phase was extracted with EA (5 v). All of the EA phase was combined and concentrated to no obvious fraction under vacuum at 40±5° C. The residue was purified through silica gel (100-200 mesh, 10 wt.) and eluted with (EA:DCM=0 to 1:10). The pure 8.06 fraction was collected and concentrated under vacuum at 40±5° C. to dry. (34 g, 82.4%)

### Preparation of Compound 8.07

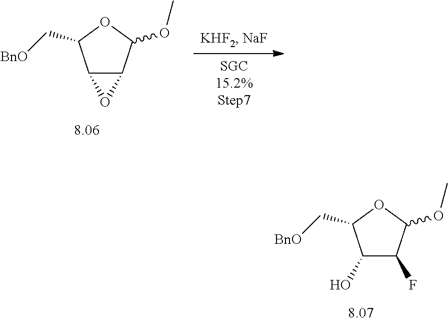

8.06 in DCM (10 v) was dissolved in an eggplant-shaped flask. The solution was concentrated under vacuum at 35±5° C. to no obvious fraction. DCM (10 v) was charged to the residue, and the solution was concentrated under vacuum at 35±5° C. to no obvious fraction. Ethane-1,2-diol (10 v) was charged to the residue. KF was sampled and determined to be less than 0.5%. The mixture was transferred to a 4-necked round bottom flask in an N₂ atmosphere. KHF₂ (3.0 eq.) and NaF (5.6 eq.) were charged to the mixture. The reaction was heated to 165±5° C. and stirred for 2 h. TLC (EA:DCM=1:5) showed no remaining 8.06. The reaction was cooled to 25±5° C. Saturated NaHCO₃ solution (50 v) was added dropwise to the reaction mixture. The organic material was extracted with DCM (20 v*4). All of the DCM solution was combined and concentrated to no fraction under vacuum at 35±5° C. The residue was purified through silica gel (100-200 mesh, 10 wt.) and eluted with a solution of (EA:PE=1:20 to 1:1). The pure 8.07 fraction was collected and concentrated to dry under vacuum at 40±5° C. (5.6 g, 15.2%)

### Preparation of Compound 8.08

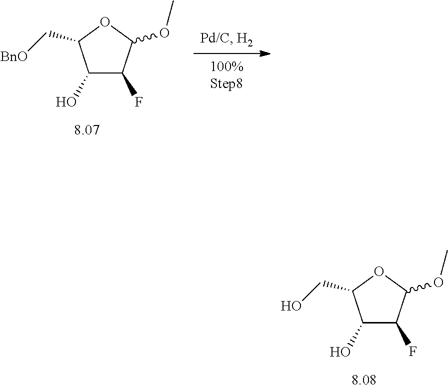 [SMILES: COC1O[C@@H](COCc2ccccc2)[C@@H](O)[C@@H]1F ; "100%Step8" ; "Pd/C, H2" ; COC1O[C@@H](CO)[C@@H](O)[C@@H]1F]

8.07 in EtOH (20 v) was charged into a 4-necked round bottom flask under stirring conditions. The flask was placed under vacuum to ≤−0.08 MPa and then placed under nitrogen atmosphere (repeated three times). Then 10% anhydrous Pd/C (0.2 wt.) was charged to the reaction solution under N₂ protection. Once again, the system was placed under vacuum to ≤−0.08 MPa and then placed under nitrogen atmosphere (repeated three times). The flask was then inflated to an H₂ atmosphere, and the reaction was stirred for 16 h at least at 255° C., keeping the H₂ atmosphere. TLC (EA:PE=1:1) showed no remaining 8.07. The reaction mixture was filtered through Celite (1 wt.) under an N₂ atmosphere. The filter cake was rinsed with EtOH (5 v). The filtrate was combined and concentrated to no fraction under vacuum at 45±5° C. EtOH contained in the residue was swapped with DCM (5 v*5) and was dried under vacuum at 45±5° C. The dry residue was collected as 8.08 (in theory yield).

### Preparation of Compound 8.09

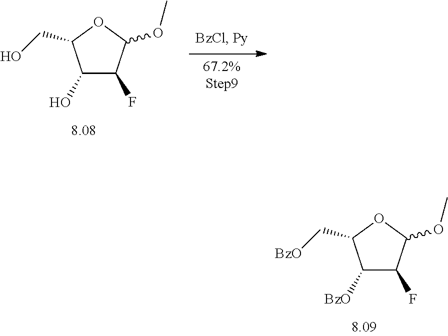

8.08 in Py (26 v) was charged to a 4-necked round bottom flask under an N₂ atmosphere under stirring conditions. The solution was cooled to 5±5° C., and Bz-Cl (2.5 eq.) was added dropwise to the mixture. The reaction was stirred for 2 h at 5±5° C. TLC (EA:PE=1:3) showed no remaining 8.08. The reaction mixture was diluted with DCM (100 v) and washed with H₂O (30 v*2). The organic phase was washed with 10% citric acid solution (30 v*2) and with H₂O (30 v*2). Silica gel (100-200 mesh, 1.5 wt.) was charged to the organic phase. The organic phase was then concentrated to dry under vacuum at 35±5° C. The residue was purified through silica gel (100-200 mesh, 10 wt.) and eluted with a solution of (EA:PE=1:100 to 1:25). The pure 8.09 fraction was collected and concentrated to dry under vacuum at 40±5° C. (5.5 g, 67.2%)

### Preparation of Compound 8.10

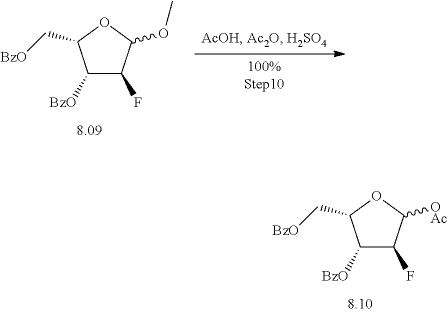

ACOH (9 wt.) was charged to a 3-necked round bottom flask in an N₂ atmosphere under stirring conditions. Ac₂O (9 eq.) was charged to the flask under N₂ protection. The solution was stirred for 5 min. The solution was then charged to dissolve 8.09 resulting in a clear solution. The clear solution was transferred to a 3-necked round bottom flask under N₂ protection under stirring conditions. The reaction was cooled to 5±5° C., then 98% H₂SO₄ (3 eq.) was added dropwise to the mixture at 5±5° C. The reaction was heated to 30±5° C. and stirred for 3 h. TLC (EA:PE=1:5) showed no remaining 8.09. The reaction mixture was added dropwise to saturated NaHCO₃ (500 v) at 5±5° C. The mixture was extracted with DCM (150 v*3). The DCM solution was combined and washed with H₂O (150 v*2). The organic phase was concentrated to no obvious fraction under vacuum at 35±5° C. The water contained in the residue was swapped with DCM (10 v*5) until KF<0.1%. The dry residue was collected as crude 8.10 (in theory yield).

### Preparation of Compounds 8.11-A and 8.11-B

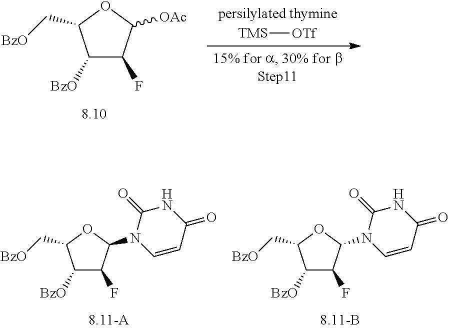

8.10 crude (1 eq.) was dissolved in DCE (20 v) in an N₂ atmosphere. KF was sampled and determined to be less than 0.1%. The solution was transferred to a 4-necked round bottom flask in an N₂ atmosphere under stirring conditions. Uracil (1.1 eq.), HMDS (3.0 eq.), DCE (10 v), and (NH₄)₂SO₄ (0.022 eq.) were charged in turn to another 4-necked round bottom flask under N₂ protection under stirring conditions and heated to 85±5° C. (suspension solution). The solution was stirred for 3 h at 85±5° C., resulting in a clear solution. The clear solution was cooled to 30±5° C. and charged to the 8.10-in-DCE solution under an N₂ atmosphere. TMSOTf (3.3 eq.) was added dropwise to the mixed solution, which was then heated to 85±5° C. and stirred for 3 h. The solution was sampled for LCMS, and no remaining 8.10 was observed. The reaction was cooled to 25±5° C., diluted with DCM (100 v), washed with saturated NaHCO₃ solution (100 v*2), and washed with H₂O (100 v*2). The collected organic phase was concentrated to no fraction under vacuum at 30±5° C. to get a residue. The residue was purified by reverse phase chromatography (5% 8.11 sample in DMSO, C18, Agela-1(HP-Flash-53), ACN-0.05% TFA in water, 30% for 30 min, then 35% to 55% for 40 min). The pure 8.11-A fraction (˜500 v) and 8.11-B fraction (˜1000 v) were collected.

The 8.11-A fraction was extracted with DCM (500 v*2), and this solution was washed with H₂O (200 v*1). The solution was concentrated to no fraction under vacuum at 35±5° C., leaving residue 8.11-A. (1.0 g, 15%) The 8.11-B fraction was extracted with DCM (1000 v*2), and this solution was washed with H₂O (400 v*1). The solution was concentrated to no fraction under vacuum at 35±5° C., leaving residue 8.11-B. (2.0 g, 30%)

### Preparation of Compound 8.12-A

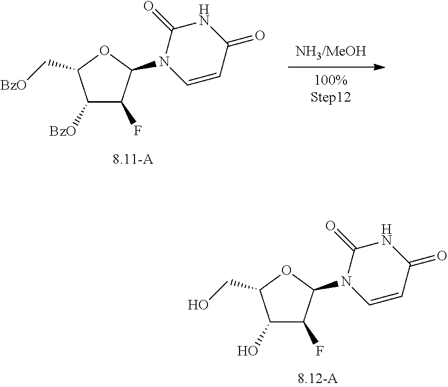

8.11-A (1 eq.) was dissolved in MeOH (58 v) under stirring conditions. NH₃ (aq., 46 wt.) was charged to the solution. The reaction was stirred for 12 h at a temperature of at least 25±5° C. The solution was sampled for LCMS, and no remaining 8.11-A was observed. The reaction solution was concentrated to no obvious fraction under vacuum at 55±5° C. H₂O was removed by azeotropic distillation with MeOH (10 v*5). Residual MeOH was swapped with DCM (20 v*5) under vacuum at 35±5° C. Py (20 v) and DCM (10 v) were charged to the residue. The mixture was concentrated to 20 v under vacuum at 35±5° C. Twice more, DCM (10 v) was charged to the residue, and the mixture was concentrated to 20 v under vacuum at 35±5° C. KF was sampled and determined to be less than 0.05%. The solution was collected as 8.12-A (in theory yield).

### Preparation of Compound 8.12-B

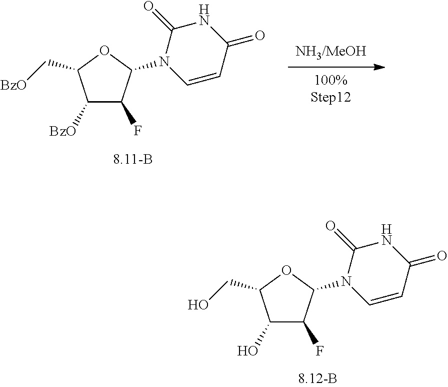

8.11-B (1 eq.) was dissolved in MeOH (58 v) under stirring conditions. NH₃ (aq., 46 wt.) was charged to the solution. The reaction was stirred for 12 h at a temperature of at least 25±5° C. The solution was sampled for LCMS, and no remaining 8.11-B was observed. The reaction solution was concentrated to no obvious fraction under vacuum at 55±5° C. H₂O was removed by azeotropic distillation with MeOH (10 v*5). Residual MeOH was swapped with DCM (20 v*5) under vacuum at 35±5° C. Py (20 v) and DCM (10 v) were charged to the residue. The mixture was concentrated to 20 v under vacuum at 35±5° C. Twice more, DCM (10 v) was charged to the residue, and the mixture was concentrated to 20 v under vacuum at 35±5° C. KF was sampled and determined to be less than 0.05%. The solution was collected as 8.12-B (in theory yield).

### Preparation of Compound 8.13-A

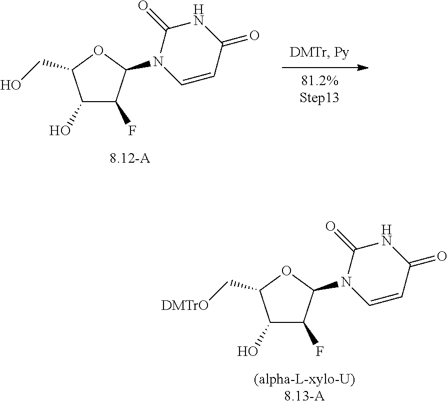

8.12-A in a Py (33 v) solution was transferred to a 4-necked round bottom flask under an N₂ atmosphere under stirring conditions. The reaction was cooled to 5±5° C., and DMTr (0.35 eq. each time, 3 times, total 1.05 eq.) was charged batch-wise at 5±5° C. The reaction was heated to 25±5° C. and stirred for 2 h. TLC (DCM:MeOH=10:1) showed no remaining 8.12-A. The reaction solution was diluted with DCM (100 v), washed with saturated NaHCO₃ (100 v*1), washed with H₂O (100 v*3), and washed with saturated NaCl solution (100 v*1). Silica gel (1.5 wt.) was charged to the organic phase. The mixture was concentrated to dry under vacuum at 35±5° C. leaving a residue. The residue was purified through silica gel (100 mesh, 50 wt.), eluted with PE containing 0.5% TEA, then eluted with a solution of (PE:DCM=1:1) containing 0.5% TEA to remove DMTr and Py, and then eluted with a solution of (DCM:EA=5) containing 0.5% TEA. The target fraction was concentrated to dry under vacuum at 40±5° C., leaving pure residue 8.13-B. (0.98 g, 81.2%)

### Preparation of Compound 8.13-B

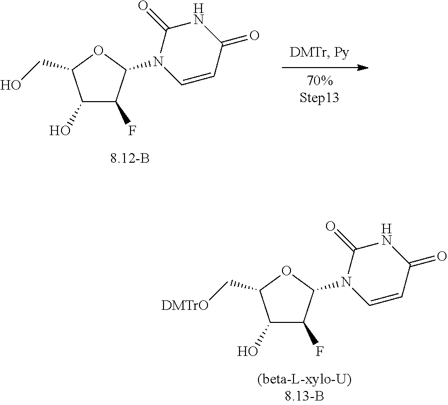

8.12-B in a Py (33 v) solution was transferred to a 4-necked round bottom flask under an N₂ atmosphere under stirring conditions. The reaction was cooled to 5±5° C., and DMTr (0.35 eq. each time, 3 times, total 1.05 eq.) was charged batch-wise at 5±5° C. The reaction was heated to 25±5° C. and stirred for 2 h. TLC (DCM:MeOH=10:1) showed no remaining 8.12-B. The reaction solution was diluted with DCM (100 v), washed with saturated NaHCO₃ (100 v*1), washed with H₂O (100 v*3), and washed with saturated NaCl solution (100 v*1). Silica gel (1.5 wt.) was charged to the organic phase. The mixture was concentrated to dry under vacuum at 35±5° C. leaving a residue. The residue was purified through silica gel (100 mesh, 50 wt.), eluted with PE containing 0.5% TEA, then eluted with a solution of (PE:DCM=1:1) containing 0.5% TEA to remove DMTr and Py, and then eluted with a solution of (DCM:EA=5) containing 0.5% TEA. The target fraction was concentrated to dry under vacuum at 40±5° C., leaving pure residue 8.13-B. (1.69 g, 70%)

### Example 27: Synthesis of Amidites of 2′-Substituted Stereo-Non-Standard Nucleosides Comprising 2′-fluoro-α-D-lyxosyl or 2′-fluoro-β-D-lyxosyl Sugar Moieties

The amidites of 2′-substituted stereo-non-standard nucleosides comprising 2′-fluoro-α-D-lyxosyl or 2′-fluoro-β-D-lyxosyl sugar moieties can be synthesized according to the scheme below.

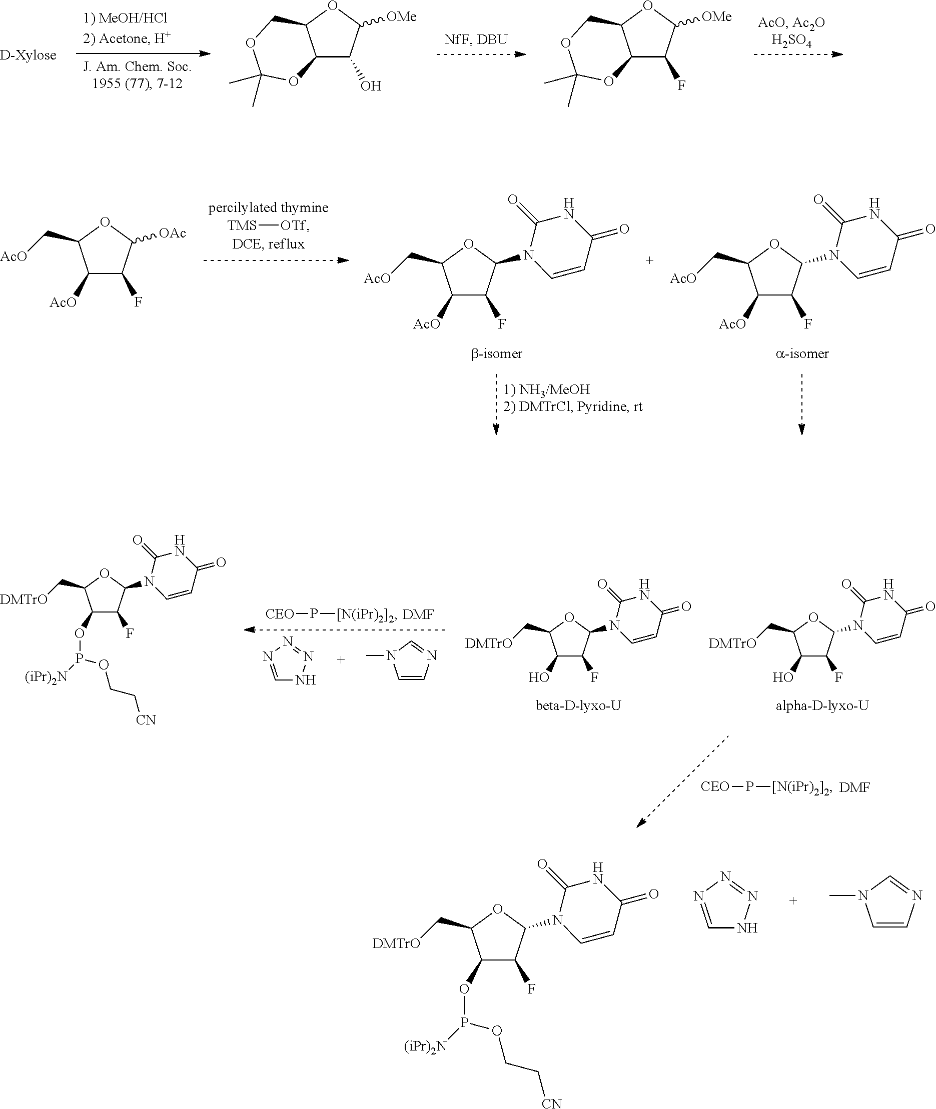

Steps in this synthesis have been previously described, see, e.g., Baker, JACS, 1955.

### Example 28: Synthesis of Amidites of 2′-Substituted Stereo-Non-Standard Nucleosides Comprising 2′-fluoro-α-L-lyxosyl or 2′-fluoro-β-L-lyxosyl Sugar Moieties

The amidites of 2′-substituted stereo-non-standard nucleosides comprising 2′-fluoro-α-L-lyxosyl or 2′-fluoro-(3-L-lyxosyl sugar moieties can be synthesized according to the scheme below:

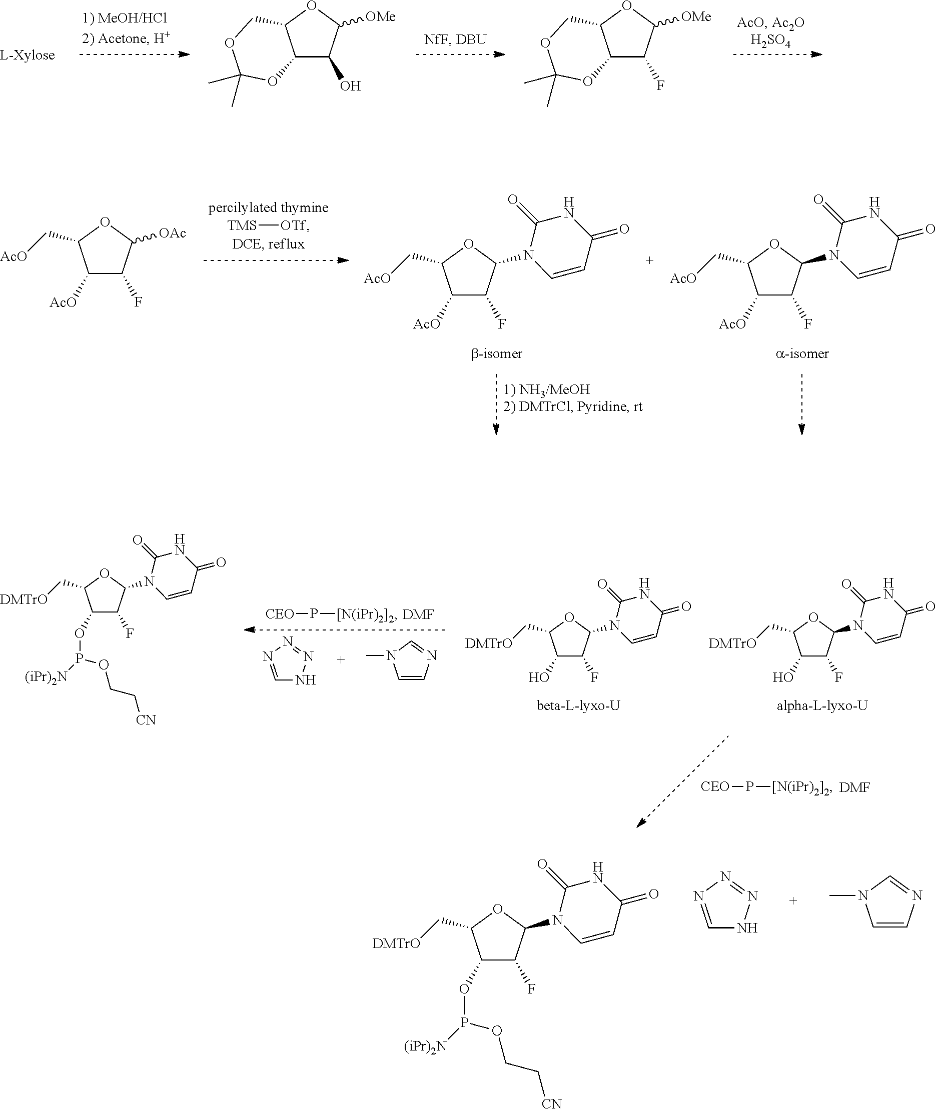

### Example 29: Synthesis of an Amidite of a 2′-Substituted Stereo-Non-Standard Nucleoside Comprising a 2′-O-methyl-α-L-arabinosyl Sugar Moiety

Compound 9a, the amidite of a 2′-substituted stereo-non-standard nucleoside comprising a 2′-O-methyl-α-L-arabinosyl sugar moiety was synthesized according to the scheme below:

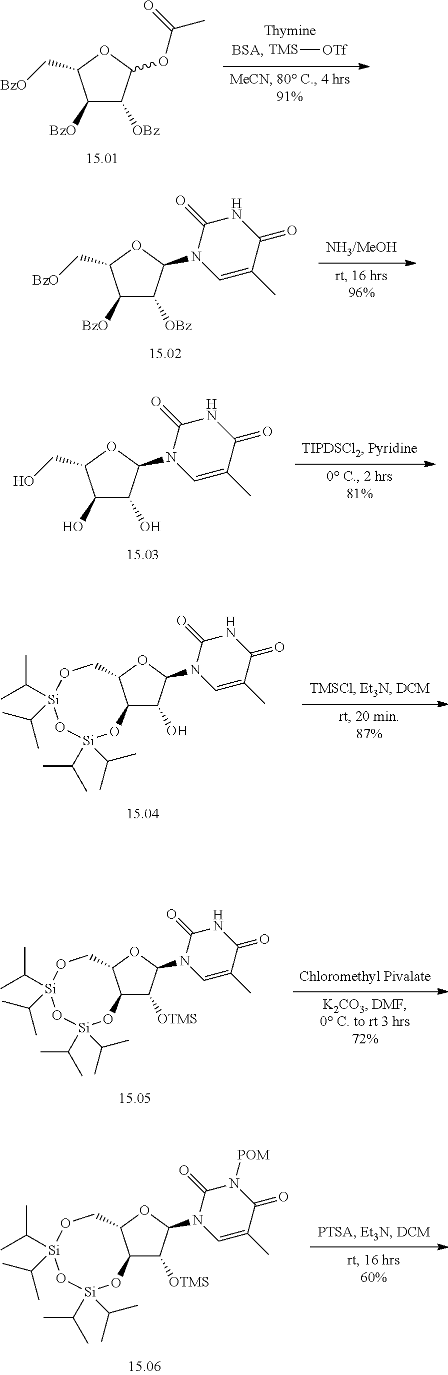

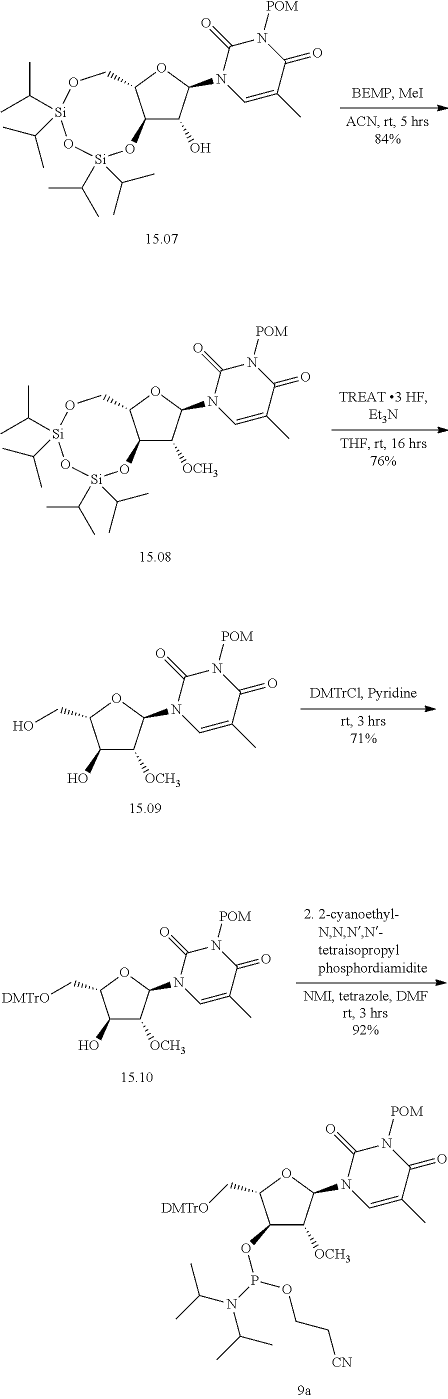

Steps of this synthesis have been previously described, see, e.g., Grotli, Tetrahedron, 1997; Grotli, Tetrahedron, 1999.

### Preparation of Compound 15.02

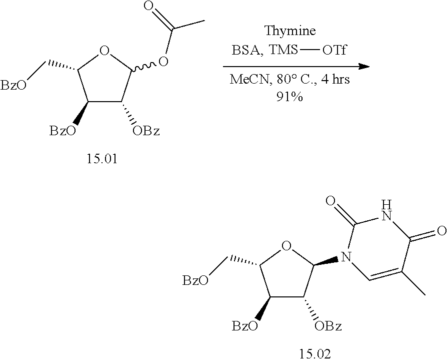

Thymine (5.25 g, 3.49 mmol, 1.5 eq) and N,O-Bis(trimethylsilyl)acetamide (20.4 mL, 83.4 mmol, 3.0 eq) were added to a solution of [(2S,3S,4R)-5-acetoxy-3,4-dibenzoyloxy-tetrahydrofuran-2-yl]methyl benzoate (15.01) (14.0 g, 27.8 mmol) in acetonitrile (140 mL). After heating at 80° C. for 15 minutes to obtain a clear solution, trimethylsilyl trifluoromethanesulfonate (3.53 mL, 36.1 mmol, 1.3 eq) was added, and the reaction was stirred overnight at 80° C. The reaction was concentrated under reduced pressure and diluted with ethyl acetate. The organics were washed with saturated sodium bicarbonate solution and brine, followed by concentration to an oil under reduced pressure. Purification by Biotage (Si, 100 g col, 50% ethyl acetate/hexanes) afforded the desired product (15.02) as a white solid. (14.5 g, 91% yield)

### Preparation of Compound 15.03

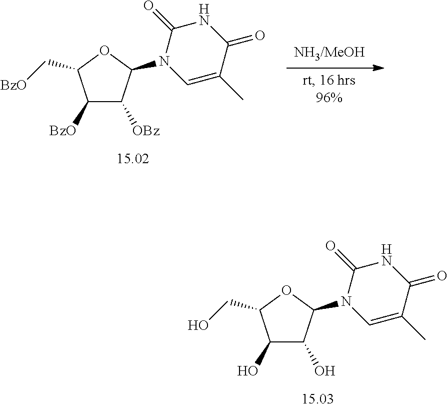

NH₃ (7.00 M, 30 mL, 210 mmol) in methanol was added to [(2S,3S,4R,5R)-3,4-dibenzoyloxy-5-(5-methyl-2,4-dioxo-pyrimidin-1-yl)tetrahydrofuran-2-yl]methyl benzoate (15.02) (14.5 g, 25.4 mmol). The reaction was heated at 45° C. for 16 hours. The reaction was concentrated to an oil and purification by Biotage (Si, 25 g col, 0-20% methanol/dichloromethane) afforded the desired product (15.03) as a white solid. (6.30 g, 96% yield)

### Preparation of Compound 15.04

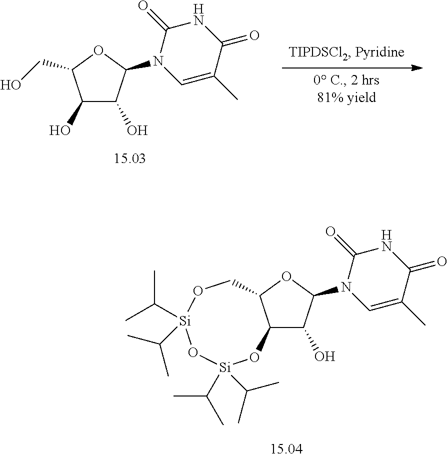 [SMILES: Cc1cn([C@@H]2O[C@@H](CO)[C@H](O)[C@H]2O)c(=O)[nH]c1=O ; "0° C., 2 hrs81% yield" ; "TIPDSCl2, Pyridine" ; Cc1cn([C@@H]2O[C@H]3CO[Si](C(C)C)(C(C)C)O[Si](C(C)C)(C(C)C)O[C@@H]3[C@H]2O)c(=O)[nH]c1=O]

1-((2R,3R,4R,5S)-3,4-dihydroxy-5-(hydroxymethyl)tetrahydrofuran-2-yl)-5-methylpyrimidine-2,4(1H,3H)-dione (15.03) (6.30 g, 24.4 mmol) was dissolved in pyridine (50 mL) then cooled down with an ice bath to 0° C. 1,3-Dichloro-1,1,3,3-tetraisopropyldisiloxane (7.73 mL, 24.2 mmol, 0.99 eq) was added dropwise. The reaction was stirred for 2 hours. TLC in EtOAc/hexane (6/4) indicated that the reaction was complete. The reaction was quenched by slowly adding water (10 mL) at 0° C. and stirred for 10 minutes. The reaction solution was diluted with EtOAc (50 mL), transferred to a separatory funnel. The reaction solution was washed first with plain DI water, sat. NaHCO₃ solution, and sat. brine. The organic layer was finally dried over Na₂SO₄ and filtered and concentrated to afford a crude oil. Purification by Biotage (Si, 100 g col, 6% EtOAc/hexane) to afford the desired product (15.04) as a white solid. (10 g, 68% yield)

### Preparation of Compound 15.05

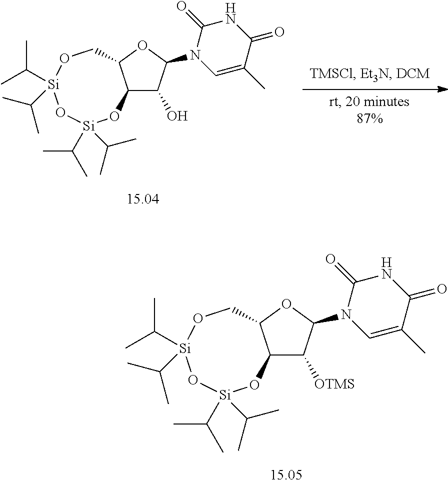

Compound 1-((6aS,8R,9R,9aR)-9-hydroxy-2,2,4,4-tetraisopropyltetrahydro-6H-furo[3,2-f][1,3,5,2,4]trioxadisilocin-8-yl)-5-methylpyrimidine-2,4(1H,3H)-dione (15.04) (10 g, 20 mmol) was dissolved in dry dichloromethane (100 mL). Triethylamine (9.74 mL, 69.90 mmol, 3.5 eq) and chloro(trimethyl)silane (6.34 mL, 49.90 mmol 2.5 eq) were added dropwise at room temperature under nitrogen. The reaction mixture was left at room temperature for 20 minutes at room temperature. TLC in hexane/EtOAc (8/2) indicated reaction was completed. The reaction mixture was poured into vigorously stirred 1 M NaHCO₃ solution (50 mL). The organic layer was separated, dried (using Na₂SO₄), and filtered and evaporated to dryness under reduced pressure. Without any further purification, the crude material was dried under high vacuum and used for the next step. (10.0 g, 87% yield)

### Preparation of Compound 15.06

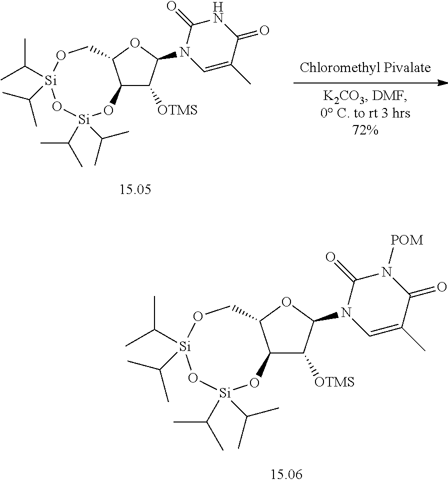

Compound 5-methyl-1-((6aS,8R,9R,9aS)-2,2,4,4-tetraisopropyl-9-((trimethylsilyl)oxy)tetrahydro-6H-furo[3,2-f][1,3,5,2,4]trioxadisilocin-8-yl)pyrimidine-2,4(1H,3H)-dione (15.05) (10 g, 17.50 mmol) was dissolved in dry dimethylformamide (70 mL) and potassium carbonate (6.34 mL, 87.30 mmol, 5 eq). Chloromethyl pivalate (6.6.29 mL, 43.60 mmol 2.5 eq) was added dropwise at room temperature under nitrogen. The reaction mixture was stirred at room temperature overnight. TLC in hexane/EtOAc (8/2) indicated reaction was completed. The solvent was removed under reduced pressure, and the crude material was dissolved in 50 mL ethyl acetate and the solution was washed with 1 M NaHCO₃ solution (50 mL). The organic layer was separated, dried (using Na₂SO₄), filtered, and evaporated to dryness under reduced pressure. Purification by Biotage (Si, 100 g col, 80% hexane/EtOAc) afforded the desired product (15.06) as a colorless oil. (8.70 g, 72% yield)

### Preparation of Compound 15.07

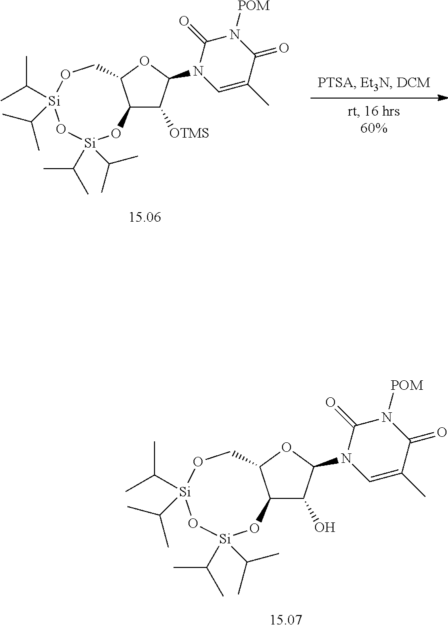

Compound (5-methyl-2,6-dioxo-3-((6aS,8R,9R,9aS)-2,2,4,4-tetraisopropyl-9-((trimethylsilyl)oxy)tetrahydro-6H-furo[3,2-f][1,3,5,2,4]trioxadisilocin-8-yl)-3,6-dihydropyrimidin-1(2H)-yl)methyl pivalate (15.06) (8.70 g, 12.10 mmol) was dissolved in dry dichloromethane (80 mL) and treated with solution of p-toluenesulfonic acid (PTSA) (5.22 g, 30.30 mmol, 2.5 eq) in tetrahydrofuran (20 mL). The reaction was stirred under nitrogen for 20 minutes. TLC in hexane/EtOAc (8/2) indicated reaction was completed. Reaction was quenched by addition of triethylamine to pH 7. The reaction mixture was poured into vigorously stirred 1 M NaHCO₃ solution (50 mL). The organic layer was separated, dried (using Na₂SO₄), and filtered and evaporated to dryness under reduced pressure. The crude material was purified by Biotage (Si, 220 g col, 5-15% EtOAc hexane) to afford the desired product (15.07) as a white solid. (4.80 g, 64% yield)

### Preparation of Compound 15.08

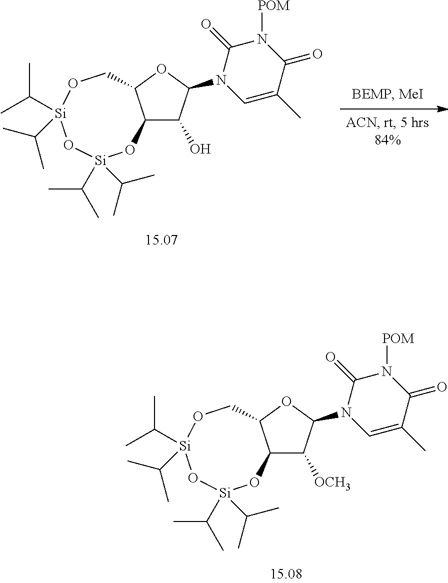

Compound (3-((6aS,8R,9R,9aR)-9-hydroxy-2,2,4,4-tetraisopropyltetrahydro-6H-furo[3,2-f][1,3,5,2,4]trioxadisilocin-8-yl)-5-methyl-2,6-dioxo-3,6-dihydropyrimidin-1(2H)-yl)methyl pivalate (15.07) (4.70 g, 7.64 mmol) was dissolved in anhydrous acetonitrile (38 mL) under nitrogen. 2-(tert-butylimino)-N,N-diethyl-1,3-dimethyl-1,3,215-diazaphosphinan-2-amine (BEMP) (4.19 g, 15.30 mmol, 2 eq) was added at room temperature followed immediately by Iodomethane (3.25 g, 22.90 mmol, 3.0 eq). The reaction mixture was stirred at room temperature for 5 hours. TLC in hexane/EtOAc (8/2) indicated reaction was completed. The reaction mixture was quenched with methanol (3 mL) and poured into a separatory funnel and washed with plain DI water. The organic layer was extracted with ethyl acetate and washed with sat. NaHCO₃ and sat. brine. The organic layer was dried over NaSO₄ for 10 minutes. The organic material was filtered and evaporated to dryness under reduced pressure. The crude material was purified by Biotage (Si, 220 g col, 5-20% EtOAc/hexane) to afford the desired product (15.08) as a colorless oil. (4.30 g, 84% yield)

### Preparation of Compound 15.09

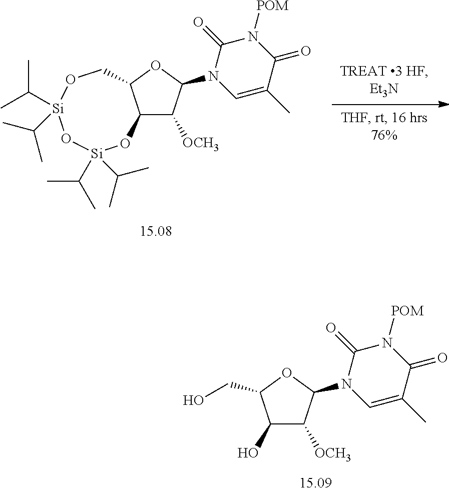

TEA (2.38 mL, 17.10 mmol, 2.5 eq) was added to a solution of (5-methyl-2,6-dioxo-3-((6aS,8R,9R,9aS)-2,2,4,4-tetraisopropyl-9-methoxytetrahydro-6H-furo[3,2-f][1,3,5,2,4]trioxadisilocin-8-yl)-3,6-dihydropyrimidin-1(2H)-yl)methyl pivalate (15.08) (4.30 g, 6.84 mmol) in THF (34 mL). The reaction was cooled to 0° C. with an ice bath under an atmosphere of nitrogen. Triethylamine trihydrofluoride (5.57 mL, 34.2 mmol, 5 eq) was added slowly at 0° C. and then the reaction was warmed to room temperature and stirred overnight. TLC in hexane/EtOAc (8/2) indicated reaction was completed. Reaction was stopped by adding 2.5 mL of Et₃N, and the solvent was evaporated to dryness under reduced pressure to obtain a white solid. The crude material was dissolved in EtOAc and washed with plain DI water. The aqueous layer was removed, and the organic layer was washed with sat. NaHCO₃ and sat. brine solution. The organic material was dried over NaSO₄, and filtered and evaporated to dryness under reduced pressure. The crude material was purified by Biotage (Si, 10 g col, 0-8% methanol/dichloromethane) to afford the desired product (15.09) as a white solid. (2 g, 76% yield)

### Preparation of Compound 15.10

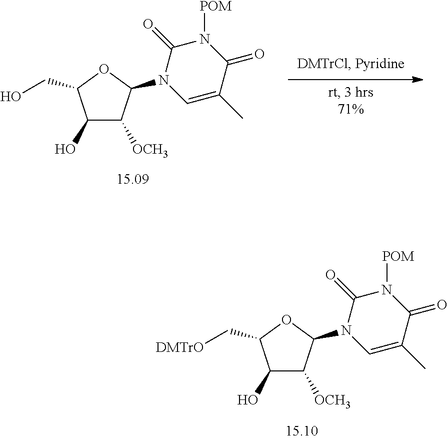

DMTrCl (1.49 g, 4.49 mmol) was added to a solution of (3-((2R,3R,4S,5S)-4-hydroxy-5-(hydroxymethyl)-3-methoxytetrahydrofuran-2-yl)-5-methyl-2,6-dioxo-3,6-dihydropyrimidin-1(2H)-yl)methyl pivalate (15.09) (1.7 g, 4.40 mmol) in pyridine (15 mL) at room temperature and stirred for 3 hours. TLC in EtOAc/hexane (4/6) indicated the reaction was completed. The solution was transferred into a separatory funnel and diluted with ethyl acetate (50 mL) and washed with DI water (2×50 mL). The aqueous layer was removed. The organic layer was washed with sat. NaHCO₃, sat. brine dried over Na₂SO₄, filtered, and evaporated solvent under reduced pressure to obtain crude material. The crude material was dissolved in DCM and loaded to silica gel chromatography. Biotage (Si, 50 g col, 40-100% EtOAc/hexane+1% Et₃N) afforded the desired product (15.10) as a white solid. (2.37 g, 71% yield)

### Preparation of Compound 9a

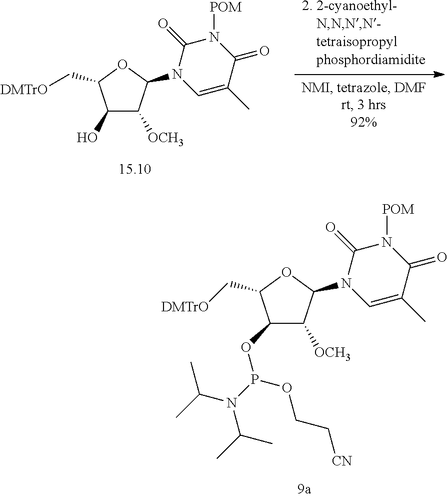

1H-Tetrazole (190 mg, 2.75 mmol, 0.8 eq) and 1-methylimidazole (68.2 μL, 860 μmol, 0.25 eq) were added to a solution of (3-((2R,3R,4S,5S)-5-((bis(4-methoxyphenyl)(phenyl)methoxy)methyl)-4-hydroxy-3-methoxytetrahydrofuran-2-yl)-5-methyl-2,6-dioxo-3,6-dihydropyrimidin-1(2H)-yl)methyl pivalate (15.10) (2.37 g, 3.44 mmol) in anhydrous dimethylformamide (35 mL) at room temperature under an atmosphere of nitrogen. 3-bis(diisopropylamino)phosphanyloxypropanenitrile (1.64 mL, 5.165 mmol, 1.5 eq) was then added dropwise, and the reaction was stirred at room temperature for 3 hours. TLC in hexane/EtOAc (1/1) indicated reaction was completed. The reaction solution was transferred to a separatory funnel and diluted by adding a 3:1 mixture of toluene/hexanes (30 mL). The organic layer was washed (4×30 mL) with a 3:2 mixture of DMF/H₂O. The organic layer was washed with saturated sodium bicarbonate solution, brine, dried over solid sodium sulfate and concentrated under reduced pressure to a white foam. Purification by Biotage (Si, 25 g col, 50-70% ethyl acetate/hexanes+1% triethylamine) afforded the desired product (9a) as a white solid. (2.80 g, 92% yield)

### Example 30: Synthesis of an Amidite of a 2′-Substituted Stereo-Non-Standard Nucleoside Comprising a 2′-O-methyl-β-D-arabinosyl Sugar Moiety

Compound 16.10, the amidite of a 2′-substituted stereo-non-standard nucleoside comprising 2′-O-methyl-β-D-arabinosyl sugar moiety was synthesized according to the scheme below:

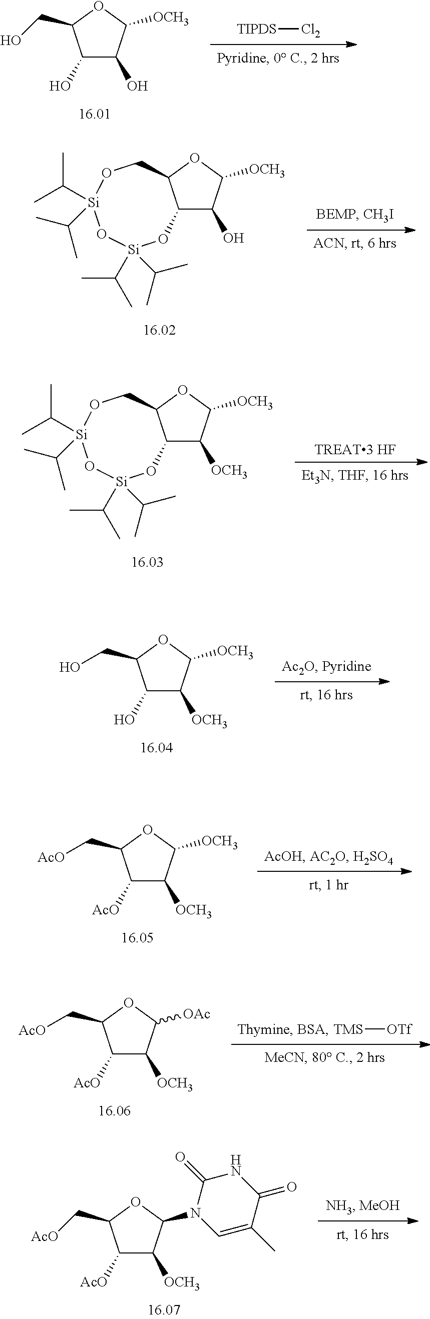

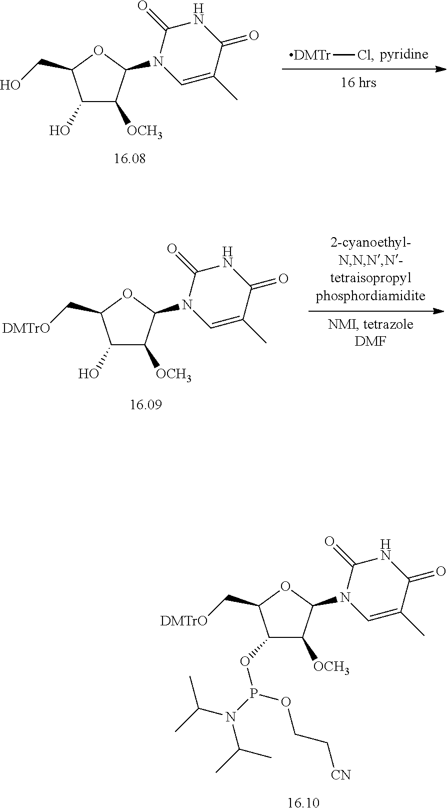

Compound 16.10 was synthesized according to previously described methods (Gotfredsen, C. H. et al., Bioorganic & Medicinal Chemistry, Vol. 4, No. 8, pp. 1217-1225, 1996; Grotli, M. et al., Tetrahedron. Vol. 55, pp. 4299-4314, 1999).

### Example 31: Synthesis of an Amidite of a 2′-Substituted Stereo-Non-Standard Nucleoside Comprising a 2′-O-methyl-β-D-xylosyl Sugar Moiety

Compound 17.07, the amidite of a 2′-substituted stereo-non-standard nucleoside comprising 2′-O-methyl-β-D-xylosyl sugar moiety was synthesized according to the scheme below:

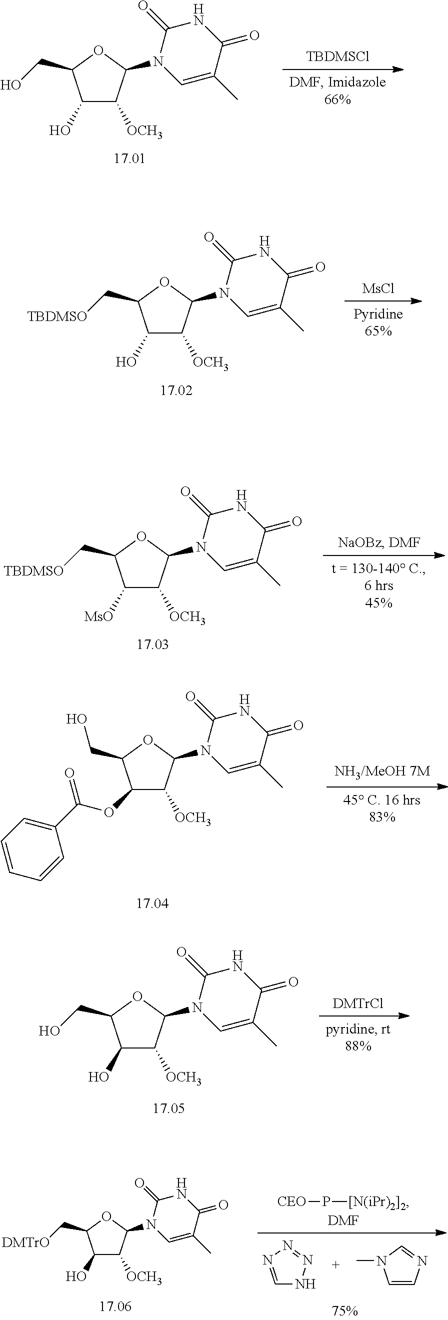

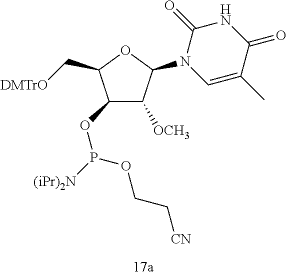

Steps of this synthesis have been previously described, see, e.g., Yung, JACS, 1961.

### Preparation of Compound 17.02

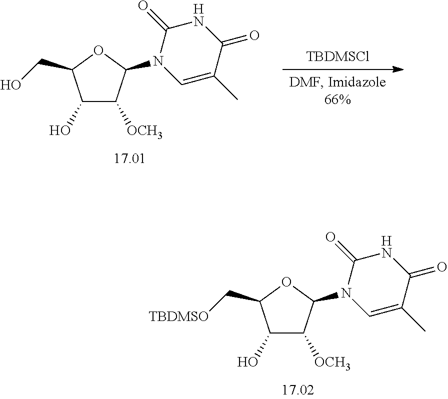 [SMILES: CO[C@@H]1[C@H](O)[C@@H](CO)O[C@H]1n1cc(C)c(=O)[nH]c1=O ; "TBDMSCl" ; "DMF, Imidazole66%" ; CO[C@@H]1[C@H](O)[C@@H](CO[Si](C)(C)C(C)(C)C)O[C@H]1n1cc(C)c(=O)[nH]c1=O]

Compound 1-((2R,3R,4R,5R)-4-hydroxy-5-(hydroxymethyl)-3-methoxytetrahydrofuran-2-yl)-5-methylpyrimidine-2,4(1H,3H)-dione (17.01) (5.0 g, 18.44 mmol) was dissolved in anhydrous dimethylformamide (100 mL), and the solution was stirred under nitrogen. 1H-Imidazole (2.50 g, 36.7 mmol, 2 eq.) was added, then the solution was cooled in an ice bath to 0° C. Tert-butylchlorodimethylsilane (2.49 g, 16.5 mmol, 1.0 eq) in a solution of anhydrous dimethylformamide (10 mL) was added dropwise. The ice bath was removed, and the reaction was warmed up at room temperature stirred for 3 hours. TLC in hexane/EtOAc (6/4) indicated reaction was completed.

The solution was cooled in an ice bath to 0° C., and the reaction was slowly quenched by adding 30 mL of water. The solution was transferred to a separatory funnel, and washed with plain DI water. The product was extracted with ethyl acetate. The aqueous layer was removed from the organic layer. The organic layer was washed with sat. NaHCO₃ and sat. brine, dried over Na₂SO₄, and filtered and evaporated solvent to obtain a crude oil. The crude material was dissolved in dichloromethane and loaded to a plug of silica gel (50 g) and eluted with EtOAc/hexane (6/4) to obtain the product (17.02) at 6.70 g, 66% yield.

### Preparation of Compound 17.03

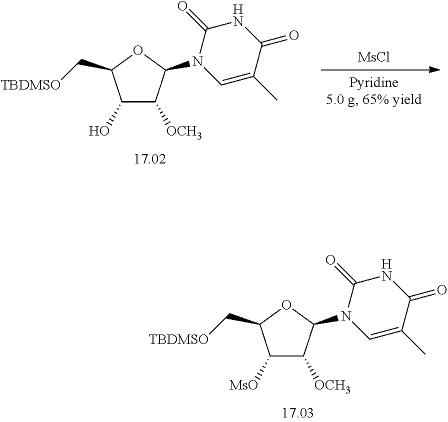 [SMILES: "MsCl" ; CO[C@@H]1[C@H](O)[C@@H](CO[Si](C)(C)C(C)(C)C)O[C@H]1n1cc(C)c(=O)[nH]c1=O ; "Pyridine5.0 g, 65% yield" ; CO[C@@H]1[C@H](OS(C)(=O)=O)[C@@H](CO[Si](C)(C)C(C)(C)C)O[C@H]1n1cc(C)c(=O)[nH]c1=O]

Compound 1-((2R,3R,4R,5R)-5-(((tert-butyldimethylsilyl)oxy)methyl)-4-hydroxy-3-methoxytetrahydrofuran-2-yl)-5-methylpyrimidine-2,4(1H,3H)-dione (17.02) (5.0 g, 18.44 mmol) was dissolved in anhydrous pyridine (50 mL), and the solution was stirred under nitrogen. The solution was cooled with an ice bath to 0° C. and methanesulfonyl chloride (0.933 mL, 12.1 mmol, 1.0 eq) was added dropwise. The ice bath was removed, and the reaction was warned up to room temperature and stirred overnight. The next day, TLC in hexane/EtOAc (6/4) indicated reaction was completed.

The reaction was slowly quenched by adding the reaction solution into 50 mL ice crystals while stirring vigorously. The solution was transferred to a separatory funnel, and the product was extracted with ethyl acetate (50 mL). The organic layer was washed with sat. NaHCO₃ and sat. brine. The organic layer was finally dried over Na₂SO₄ and filtered. The solvent was evaporated to obtain a crude oil. The crude material was dissolved in a minimum amount of dichloromethane, and 500 mL of hexane was slowly added to obtain white precipitate. The solid was collected and dried under high vacuum to obtain the product (17.03) at 5.0 g, 89% yield.

### Preparation of Compound 17.04

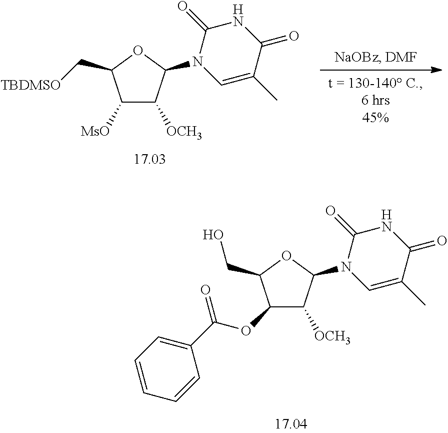

Compound (2R,3R,4R,5R)-2-(((tert-butyldimethylsilyl)oxy)methyl)-4-methoxy-5-(5-methyl-2,4-dioxo-3,4-dihydropyrimidin-1(2H)-yl)tetrahydrofuran-3-yl methanesulfonate (17.03) (4.0 g, 8.61 mmol) was added to a solution of sodium benzoate (14.27 g, 99.0 mmol, 11.5 eq) in anhydrous dimethylformamide (200 mL). Using a mechanical stirrer, the reaction was stirred for 6 hours at 130° C. to 140° C. TLC in DCM/MeOH (9/1) indicated reaction was completed with minor side product. The mixture was cooled down to room temperature, diluted with DI water (200 mL). The product was extracted with ethyl acetate (3×50 mL). The organic layer was washed with sat. NaHCO₃ and sat. brine. The organic layer was dried over Na₂SO₄ and filtered. The solvent was evaporated to obtain crude oil. Purification by Biotage (Si, 50 g col, 0-10% MeOH/DCM) afforded the desired product (17.04) as a white solid. (1.35 g, 45% yield)

### Preparation of Compound 17.05

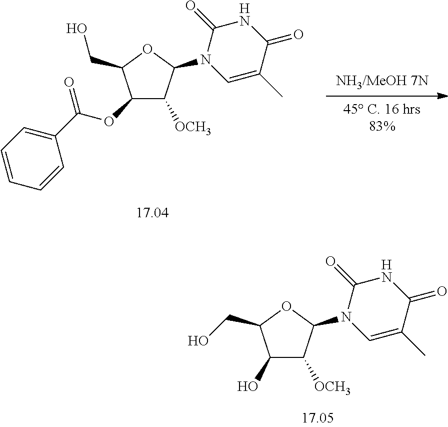 [SMILES: CO[C@@H]1[C@@H](OC(=O)c2ccccc2)[C@@H](CO)O[C@H]1n1cc(C)c(=O)[nH]c1=O ; "45° C. 16 hrs83%" ; "NH3/MeOH 7N" ; CO[C@@H]1[C@@H](O)[C@@H](CO)O[C@H]1n1cc(C)c(=O)[nH]c1=O]

(2R,3S,4R,5R)-2-(hydroxymethyl)-4-methoxy-5-(5-methyl-2,4-dioxo-3,4-dihydropyrimidin-1(2H)-yl)tetrahydrofuran-3-yl benzoate (17.04) (1.33 g, 3.53 mmol) in a 7 N NH₃ methanol solution (20 mL) was heated at 45° C. overnight. The next day, TLC in DCM/MeOH (9/1) indicated reaction was completed. Solvent was evaporated under reduced pressure. Material was dissolved in DCM/MeOH (95/5) and load to column. Biotage (Si, 10 g col, 0-10% DCM/MeOH) afforded the desired product (17.05) as a white solid. (800 mg, 83% yield)

### Preparation of Compound 17.06

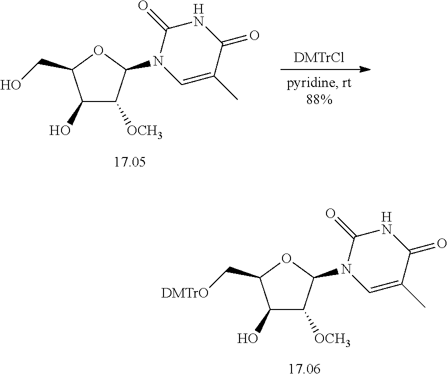

DMTrCl (1.10 g, 3.23 mmol, 1.10 eq) was added to a solution of 1-((2R,3R,4S,5R)-4-hydroxy-5-(hydroxymethyl)-3-methoxytetrahydrofuran-2-yl)-5-methylpyrimidine-2,4(1H,3H)-dione (17.05) (800 mg, 2.94 mmol), in pyridine (10 mL) at room temperature and stirred for 3 hours. TLC in EtOAc/hexane (9/12) indicated reaction was completed. Solution was transferred into a separatory funnel and diluted with ethyl acetate (50 mL) and washed with DI water (2×50 mL). The aqueous layer was removed. The organic layer was washed with sat. NaHCO₃, sat. brine, then dried over Na₂SO₄ and filtered. The solvent was evaporated under reduced pressure to obtain crude material. The crude material was dissolved in DCM and loaded to silica gel chromatography. Biotage (Si, 50 g col, 0-5% MeOH/DCM+1% Et₃N) afforded the desired product (17.06) as a white solid. (1.5 g, 89% yield)

### Preparation of Compound 17a

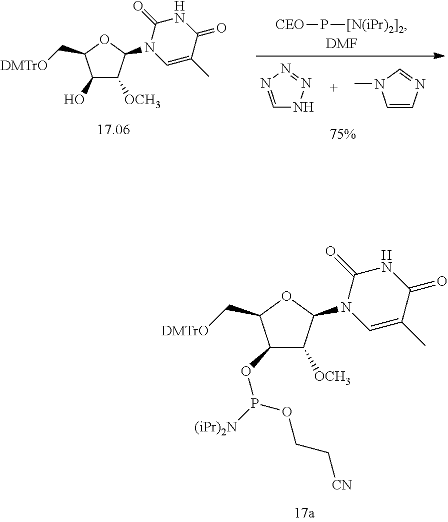

1H-Tetrazole (144.20 mg, 2.09 mmol, 0.8 eq) and 1-methylimidazole (46.82 μL, 587.33 μmol, 0.25 eq) were added to a solution of 1-((2R,3R,4S,5R)-5-((bis(4-methoxyphenyl)(phenyl)methoxy)methyl)-4-hydroxy-3-methoxytetrahydrofuran-2-yl)-5-methylpyrimidine-2,4(1H,3H)-dione (17.06) (1.5 g, 2.61 mmol) in anhydrous dimethylformamide (15 mL) at room temperature under an atmosphere of nitrogen. 3-bis(diisopropylamino)phosphanyloxypropanenitrile (1.24 mL, 3.92 mmol, 1.5 eq) was then added dropwise and the reaction was stirred at room temperature for 3 hours. The reaction solution was transferred to a separatory funnel and diluted by adding a 3:1 mixture of toluene/hexanes (30 mL). The organic layer was washed (4×30 mL) with a 3:2 mixture of DMF/H₂O. The organic layer was then washed with saturated sodium bicarbonate solution and brine, dried over solid sodium sulfate, and concentrated under reduced pressure to a white foam. Purification by Biotage (Si, 25 g col, 50-70% ethyl acetate/hexanes+1% triethylamine) afforded the desired product (17a) as a white solid (1.5 g, 75% yield)

### Example 32: Synthesis of an Amidite of a 2′-Substituted Stereo-Non-Standard Nucleoside Comprising a 2′-O-methyl-α-D-arabinosyl Sugar Moiety

Compound 18.10, the amidite of a 2′-substituted stereo-non-standard nucleoside comprising 2′-O-methyl-α-D-arabinosyl sugar moiety was synthesized according to the scheme below. These methods have been previously described (Gotfredsen, C. H. et al., Bioorganic & Medicinal Chemistry, Vol. 4, No. 8, pp. 1217-1225, 1996; Grotli, M. et al., Tetrahedron. Vol. 55, pp. 4299-4314, 1999).

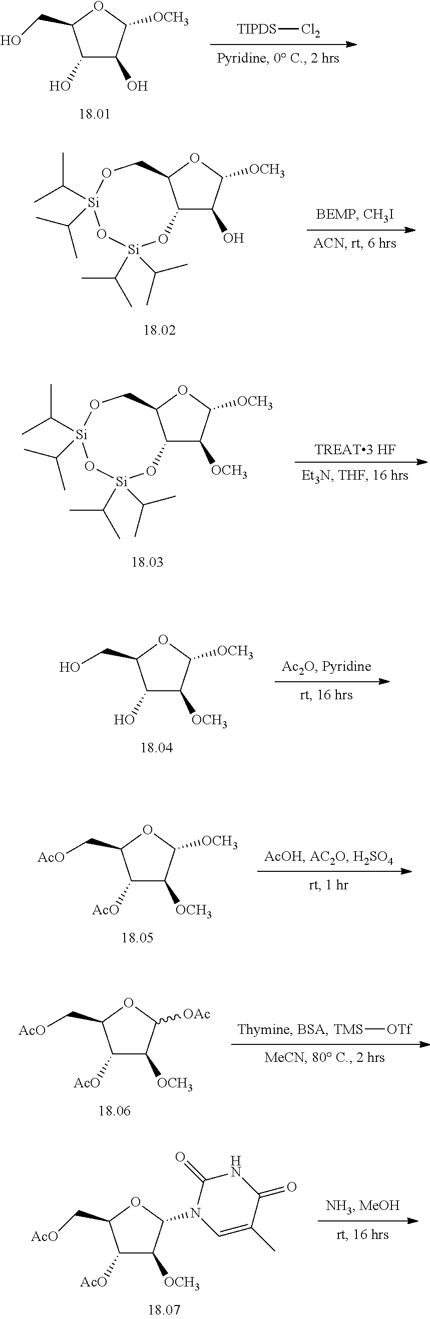

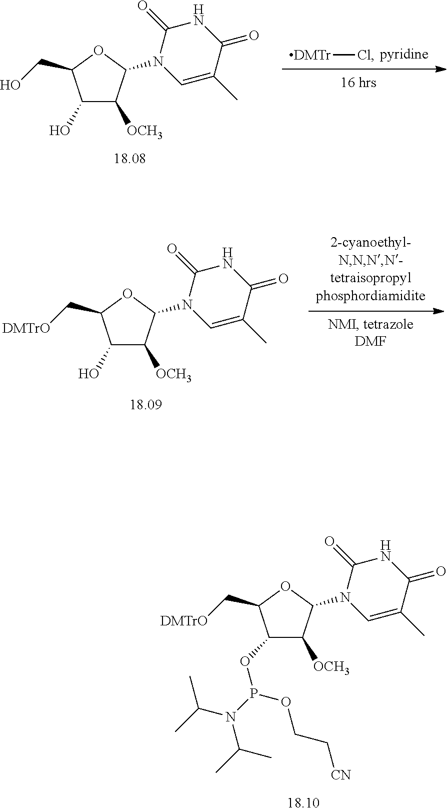

### Example 33: Synthesis of an Amidite of a Stereo-Non-Standard Nucleoside Comprising a 2′-α-L-Deoxyribosyl Sugar Moiety

Compound 1a, the amidite of a stereo-non-standard nucleoside comprising 2′-α-L-deoxyribosyl sugar moiety was synthesized according to the scheme below.

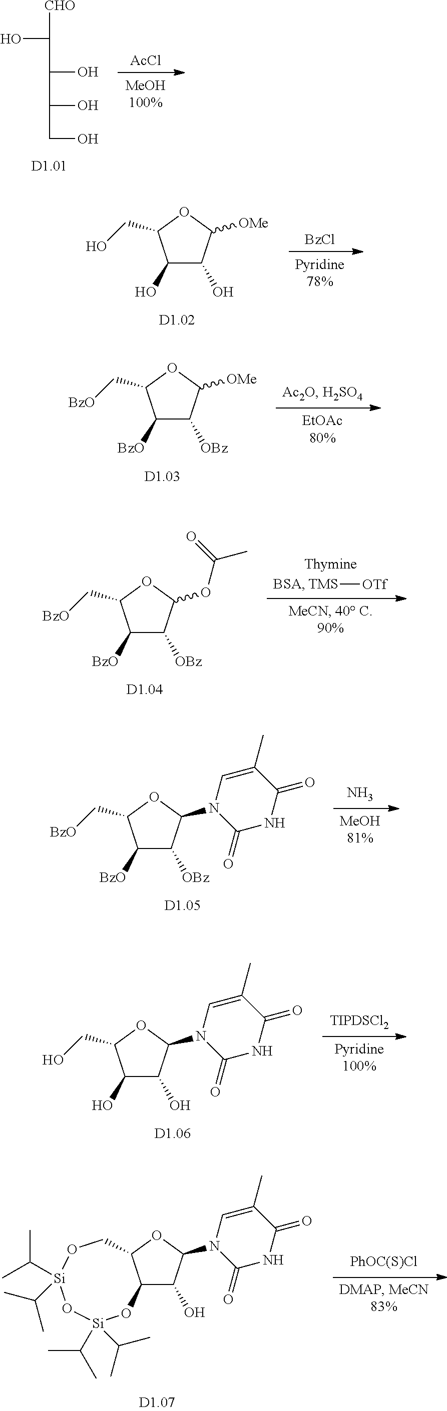

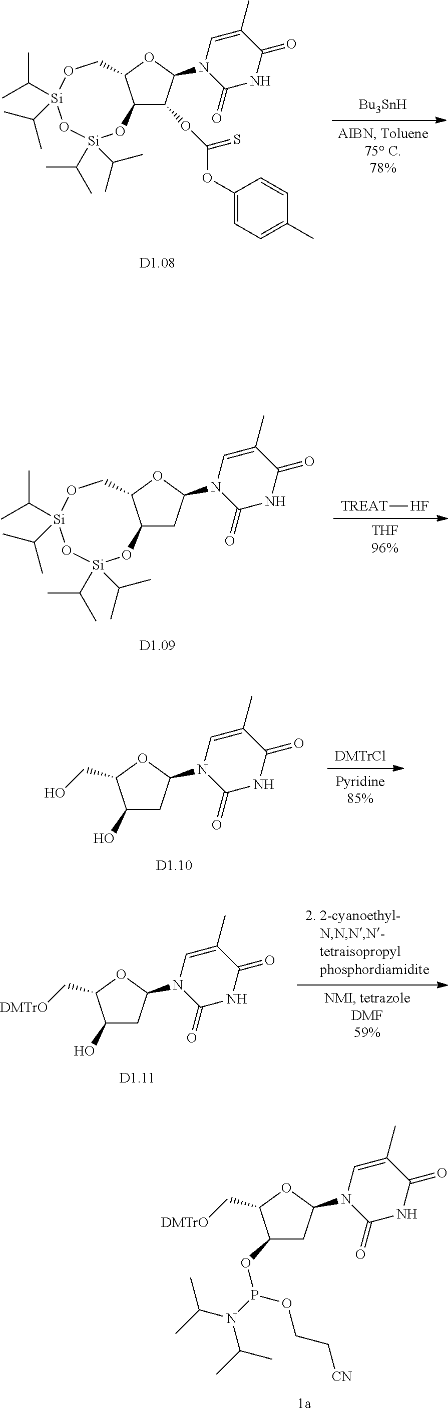

Steps of this synthesis and similar syntheses have been previously described, see, e.g., Dangerfield, et. al., Carbohydrate Res., 2010; Callam, et al., J. Chem. Educ., 2001; Czernecki, et al., Synthesis, 1991; Asseline, et al., Nuc. Acids Res., 1991; Pankiewicz, et al., J. Org. Chem., 1982.

### Preparation of Compound D1.02

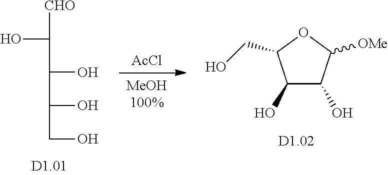 [SMILES: "AcCl" ; O=CC(O)C(O)C(O)CO ; "MeOH100%" ; COC1O[C@@H](CO)[C@H](O)[C@H]1O]

Acetyl chloride (13.3 M, 2.50 mL, 33.3 mmol) was added dropwise to methanol (30.0 mL) at 0° C. The methanolic hydrogen chloride solution was then added slowly to a solution of 2,3,4,5-tetrahydroxypentanal (D1.01) (1.00 g, 6.66 mmol) in methanol (100.0 mL). After 3 hours of stirring at room temperature the reaction was neutralized by addition of pyridine (20 mL) and evaporated to provide the desired compound (D1.02) as an oil. The oil was dried on high vacuum overnight and used in next step with no further purification.

(2S,3R,4R)-2-(hydroxymethyl)-5-methoxy-tetrahydrofuran-3,4-diol (5.47 g, 33.3 mmol, yield: 100%)

¹H NMR (300 MHz, METHANOL-d4) δ 4.75-4.79 (m, 2H), 3.48-4.03 (m, 10H), 3.36-3.43 (m, 6H)

¹³C NMR (75 MHz, METHANOL-d4) δ 109.0, 102.5, 84.0, 82.9, 81.8, 77.5, 77.2, 75.4, 64.0, 61.6, 54.1, 53.8

LCMS: No ionization

### Preparation of Compound D1.03

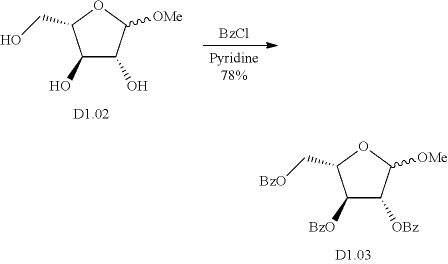

(2S,3R,4R)-2-(hydroxymethyl)-5-methoxy-tetrahydrofuran-3,4-diol (D1.02) (5.47 g, 33.3 mmol) was dissolved in pyridine (40.00 mL) and cooled to 0° C. Benzoyl chloride (31.0 mL, 267 mmol) was added slowly. The reaction was warmed to room temperature and stirred overnight. Water was then added, and the reaction mixture was extracted with dichloromethane. The combined organic extracts were washed with 10% hydrochloric acid (aq), (3×300 mL) and evaporated under reduced pressure. The crude reaction mixture was purified by Biotage (Si, 220 g col, 0-20% ethyl acetate/hexanes) to give the desired product (D1.03) as a clear colorless oil.

[(2S,3S,4R)-3,4-dibenzoyloxy-5-methoxy-tetrahydrofuran-2-yl]methyl benzoate (12.4 g, 26.0 mmol, yield: 78.1%)

¹H NMR (300 MHz, CHLOROFORM-d) δ 7.97-8.14 (m, 6H), 7.52-7.64 (m, 2H), 7.35-7.52 (m, 5H), 7.25-7.34 (m, 2H), 5.59 (td, J=0.90, 5.12 Hz, 1H), 5.52 (d, J=1.41 Hz, 1H), 5.19 (s, 1H), 4.81-4.89 (m, 1H), 4.66-4.75 (m, 1H), 4.58 (dt, J=3.52, 4.90 Hz, 1H), 3.50 (s, 3H)

¹³C NMR (75 MHz, CHLOROFORM-d) δ 166.2, 165.8, 165.5, 133.5, 133.5, 133.1, 130.0, 129.9, 129.8, 129.1, 129.1, 128.5, 128.5, 128.3, 106.9, 82.2, 80.9, 78.0, 63.7, 55.0

LCMS: M+Na=499.1

### Preparation of Compound D1.04

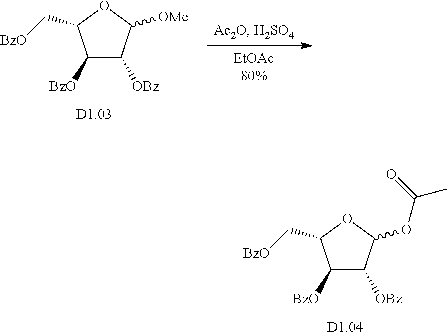

[(2S,3S,4R)-3,4-dibenzoyloxy-5-methoxy-tetrahydrofuran-2-yl]methyl benzoate (D1.03) (15.9 g, 33.4 mmol) was dissolved in ethyl acetate (95.0 mL), then acetic anhydride (10.3 mL, 110 mmol) was added followed by sulfuric acid (0.356 mL, 6.67 mmol). After 3 hours stirring at room temperature, the reaction was diluted with saturated aqueous sodium bicarbonate solution (100 mL) and ethyl acetate (100 mL). The aqueous layer was extracted with ethyl acetate. The combined organics were washed with saturated sodium bicarbonate solution (aq), water and brine, followed by concentration under reduced pressure to give a crude oil. Purification by Biotage (Si, 10 g col, 0-20% ethyl acetate/hexanes) afforded the desired product (D1.04) as a white foam.

[(2S,3S,4R)-5-acetoxy-3,4-dibenzoyloxy-tetrahydrofuran-2-yl]methyl benzoate (13.5 g, 26.8 mmol, yield: 80.4%)

¹H NMR (300 MHz, CHLOROFORM-d) δ 8.00-8.11 (m, 7H), 7.56-7.65 (m, 3H), 7.38-7.52 (m, 6H), 7.26-7.34 (m, 2H), 6.49 (s, 1H), 5.66 (d, J=1.15 Hz, 1H), 5.62-5.65 (m, 1H), 4.66-4.84 (m, 3H), 2.19 (s, 3H)

LCMS: M+Na=527.1

### Preparation of Compound D1.05

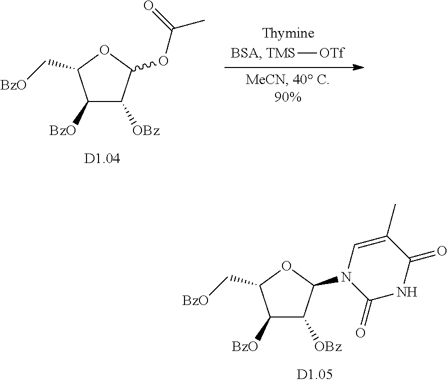

Thymine (0.440 g, 3.49 mmol) and N,O-Bis(trimethylsilyl)acetamide (2.33 mL, 9.54 mmol) were added to a solution of [(2S,3S,4R)-5-acetoxy-3,4-dibenzoyloxy-tetrahydrofuran-2-yl]methylbenzoate (D1.04) (1.60 g, 3.17 mmol) in acetonitrile (16.0 mL). After heating at 40° C. for 15 minutes to obtain a clear solution, trimethylsilyl trifluoromethanesulfonate (0.746 mL, 4.12 mmol) was added, and the reaction was stirred overnight at 40° C. The reaction was concentrated under reduced pressure and diluted with ethyl acetate. The organics were washed with saturated sodium bicarbonate solution and brine, followed by concentration to an oil under reduced pressure. Purification by Biotage (Si, 100 g col, 0-50% ethyl acetate/hexanes) afforded the desired product (D1.05) as a white solid.

[(2S,3S,4R,5R)-3,4-dibenzoyloxy-5-(5-methyl-2,4-dioxo-pyrimidin-1-yl)tetrahydrofuran-2-yl]methyl benzoate (1.63 g, 2.86 mmol, yield: 90.1%)

¹H NMR (300 MHz, CHLOROFORM-d) δ 8.20 (s, 1H), 7.99-8.13 (m, 6H), 7.36-7.65 (m, 9H), 7.29 (d, J=1.28 Hz, 1H), 6.27 (d, J=3.20 Hz, 1H), 5.93 (t, J=2.94 Hz, 1H), 5.72-5.83 (m, 1H), 4.93-5.02 (m, 1H), 4.61-4.83 (m, 2H), 1.93 (d, J=1.15 Hz, 3H)

¹³C NMR (75 MHz, CHLOROFORM-d) δ 166.1, 165.3, 165.2, 163.8, 150.2, 136.0, 134.0, 133.9, 133.4, 130.0, 129.8, 129.4, 128.7, 128.7, 128.5, 128.5, 128.4, 111.3, 91.1, 83.5, 80.5, 63.8, 12.6

LCMS: M+H=571.2

### Preparation of Compound D1.06

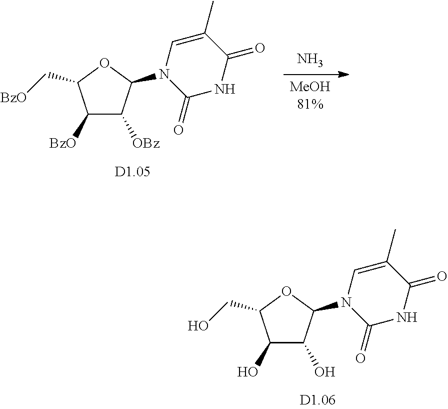

NH₃ (7.00 M, 8.26 mL, 57.8 mmol) in methanol was added to a solution of [(2S,3S,4R,5R)-3,4-dibenzoyloxy-5-(5-methyl-2,4-dioxo-pyrimidin-1-yl)tetrahydrofuran-2-yl]methyl benzoate (D1.05) (11.0 g, 19.3 mmol) was dissolved in methanol (80.0 mL). The reaction was heated at 40° C. for 16 hours and then stirred at room temperature for 72 hours. The reaction was concentrated to an oil and purification by Biotage (Si, 25 g col, 0-20% methanol/dichloromethane) afforded the desired product (D1.06) as a white solid.

1-[(2R,3R,4R,5S)-3,4-dihydroxy-5-(hydroxymethyl)tetrahydrofuran-2-yl]-5-methyl-pyrimidine-2,4-dione (4.05 g, 15.7 mmol, yield: 81.3%)

¹H NMR (300 MHz, DMSO-d6) δ 11.28 (s, 1H), 7.60 (d, J=1.28 Hz, 1H), 5.72 (d, J=4.99 Hz, 1H), 5.64 (d, J=5.38 Hz, 1H), 5.44 (d, J=4.48 Hz, 1H), 4.90 (t, J=5.57 Hz, 1H), 3.85-4.15 (m, 3H), 3.40-3.62 (m, 2H), 1.79 (d, J=1.15 Hz, 3H)

¹³C NMR (75 MHz, DMSO-d6) δ 163.8, 150.6, 137.0, 109.1, 89.2, 85.7, 79.1, 74.6, 61.1, 12.1 LCMS: M+H=259.0

### Preparation of Compound D1.07

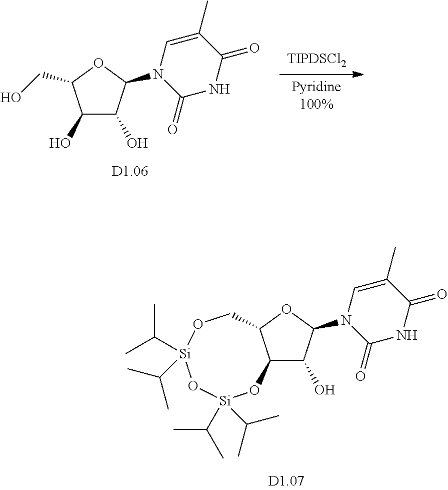 [SMILES: Cc1cn([C@@H]2O[C@@H](CO)[C@H](O)[C@H]2O)c(=O)[nH]c1=O ; "Pyridine100%" ; "TIPDSCl2" ; Cc1cn([C@@H]2O[C@H]3CO[Si](C(C)C)(C(C)C)O[Si](C(C)C)(C(C)C)O[C@@H]3[C@H]2O)c(=O)[nH]c1=O]

1-[(2R,3R,4R,5S)-3,4-dihydroxy-5-(hydroxymethyl)tetrahydrofuran-2-yl]-5-methyl-pyrimidine-2,4-dione (D1.06) (3.92 g, 15.2 mmol) was dissolved in pyridine (50 mL) and evaporated to dryness under reduced pressure at 60° C. three times to dry the starting material. This was then dissolved in pyridine (50.5 mL) and 1,3-dichloro-1,1,3,3-tetraisopropyldisiloxane (5.83 mL, 18.2 mmol) was added. The reaction was stirred at room temperature for 30 mins before concentration to an oil under reduced pressure. The oil was dissolved in ethyl acetate and the organics were washed with 10% HCl (aq), water, saturated sodium bicarbonate solution, water, brine, then concentrated to afford the desired product (D1.07) as a white amorphous solid.

1-[(6aS,8R,9R,9aR)-9-hydroxy-2,2,4,4-tetraisopropyl-6a,8,9,9a-tetrahydro-6H-furo[3,2-f][1,3,5,2,4]trioxadisilocin-8-yl]-5-methyl-pyrimidine-2,4-dione (7.61 g, 15.2 mmol, yield: 100%)

¹H NMR (300 MHz, CHLOROFORM-d) δ 8.89 (s, 1H), 7.32 (d, J=1.15 Hz, 1H), 5.50 (d, J=4.48 Hz, 1H), 4.37-4.44 (m, 1H), 4.28-4.36 (m, 1H), 4.05 (dd, J=3.33, 4.99 Hz, 2H), 3.92-4.01 (m, 1H), 3.84 (d, J=2.56 Hz, 1H), 1.95 (d, J=1.15 Hz, 3H), 0.96-1.17 (m, 28H)

¹³C NMR (75 MHz, CHLOROFORM-d) δ 164.3, 151.5, 149.6, 136.2, 134.7, 123.8, 110.8, 92.0, 83.4, 82.0, 76.0, 61.6, 60.4, 17.5, 17.3, 17.2, 17.0, 17.0, 16.9, 16.9, 13.0, 12.5

LCMS: M+H=501.2

### Preparation of Compound D1.08

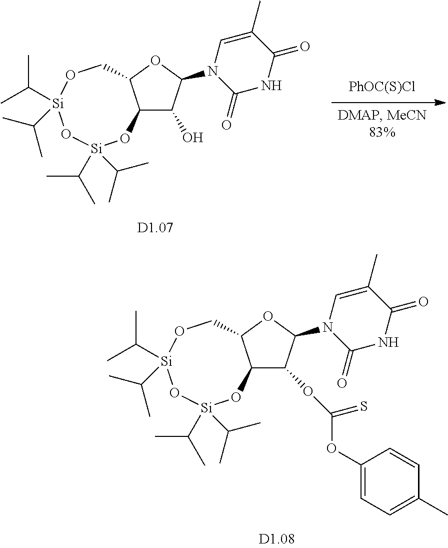 [SMILES: "PhOC(S)Cl" ; Cc1cn([C@@H]2O[C@H]3CO[Si](C(C)C)(C(C)C)O[Si](C(C)C)(C(C)C)O[C@@H]3[C@H]2O)c(=O)[nH]c1=O ; "DMAP, MeCN83%" ; Cc1ccc(OC(=S)O[C@@H]2[C@H]3O[Si](C(C)C)(C(C)C)O[Si](C(C)C)(C(C)C)OC[C@@H]3O[C@H]2n2cc(C)c(=O)[nH]c2=O)cc1]

1-[(6aS,8R,9R,9aR)-9-hydroxy-2,2,4,4-tetraisopropyl-6a,8,9,9a-tetrahydro-6H-furo[3,2-f][1,3,5,2,4]trioxadisilocin-8-yl]-5-methyl-pyrimidine-2,4-dione (D1.07) (2.84 g, 0.00567 mol) and 4-dimethylaminopyridine (1.39 g, 0.0113 mol) were dissolved in anhydrous acetonitrile (56.8 mL) followed by slow addition of O-4-methylphenyl chlorothioformate (0.951 mL, 0.00624 mol). The reaction was stirred at room temperature for 72 hours. The solvents were removed under reduced pressure and the residue was partitioned between ethyl acetate and water. The aqueous layer was extracted with ethyl acetate and the combined organics were washed with 10% HCl (aq), water, saturated sodium bicarbonate solution, water and brine. The organic fractions were dried over magnesium sulfate and concentrated. Purification by Biotage (Si, 100 g col, 0-40% ethyl acetate/hexanes) afforded the desired product (D1.08) as a white solid.

1-[(6aS,8R,9R,9aS)-2,2,4,4-tetraisopropyl-9-(4-methylphenoxy)carbothioyloxy-6a,8,9,9a-tetrahydro-6H-furo[3,2-f][1,3,5,2,4]trioxadisilocin-8-yl]-5-methyl-pyrimidine-2,4-dione (3.06 g, 0.00470 mol, yield: 82.9%)

¹H NMR (300 MHz, CHLOROFORM-d) δ 8.21 (s, 1H), 7.09-7.24 (m, 3H), 6.86-7.04 (m, 2H), 6.27 (dd, J=5.18, 6.98 Hz, 1H), 5.93 (d, J=5.25 Hz, 1H), 4.74 (dd, J=7.04, 8.19 Hz, 1H), 4.35 (td, J=3.89, 7.97 Hz, 1H), 3.96-4.06 (m, 2H), 2.36 (s, 3H), 1.94 (d, J=1.15 Hz, 3H), 1.00-1.18 (m, 28H)

¹³C NMR (75 MHz, CHLOROFORM-d) δ 194.8, 163.6, 151.3, 150.3, 136.6, 136.6, 130.1, 121.2, 111.6, 88.3, 87.5, 83.6, 74.0, 61.7, 21.0, 17.4, 17.3, 17.3, 17.3, 17.0, 16.9, 13.5, 13.1, 12.8, 12.6, 12.4

LCMS: no ionization

### Preparation of Compound D1.09

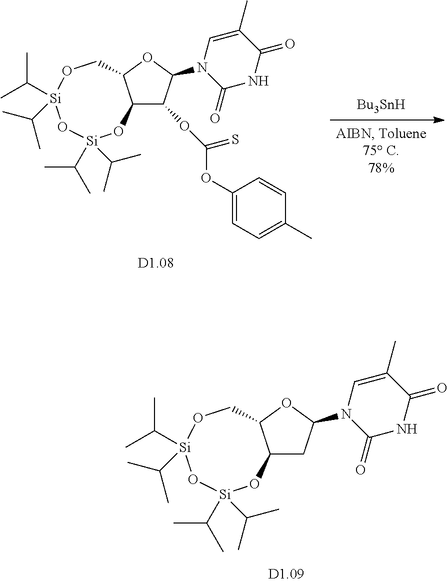

Azobisisobutyronitrile (AIBN) (0.0101 g, 0.0615 mmol) and tributyltin hydride (0.894 g, 3.07 mmol) in toluene (2 mL) were added dropwise to a degassed (with nitrogen) solution of 1-[(6aS,8R,9R,9aS)-2,2,4,4-tetraisopropyl-9-(4-methylphenoxy)carbothioyloxy-6a,8,9,9a-tetrahydro-6H-furo[3,2-f][1,3,5,2,4]trioxadisilocin-8-yl]-5-methyl-pyrimidine-2,4-dione (D1.08) (0.200 g, 0.307 mmol) in toluene (4 mL) at 80° C. The solution was heated at 80° C. for 1 hour before being cooled to room temperature and removal of the solvents under reduced pressure. Purification by Biotage (Si, 50 g col, 0-40% ethyl acetate/hexanes) afforded the desired product (D1.09) as a white solid.

1-[(6aS,8R,9aR)-2,2,4,4-tetraisopropyl-6a,8,9,9a-tetrahydro-6H-furo[3,2-f][1,3,5,2,4]trioxadisilocin-8-yl]-5-methyl-pyrimidine-2,4-dione (0.116 g, 0.239 mmol, yield: 77.9%)

¹H NMR (300 MHz, CHLOROFORM-d) δ 8.15 (s, 1H), 7.48 (d, J=1.28 Hz, 1H), 6.18 (dd, J=5.70, 6.72 Hz, 1H), 4.49-4.65 (m, 1H), 3.99-4.10 (m, 2H), 3.73-3.86 (m, 1H), 2.80 (td, J=7.20, 14.02 Hz, 1H), 2.16 (td, J=6.11, 14.02 Hz, 1H), 1.95 (d, J=1.28 Hz, 3H), 0.99-1.15 (m, 28H)

¹³C NMR (75 MHz, CHLOROFORM-d) δ 163.9, 150.7, 135.5, 111.0, 85.9, 84.6, 72.4, 63.1, 39.9, 17.5, 17.3, 17.0, 17.1, 13.3, 13.3, 12.9, 12.6, 12.5

LCMS: no ionization

### Preparation of Compound D1.10

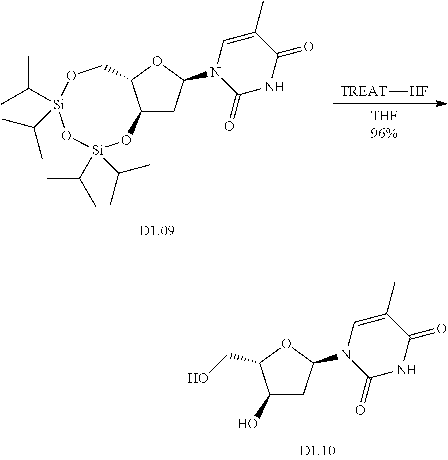

TEA (0.0812 mL, 0.583 mmol) was added to a solution of 1-[(6aS,8R,9aR)-2,2,4,4-tetraisopropyl-6a,8,9,9a-tetrahydro-6H-furo[3,2-f][1,3,5,2,4]trioxadisilocin-8-yl]-5-methyl-pyrimidine-2,4-dione (D1.09) (0.113 g, 0.233 mmol) in THF (1.16 mL). The reaction was cooled to 0° C. with an ice bath under an atmosphere of nitrogen. Triethylamine trihydrofluoride (0.190 mL, 1.17 mmol) was added slowly at 0° C., and then the reaction was warmed to room temperature and stirred for 1.5 hours. The solvents were removed under reduced pressure and purification by Biotage (Si, 10 g col, 0-10% methanol/dichloromethane) afforded the desired product (D1.10) as a white gummy solid.

1-[(2R,4R,5S)-4-hydroxy-5-(hydroxymethyl)tetrahydrofuran-2-yl]-5-methyl-pyrimidine-2,4-dione (54.0 mg, 0.000223 mol, yield: 95.6%)

¹H NMR (300 MHz, METHANOL-d4) δ 7.79 (d, J=1.02 Hz, 1H), 6.21 (dd, J=2.82, 7.42 Hz, 1H), 4.38 (td, J=2.13, 6.11 Hz, 1H), 4.30 (dt, J=2.11, 4.32 Hz, 1H), 3.49-3.70 (m, 2H), 2.69 (ddd, J=6.34, 7.52, 14.31 Hz, 1H), 2.07 (td, J=2.48, 14.50 Hz, 1H), 1.90 (d, J=0.90 Hz, 3H)

¹³C NMR (75 MHz, METHANOL-d4) δ 166.7, 152.5, 138.9, 110.7, 91.0, 88.1, 72.5, 63.5, 41.7, 12.6

LCMS: M+H=243.1 and M+Na=265.1

### Preparation of Compound D1.11

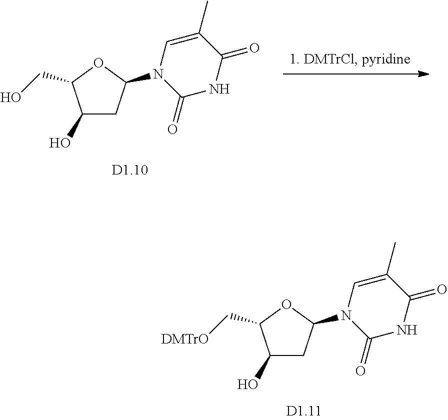

DMTrCl (73.9 mg, 0.218 mmol) was added to a solution of 1-[(2R,4R,5S)-4-hydroxy-5-(hydroxymethyl)tetrahydrofuran-2-yl]-5-methyl-pyrimidine-2,4-dione (D1.10) (732 mg, 3.02 mmol) in pyridine (10.1 mL) at room temperature and stirred for 2 hours. The reaction was quenched with the addition of methanol (0.5 mL), followed by dilution of the reaction with water and ethyl acetate. The aqueous layer was extracted with ethyl acetate. The combined organics were washed with water, saturated sodium bicarbonate, and brine. Followed by removal of the solvents under reduced pressure. Purification by Biotage (Si, 100 g col, 0-80% ethyl acetate/hexanes) afforded the desired product (D1.11) as a white solid.

1-[(2R,4R,5S)-5-[[bis(4-methoxyphenyl)-phenyl-methoxy]methyl]-4-hydroxy-tetrahydrofuran-2-yl]-5-methyl-pyrimidine-2,4-dione (1394 mg, 2.56 mmol, yield: 84.7%)

¹H NMR (300 MHz, CHLOROFORM-d) δ 8.22 (s, 1H), 7.49 (d, J=1.28 Hz, 1H), 7.36-7.44 (m, 2H), 7.27-7.34 (m, 6H), 7.23 (d, J=7.04 Hz, 1H), 6.78-6.91 (m, 4H), 6.20 (dd, J=2.50, 7.87 Hz, 1H), 4.30-4.51 (m, 2H), 3.80 (s, 6H), 3.11-3.31 (m, 2H), 2.82 (ddd, J=6.40, 7.97, 14.69 Hz, 1H), 2.62 (br s, 1H), 2.14 (br d, J=14.72 Hz, 1H), 1.93 (d, J=1.02 Hz, 3H)

LCMS: M−H=543.3

### Preparation of Compound 1a

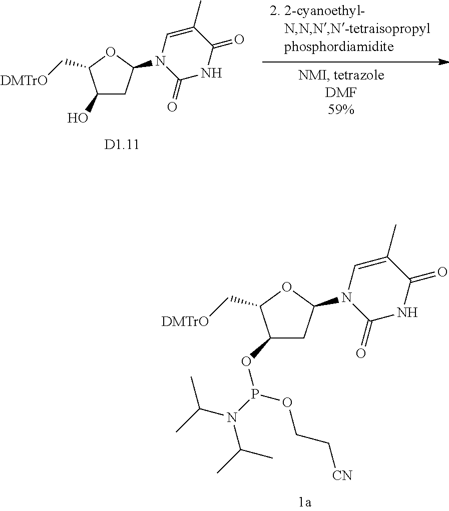

1H-tetrazole (0.157 g, 2.25 mmol) and 1-methylimidazole (0.0557 mL, 0.702 mmol) were added to a solution of 1-[(2R,4R,5S)-5-[[bis(4-methoxyphenyl)-phenyl-methoxy]methyl]-4-hydroxy-tetrahydrofuran-2-yl]-5-methyl-pyrimidine-2,4-dione (D1.11) (1.53 g, 2.81 mmol) in DMF (22.3 mL) at room temperature under an atmosphere of nitrogen. 3-Bis(diisopropylamino)phosphanyloxypropanenitrile (1.34 mL, 4.21 mmol) was then added dropwise and the reaction was stirred at room temperature for 90 minutes. Water (1.0 mL) was added to quench the reaction. A 3:1 mixture of toluene/hexanes (80 mL) was added and the organic layer was washed four times with a 3:2 mixture of DMF/H₂O (50 mL). The organics were then washed with saturated sodium bicarbonate solution, brine, dried over solid sodium sulfate and concentrated under reduced pressure to a white foam. Purification by Biotage (Si, 50 g col, 0-60% ethyl acetate/hexanes) afforded the desired product (1a) as a white amorphous solid.

3-[[(2S,3R,5R)-2-[[bis(4-methoxyphenyl)-phenyl-methoxy]methyl]-5-(5-methyl-2,4-dioxo-pyrimidin-1-yl)tetrahydrofuran-3-yl]oxy-(diisopropylamino)phosphanyl]oxypropanenitrile (1.23 g, 1.65 mmol, yield: 58.8%)

¹H NMR (300 MHz, CHLOROFORM-d) δ 8.11 (br s, 1H), 7.52 (dd, J=1.28, 4.86 Hz, 1H), 7.38-7.46 (m, 2H), 7.27-7.36 (m, 6H), 7.24 (dd, J=1.86, 7.36 Hz, 1H), 6.77-6.93 (m, 4H), 6.31 (td, J=2.24, 7.30 Hz, 1H), 4.43-4.66 (m, 2H), 3.80 (d, J=0.90 Hz, 6H), 3.61-3.76 (m, 2H), 3.43-3.58 (m, 2H), 3.08-3.34 (m, 2H), 2.67-2.90 (m, 1H), 2.51 (td, J=6.18, 19.01 Hz, 2H), 2.15-2.37 (m, 1H), 1.88-1.98 (m, 3H), 1.04-1.19 (m, 12H)

¹³C NMR (75 MHz, CHLOROFORM-d) δ 164.1, 164.0, 158.6, 150.4, 150.4, 144.5, 144.4, 136.5, 136.4, 135.7, 135.6, 135.6, 135.5, 130.0, 128.1, 128.1, 127.9, 127.0, 117.3, 113.2, 109.8, 109.7, 88.0, 87.9, 87.8, 87.7, 87.4, 87.1, 86.6, 86.6, 74.9, 74.6, 74.6, 63.9, 63.8, 60.4, 58.4, 58.2, 58.1, 58.0, 55.3, 43.4, 43.2, 40.9, 40.8, 40.2, 24.6, 24.5, 24.4, 24.4, 24.3, 21.1, 20.3, 20.2, 14.2, 12.7

³¹P NMR (121 MHz, CHLOROFORM-d) δ 149.89 (s, 1P), 149.47 (s, 1P)

LCMS: M−H=743.4

### Example 34: Synthesis of an Amidite of a Stereo-Non-Standard Nucleoside Comprising a 2′-α-L-Deoxyribosyl Sugar Moiety

Compound 2a, the amidite of a stereo-non-standard nucleoside comprising 2′-α-L-deoxyribosyl sugar moiety was synthesized according to the scheme below. A similar synthesis has been previously reported (WO 9945935).

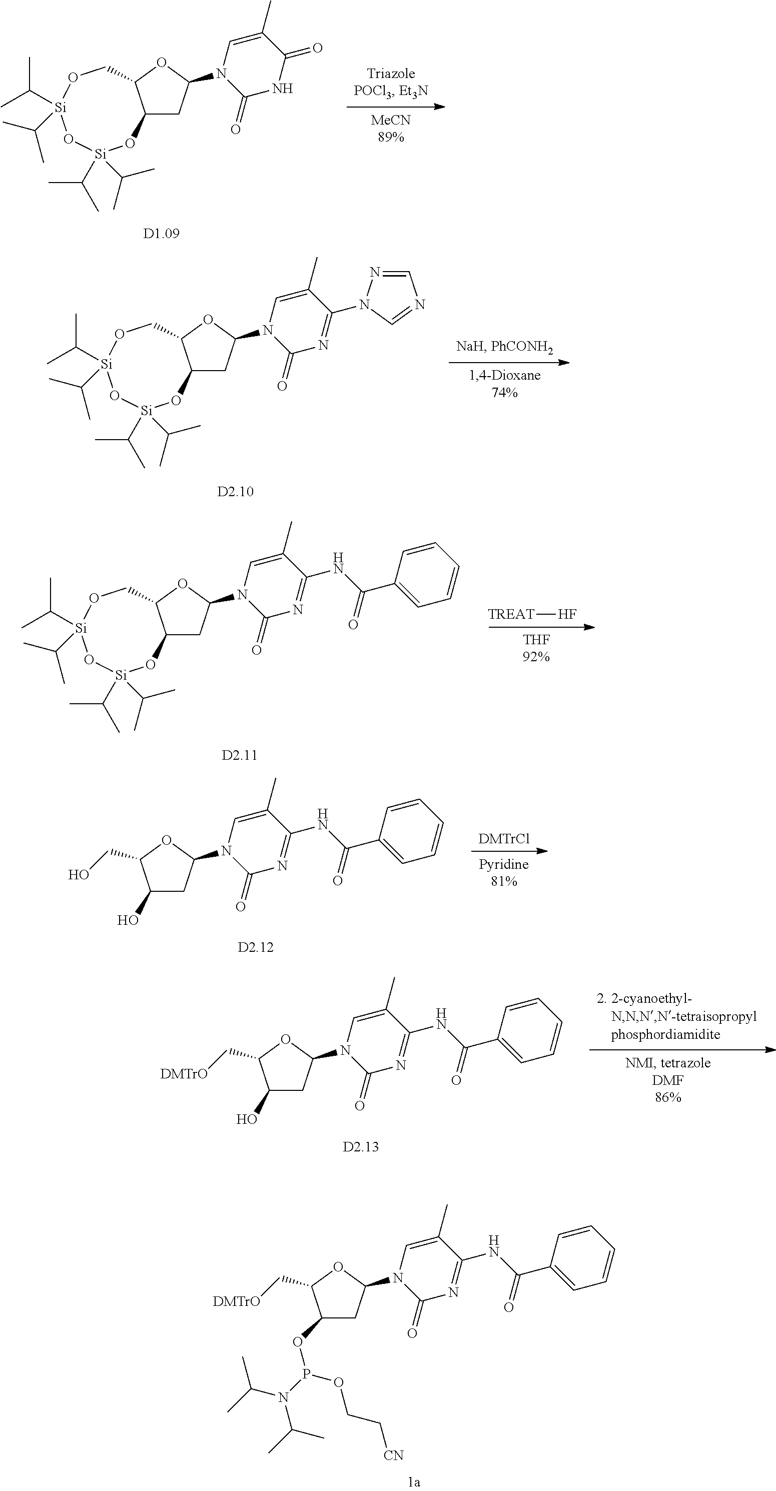

### Preparation of Compound D2.10

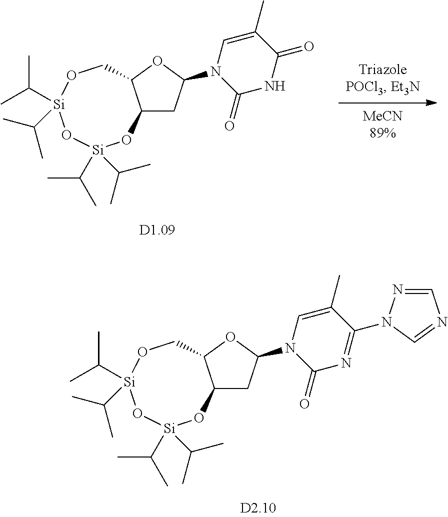 [SMILES: Cc1cn([C@H]2C[C@H]3O[Si](C(C)C)(C(C)C)O[Si](C(C)C)(C(C)C)OC[C@@H]3O2)c(=O)[nH]c1=O ; "MeCN89%" ; "TriazolePOCl3, Et3N" ; Cc1cn([C@H]2C[C@H]3O[Si](C(C)C)(C(C)C)O[Si](C(C)C)(C(C)C)OC[C@@H]3O2)c(=O)nc1-n1cncn1]

POCl₃ (2.53 mL, 27.6 mmol) was added dropwise to a suspension of 1,2,4-1H-triazole (7.16 g, 104 mmol) in acetonitrile (69.0 mL) under an atmosphere of nitrogen at 0° C., followed by dropwise addition of triethylamine (19.3 mL, 138 mmol). After 30 minutes at 0° C. a solution of 1-[(6aS,8R,9aR)-2,2,4,4-tetraisopropyl-6a,8,9,9a-tetrahydro-6H-furo[3,2-f][1,3,5,2,4]trioxadisilocin-8-yl]-5-methyl-pyrimidine-2,4-dione (D1.09) (3.35 g, 6.91 mmol) in THF (10.00 mL) was added dropwise. This was stirred at room temperature overnight. The reaction was concentrated to small volume under reduced pressure, diluted with ethyl acetate, and the organic layer was washed with aqueous saturated sodium bicarbonate (2×), water, and brine, then concentrated to a yellow oil. Purification by column on Biotage (Si, 25 g col, 0-60% ethyl acetate/hexanes) afforded the desired product (D2.10) as a white amorphous solid.

1-[(6aS,8R,9aR)-2,2,4,4-tetraisopropyl-6a,8,9,9a-tetrahydro-6H-furo[3,2-f][1,3,5,2,4]trioxadisilocin-8-yl]-5-methyl-4-(1,2,4-triazol-1-yl)pyrimidin-2-one (3.29 g, 6.14 mmol, yield: 88.9%)

¹H NMR (300 MHz, CHLOROFORM-d) δ 9.29 (s, 1H), 8.12 (s, 1H), 8.00 (d, J=0.77 Hz, 1H), 6.12 (dd, J=5.50, 6.27 Hz, 1H), 4.53-4.68 (m, 1H), 4.05-4.24 (m, 2H), 3.85-3.99 (m, 1H), 3.08 (td, J=6.93, 13.92 Hz, 1H), 2.49 (d, J=0.77 Hz, 3H), 2.20 (ddd, J=5.50, 6.88, 13.99 Hz, 1H), 0.95-1.21 (m, 28H)

LCMS: M+H=536.2

### Preparation of Compound D2.11

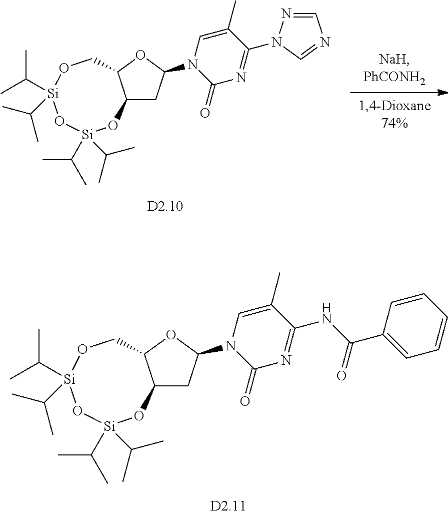

1,4-Dioxane (1.96 mL) was added to NaH (60.0%, 63.3 mg, 1.58 mmol) in a flask under an atmosphere of nitrogen at room temperature. A suspension of benzamide (192 mg, 1.58 mmol) in 1,4-dioxane (1.00 mL) was added to the flask and the reaction was stirred for 1 hour at room temperature. A solution of 1-[(6aS,8R,9aR)-2,2,4,4-tetraisopropyl-6a,8,9,9a-tetrahydro-6H-furo[3,2-f][1,3,5,2,4]trioxadisilocin-8-yl]-5-methyl-4-(1,2,4-triazol-1-yl)pyrimidin-2-one (D2.10) (212 mg, 0.396 mmol) in 1,4-dioxane (1.00 mL) was added to the reaction flask and the reaction was stirred for 2 hours at room temperature. The reaction was quenched by addition of saturated aqueous ammonium chloride solution, and the aqueous layer was extracted with ethyl acetate. The combined organics were washed with brine, dried over magnesium sulfate, and concentrated to a crude solid. Purification by column on Biotage (Si, 25 g col, 0-10% ethyl acetate/hexanes) afforded the desired product (D2.11) as a white solid.

N-[1-[(6aS,8R,9aR)-2,2,4,4-tetraisopropyl-6a,8,9,9a-tetrahydro-6H-furo[3,2-f][1,3,5,2,4]trioxadisilocin-8-yl]-5-methyl-2-oxo-pyrimidin-4-yl]benzamide (173 mg, 0.294 mmol, yield: 74.4%)

¹H NMR (300 MHz, CHLOROFORM-d) δ 8.27-8.38 (m, 2H), 7.61 (d, J=1.02 Hz, 1H), 7.49-7.56 (m, 1H), 7.40-7.48 (m, 2H), 6.18 (dd, J=5.70, 6.59 Hz, 1H), 4.51-4.64 (m, 1H), 4.02-4.15 (m, 2H), 3.78-3.91 (m, 1H), 2.86 (td, J=7.14, 13.89 Hz, 1H), 2.16-2.25 (m, 1H), 2.15 (d, J=1.02 Hz, 3H), 1.00-1.18 (m, 28H)

LCMS: M+H=588.3

### Preparation of Compound D2.12

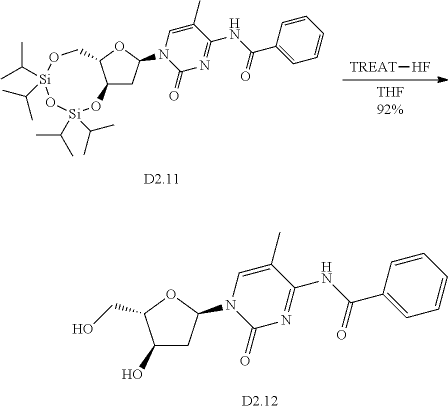

TEA (1.96 mL, 14.0 mmol) was added to a solution of N-[1-[(6aS,8R,9aR)-2,2,4,4-tetraisopropyl-6a,8,9,9a-tetrahydro-6H-furo[3,2-f][1,3,5,2,4]trioxadisilocin-8-yl]-5-methyl-2-oxo-pyrimidin-4-yl]benzamide (D2.11) (3.30 g, 5.61 mmol) in tetrahydrofuran (56.0 mL). The reaction was cooled to 0° C. under an atmosphere of nitrogen. Triethylamine trihydrofluoride (4.58 mL, 28.1 mmol) was added slowly, and then the reaction was warmed to room temperature with stirring for 3 hours. The solvents were removed under reduced pressure, and purification by Biotage (Si, 220 g col, 0-10% methanol/dichloromethane) afforded the desired product (D2.12) as a white solid.

N-[1-[(2R,4R,5S)-4-hydroxy-5-(hydroxymethyl)tetrahydrofuran-2-yl]-5-methyl-2-oxo-pyrimidin-4-yl]benzamide (1.78 g, 5.15 mmol, yield: 91.7%)

¹H NMR (300 MHz, DMSO-d6) δ 8.17 (d, J=7.17 Hz, 2H), 8.03 (d, J=1.02 Hz, 1H), 7.56-7.56 (m, 1H), 7.44-7.55 (m, 3H), 6.11 (dd, J=2.37, 7.36 Hz, 1H), 5.08-5.62 (m, 1H), 4.73-4.99 (m, 1H), 4.21-4.35 (m, 2H), 3.43 (d, J=4.48 Hz, 2H), 2.52-2.77 (m, 2H), 2.04 (d, J=0.90 Hz, 3H)

LCMS: M+H=346.2

### Preparation of Compound D2.13

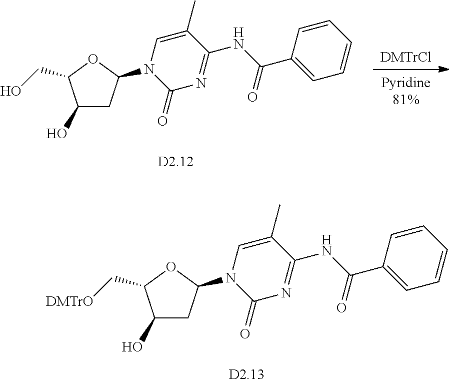

DMTrCl (1.92 g, 5.66 mmol) was added to a solution of N-[1-[(2R,4R,5S)-4-hydroxy-5-(hydroxymethyl)tetrahydrofuran-2-yl]-5-methyl-2-oxo-pyrimidin-4-yl]benzamide (D2.12) (1.78 g, 5.15 mmol) in pyridine (17.1 mL). The reaction was stirred at room temperature for 2 hours. The reaction was quenched with the addition of methanol (0.5 mL), followed by dilution with water and ethyl acetate. The aqueous layer was extracted with ethyl acetate. The combined organics were washed with water, saturated sodium bicarbonate, brine, then concentrated under reduced pressure. Purification by Biotage (Si, 220 g col, 0-60% ethyl acetate/hexanes) afforded the desired product (D2.13) as a pale yellow solid.

N-[1-[(2R,4R,5S)-5-[[bis(4-methoxyphenyl)-phenyl-methoxy]methyl]-4-hydroxy-tetrahydrofuran-2-yl]-5-methyl-2-oxo-pyrimidin-4-yl]benzamide (2.70 g, 4.17 mmol, yield: 81.0%)

¹H NMR (300 MHz, CHLOROFORM-d) δ 8.26-8.39 (m, 2H), 7.65 (d, J=1.15 Hz, 1H), 7.49-7.56 (m, 1H), 7.39-7.48 (m, 4H), 7.27-7.36 (m, 6H), 7.18-7.25 (m, 1H), 6.76-6.93 (m, 4H), 6.24 (dd, J=2.37, 7.74 Hz, 1H), 4.45 (t, J=3.84 Hz, 2H), 3.80 (s, 6H), 3.11-3.33 (m, 2H), 2.76-2.97 (m, 1H), 2.44 (d, J=3.71 Hz, 1H), 2.18 (br d, J=14.72 Hz, 1H), 2.13 (d, J=1.02 Hz, 3H)

LCMS: M−H=648.36

### Preparation of Compound 2a

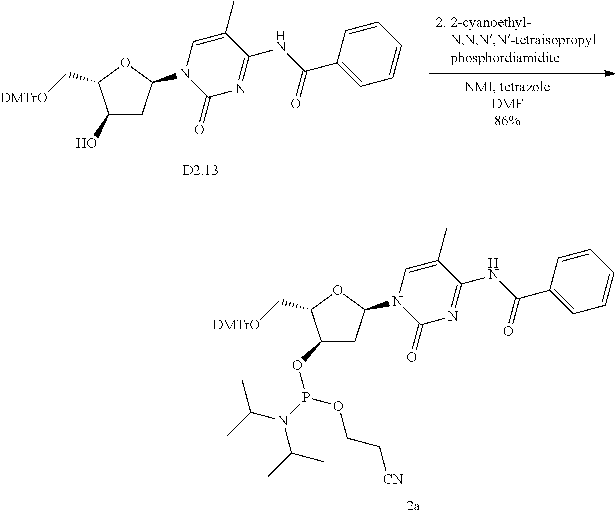

1H-Tetrazole (0.234 g, 3.33 mmol) and 1-methylimidazole (0.0827 mL, 1.04 mmol) were added to a solution of N-[1-[(2R,4R,5S)-5-[[bis(4-methoxyphenyl)-phenyl-methoxy]methyl]-4-hydroxy-tetrahydrofuran-2-yl]-5-methyl-2-oxo-pyrimidin-4-yl]benzamide (D2.13) (2.70 g, 4.17 mmol) in DMF (41.6 mL), followed by dropwise addition of 3-bis(diisopropylamino)phosphanyloxypropanenitrile (1.99 mL, 6.25 mmol) and stirred at room temperature for 90 minutes. Water (1.0 mL) was added to quench the reaction. A 3:1 mixture of toluene/hexanes (80 mL) was added, and the organic layer was washed four times with a 3:2 mixture of DMF/H₂O (50 mL). The organics were then washed with saturated sodium bicarbonate solution, brine, dried over solid sodium sulfate, then concentrated under reduced pressure. Purification by Biotage (Si, 220 g col, 0-50% ethyl acetate/hexanes) (loaded with a small amount of EtOAc) afforded the desired product (2a) as a white amorphous solid.

N-[1-[(2R,4R,5S)-5-[[bis(4-methoxyphenyl)-phenyl-methoxy]methyl]-4-[2-cyanoethoxy-(diisopropylamino)phosphanyl]oxy-tetrahydrofuran-2-yl]-5-methyl-2-oxo-pyrimidin-4-yl]benzamide (3.03 g, 3.57 mmol, yield: 85.7%)

¹H NMR (300 MHz, CHLOROFORM-d) δ 8.27-8.41 (m, 2H), 7.67 (d, J=0.90 Hz, 1H), 7.51 (d, J=7.30 Hz, 1H), 7.38-7.48 (m, 4H), 7.27-7.35 (m, 6H), 7.19-7.26 (m, 1H), 6.80-6.91 (m, 4H), 6.24-6.38 (m, 1H), 4.46-4.74 (m, 2H), 3.80 (d, J=0.77 Hz, 6H), 3.60-3.77 (m, 2H), 3.39-3.59 (m, 2H), 3.09-3.35 (m, 2H), 2.68-2.90 (m, 1H), 2.45-2.58 (m, 2H), 2.22-2.45 (m, 1H), 2.13 (t, J=1.15 Hz, 3H), 1.00-1.19 (m, 12H)

¹³C NMR (75 MHz, CHLOROFORM-d) δ 160.2, 158.6, 148.0, 144.5, 144.4, 137.8, 137.4, 135.7, 135.6, 135.5, 135.5, 132.3, 132.3, 130.0, 129.9, 128.1, 128.1, 128.0, 127.0, 117.4, 117.3, 110.5, 113.3, 88.4, 88.4, 88.3, 88.3, 88.2, 87.9, 86.7, 86.7, 74.8, 63.9, 63.8, 60.4, 58.2, 55.3, 43.3, 41.0, 40.9, 40.3, 24.6, 24.5, 24.5, 24.2, 21.1, 20.3, 14.2, 13.8

³¹P NMR (121 MHz, CHLOROFORM-d) δ 149.98 (s, 1P), 149.26 (s, 1P)

LCMS: M−H=846.5

### Example 35: Design and Synthesis of Stereo-Non-Standard Nucleoside, α-L-Deoxyribose

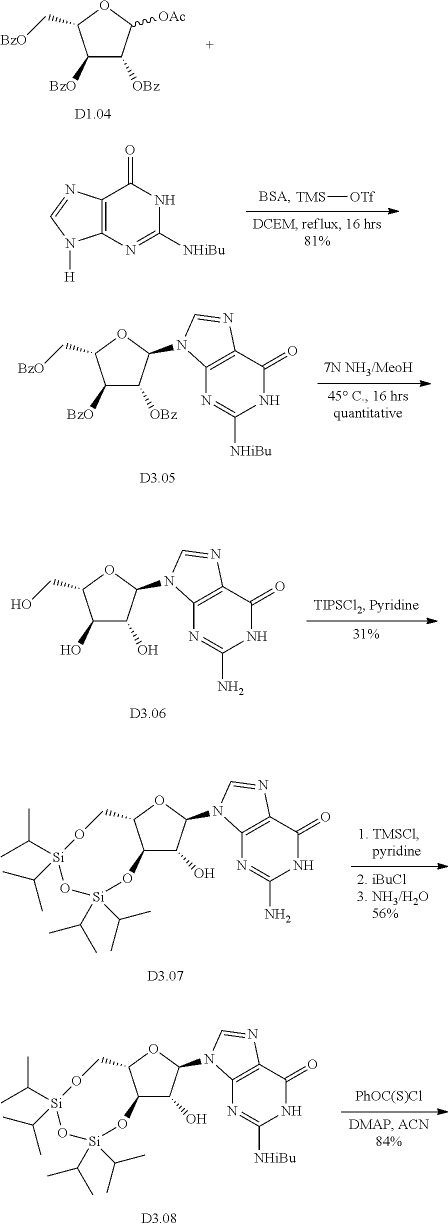

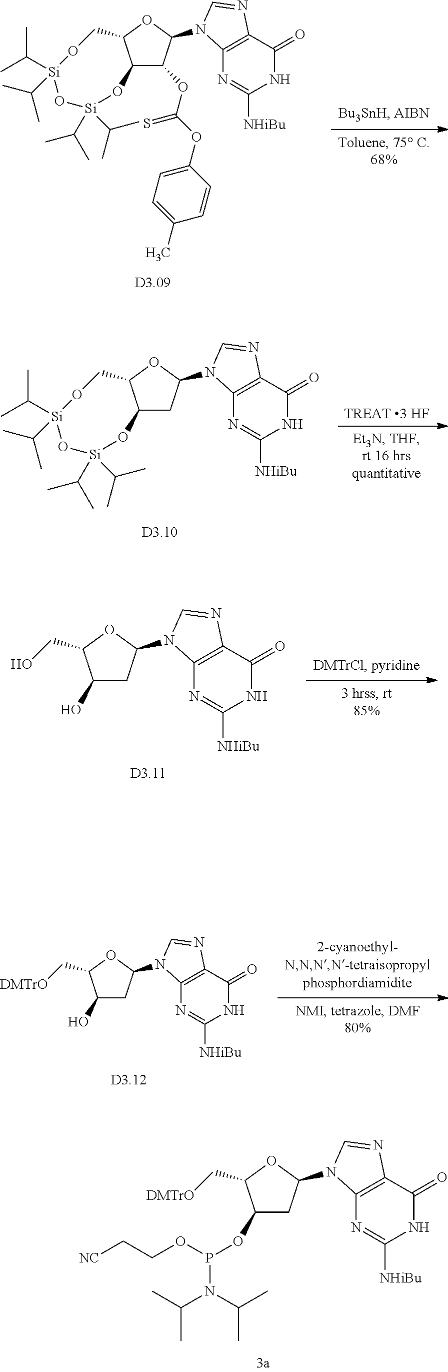

### Preparation of Compound D3.05

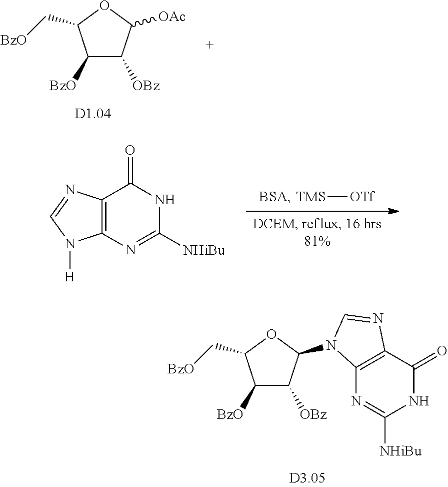

2-(isobutylamino)-1,9-dihydro-6H-purin-6-one (23 g, 119 mmol) and [(2S,3S,4R)-5-acetoxy-3,4-dibenzoyloxy-tetrahydrofuran-2-yl]methyl benzoate (D1.04) (40 g, 79.3 mmol) was co-evaporated (4×50 mL) with toluene at 60° C. then suspended in anhydrous dichloroethane (800 mL). Next N,O-Bis(trimethylsilyl)acetamide (75.5 mL, 317 mmol) was added to the reaction. Reflux at 80° C. for 1 hour led to a clear solution. The reaction solution was cooled with an ice bath to 5° C. Trimethylsilyl trifluoromethanesulfonate (23 mL, 127 mmol) was added, and the reaction was stirred overnight at 80° C. The next day, the reaction was concentrated under reduced pressure and diluted with ethyl acetate. The organic material was washed with plain DI water first, then with saturated sodium bicarbonate solution to pH 7, and then with saturated brine solution. The material was concentrated to an oil under reduced pressure. Purification by silica gel glass chromatography (Silica gel 1000 mL 6/4 diethyl ether/hexanes) afforded the desired product (D3.05) as a white solid. (43.0 g crude, 81% yield)

### Preparation of Compound D3.06

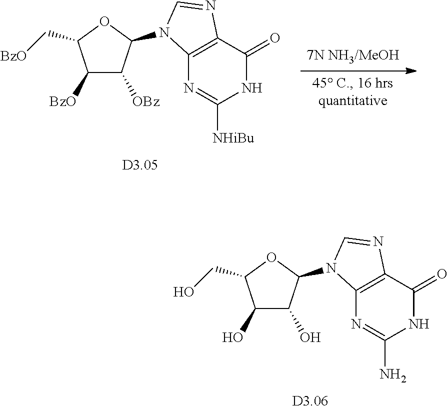

9-((2R,3R,4R,5S)-3,4-Dihydroxy-5-(hydroxymethyl)tetrahydrofuran-2-yl)-2-(isobutylamino)-1,9-dihydro-6H-purin-6-one (D3.05) (43.0 g, 6560 mmol) was suspend in methanol (50.0 mL) and cooled to −20° C. Then NH₃/MeOH (7.00 M, 150 mL) was added, and the reaction was heated at 45° C. for 16 hours. The next day, the solution was concentrated to an oil. The crude oil was suspended in EtOAc (100 mL) to obtain a white precipitate. The solid was filtered and rinsed with fresh EtOAc. The crude solid was dried under high vacuum to obtain product D3.06. (20 g, quantitative % yield)

### Preparation of Compound D3.07

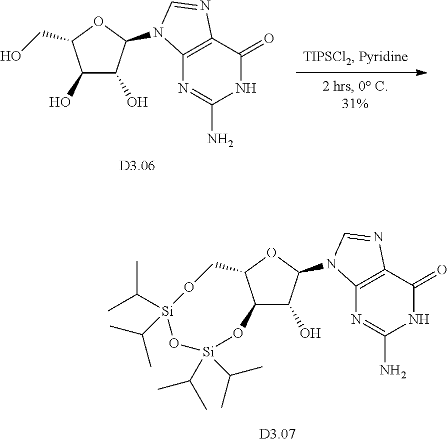 [SMILES: Nc1nc2c(ncn2[C@@H]2O[C@@H](CO)[C@H](O)[C@H]2O)c(=O)[nH]1 ; "2 hrs, 0° C.31%" ; "TIPSCl2, Pyridine" ; CC(C)[Si]1(C(C)C)OC[C@@H]2O[C@@H](n3cnc4c(=O)[nH]c(N)nc43)[C@H](O)[C@H]2O[Si](C(C)C)(C(C)C)O1]

2-Amino-9-((2R,3R,4R,5S)-3,4-dihydroxy-5-(hydroxymethyl)tetrahydrofuran-2-yl)-1,9-dihydro-6H-purin-6-one (D3.06) (20 g, 76.60 mmol) was dissolved in pyridine (400 mL) under nitrogen. The solution was cooled with an ice bath, and 1,3-dichloro-1,1,3,3-tetraisopropyldisiloxane (23.30 mL, 63.60 mmol, 0.90 eq.) was added dropwise. The reaction was warmed up slowly to about 10° C. for 2 hours. TLC in EtOAc/hexane (8/2) indicated reaction was completed. The reaction was cooled down with an ice bath to 0° C. and was quenched by adding slowly DI water (20 mL) and concentrated to an oil under reduced pressure. The oil was dissolved in ethyl acetate, and the organics were washed with 10% HCl (aq), water, saturated sodium bicarbonate solution, water, and brine, then concentrated to afford the desired product as a colorless oil. The crude oil was suspended in hexane to obtain white precipitate (D3.07). (Final weight 14.40 g crude, 31% yield)

### Preparation of Compound D3.08

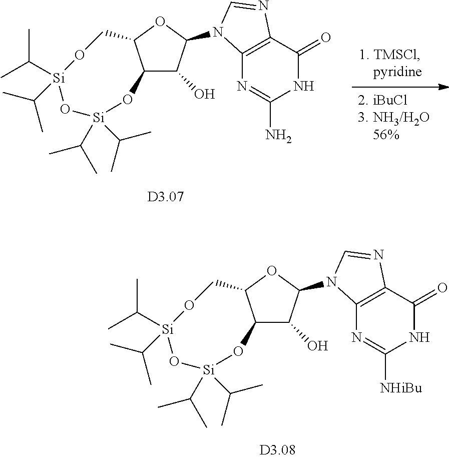

2-Amino-9-((6aS,8R,9R,9aR)-9-hydroxy-2,2,4,4-tetraisopropyltetrahydro-6H-furo[3,2-f][1,3,5,2,4]trioxadisilocin-8-yl)-1,9-dihydro-6H-purin-6-one (D3.07) (14.20 g, 27.10 mmol) was dissolved in pyridine (100 mL) under nitrogen. The solution was cooled with an ice bath, and trimethylsilyl chloride (13.20 mL, 135 mmol, 5 eq.) was added dropwise. The ice bath was removed, and the reaction was stirred for 1 hour at room temperature. The reaction was again cooled with an ice bath, and isobutyryl chloride (13.40 g, 135 mmol, 5 eq.) was added dropwise. After the addition, the reaction was warmed up slowly to room temperature and stirred overnight. The next day, the reaction was cooled with an ice bath and water (40 mL) was added dropwise, keeping the temperature below 7° C. The reaction was then stirred at room temperature for 1 hour. The reaction was cooled again, and NH₄₀H (55 mL) was added dropwise to the reaction. The reaction was stirred for another 30 minutes. Most of the NH₄₀H was evaporated at room temperature to obtain mostly water and product. The remaining solution was diluted with EtOAc, and the organic material was washed with plain water (100 mL). The aqueous layer was removed, and the organic material was washed with sat. NaHCO₃ and sat. brine, then dried over Na₂SO₄. The salt was filtered out, and the solvent was evaporated to obtain crude material. The crude material was dissolved and purified by Biotage column (100 g, eluted with DCM/MeOH (97/3)+1% Et₃N) to obtain product D3.08. (9.0 g, 56% yield)

### Preparation of Compound D3.09

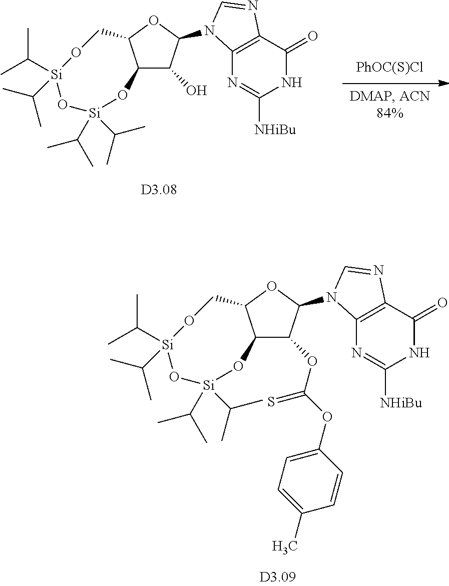

9-((6aS,8R,9R,9aR)-9-hydroxy-2,2,4,4-tetraisopropyltetrahydro-6H-furo[3,2-f][1,3,5,2,4]trioxadisilocin-8-yl)-2-(isobutylamino)-1,9-dihydro-6H-purin-6-one (D3.08) (7.80 g, 131 mmol) and 4-dimethylaminopyridine (3.20 g, 262 mmol, 2 eq.) were dissolved in anhydrous acetonitrile (131 mL). Some anhydrous THF 50 mL was added to dissolved the nucleoside. Then O-4-methylphenyl chlorothioformate (2.69 mL, 144 mmol, 1.2 eq.) was added slowly to the reaction. The reaction was stirred at room temperature for 16 hours. The next day, reaction was TLC in DCM/MeOH (95/5) The solvents were removed under reduced pressure, and the residue was partitioned between ethyl acetate and water. The aqueous layer was extracted with ethyl acetate, and the combined organics were washed with 10% HCl (aq), water, saturated sodium bicarbonate solution, water, and brine. The organic fractions were dried over magnesium sulfate and concentrated. Purification by Biotage (Si, 100 g col, eluted with 0-3% MeOH/DCM) afforded the desired product (D3.09) as a white solid. (8.24 g, 84% yield)

### Preparation of Compound D3.10

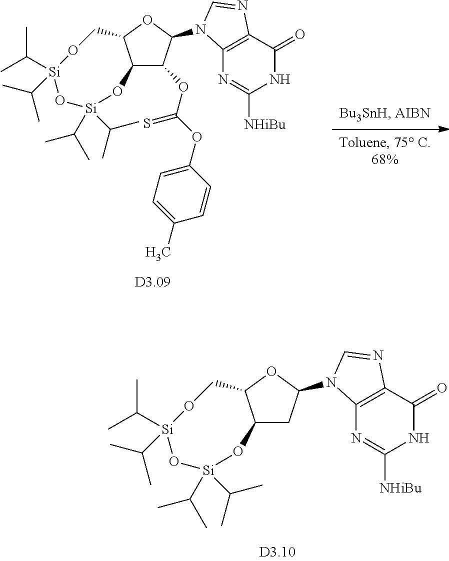

Azobisisobutyronitrile (AIBN) (0.267 g, 1.80 mmol, 0.2 eq) and tributyltin hydride (24.10 mL, 89.4 mmol 10 eq.) in toluene (40 mL) were degassed for 30 minutes with nitrogen then added dropwise to a degassed (with nitrogen) solution of compound O-((6aS,8R,9R,9aS)-8-(2-(isobutylamino)-6-oxo-1,6-dihydro-9H-purin-9-yl)-2,2,4,4-tetraisopropyltetrahydro-6H-furo[3,2-f][1,3,5,2,4]trioxadisilocin-9-yl)O-(p-tolyl) carbonothioate (D3.09) (6.67 g, 8.94 mmol) in toluene (140 mL) at 80° C. (degassed for 30 minutes with nitrogen). The solution was heated to 80° C. for 1 hour before being cooled to room temperature. The solvents were removed under reduced pressure. The reaction was monitored with TLC in EtOAc/hexane (7/3). Purification by Biotage (Si, 100 g col, 70% ethyl acetate/hexanes) afforded the desired product (D3.10) as a white solid. (3.54 g, 68% yield)

### Preparation of Compound D3.11

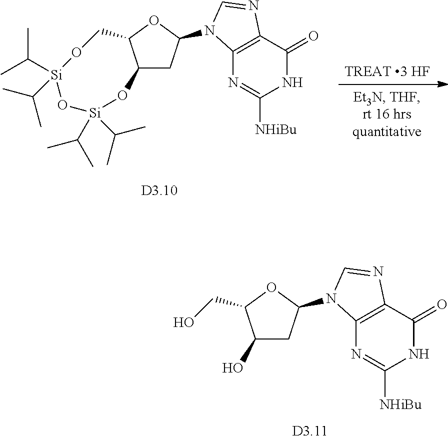

TEA (2.13 mL, 15.40 mmol, 2.5 eq.) was added to a solution of 2-(isobutylamino)-9-((6aS,8R,9aR)-2,2,4,4-tetraisopropyltetrahydro-6H-furo[3,2-f][1,3,5,2,4]trioxadisilocin-8-yl)-1,9-dihydro-6H-purin-6-one (D3.10) (3.54 g, 6.11 mmol) in THF (30 mL). The reaction was cooled to 0° C. with an ice bath under an atmosphere of nitrogen. Triethylamine trihydrofluoride (4.98 mL, 30.5 mmol, 5 eq.) was added slowly at 0° C., and then the reaction was warmed to room temperature and stirred for 16 hours. The solvents were removed under reduced pressure and purification by plug of silica gel (50 g); elution with a 5-10% methanol/dichloromethane solution afforded the desired product (D3.11) as a white solid. (3.7 g, 100+% yield)

### Preparation of Compound D3.12

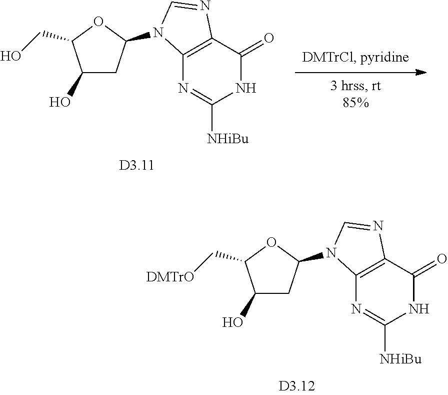

DMTrCl (4.36 g, 13.20 mmol, 1.2 eq.) was added to a solution of 9-((2R,4R,5S)-4-hydroxy-5-(hydroxymethyl)tetrahydrofuran-2-yl)-2-(isobutylamino)-1,9-dihydro-6H-purin-6-one (D3.11) (3.70 g, 110 mmol) in pyridine (30 mL) at room temperature and stirred for 2 hours. The reaction was quenched with the addition of methanol (2 mL), followed by dilution of the reaction with water and ethyl acetate. The aqueous layer was extracted with ethyl acetate. The combined organics were washed with water, saturated sodium bicarbonate, and brine. The organic material was dried over Na₂SO₄ for 10 minutes, and the salts were filtered out. The filtrate was evaporated to obtain crude material. The crude material was dissolved in DCM and loaded to Biotage (Si, 100 g col, 0-5% methanol/dichloromethane) to afford the desired product (D3.12) as a white solid. (3.30 g, 85% yield)

### Preparation of Compound 3a

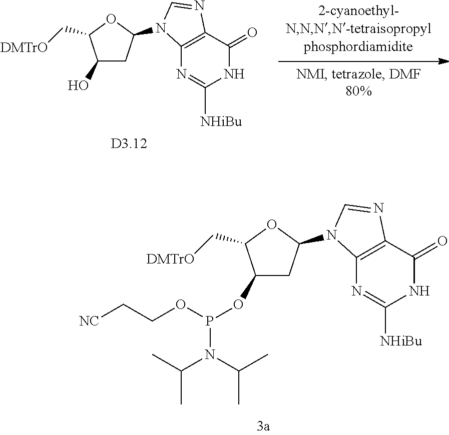

1H-Tetrazole (0.294 g, 4.25 mmol, 0.8 eq.) and 1-methylimidazole (0.105 mL, 1.33 mmol, 0.25 eq.) were added to a solution of 9-((2R,4R,5S)-5-((bis(4-methoxyphenyl)(phenyl)methoxy)methyl)-4-hydroxytetrahydrofuran-2-yl)-2-(isobutylamino)-1,9-dihydro-6H-purin-6-one (D3.12) (3.40 g, 5.33 mmol) in DMF (40 mL) at room temperature under an atmosphere of nitrogen. 3-Bis(diisopropylamino)phosphanyloxypropanenitrile (2.53 mL, 7.97 mmol, 1.5 eq.) was then added dropwise and the reaction was stirred at room temperature for 90 minutes. Water (1.0 mL) was added to quench the reaction. A 3:1 mixture of toluene/hexanes (80 mL) was added, and the organic layer was washed four times with a 3:2 mixture of DMF/H₂O (50 mL). The organics were then washed with saturated sodium bicarbonate solution, brine, dried over solid sodium sulfate, and concentrated under reduced pressure to a white foam. Purification by a plug of silica gel (50 g, eluted with 100% EtOAc) afforded the desired product (3a) as a white amorphous solid. (3.54 g, 80% yield)

### Example 36: Design and Synthesis of Stereo-Non-Standard Nucleoside, α-L-Deoxyribose

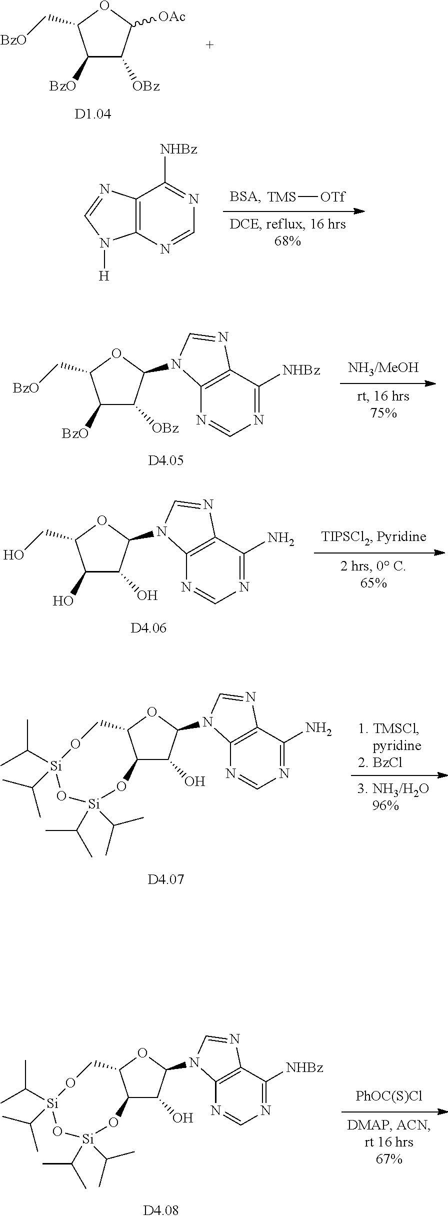

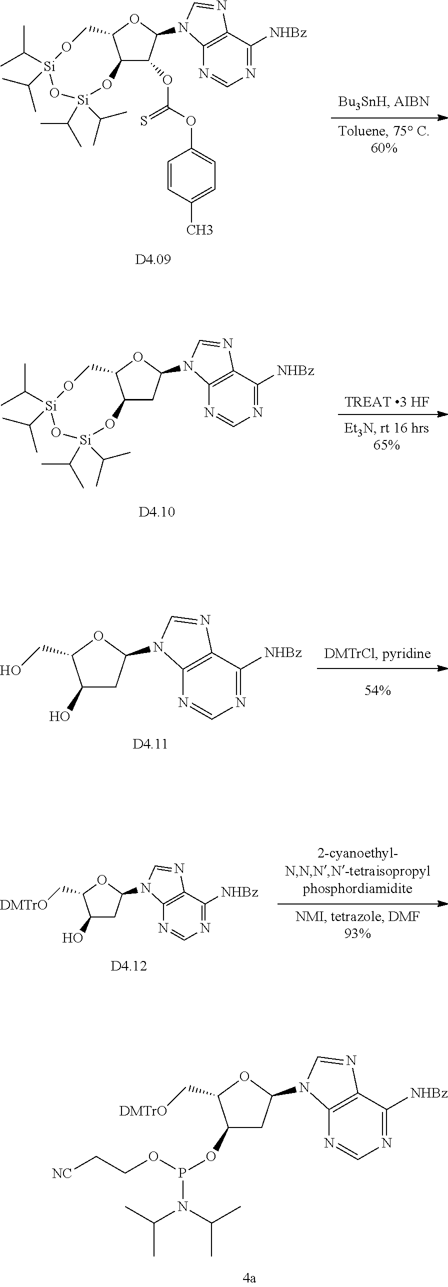

### Preparation of Compound D4.05

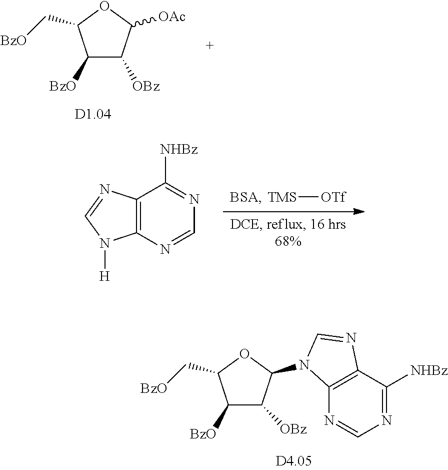

N-(9H-purin-6-yl)benzamide (23.40 g, 97.30 mmol, 1.30 eq.) and [(2S,3S,4R)-5-acetoxy-3,4-dibenzoyloxy-tetrahydrofuran-2-yl]methyl benzoate (D1.04) (38 g, 75.3 mmol) was first co-evaporated 4×(50 mL) with toluene at 60° C. The mixture was suspended in anhydrous dichloroethane (800 mL), and N,O-Bis(trimethylsilyl)acetamide (73.7 mL, 301 mmol, 4 eq.) was added to the reaction mixture. Reflux at 80° C. for 1 hour resulted in a clear solution. The reaction solution was cooled down with an ice bath to 5° C. Trimethylsilyl trifluoromethanesulfonate (21.80 mL, 121 mmol, 1.6 eq.) was added, and the reaction was stirred to reflux overnight. The next day, the reaction was concentrated under reduced pressure. The crude oil was diluted with ethyl acetate (200 mL). The organic material with plain DI water (200 mL), followed with saturated sodium bicarbonate solution to pH 7, then saturated brine solution. The organic material was dried over N₂SO₄ for 10 minutes and filtered, then solvent was concentrated under reduced pressure. Purification by Biotage (Si, 320 g col, eluted with 0-5% dichloromethane/methanol) afforded the desired product (D4.05) as a white solid (35.18 g, 68% yield).

### Preparation of Compound D4.06

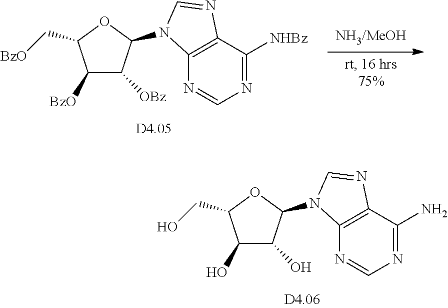

(2R,3R,4S,5S)-2-(6-Benzamido-9H-purin-9-yl)-5-((benzoyloxy)methyl)tetrahydrofuran-3,4-diyl dibenzoate (D4.05) (43.0 g, 58.50 mmol) was suspended in methanol (50.0 mL) and cooled to −20° C. A 7 N NH₃/MeOH (150 mL) solution was added to the reaction, then the reaction was heated overnight at 45° C. The next day, the solution was concentrated to an oil, and the material was suspended in EtOAc (100 mL) to obtain a white precipitate. The solid was filtered and rinsed with fresh EtOAc. The solid was dried under high vacuum to obtain product D4.06 (11.70 g, 75% yield).

### Preparation of Compound D4.07

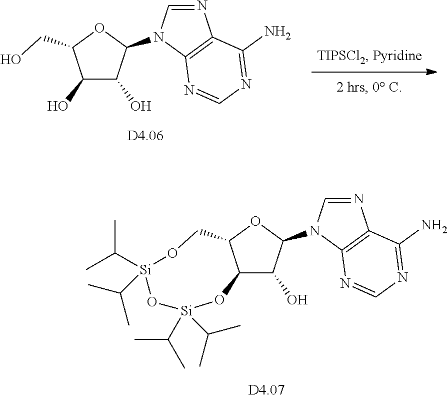

(2R,3R,4R,5S)-2-(6-Amino-9H-purin-9-yl)-5-(hydroxymethyl)tetrahydrofuran-3,4-diol (D4.06) (11.76 g, 43.78 mmol) was dissolved in pyridine (400 mL) under nitrogen. The solution was cooled with an ice bath to 0° C., and 1,3-dichloro-1,1,3,3-tetraisopropyldisiloxane (12.66 mL, 39.60 mmol, 0.90 eq.) was added dropwise. The reaction was warmed up slowly to about 10° C. for 2 hours. TLC in EtOAc/hexane (8/2) indicated reaction was completed. The reaction was cooled down with an ice bath to 0° C. and then was quenched by slowly adding DI water (20 mL). The solution was concentrated to an oil under reduced pressure. The oil was dissolved in ethyl acetate, and the organics were washed with 10% HCl (aq), water, saturated sodium bicarbonate solution, water, and brine. The solution was concentrated to afford the desired product (D4.07) as a colorless oil. The crude oil was suspended in hexane to obtain a white precipitate (final weight 13.90 g crude, 62% yield).

### Preparation of Compound D4.08

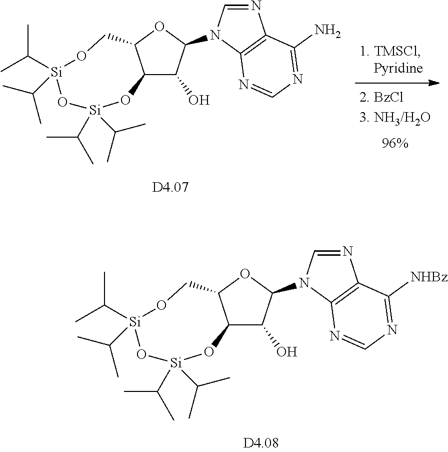

Compound (6aS,8R,9R,9aR)-8-(6-amino-9H-purin-9-yl)-2,2,4,4-tetraisopropyltetrahydro-6H-furo[3,2-f][1,3,5,2,4]trioxadisilocin-9-ol (D4.07) (7.90 g, 15.50 mmol) was dissolved in pyridine (100 mL) under nitrogen. Cooled solution with an ice bath and added trimethylsilyl chloride (13.80 mL, 108 mmol, 5 eq.) dropwise. Removed the ice bath and let reaction stir for 1 hr at rt. Cooled reaction again with an ice bath and added dropwise benzoyl chloride (9 mL, 77.50 mmol, 5 eq.) after the addition, let reaction warm up slowly to rt and continue stir reaction overnight. The next day, cooled reaction with an ice bath and added water 150 mL dropwise and keep temperature below 7° C. Let reaction stir at rt for 1 hour. Cooled reaction again and added dropwise NH₄OH (100 mL) to reaction. Reaction was stirred for another 30 minutes. Evaporated most of the NH₄OH at room temperature to obtain mostly water and product.

Remaining solution was diluted with EtOAc and wash the organic with plain water 100 (mL) aqueous layer was removed and organic continue to wash organic with sat. NaHCO₃, sat. brine and finally dry organic over Na₂SO₄ filtered salt and evaporated solvent to obtain crude material. The crude material was dissolved and purified by Biotage (Si, 100 g col, eluted with 0-5% dichloromethane/methanol) afforded the desired product (D4.08) as a white solid (9.20 g, 96% yield).

### Preparation of Compound D4.09

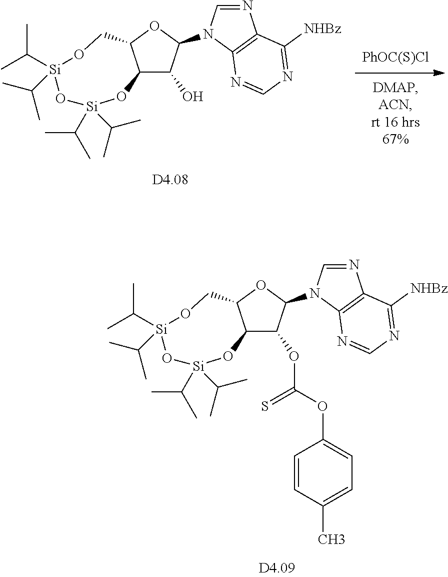

N-(9-((6aS,8R,9R,9aR)-9-Hydroxy-2,2,4,4-tetraisopropyltetrahydro-6H-furo[3,2-f][1,3,5,2,4]trioxadisilocin-8-yl)-9H-purin-6-yl)benzamide (D4.08) (8.0 g, 130 mmol) and 4-dimethylaminopyridine (3.18 g, 261 mmol, 2 eq.) were dissolved in anhydrous acetonitrile (131 mL). Some anhydrous THF (50 mL) was added to help dissolve the nucleoside. Then 0-4-methylphenyl chlorothioformate (2.18 mL, 143 mmol, 1.2 eq.) was added slowly. The reaction was stirred at room temperature for 16 hours. The solvents were removed under reduced pressure, and the residue was partitioned between ethyl acetate and water. The product was extracted from the aqueous layer with ethyl acetate (2×), and the combined organics were washed with 10% HCl (aq), water, saturated sodium bicarbonate solution, water, and brine. The organic fraction was dried over magnesium sulfate and concentrated. Purification by Biotage (Si, 100 g col, eluted with 0-3% methanol/DCM) afforded the desired product (D4.09) as a white solid (6.67 g, 67% yield).

### Preparation of Compound D4.10

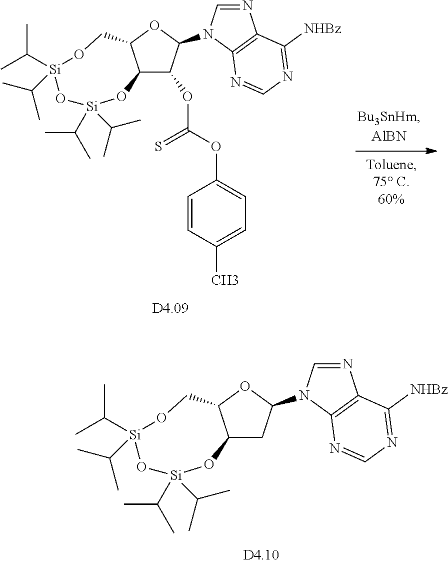

Azobisisobutyronitrile (AIBN) (0.287 g, 1.75 mmol, 0.2 eq) and tributyltin hydride (23.50 mL, 87.30 mmol 10 eq.) in toluene (40 mL) (Note: solution was degassed for 30 minutes with nitrogen) were added dropwise to a degassed (with nitrogen) solution of O-((6aS,8R,9R,9aS)-8-(6-benzamido-9H-purin-9-yl)-2,2,4,4-tetraisopropyltetrahydro-6H-furo[3,2-f][1,3,5,2,4]trioxadisilocin-9-yl)O-(p-tolyl) carbonothioate (D4.09) (6.67 g, 8.73 mmol) in toluene (140 mL) at 80° C. The solution was heated at 80° C. for 1 hour before being cooled to room temperature. The solvents were removed under reduced pressure. The reaction was monitored with TLC in EtOAc/hexane (7/3). Purification by Biotage (Si, 100 g col, 70% ethyl acetate/hexanes) afforded the desired product (D4.10) as a white solid (3.0 g, 60% yield).

### Preparation of Compound D4.11

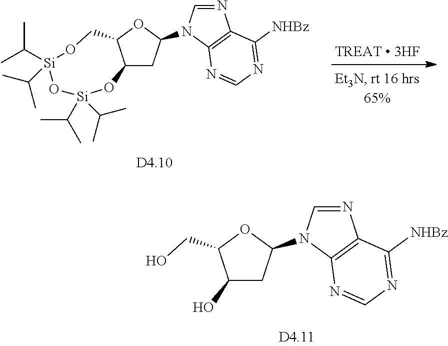

TEA (1.36 mL, 9.80 mmol, 2.5 eq.) was added to a solution of N-(9-((6aS,8R,9aR)-2,2,4,4-tetraisopropyltetrahydro-6H-furo[3,2-f][1,3,5,2,4]trioxadisilocin-8-yl)-9H-purin-6-yl)benzamide (D4.10) (2.34 g, 3.91 mmol) in THF (30 mL). The reaction was cooled to 0° C. with an ice bath under an atmosphere of nitrogen. Triethylamine trihydrofluoride (3.19 mL, mmol, 5 eq.) was added slowly at 0° C., and then the reaction was warmed to room temperature and stirred for 16 hours. The solvents were removed under reduced pressure and purification by plug of silica gel (50 g). Elution with 5-10% methanol/dichloromethane) afforded the desired product (D4.11) as a white solid (0.90 g, 65% yield).

### Preparation of Compound D4.12

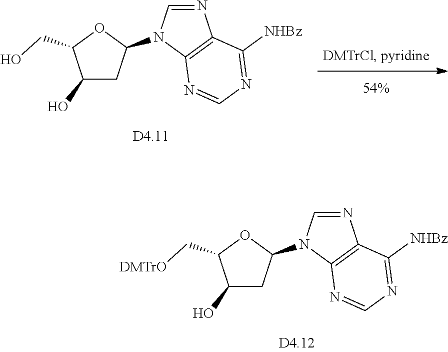

DMTrCl (1.1 g, 3.04 mmol, 1.2 eq.) was added to a solution of N-(9-((2R,4R,5S)-4-hydroxy-5-(hydroxymethyl)tetrahydrofuran-2-yl)-9H-purin-6-yl)benzamide (D4.11) (0.90 g, 2.53 mmol) in pyridine (20 mL) at room temperature and stirred for 2 hours. The reaction was quenched with the addition of methanol (2 mL), followed by dilution of the reaction with water and ethyl acetate. The aqueous layer was extracted with ethyl acetate. The combined organics were washed with water, saturated sodium bicarbonate, and brine. The organic material was dried over Na₂SO₄ for 10 minutes, and the salts were filtered out. The filtrate was evaporated to obtain crude material, which was dissolved in DCM and loaded to Biotage (Si, 50 g col, 0-5% methanol/dichloromethane) to afford the desired product (D4.12) as a white solid (0.90 g, 54% yield).

### Preparation of Compound 4a

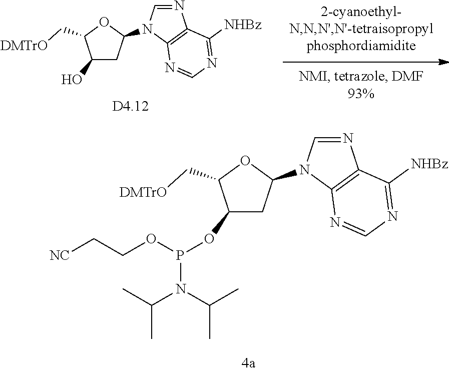

1H-Tetrazole (0.075 g, 1.09 mmol, 0.8 eq.) and 1-methylimidazole (0.0271 mL, 0.342 mmol, 0.25 eq.) were added to a solution of N-(9-((2R,4R,5S)-5-((bis(4-methoxyphenyl)(phenyl)methoxy)methyl)-4-hydroxytetrahydrofuran-2-yl)-9H-purin-6-yl)benzamide (D4.12) (0.90 g, 1.37 mmol) in DMF (10 mL) at room temperature under an atmosphere of nitrogen. 3-Bis(diisopropylamino)phosphanyloxypropanenitrile (0.652 mL, 2.05 mmol, 1.5 eq.) was then added dropwise, and the reaction was stirred at room temperature for 90 minutes. Water (1.0 mL) was added to quench the reaction. A 3:1 mixture of toluene/hexanes (80 mL) was added, and the organic layer was washed four times with a 3:2 mixture of DMF/H₂O (50 mL). The organics were then washed with saturated sodium bicarbonate solution, brine, dried over solid sodium sulfate, and concentrated under reduced pressure to a white foam. Purification by a plug of silica gel (30 g) and elution with EtOAc/hexane (9/1) afforded the desired product (4a) as a white amorphous solid (1.10 g, 93% yield).

### Example 37: Synthesis of an Amidite of a Stereo-Non-Standard Nucleoside, β-D-Deoxyxylose

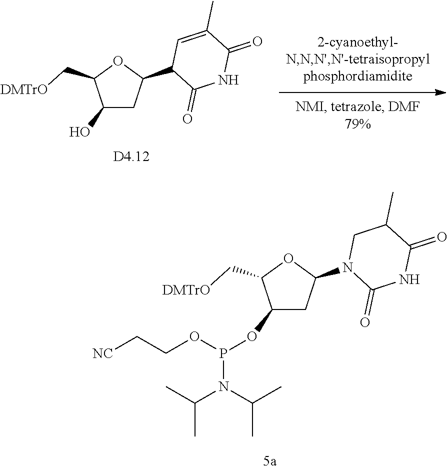

Synthesis of compound D5.01 has been previously described, see, e.g., Poopeiko, et al., Biorg. Med. Chem. Letters, 2003.

### Preparation of Compound 5a

1H-Tetrazole (0.5647 g, 7.92 mmol 0.8 eq.) and 1-methylimidazole (0.196 mL, 2.47 mmol, 0.25 eq) were added to a solution of 1-((2R,4R,5R)-5-((bis(4-methoxyphenyl)(phenyl)methoxy)methyl)-4-hydroxytetrahydrofuran-2-yl)-5-methylpyrimidine-2,4(1H,3H)-dione (D5.01) (5.31 g, 9.90 mmol) in DMF (51 mL) at room temperature under an atmosphere of nitrogen. 3-Bis(diisopropylamino)phosphanyloxypropanenitrile (4.72 mL, 14.80 mmol, 1.5 eq.) was then added dropwise, and the reaction was stirred at room temperature for 90 minutes. Water (1.0 mL) was added to quench the reaction. A 3:1 mixture of toluene/hexanes (80 mL) was added, and the organic layer was washed four times with a 3:2 mixture of DMF/H₂O (50 mL). The organics were then washed with saturated sodium bicarbonate solution, brine, dried over solid sodium sulfate and concentrated under reduced pressure to obtain crude oil. Crude material was dissolved in DCM+1% Et₃N and load to a plug of silica gel (50 g). Note: The silica gel was first treated with EtOAc/hexane (1/1)+1% Et₃N before loading the material. Elution with EtOAc/hexane (1/1)+1% Et₃N yielded product 5a (5.80 g, 79% yield).

### Example 38: Synthesis of an Amidite of a Stereo-Non-Standard Nucleoside, β-D-Deoxyxylose

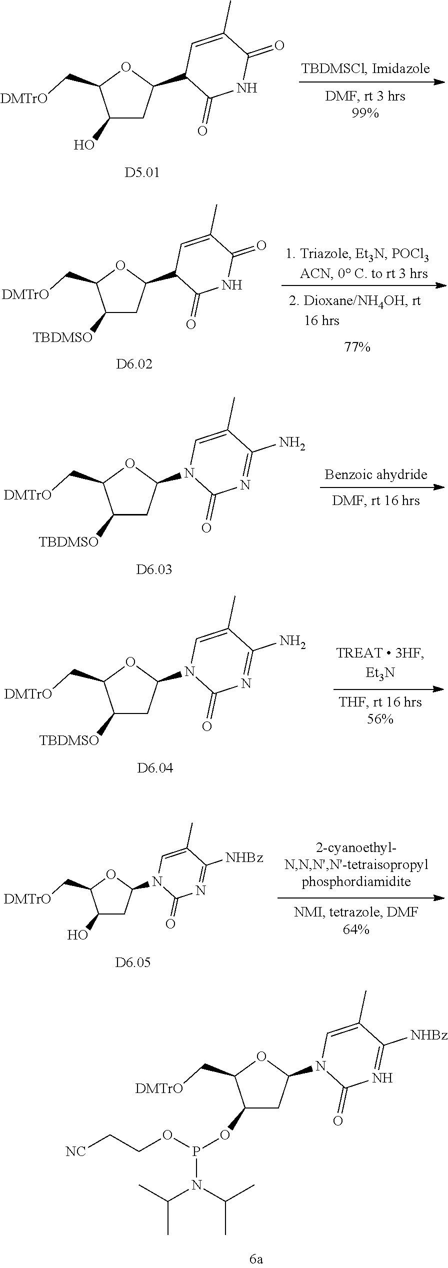

### Preparation of Compound D6.02

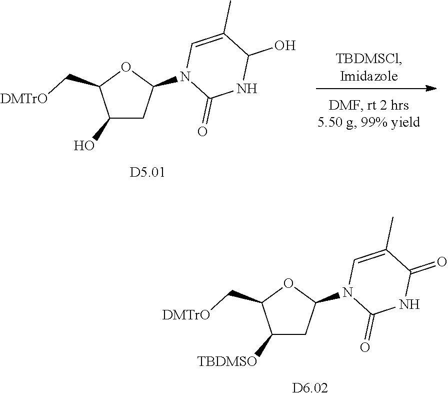

Compound 1-[(2R,3R,4R,5S)-3,4-dihydroxy-5-(hydroxymethyl)tetrahydrofuran-2-yl]-5-methyl-pyrimidine-2,4-dione (D5.01) (4.56 g, 8.37 mmol) was dissolved in anhydrous dimethylformamide (40 mL), and the solution was stirred under nitrogen. 1H-imidazole (1.44 g, 16.7 mmol, 2 eq.) was added, and the solution was cooled with an ice bath to 0° C. Tert-butylchlorodimethylsilane (1.40 g, 16.7 mmol, 2 eq) in a solution of anhydrous dimethylformamide (10 mL) was added dropwise to the reaction. The ice bath was removed, and the reaction was warmed up to room temperature and continued to stir for 3 hours. TLC in hexane/EtOAc (6/4) indicated reaction was completed. The solution was cooled with an ice bath to 0° C., and the reaction was slowly quenched by adding 30 mL of water. The solution was transferred to a separatory funnel and washed with plain DI water. The product was extracted with ethyl acetate. The aqueous layer was removed from the organic, and the organic layer was washed with saturated NaHCO₃ and saturated brine. The organic material was dried over Na₂SO₄, filtered, and evaporated solvent to obtain a crude oil. The crude material was dissolved in dichloromethane, loaded to a plug of silica gel, and eluted with EtOAc/hexane (6/4) to obtain the product D6.02 (5.50 g, 99% yield).

### Preparation of Compound D6.03

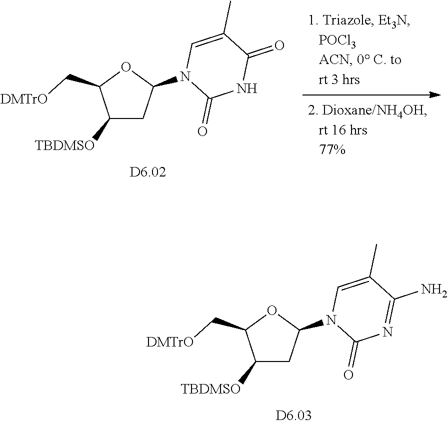

POCl₃ (6.45 mL, 70.40 mmol, 8 eq) was added dropwise to a suspension of 1,2,4-1H-triazole (20.7 g, 299 mmol, 34 eq.) in acetonitrile (200 mL) under an atmosphere of nitrogen at 0° C. The ice bath was then removed, and the reaction was stirred at room temperature for 20 minutes. The reaction was cooled down in an ice bath again, triethylamine (49.10 mL, 352 mmol, 40 eq.) was added dropwise to the reaction. The ice bath was removed, and the reaction was stirred for 30 minutes. The reaction was cooled down to 0° C. and a solution of (2R,3R,5R)-2-((bis(4-methoxyphenyl)(phenyl)methoxy)methyl)-5-(5-methyl-2,4-dioxo-3,4-dihydropyrimidin-1(2H)-yl)tetrahydrofuran-3-yl (2-cyanoethyl) diisopropylphosphoramidite (D6.02) (5.80 g, 8.80 mmol) in acetonitrile (20 mL) was added dropwise to the reaction. This was stirred at room temperature overnight. The reaction was concentrated to a small volume under reduced pressure then diluted with ethyl acetate. The organic layer was washed with aqueous saturated sodium bicarbonate (2×), water, and brine, then concentrated to a yellow oil to afford the desired crude material. Without any further purification, the crude material was suspended in dioxane/NH₄₀H (30 mL/10 mL). The solution was stirred at room temperature for 2 hours. TLC in EtOAc/hexane (8/2) indicated reaction was completed. The solvent was concentrated under reduced pressure, and the remaining oil was diluted with ethyl acetate and washed with 1×200 mL plain DI water, 1×200 mL sat. NaHCO₃, and 1×200 mL sat. brine. The organic material was dried over Na₂SO₄, filtered, and concentrated under reduced pressure to obtain a crude oil. The crude material was dissolved in DCM and loaded to a plug of silica gel for purification. Elution with dichloromethane/methanol (95/5) gave the product D6.03 (8.83 g, 77% yield).

### Preparation of Compound D6.04

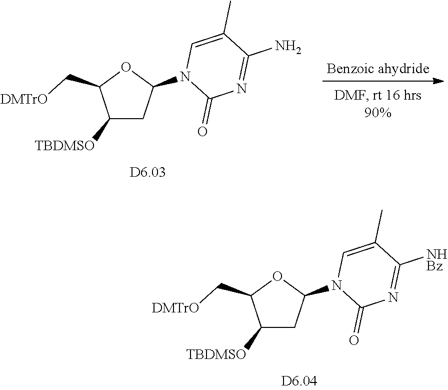

4-Amino-1-((2R,4R,5R)-5-((bis(4-methoxyphenyl)(phenyl)methoxy)methyl)-4-((tert-butyldimethylsilyl)oxy)tetrahydrofuran-2-yl)-5-methylpyrimidin-2(1H)-one (D6.03) (5.30 g, 8.03 mmol) was dissolved in anhydrous dimethylformamide (30 mL) and stirred under nitrogen at room temperature. Benzoic anhydride (2.0 g, 8.83 mmol, 1.1 3q.) was added to the reaction. The reaction was stirred at room temperature overnight. The next day, TLC in EtOAc/hexane (6/4) indicated reaction was completed. The reaction was cooled down with an ice bath to 0° C. About 20 mL of water was slowly added followed with EtOAc. The mixture was stirred for 10 minutes. The solution was transferred to a separatory funnel and washed with plain DI water, and the product was extracted with EtOAc. The aqueous layer was removed, and the organic material was washed with sat. NaHCO₃ and sat. brine solution. The organic material was dried over Na₂SO₄ for 10 minutes then the salts were filtered out. The solvent was concentrated under reduced pressure to obtain a crude oil. The crude material was dissolved in DCM and loaded to a plug of silica gel and eluted with hexane/EtOAc (6/4). The fractions with product were combined and concentrated under reduced pressure to obtain the product D6.04 (5.33 g, 90% yield).

### Preparation of Compound D6.05

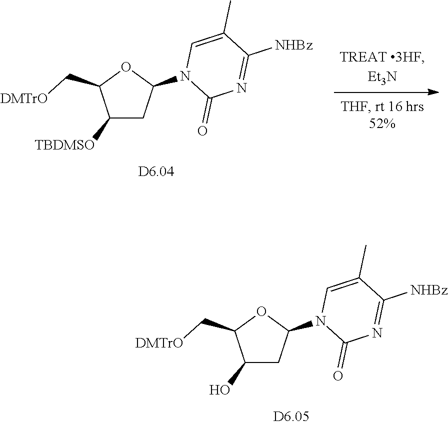

TEA (0.88 mL, 6.36 mmol, 2.5 eq.) was added to a solution of N-(1-((2R,4R,5R)-5-((bis(4-methoxyphenyl)(phenyl)methoxy)methyl)-4-((tert-butyldimethylsilyl)oxy)tetrahydrofuran-2-yl)-5-methyl-2-oxo-1,2-dihydropyrimidin-4-yl)benzamide (D6.04) (1.50 g, 2.89 mmol) in tetrahydrofuran (10.0 mL). The reaction was cooled to 0° C. under an atmosphere of nitrogen. Triethylamine trihydrofluoride (2.08 mL, 12.77 mmol, 5 eq.) was added slowly and then the reaction was warmed to room temperature and stirred for 16 hours. The solvents were removed under reduced pressure and purification by Biotage (Si, 20 g col, 70% EtOAc/hexane) afforded the desired product (D6.05) as a white solid (0.66 g, 52% yield).

### Preparation of Compound 6a

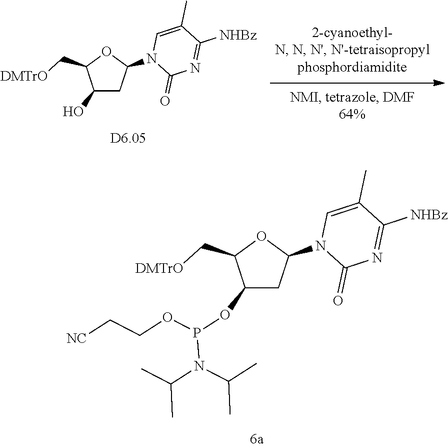

1H-Tetrazole (0.0561 g, 0.813 mmol, 0.8 eq.) and 1-methylimidazole (0.0201 mL, 0.254 mmol) were added to a solution of N-(1-((2R,4R,5R)-5-((bis(4-methoxyphenyl)(phenyl)methoxy)methyl)-4-hydroxytetrahydrofuran-2-yl)-5-methyl-2-oxo-1,2-dihydropyrimidin-4-yl)benzamide (D6.05) (0.66 g, 1.02 mmol) in DMF (10 mL), followed by dropwise addition of 3-bis(diisopropylamino)phosphanyloxypropanenitrile (0.484 mL, 1.52 mmol, 1.5 eq.). The reaction was stirred at room temperature for 2 hours. Water (1.0 mL) was added to quench the reaction. A 3:1 mixture of toluene/hexanes (20 mL) was added and the organic layer was washed four times with a 3:2 mixture of DMF/H₂O (20 mL). The organics were then washed with saturated sodium bicarbonate solution, brine, dried over solid sodium sulfate and concentrated under reduced pressure. Purification by Biotage (Si, 20 g col, 40% ethyl acetate/hexanes+1% Et₃N) (loaded with a small amount of DCM) afforded the desired product (6a) as a white amorphous solid. (55.0 g, 64% yield)

### Example 39: Synthesis of an Amidite of a Stereo-Non-Standard Nucleoside, β-D-Deoxyxylose

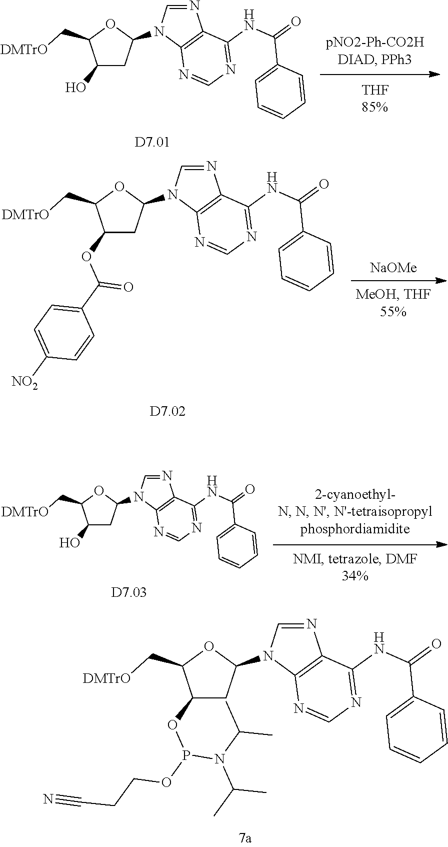

Steps of this synthesis have been previously described, see, e.g., Lavandera, et al., Tetrahedron, 2003; Chen, et al., Nuc. Acids Res., 1995.

### Preparation of Compound D7.02

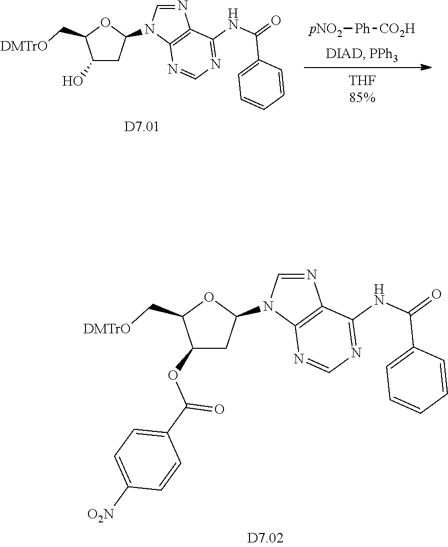

4-Nitrobenzoic acid (4.07 g, 24.3 mmol) and triphenyl phosphine (6.38 g, 24.3 mmol) were added to a solution of N-[9-[(2R,4S,5R)-5-[[bis(4-methoxyphenyl)-phenyl-methoxy]methyl]-4-hydroxy-tetrahydrofuran-2-yl]purin-6-yl]benzamide (D7.01) (8.00 g, 12.2 mmol) in THF (70.0 mL) at room temperature under an atmosphere of nitrogen. The reaction was cooled to 0° C. in an ice bath before dropwise addition of diisopropyl azodicarboxylate (4.71 mL, 24.3 mmol) in THF (10.00 mL). The reaction was stirred for 30 minutes at 0° C. and then warmed to room temperature for 60 minutes. The reaction mixture was diluted with water, ethyl acetate, and saturated sodium bicarbonate solution. The aqueous layer was extracted with ethyl acetate. The combined organic fractions were washed with brine and then concentrated under reduced pressure. Purification by Biotage (Si, 50 g col, 0-100 ethyl acetate/hexanes) afforded the desired product (D7.02) as an off-white foam.

[(2R,3R,5R)-5-(6-benzamidopurin-9-yl)-2-[[bis(4-methoxyphenyl)-phenyl-methoxy]methyl]tetrahydrofuran-3-yl] 4-nitrobenzoate (8.35 g, 10.3 mmol, yield: 85.1%)

¹H NMR (300 MHz, CHLOROFORM-d) δ 8.89 (s, 1H), 8.63 (s, 1H), 8.12-8.25 (m, 3H), 8.00 (dd, J=1.54, 7.04 Hz, 2H), 7.54 (dd, J=1.22, 7.10 Hz, 5H), 7.33-7.37 (m, 2H), 7.19 (br dd, J=3.33, 5.50 Hz, 7H), 6.65-6.73 (m, 4H), 6.54 (dd, J=2.62, 6.46 Hz, 1H), 5.95 (t, J=3.33 Hz, 1H), 4.57-4.70 (m, 1H), 3.74 (s, 3H), 3.73 (s, 3H), 3.68 (dd, J=5.63, 9.22 Hz, 1H), 3.46 (dd, J=7.30, 9.22 Hz, 1H), 3.06 (s, 2H)

¹³C NMR (75 MHz, CHLOROFORM-d) δ 164.6, 163.3, 158.6, 158.5, 152.5, 151.4, 150.6, 149.5, 144.2, 140.7, 135.3, 135.0, 133.4, 133.2, 132.9, 131.9, 131.9, 131.9, 130.4, 130.0, 130.0, 129.9, 128.9, 127.9, 127.0, 123.7, 113.1, 113.1, 86.6, 84.6, 82.5, 73.4, 60.8, 55.2, 55.2, 55.1, 39.0

LCMS: M+H=807.3

### Preparation of Compound D7.03

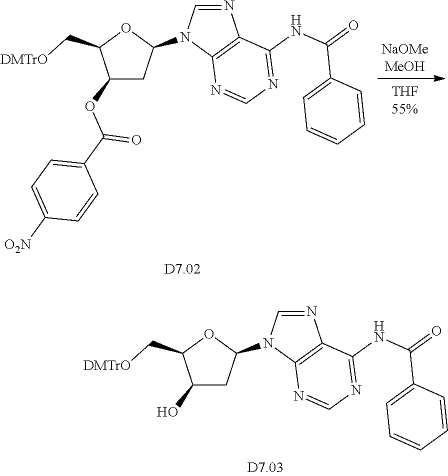

[(2R,3R,5R)-5-(6-benzamidopurin-9-yl)-2-[[bis(4-methoxyphenyl)-phenyl-methoxy]methyl]tetrahydrofuran-3-yl] 4-nitrobenzoate (D7.02) (8.35 g, 10.3 mmol) was dissolved in THF (69.1 mL) and then cooled to 0° C. in an ice bath. Sodium methoxide (0.500 M, 20.7 mL, 10.3 mmol) in methanol was added, and the reaction was stirred for 45 minutes at 0° C. The reaction mixture was diluted with water and ethyl acetate. The aqueous layer was extracted with ethyl acetate, followed by the combined organic fractions being washed with brine and concentrated to an oil. Purification by Biotage (Si, 220 g col, 0-100% ethyl acetate/hexanes) afforded the product (D7.03) as a white foam.

N-[9-[(2R,4R,5R)-5-[[bis(4-methoxyphenyl)-phenyl-methoxy]methyl]-4-hydroxy tetrahydrofuran-2-yl]purin-6-yl]benzamide (3.71 g, 5.64 mmol, yield: 54.5%)

¹H NMR (300 MHz, CHLOROFORM-d) δ 8.98 (s, 1H), 8.74 (s, 1H), 8.24 (s, 1H), 7.98-8.07 (m, 2H), 7.59-7.66 (m, 1H), 7.50-7.58 (m, 2H), 7.39-7.45 (m, 2H), 7.28-7.35 (m, 4H), 7.16-7.26 (m, 3H), 6.73-6.82 (m, 4H), 6.27 (dd, J=2.56, 9.22 Hz, 1H), 5.67 (d, J=8.32 Hz, 1H), 4.43-4.56 (m, 1H), 4.11 (s, 1H), 3.77 (d, J=1.92 Hz, 6H), 3.50-3.64 (m, 2H), 2.89 (ddd, J=6.02, 9.09, 15.23 Hz, 1H), 2.54 (dd, J=2.50, 15.55 Hz, 1H)

¹³C NMR (75 MHz, CHLOROFORM-d) δ 164.6, 158.5, 152.0, 150.4, 149.9, 144.6, 143.1, 135.9, 135.8, 133.5, 132.8, 130.0, 130.0, 128.9, 128.1, 127.9, 127.8, 126.8, 123.8, 113.1, 86.6, 84.4, 84.1, 71.0, 62.4, 55.2, 40.9

LCMS: M+H=658.3

### Preparation of Compound 7a

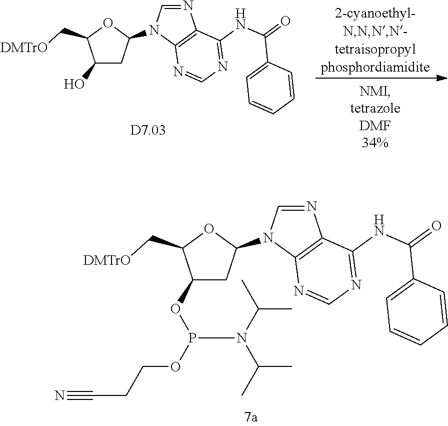

N-[9-[(2R,4R,5R)-5-[[bis(4-methoxyphenyl)-phenyl-methoxy]methyl]-4-hydroxy-tetrahydrofuran-2-yl]purin-6-yl]benzamide (D7.03) (3.71 g, 5.64 mmol) was dissolved in dry DMF (57.2 mL) under an atmosphere of nitrogen. To this was added 1H-tetrazole (0.316 g, 4.51 mmol) and 1-methylimidazole (0.112 mL, 1.41 mmol), followed by dropwise addition of 3-bis(diisopropylamino)phosphanyloxypropanenitrile (2.69 mL, 8.46 mmol). The reaction was stirred at room temperature for 90 minutes. Water (1.0 mL) was added to quench the reaction, followed by a 3:1 mixture of toluene/hexanes (80.0 mL). This organic fraction was washed four times with a 3:2 mixture of DMF/H₂O (50.0 mL). The organic fraction was then washed with saturated sodium bicarbonate solution and brine, followed by drying over sodium sulfate. The crude reaction was then concentrated to an oil under reduced pressure. Purification by Biotage (Si, 220 g col, 0-100% ethyl acetate) afforded the desired product (7a) as a white solid.

N-[9-[(2R,4R,5R)-5-[[bis(4-methoxyphenyl)-phenyl-methoxy]methyl]-4-[2-cyanoethoxy-(diisopropylamino)phosphanyl]oxy-tetrahydrofuran-2-yl]purin-6-yl]benzamide (1.65 g, 1.93 mmol, yield: 34.2%)

1H NMR (300 MHz, CHLOROFORM-d) δ 8.93 (s, 1H), 8.75-8.85 (m, 1H), 8.32 (s, 1H), 8.01 (br d, J=7.04 Hz, 2H), 7.43-7.69 (m, 5H), 7.27-7.41 (m, 5H), 7.23 (br d, J=6.78 Hz, 2H), 6.76-6.89 (m, 4H), 6.49-6.64 (m, 1H), 4.57 (br s, 1H), 4.36-4.50 (m, 1H), 3.72-3.89 (m, 6H), 3.51-3.68 (m, 2H), 3.18-3.50 (m, 4H), 2.67 (s, 2H), 2.25-2.50 (m, 2H), 0.98-1.18 (m, 6H), 0.81-0.97 (m, 6H)

¹³C NMR (75 MHz, CHLOROFORM-d) δ 171.1, 164.6, 158.5, 158.5, 158.5, 152.5, 152.4, 151.2, 149.3, 149.2, 144.7, 144.7, 142.0, 141.9, 136.1, 136.0, 135.9, 135.8, 133.9, 132.7, 130.2, 130.1, 130.0, 128.8, 128.3, 128.2, 127.9, 127.8, 126.9, 126.8, 123.0, 117.5, 113.1, 113.1, 86.5, 86.5, 84.8, 84.7, 84.6, 84.2, 72.1, 71.9, 63.5, 63.2, 60.4, 58.3, 58.0, 57.8, 55.3, 55.2, 43.2, 43.1, 43.0, 42.9, 40.9, 40.9, 24.6, 24.5, 24.5, 24.4, 24.3, 24.2, 21.0, 20.2, 20.1, 14.2 ³¹P NMR (121 MHz, CHLOROFORM-d) δ 150.94 (s, 1P), 147.91 (s, 1P)

LCMS: M−H=856.5

### Example 40: Synthesis of an Amidite of a Stereo-Non-Standard Nucleoside, β-D-Deoxyxylose

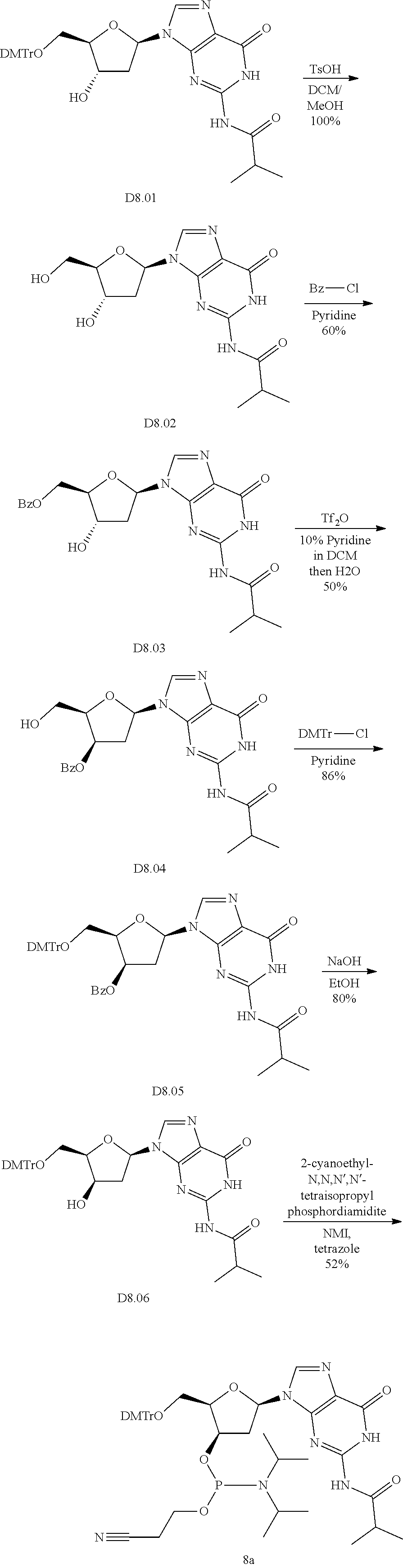

Steps of this synthesis have been previously described, see, e.g., Chen, et al., Nuc. Acids Res., 1995.

### Preparation of Compound D8.02

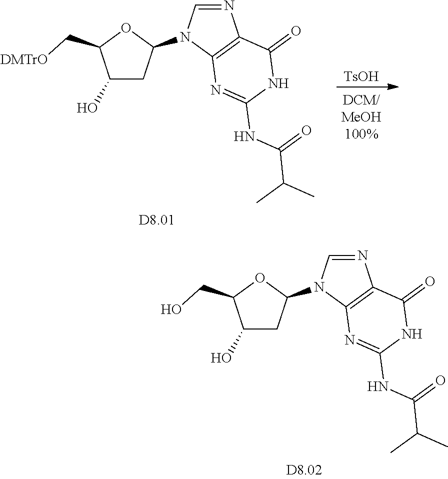

N-[9-[(2R,4S,5R)-5-[[bis(4-methoxyphenyl)-phenyl-methoxy]methyl]-4-hydroxy-tetrahydrofuran-2-yl]-6-oxo-1H-purin-2-yl]-2-methyl-propanamide (D8.01) (50.0 g, 78.2 mmol) was dissolved in DCM/methanol (3:1, 1560 mL) and cooled to 0° C. TsOH (17.8 g, 93.8 mmol) was added and the orange reaction mixture was stirred at 0° C. After 60 minutes, sodium carbonate (9.94 g, 93.8 mmol) was added at 0° C. and stirred until the orange color disappeared. The solvents were removed under reduced pressure. Dichloromethane was added to the crude reaction and the white precipitate was isolated and dried under high vacuum. The crude desired product (D8.02) was isolated as a white solid.

N-[9-[(2R,4S,5R)-4-hydroxy-5-(hydroxymethyl)tetrahydrofuran-2-yl]-6-oxo-1H-purin-2-yl]-2-methyl-propanamide (28.7 g, 85.1 mmol, yield: 109%)

¹H NMR (300 MHz, DMSO-d6) δ 12.08 (br s, 1H), 11.68 (br s, 1H), 8.24 (s, 1H), 6.21 (dd, J=6.02, 7.42 Hz, 1H), 5.31 (d, J=3.84 Hz, 1H), 4.95 (t, J=5.44 Hz, 1H), 4.37 (dd, J=2.88, 5.70 Hz, 1H), 3.79-3.91 (m, 1H), 3.45-3.63 (m, 2H), 2.77 (quin, J=6.82 Hz, 1H), 2.53-2.62 (m, 1H), 2.19-2.28 (m, 1H), 1.12 (d, J=6.78 Hz, 6H)

¹³C NMR (75 MHz, DMSO-d6) δ 180.1, 154.8, 148.3, 148.0, 137.8, 137.4, 120.1, 87.7, 82.9, 70.4, 61.4, 34.7, 18.8

LCMS: M+H=138.1

### Preparation of Compound D8.03

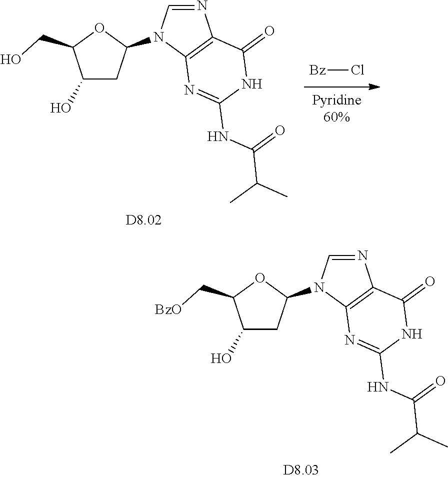

Crude N-[9-[(2R,4S,5R)-4-hydroxy-5-(hydroxymethyl)tetrahydrofuran-2-yl]-6-oxo-1H-purin-2-yl]-2-methyl-propanamide (D8.02) (26.4 g, 78.3 mmol) was suspended in pyridine (780 mL) under an atmosphere of nitrogen. Benzoyl chloride (9.08 mL, 78.3 mmol) was added dropwise to the reaction and was stirred at room temperature for 1 hr. The solvents were removed under reduced pressure, and the crude mixture was separated between dichloromethane and water. The organic phase was collected and washed with water (3 times) and brine. The crude reaction was then dried over sodium sulfate and concentrated under reduced pressure. Purification by Biotage (Si, 330 g col, 0-10% methanol/dichloromethane) afforded the desired product (D8.03) as a white solid.

[(2R,3S,5R)-3-hydroxy-5-[2-(2-methylpropanoylamino)-6-oxo-1H-purin-9-yl]tetrahydrofuran-2-yl]methyl benzoate (20.7 g, 46.9 mmol, yield: 59.9%)

¹H NMR (300 MHz, DMSO-d6) δ 12.08 (br s, 1H), 11.64 (br s, 1H), 8.19 (s, 1H), 7.85-8.01 (m, 2H), 7.62-7.73 (m, 1H), 7.47-7.57 (m, 2H), 6.26 (t, J=6.66 Hz, 1H), 5.54 (d, J=4.10 Hz, 1H), 4.54-4.62 (m, 1H), 4.37-4.54 (m, 2H), 4.05-4.19 (m, 1H), 2.76 (quin, J=6.56 Hz, 2H), 2.39 (ddd, J=4.42, 6.43, 13.35 Hz, 1H), 1.12 (d, J=6.78 Hz, 6H)

LCMS: M+H=442.2

### Preparation of Compound D8.04

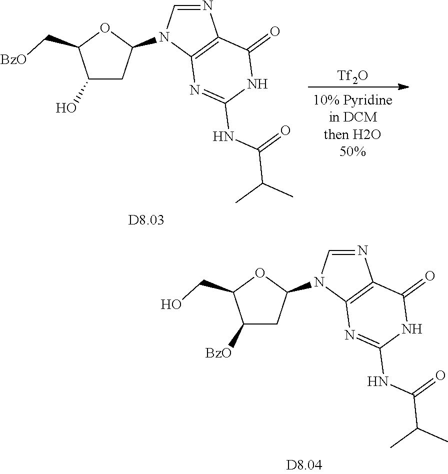

2R,3S,5R)-3-hydroxy-5-[2-(2-methylpropanoylamino)-6-oxo-1H-purin-9-yl]tetrahydrofuran-2-yl]methyl benzoate (D8.03) (10.0 g, 0.0227 mol) was dissolved in 10% pyridine in dichloromethane (164 mL) and cooled to −35° C. in an acetone/dry ice bath under an atmosphere of nitrogen. Trifluoromethanesulfonic anhydride (5.72 mL, 0.0340 mol) was added dropwise. After completion of addition the reaction mixture was warmed to 0° C. and stirred for 45 minutes before the addition of water (4.92 mL, 0.273 mol). The reaction was then warmed to room temperature overnight. The solvents were removed under reduced pressure. Equal volumes of water (150 mL) and ethyl acetate (150 mL) were added to the crude reaction, and this was shaken in a separation funnel. The white precipitate which formed was collected and dried under high vacuum affording the desired product (D8.04) as a white solid.

[(2R,3R,5R)-2-(hydroxymethyl)-5-[2-(2-methylpropanoylamino)-6-oxo-1H-purin-9-yl]tetrahydrofuran-3-yl]benzoate (5.24 g, 0.0119 mol, yield: 52.4%)

¹H NMR (300 MHz, DMSO-d6) δ 12.05 (s, 1H), 11.71 (s, 1H), 8.19 (s, 1H), 7.78-7.89 (m, 2H), 7.60-7.75 (m, 1H), 6.25 (dd, J=2.24, 7.62 Hz, 1H), 5.69 (t, J=4.16 Hz, 1H), 4.28-4.41 (m, 1H), 3.67-3.86 (m, 2H), 3.01 (dq, J=5.57, 7.57 Hz, 1H), 2.67-2.86 (m, 2H), 1.11 (d, J=6.91 Hz, 6H)

LCMS: M+H=442.2

### Preparation of Compound D8.05

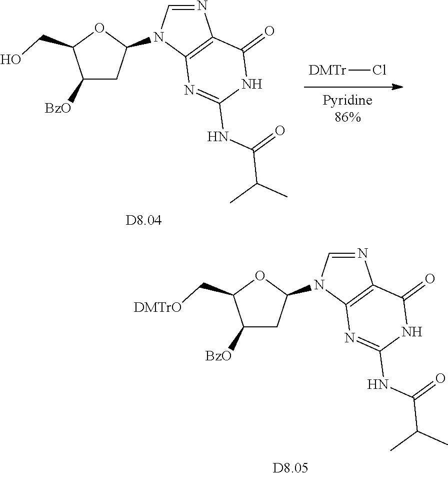

DMTrCl (3.68 g, 10.9 mmol) was added to a solution of [(2R,3R,5R)-2-(hydroxymethyl)-5-[2-(2-methylpropanoylamino)-6-oxo-1H-purin-9-yl]tetrahydrofuran-3-yl]benzoate (D8.04) (4.00 g, 9.06 mmol) in pyridine (30.2 mL), and the reaction was stirred at room temperature for 2 hours. The reaction was concentrated to an oil, and purification by Biotage (Si, 10 g col, 0-100% ethyl acetate/hexanes) afforded the desired product (D8.05) as a white solid.

[(2R,3R,5R)-2-[[bis(4-methoxyphenyl)-phenyl-methoxy]methyl]-5-[2-(2-methylpropanoylamino)-6-oxo-1H-purin-9-yl]tetrahydrofuran-3-yl]benzoate (5.79 g, 7.78 mmol, yield: 85.9%)

¹H NMR (300 MHz, DMSO-d6) δ 12.05 (br s, 1H), 11.73 (br s, 1H), 7.95 (s, 1H), 7.57-7.74 (m, 3H), 7.40-7.51 (m, 2H), 7.26-7.37 (m, 2H), 7.10-7.25 (m, 7H), 6.73 (t, J=8.58 Hz, 4H), 6.28 (dd, J=2.37, 7.62 Hz, 1H), 5.82 (t, J=4.22 Hz, 1H), 4.50-4.63 (m, 1H), 3.68 (d, J=4.99 Hz, 6H), 3.27 (d, J=6.02 Hz, 2H), 2.96-3.11 (m, 1H), 2.83-2.93 (m, 1H), 2.77 (quin, J=6.78 Hz, 1H), 1.14-1.14 (m, 1H), 1.11 (dd, J=0.90, 6.78 Hz, 5H)

LCMS: M+H=744.3

### Preparation of Compound D8.06

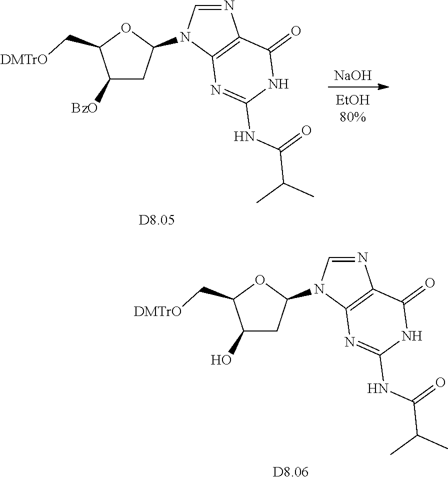

[(2R,3R,5R)-2-[[bis(4-methoxyphenyl)-phenyl-methoxy]methyl]-5-[2-(2-methylpropanoylamino)-6-oxo-1H-purin-9-yl]tetrahydrofuran-3-yl]benzoate (D8.05) (5.45 g, 0.00733 mol) was dissolved in a 1:1:1 mixture of THF (54.5 mL):1,4-dioxane (54.5 mL):methanol (54.5 mL). The reaction was cooled to 0° C., and to this was added 1 N NaOH (54.5 mL). The reaction was stirred at 0° C. for 2 hours. The reaction was then diluted with ethyl acetate and water. The aqueous fraction was extracted with ethyl acetate. The combined organic fractions were washed with brine and dried over sodium sulfate. Purification by column on Biotage (Si, 10 g col, 0-5% methanol/methanol) afforded the desired product (D8.06) as a white solid.

N-[9-[(2R,4R,5R)-5-[[bis(4-methoxyphenyl)-phenyl-methoxy]methyl]-4-hydroxy-tetrahydrofuran-2-yl]-6-oxo-1H-purin-2-yl]-2-methyl-propanamide (3.73 g, 0.00583 mol, yield: 79.6%)

¹H NMR (300 MHz, DMSO-d6) δ 12.11 (br s, 1H), 11.76 (br s, 1H), 8.01 (s, 1H), 7.36-7.47 (m, 2H), 7.17-7.33 (m, 7H), 6.82 (dd, J=8.96, 10.37 Hz, 4H), 6.22 (d, J=6.53 Hz, 1H), 5.30 (d, J=3.58 Hz, 1H), 4.34 (br d, J=3.58 Hz, 1H), 4.15-4.27 (m, 1H), 3.72 (d, J=2.56 Hz, 6H), 3.35-3.40 (m, 1H), 3.18 (dd, J=2.69, 9.98 Hz, 1H), 2.77 (br d, J=6.91 Hz, 2H), 2.28 (br d, J=14.59 Hz, 1H), 1.12 (dd, J=1.79, 6.78 Hz, 6H)

LCMS: M+H=640.3

### Preparation of Compound 8a

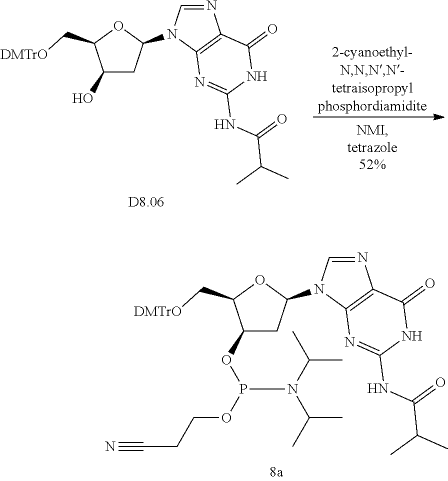

N-[9-[(2R,4R,5R)-5-[[bis(4-methoxyphenyl)-phenyl-methoxy]methyl]-4-hydroxy-tetrahydrofuran-2-yl]-6-oxo-1H-purin-2-yl]-2-methyl-propanamide (D8.06) (3.00 g, 4.69 mmol) was dissolved in dry DMF (46.8 mL) under an atmosphere of nitrogen. To this was added 1H-tetrazole (0.263 g, 3.75 mmol) and 1-methylimidazole (0.0930 mL, 1.17 mmol), followed by dropwise addition of 3-bis(diisopropylamino)phosphanyloxypropanenitrile (2.23 mL, 7.03 mmol). This was stirred at room temperature overnight. Water (1.0 mL) was added to quench the reaction, followed by a 3:1 mixture of toluene/hexanes (80.0 mL). This organic fraction was washed four times with a 3:2 mixture of DMF/H₂O (50.0 mL). The organic fraction was then washed with saturated sodium bicarbonate solution and brine, followed by drying over sodium sulfate. The crude reaction was then concentrated to an oil under reduced pressure. Purification by Biotage (Si, 50 g col, 0-100% ethyl acetate) afforded the desired product (8a) as a white solid.

N-[9-[(2R,4R,5R)-5-[[bis(4-methoxyphenyl)-phenyl-methoxy]methyl]-4-[2-cyanoethoxy-(diisopropylamino)phosphanyl]oxy-tetrahydrofuran-2-yl]-6-oxo-1H-purin-2-yl]-2-methyl-propanamide (2.03 g, 2.42 mmol, yield: 51.5%)

¹H NMR (300 MHz, CHLOROFORM-d) δ 12.03 (br s, 1H), 8.58-8.87 (m, 1H), 8.02 (d, J=9.47 Hz, 1H), 7.41-7.53 (m, 2H), 7.30-7.38 (m, 4H), 7.27-7.30 (m, 1H), 7.24 (br d, J=7.42 Hz, 2H), 6.69-6.88 (m, 4H), 6.11-6.26 (m, 1H), 4.52 (br d, J=4.10 Hz, 1H), 4.30-4.42 (m, 1H), 3.20-3.71 (m, 7H), 2.23-2.88 (m, 6H), 1.15-1.24 (m, 10H), 1.06 (dd, J=6.72, 15.30 Hz, 6H), 0.86-0.96 (m, 5H)

¹³C NMR (75 MHz, CHLOROFORM-d) δ 178.7, 178.6, 158.5, 158.5, 155.8, 155.8, 147.9, 147.7, 147.5, 147.3, 144.7, 138.0, 137.9, 136.0, 135.8, 135.8, 130.1, 130.1, 130.0, 128.3, 128.2, 127.8, 126.9, 126.9, 121.2, 121.0, 117.7, 117.5, 113.1, 113.1, 86.6, 86.5, 84.4, 84.4, 84.2, 84.0, 83.1, 79.5, 74.1, 73.8, 72.4, 72.2, 69.8, 63.8, 63.7, 57.6, 55.3, 55.2, 45.6, 43.3, 43.2, 43.1, 40.7, 43.0, 38.9, 36.4, 34.4, 34.1, 28.3, 24.7, 24.6, 24.5, 24.4, 23.1, 22.3, 21.7, 21.3, 20.3, 20.2, 19.2, 19.1, 19.0, 19.7, 18.8, 18.8, 17.8

³¹P NMR (121 MHz, CHLOROFORM-d) δ 151.47 (s, 1P), 146.99 (s, 1P)

LCMS: M−H=838.5

### Example 41: Synthesis of an Amidite of a Stereo-Non-Standard Nucleoside, α-L-Deoxyxylose

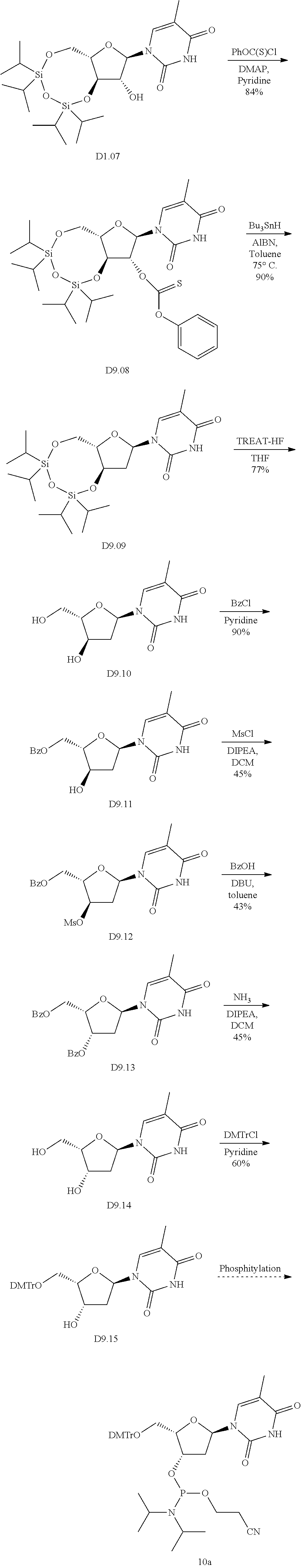

Steps of this synthesis have been previously described, see, e.g., Chatelain, Eur. J Med. Chem, 2013; Foldesi, et al., Nucleosides Nucleotides Nucleic Acids, 2007; Shi, et al., Tetrahedron Asymmetry, 2010.

### Preparation of Compound D9.08

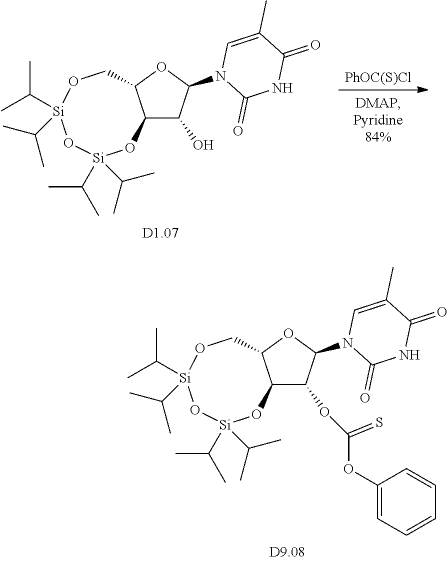 [SMILES: "PhOC(S)Cl" ; Cc1cn([C@@H]2O[C@H]3CO[Si](C(C)C)(C(C)C)O[Si](C(C)C)(C(C)C)O[C@@H]3[C@H]2O)c(=O)[nH]c1=O ; Cc1cn([C@@H]2O[C@H]3CO[Si](C(C)C)(C(C)C)O[Si](C(C)C)(C(C)C)O[C@@H]3[C@H]2OC(=S)Oc2ccccc2)c(=O)[nH]c1=O]

1-[(6aS,8R,9R,9aR)-9-Hydroxy-2,2,4,4-tetraisopropyl-6a,8,9,9a-tetrahydro-6H-furo[3,2-f][1,3,5,2,4]trioxadisilocin-8-yl]-5-methyl-pyrimidine-2,4-dione (D1.07) (15.0 g, 0.0300 mol) and 4-dimethylaminopyridine (7.32 g, 0.0599 mol) were dissolved in anhydrous acetonitrile (300 mL) followed by slow addition of O-4-methylphenyl chlorothioformate (5.02 mL, 0.0330 mol). The reaction was stirred at room temperature for 72 hours. The solvents were removed under reduced pressure and the residue was partitioned between ethyl acetate and water. The aqueous layer was extracted with ethyl acetate and the combined organics were washed with 10% HCl (aq), water, saturated sodium bicarbonate solution, water and brine. The organic fractions were dried over magnesium sulfate and concentrated. Purification by Biotage (Si, 100 g col, 0-40% ethyl acetate/hexanes) afforded the desired product (D9.08) as a white solid.

1-[(6aS,8R,9R,9aS)-2,2,4,4-tetraisopropyl-9-(4-methylphenoxy)carbothioyloxy-6a,8,9,9a-tetrahydro-6H-furo[3,2-f][1,3,5,2,4]trioxadisilocin-8-yl]-5-methyl-pyrimidine-2,4-dione (16.4 g, 0.0251 mol, yield: 83.9%)

¹H NMR (300 MHz, CHLOROFORM-d) δ 8.89 (s, 1H), 7.12-7.24 (m, 3H), 6.90-7.02 (m, 2H), 6.29 (dd, J=5.25, 6.91 Hz, 1H), 5.94 (d, J=5.12 Hz, 1H), 4.74 (dd, J=7.04, 8.06 Hz, 1H), 4.31-4.45 (m, 1H), 3.94-4.07 (m, 2H), 2.36 (s, 3H), 1.94 (d, J=1.15 Hz, 3H), 1.03-1.15 (m, 28H)

¹³C NMR (75 MHz, CHLOROFORM-d) δ 194.8, 163.6, 151.3, 150.3, 136.6, 136.6, 130.1, 121.2, 111.6, 88.3, 87.5, 83.6, 74.0, 61.7, 21.0, 17.4, 17.3, 17.3, 17.3, 17.0, 16.9, 13.5, 13.1, 12.8, 12.6, 12.4

LCMS: no ionization

### Preparation of Compound D9.09

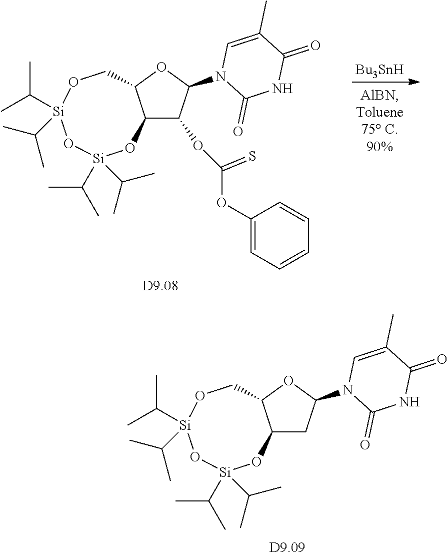 [SMILES: "Bu3SnH" ; Cc1cn([C@@H]2O[C@H]3CO[Si](C(C)C)(C(C)C)O[Si](C(C)C)(C(C)C)O[C@@H]3[C@H]2OC(=S)Oc2ccccc2)c(=O)[nH]c1=O ; Cc1cn([C@H]2C[C@H]3O[Si](C(C)C)(C(C)C)O[Si](C(C)C)(C(C)C)OC[C@@H]3O2)c(=O)[nH]c1=O]

Azobisisobutyronitrile (AIBN) (0.825 g, 5.03 mmol) and tributyltin hydride (73.2 g, 251 mmol) in toluene (475 mL) were added dropwise to a degassed (with nitrogen) solution of 1-[(6aS,8R,9R,9aS)-2,2,4,4-tetraisopropyl-9-(4-methylphenoxy)carbothioyloxy-6a,8,9,9a-tetrahydro-6H-furo[3,2-f][1,3,5,2,4]trioxadisilocin-8-yl]-5-methyl-pyrimidine-2,4-dione (D9.08) (16.4 g, 25.1 mmol) in toluene (475 mL) at 80° C. The solution was heated at 80° C. for 1 hour before being cooled to room temperature and removal of the solvents under reduced pressure. Purification by Biotage (Si, 50 g col, 0-40% ethyl acetate/hexanes) afforded the desired product (D9.09) as a white solid.

1-[(6aS,8R,9aR)-2,2,4,4-tetraisopropyl-6a,8,9,9a-tetrahydro-6H-furo[3,2-f][1,3,5,2,4]trioxadisilocin-8-yl]-5-methyl-pyrimidine-2,4-dione (11.0 g, 22.6 mmol, yield: 90.0%)

¹H NMR (300 MHz, CHLOROFORM-d) δ 9.28 (s, 1H), 7.47 (d, J=1.28 Hz, 1H), 6.20 (dd, J=5.76, 6.66 Hz, 1H), 4.48-4.64 (m, 1H), 3.98-4.11 (m, 2H), 3.75-3.88 (m, 1H), 2.73-2.89 (m, 1H), 2.16 (ddd, J=5.89, 6.78, 13.83 Hz, 1H), 1.96 (d, J=1.15 Hz, 3H), 0.99-1.12 (m, 28H)

¹³C NMR (75 MHz, CHLOROFORM-d) δ 163.9, 150.7, 135.5, 111.0, 85.9, 84.6, 72.4, 63.1, 39.9, 17.5, 17.3, 17.0, 17.1, 13.3, 13.3, 12.9, 12.6, 12.5

LCMS: No ionization

### Preparation of Compound D9.10

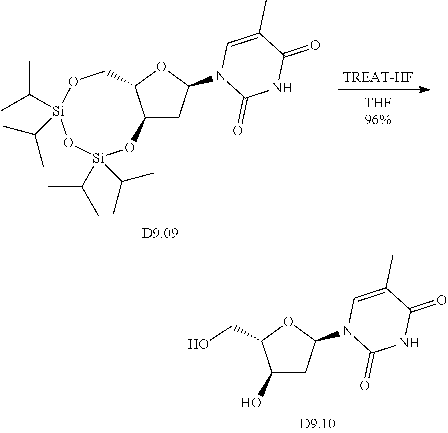 [SMILES: Cc1cn([C@H]2C[C@H]3O[Si](C(C)C)(C(C)C)O[Si](C(C)C)(C(C)C)OC[C@@H]3O2)c(=O)[nH]c1=O ; "TREAT-HF" ; Cc1cn([C@H]2C[C@@H](O)[C@H](CO)O2)c(=O)[nH]c1=O]

TEA (7.82 mL, 56.11 mmol) was added to a solution of 1-[(6aS,8R,9aR)-2,2,4,4-tetraisopropyl-6a,8,9,9a-tetrahydro-6H-furo[3,2-f][1,3,5,2,4]trioxadisilocin-8-yl]-5-methyl-pyrimidine-2,4-dione (D9.09) (10.88 g, 22.45 mmol) in THF (120.8 mL). The reaction was cooled to 0° C. with an ice bath under an atmosphere of nitrogen. Triethylamine trihydrofluoride (18.29 mL, 112.2 mmol) was added slowly at 0° C., and the reaction was warmed to room temperature and stirred for 1.5 hours. The solvents were removed under reduced pressure to give a clear colorless oil. Precipitation by addition of ethyl acetate, dichloromethane and a small amount of methanol gave the crude product as a white solid (13 g). The mother liquors from the precipitation were concentrated and purification by Biotage (Si, 25 g col, 0-100% EtOAC/hexanes then 0-10% methanol/dichloromethane) afforded the desired product as a white solid (0.200 g). Crystallization of the white solid from hot ethanol afforded the desired product as colorless crystals (3.30 g). The mother liquors from the crystallization were concentrated under reduced pressure and a second batch was isolated by crystallization from hot ethanol as colorless crystals (0.659 g).

1-[(2R,4R,5S)-4-hydroxy-5-(hydroxymethyl)tetrahydrofuran-2-yl]-5-methyl-pyrimidine-2,4-dione (4.159 g, 17.17 mmol, yield: 76.50%)

1H NMR (300 MHz, METHANOL-d4) δ 7.79 (d, J=1.02 Hz, 1H), 6.21 (dd, J=2.82, 7.42 Hz, 1H), 4.38 (td, J=2.13, 6.11 Hz, 1H), 4.30 (dt, J=2.11, 4.32 Hz, 1H), 3.49-3.70 (m, 2H), 2.69 (ddd, J=6.34, 7.52, 14.31 Hz, 1H), 2.07 (td, J=2.48, 14.50 Hz, 1H), 1.90 (d, J=0.90 Hz, 3H)

¹³C NMR (75 MHz, METHANOL-d4) δ 166.7, 152.5, 138.9, 110.7, 91.0, 88.1, 72.5, 63.5, 41.7, 12.6

LCMS: M+H=243.1 and M+Na=265.1

### Preparation of Compound D9.11

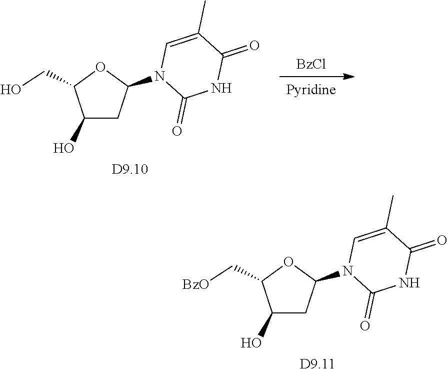

1-[(2R,4R,5S)-4-hydroxy-5-(hydroxymethyl)tetrahydrofuran-2-yl]-5-methyl-pyrimidine-2,4-dione (D9.10) (4.18 g, 17.3 mmol) was co-evaporated with dry pyridine (3×59 mL) and was dissolved in pyridine (86.0 mL). The mixture was cooled to 0° C., and benzoyl chloride (2.10 mL, 18.1 mmol) was added dropwise under a nitrogen atmosphere. The mixture was stirred for 2 h after which it was evaporated to dryness. Purification by Biotage (Si, 100 g col, 0-5% methanol/dichloromethane) afforded the desired product (D9.11) as a white solid.

[(2S,3R,5R)-3-hydroxy-5-(5-methyl-2,4-dioxo-pyrimidin-1-yl)tetrahydrofuran-2-yl]methyl benzoate (5.40 g, 15.6 mmol, yield: 90.4%)

¹H NMR (300 MHz, CHLOROFORM-d) δ 9.46 (s, 1H), 7.96-8.10 (m, 2H), 7.53-7.65 (m, 1H), 7.41-7.52 (m, 3H), 6.15 (dd, J=2.24, 7.49 Hz, 1H), 4.64-4.76 (m, 1H), 4.52 (br d, J=6.02 Hz, 1H), 4.29-4.47 (m, 2H), 3.68 (br s, 1H), 2.71 (ddd, J=6.14, 7.42, 14.98 Hz, 1H), 2.45 (br d, J=14.98 Hz, 1H), 1.88 (d, J=1.02 Hz, 3H)

¹³C NMR (75 MHz, CHLOROFORM-d) δ 166.3, 164.9, 151.0, 137.5, 133.4, 129.6, 129.4, 128.6, 109.3, 88.0, 87.0, 71.9, 64.5, 40.5, 12.4

LCMS: M+H=347.1 and M+Na=369.1

### Preparation of Compound D9.12

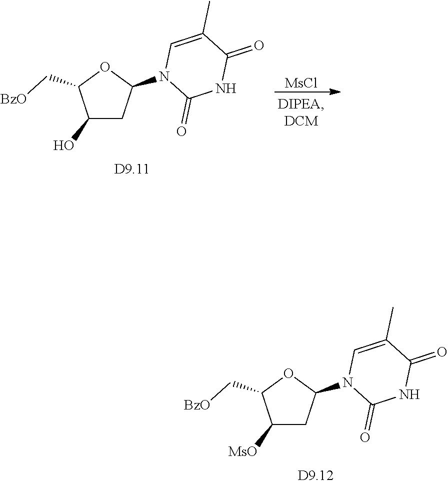

A solution of [(2S,3R,5R)-3-hydroxy-5-(5-methyl-2,4-dioxo-pyrimidin-1-yl)tetrahydrofuran-2-yl]methyl benzoate (D9.11) (3.00 g, 8.66 mmol) and triethylamine (2.41 mL, 17.3 mmol) in dichloromethane (17.34 mL) was cooled to 0° C. with an ice bath, after which methanesulfonyl chloride (1.49 g, 13.0 mmol) was added dropwise within 20 min. After the addition was finished, the ice bath was removed, and the mixture was stirred at room temperature for 2 h. The reaction was quenched with aqueous HCl solution (10%, 50 mL), and the organic phase was separated and washed with saturated aqueous K₂CO₃ solution (50 mL). This caused an emulsion (next time probably avoid the saturated K₂CO₃ solution). The aqueous phase was extracted with large quantities of dichloromethane and diluted with water. Dried over magnesium sulfate (lost some here due to spillage). This was concentrated to a small volume. A small volume of dichloromethane was added to load onto the column and a white precipitate was formed.

The dichloromethane volume was purified by Biotage (Si, 100 g col, 0-80% ethyl acetate/hexanes) afforded the desired product as a white solid—0.527 g.

The white precipitate was isolated by filtration to afford the desired product as a white solid—0.888 g.

And the filtrate from washing the precipitate was isolated by concentration under reduced pressure to afford the desired product as a white solid—0.230 g.

Products batches were combined.

[(2S,3R,5R)-5-(5-methyl-2,4-dioxo-pyrimidin-1-yl)-3-methylsulfonyloxy-tetrahydrofuran-2-yl]methyl benzoate (1.65 g, 3.88 mmol, yield: 44.7%)

¹H NMR (300 MHz, DMSO-d6) δ 11.36 (s, 1H), 7.96-8.07 (m, 2H), 7.65-7.74 (m, 1H), 7.50-7.62 (m, 3H), 6.23 (dd, J=3.58, 7.04 Hz, 1H), 5.38-5.53 (m, 1H), 4.85-5.03 (m, 1H), 4.33-4.53 (m, 2H), 3.31 (s, 3H), 2.87-3.05 (m, 1H), 2.45 (td, J=3.10, 15.04 Hz, 1H), 1.81 (d, J=0.90 Hz, 3H)

¹³C NMR (75 MHz, DMSO-d6) δ 165.4, 163.8, 150.3, 135.8, 133.5, 129.3, 129.2, 128.8, 109.2, 85.4, 82.6, 79.8, 63.7, 37.6, 12.2

LCMS: M+H=425.1

### Preparation of Compound D9.13

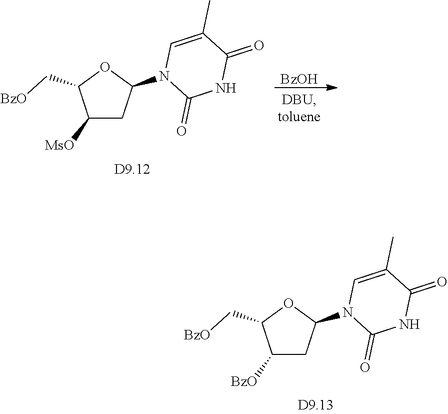

To a solution of 1,8-Diazabicyclo[5.4.0]undec-7-ene (0.703 mL, 4.71 mmol) in toluene (1.18 mL) was added benzoic acid (1.15 g, 9.42 mmol). The suspension was then stirred at room temperature for half an hour until it became a homogeneous solution, and [(2S,3R,5R)-5-(5-methyl-2,4-dioxo-pyrimidin-1-yl)-3-methylsulfonyloxy-tetrahydrofuran-2-yl]methyl benzoate (D9.12) (0.350 g, 0.825 mmol) was added. The mixture was heated to 95° C., and stirring was continued at this temperature overnight—two new peaks by LCMS and unreacted starting material.

After being cooled down to room temperature, the reaction mixture was diluted with ethyl acetate. The solution was transferred into a separatory funnel and was washed successively with 10% aqueous HCl solution, aqueous saturated NaHCO₃ solution, and brine. After the organic solution was dried over anhydrous MgSO4, the solvent was removed by distillation in vacuo to give the crude product which was purified by Biotage (Si, 100 g col, 0-70% ethyl acetate/hexanes).

[(2S,3S,5R)-3-benzoyloxy-5-(5-methyl-2,4-dioxo-pyrimidin-1-yl)tetrahydrofuran-2-yl]methyl benzoate (0.159 g, 0.353 mmol, yield: 42.8%, clear oil)

¹H NMR (300 MHz, CHLOROFORM-d) δ 9.32 (br s, 1H), 7.96-8.14 (m, 4H), 7.52-7.64 (m, 2H), 7.38-7.49 (m, 4H), 7.15 (d, J=1.28 Hz, 1H), 6.29 (t, J=6.72 Hz, 1H), 5.80-6.01 (m, 1H), 4.87-5.01 (m, 1H), 4.56-4.72 (m, 2H), 2.72-2.84 (m, 1H), 2.57-2.70 (m, 1H), 1.95 (d, J=1.15 Hz, 3H)

¹³C NMR (75 MHz, CHLOROFORM-d) δ 166.2, 165.5, 163.9, 150.3, 135.8, 133.7, 133.3, 129.8, 129.7, 129.5, 129.1, 128.6, 128.5, 111.4, 87.3, 80.4, 73.9, 62.8, 39.1, 12.6

LCMS: M+H=451.2 and M+Na=473.1

### Preparation of Compound D9.14

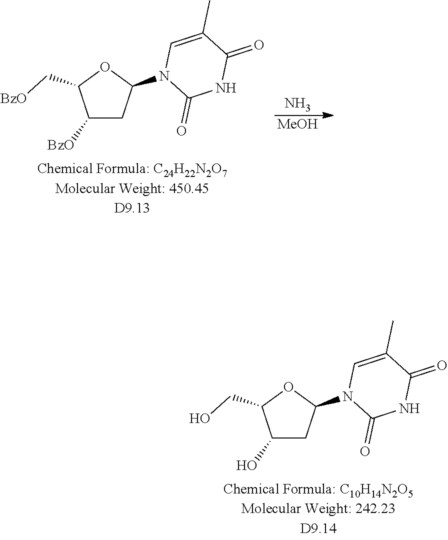

To a 7 N NH₃ in methanol (6.93 mL, 0.1 M) was added [(2S,3S,5R)-3-benzoyloxy-5-(5-methyl-2,4-dioxo-pyrimidin-1-yl)tetrahydrofuran-2-yl]methyl benzoate (D9.13) (312 mg, 0.693 mmol) and the reaction was stirred at room temperature overnight. The solvents were removed by a stream of nitrogen to give a mint-smelling crude white solid. Purification by flash chromatography (Si, 100 g col, 10% methanol/dichloromethanes) afforded the desired product (D9.14) as a clear oil.

1-[(2R,4S,5S)-4-hydroxy-5-(hydroxymethyl)tetrahydrofuran-2-yl]-5-methyl-pyrimidine-2,4-dione (134 mg, 0.55 mmol, yield: 80%)

¹H NMR (300 MHz, METHANOL-d4) δ 7.50-7.55 (m, 1H), 6.27 (dd, J=6.46, 7.36 Hz, 1H), 4.47-4.56 (m, 1H), 4.35 (ddd, J=3.39, 4.96, 6.24 Hz, 1H), 3.81 (dd, J=1.79, 5.63 Hz, 2H), 2.40-2.51 (m, 1H), 2.24-2.37 (m, 1H), 1.91 (d, J=1.15 Hz, 3H)

¹³C NMR (75 MHz, METHANOL-d4) δ 166.5, 152.3, 138.0, 111.6, 87.8, 86.1, 72.7, 62.1, 42.5, 12.4

LCMS: M+H=243.1

### Preparation of Compound D9.15

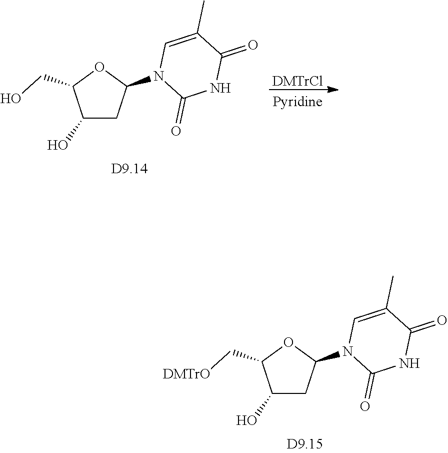

DMTrCl 154 mg, 0.455 mmol) was added to a solution of 1-((2R,4S,5S)-4-hydroxy-5-(hydroxymethyl)tetrahydrofuran-2-yl)-5-methylpyrimidine-2,4(1H,3H)-dione (D9.14) (110 mg, 0.455 mmol) in pyridine (2.27 mL, 0.2 M). The reaction was stirred at room temperature overnight. The reaction was concentrated to an oil. Purification by Biotage (Si, 10 g col, 0-80% ethyl acetate/hexanes) afforded the desired compound (D9.15) as a pale yellow solid (148 mg, 60%).

¹H NMR (300 MHz, CHLOROFORM-d) δ 8.90 (s, 1H), 7.40-7.50 (m, 2H), 7.23-7.38 (m, 7H), 7.11 (d, J=1.15 Hz, 1H), 6.80-6.93 (m, 4H), 6.24 (t, J=6.72 Hz, 1H), 4.62 (br d, J=3.58 Hz, 1H), 4.36-4.46 (m, 1H), 3.79 (s, 6H), 3.36-3.59 (m, 2H), 2.81 (d, J=3.71 Hz, 1H), 2.56 (ddd, J=1.66, 6.30, 14.05 Hz, 1H), 2.18-2.32 (m, 1H), 1.94 (d, J=1.28 Hz, 3H)

¹³C NMR (75 MHz, CHLOROFORM-d) δ 163.8, 158.7, 150.2, 144.3, 135.7, 135.4, 135.4, 129.9, 128.1, 127.9, 127.1, 113.4, 110.9, 87.3, 86.9, 82.4, 72.5, 61.9, 60.4, 55.2, 41.2, 21.1, 14.2, 12.6 LCMS: M+Na=567.2

### Preparation of Compound 10a

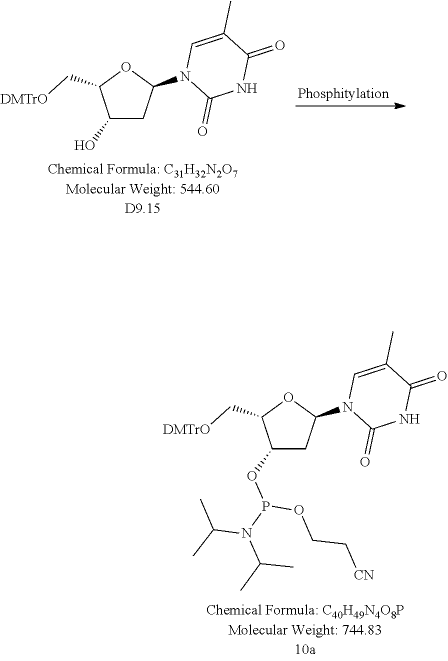

1H-Tetrazole (0.013 g, 0.188 mmol) and 1-methylimidazole (0.005 mL, 0.059 mmol) were added to a solution of 1-((2R,4S,5S)-5-((bis(4-methoxyphenyl)(phenyl)methoxy)methyl)-4-hydroxytetrahydrofuran-2-yl)-5-methylpyrimidine-2,4(1H,3H)-dione (D9.15) (0.128 g, 0.235 mmol) in DMF (2.35 mL) at room temperature under an atmosphere of nitrogen. 3-Bis(diisopropylamino)phosphanyloxypropanenitrile (0.116 mL, 0.353 mmol) was then added dropwise, and the reaction was stirred at room temperature overnight. Water (0.1 mL) was added to quench the reaction. A 3:1 mixture of toluene/hexanes was added, and the organic layer was washed four times with a 3:2 mixture of DMF/H₂O. The organics were then washed with saturated sodium bicarbonate solution, brine, dried over solid sodium sulfate, and concentrated under reduced pressure to a white foam. Purification by Biotage (Si, 25 g col, 0-60% ethyl acetate/hexanes) afforded the desired product (10a) as a white solid (82 mg, 47%).

¹H NMR (300 MHz, CHLOROFORM-d) δ 8.59 (br s, 1H), 7.46 (br d, J=7.42 Hz, 2H), 7.18-7.38 (m, 8H), 6.70-6.96 (m, 4H), 6.13-6.29 (m, 1H), 4.00-4.70 (m, 3H), 3.75-3.86 (m, 6H), 3.34-3.58 (m, 5H), 2.52-2.85 (m, 2H), 2.29-2.45 (m, 1H), 2.08-2.23 (m, 1H), 1.98 (s, 3H), 1.10 (br dd, J=6.85, 18.75 Hz, 12H)

¹³C NMR (75 MHz, CHLOROFORM-d) δ 163.7, 158.5, 158.5, 150.1, 150.1, 144.8, 136.1, 136.0, 136.0, 135.5, 135.3, 130.2, 130.1, 130.1, 128.3, 128.2, 127.8, 126.8, 126.8, 117.5, 113.1, 113.1, 110.9, 110.9, 86.4, 86.4, 83.5, 83.5, 74.1, 73.9, 73.2, 73.0, 63.3, 63.2, 58.2, 57.9, 55.3, 55.2, 45.6, 45.4, 45.3, 43.3, 43.2, 43.2, 43.1, 40.8, 24.6, 24.5, 24.5, 24.4, 24.3, 23.0, 20.5, 20.4, 20.2, 20.2, 20.1, 12.7

³¹P NMR (121 MHz, CHLOROFORM-d) δ 149.89 (s, 1P), 148.39 (s, 1P)

LCMS: M−H=743.3

### Example 42: Synthesis of an Amidite of a Stereo-Non-Standard Nucleoside, α-D-Deoxyxylose

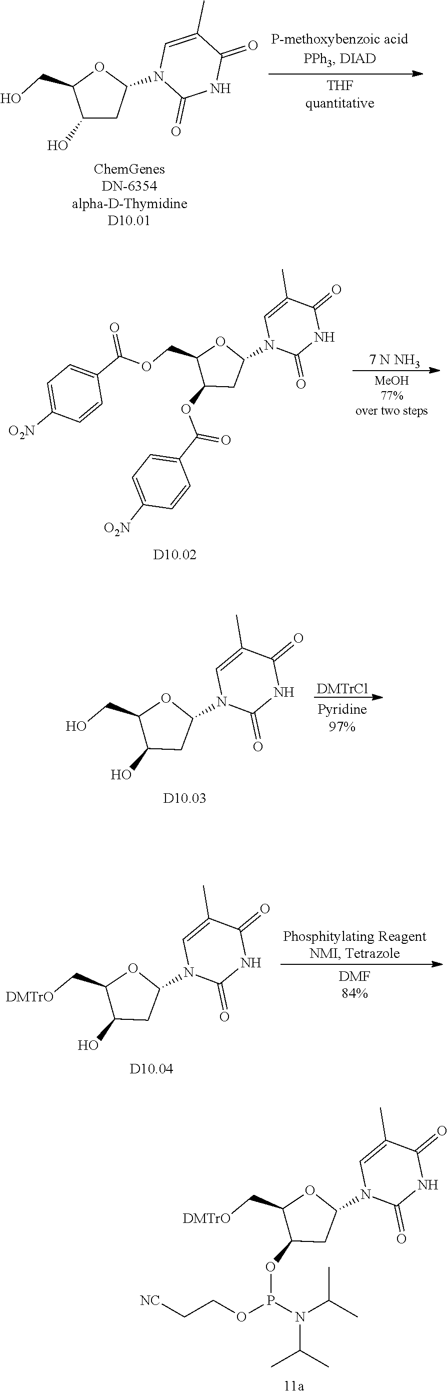

### Preparation of Compound D10.02

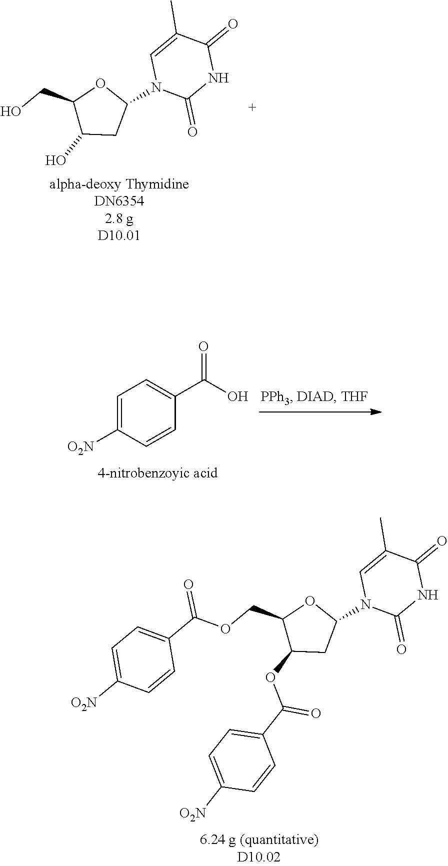 [SMILES: Cc1cn([C@@H]2C[C@H](O)[C@@H](CO)O2)c(=O)[nH]c1=O ; O=C(O)c1ccc([N+](=O)[O-])cc1 ; Cc1cn([C@@H]2C[C@@H](OC(=O)c3ccc([N+](=O)[O-])cc3)[C@@H](COC(=O)c3ccc([N+](=O)[O-])cc3)O2)c(=O)[nH]c1=O]

To a THF solution (140 mL) of alpha-deoxy thymidine (D10.01) (2.8 g, 11.56 mmol), p-nitrobenzoic acid (7.73 g, 46.24 mmol) and Ph₃P (12.13 g, 46.24 mmol), DIAD (8.96 mL, 46.24 mmol) was added dropwise at room temperature. The reaction was stirred at room temperature for 12 h and the reaction was concentrated to dryness. The residue was re-dissolved in dichloromethane. The DCM solution was washed with brine and concentrated. The crude product was purified by silica gel column chromatography and eluted with MeOH DCM solution to yield compound D10.02 (6.24 g, quantitative).

### Preparation of Compound D10.03

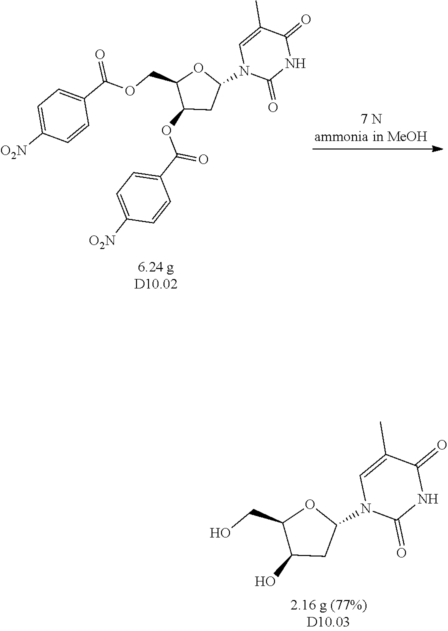 [SMILES: Cc1cn([C@@H]2C[C@@H](OC(=O)c3ccc([N+](=O)[O-])cc3)[C@@H](COC(=O)c3ccc([N+](=O)[O-])cc3)O2)c(=O)[nH]c1=O ; Cc1cn([C@@H]2C[C@@H](O)[C@@H](CO)O2)c(=O)[nH]c1=O]

D10.02 (6.24 g, 11.56 mmol) was dissolved in a 7 N ammonia MeOH solution (50 mL). The solution was stirred at room temperature for 4 h. The reaction was concentrated to dryness and the residue was purified by silica gel column chromatography and eluted with an MeOH/dichloromethane solution to yield compound D10.03 (2.16 g, 77%).

### Preparation of Compound D10.04

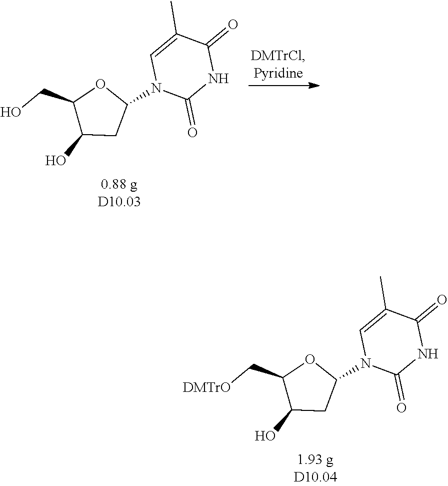

To a pyridine solution (40 mL) of D10.03 (0.88 g, 3.64 mmol) at 0° C., DMTrCl (1.54 g, 4.46 mmol) was added. The reaction mixture was warmed to room temperature and stirred for 2 h. The reaction was quenched with water, extracted with ethyl acetate. The ethyl acetate solution was concentrated to dryness. The residue was purified by silica gel column chromatography and eluted with an MeOH/dichloromethane solution to yield compound D10.04 (1.93 g, 97%).

### Preparation of Compound 11a

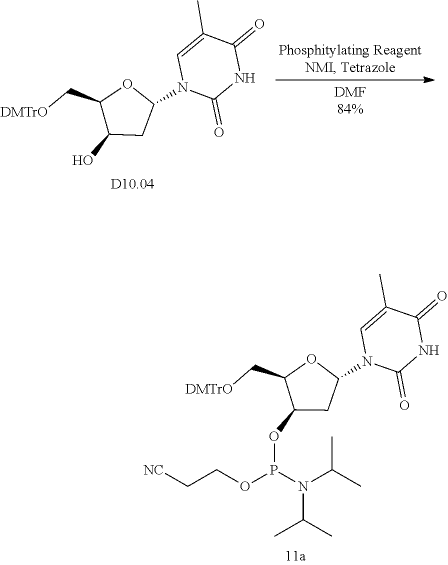

To a DMF (18 mL) solution of D10.04 (1.93 g, 3.55 mmol) and tetrazole (0.2 g, 2.84 mmol) at 0° C., 1-methylimidazole (0.071 mL, 0.89 mmol) and phosphitylating reagent (2.25 mL, 7.10 mmol) were added. The reaction was warmed to room temperature and stirred for 2 h. The reaction mixture was extracted with ethyl acetate (100 mL), washed with sat. NaHCO₃. The ethyl acetate solution was concentrated to dryness. The crude product was purified by silica gel column chromatography and eluted with an ethyl acetate/hexane solution to yield compound 11a (2.23 g, 84%).

### Example 43: Synthesis of an Amidite of a Stereo-Non-Standard Nucleoside, β-L-Deoxyxylose

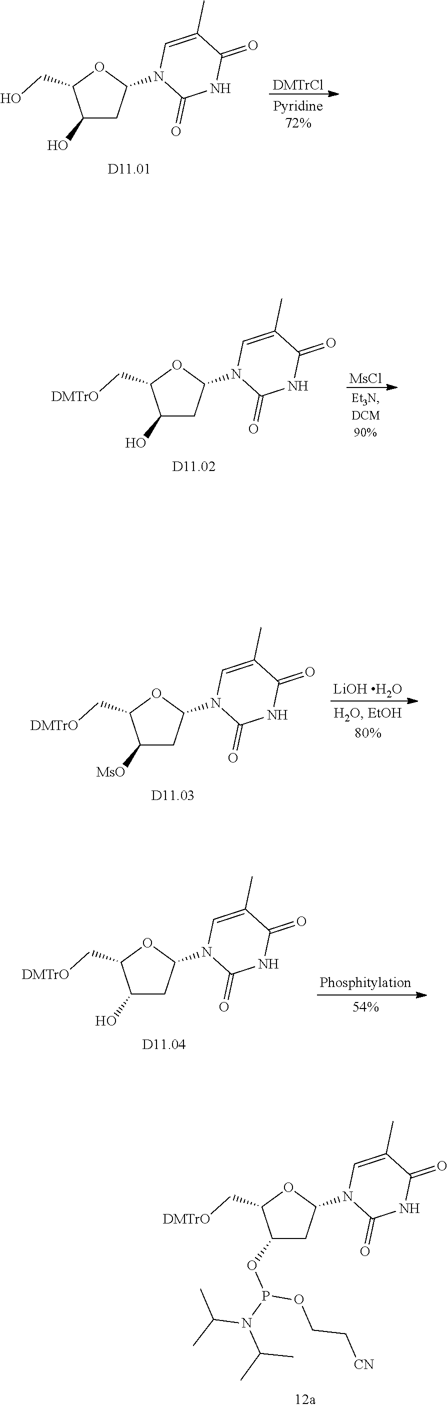

This synthesis is similar to that described in Kong, et al., Nucleosides Nucleotides Nucleic Acids, 2001.

### Preparation of Compound D11.02

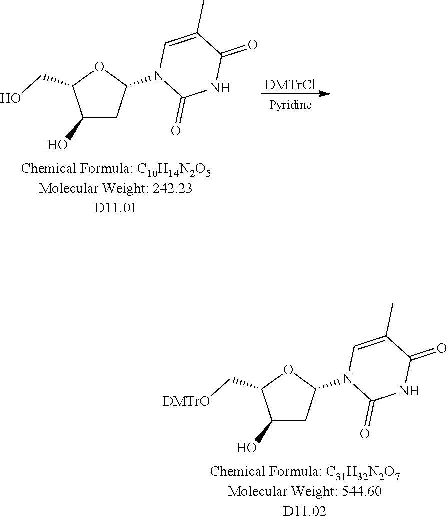

1-[(2R,4R,5S)-4-hydroxy-5-(hydroxymethyl)tetrahydrofuran-2-yl]-5-methyl-pyrimidine-2,4-dione (D11.01) (3.00 g, 12.3 mmol) was dissolved in pyridine (61.9 mL, 0.2 M). The mixture was cooled to 0° C. and benzoyl chloride (1.73 mL, 14.9 mmol) was added dropwise under a nitrogen atmosphere. The mixture was stirred for 2 h, after which it was evaporated to dryness. Purification by Biotage (Si, 100 g col, 0-5% methanol/dichloromethane) afforded the desired product (D11.02) as a white solid (4.72 g, 70%).

¹H NMR (300 MHz, METHANOL-d4) δ 7.71 (d, J=1.15 Hz, 1H), 7.39-7.51 (m, 2H), 7.21-7.38 (m, 7H), 6.80-6.91 (m, 4H), 6.34 (t, J=6.78 Hz, 1H), 4.50-4.59 (m, 1H), 4.02 (q, J=3.07 Hz, 1H), 3.76 (s, 6H), 3.34-3.46 (m, 2H), 2.28-2.41 (m, 2H), 1.40 (d, J=1.15 Hz, 3H)

¹³C NMR (75 MHz, METHANOL-d4) δ 173.0, 166.4, 160.4, 160.3, 152.3, 146.1, 137.7, 136.9, 136.8, 131.4, 129.5, 129.0, 128.1, 114.3, 111.7, 88.1, 87.9, 86.2, 72.8, 64.9, 61.6, 55.8, 41.5, 20.9, 14.5, 12.1 LCMS: M+Na=567.2

### Preparation of Compound D11.03

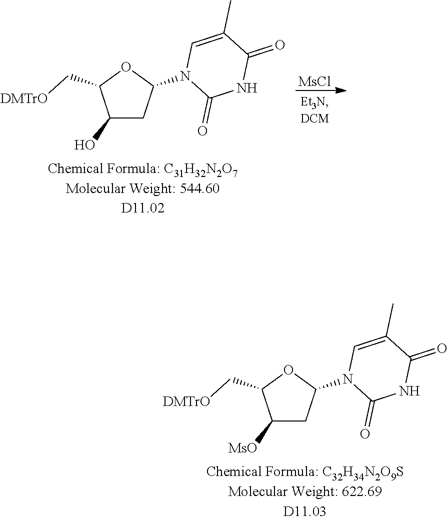

A solution 1-((2S,4R,5S)-5-((bis(4-methoxyphenyl)(phenyl)methoxy)methyl)-4-hydroxytetrahydrofuran-2-yl)-5-methylpyrimidine-2,4(1H,3H)-dione (D11.02) (3.0 g, 5.51 mmol) and triethylamine (1.53 mL, 11.1 mmol) in dichloromethane (27.5 mL) was cooled to 0° C. with an ice bath, after which methanesulfonyl chloride (0.644 mL, 8.26 mmol) was added dropwise within 20 min. After the addition was finished, the ice bath was removed, and the mixture was stirred at room temperature for 2 hr. The reaction mixture was diluted with saturated aqueous sodium carbonate solution and extracted with dichloromethane. The combined organic fractions were washed with brine and concentrated to a crude solid. Purification by Biotage (Si, 10 g col, 0-80% ethyl acetate/hexanes) afforded the desired compound (D11.03) as a white solid (3.08 g, 90.0%).

¹H NMR (300 MHz, CHLOROFORM-d) δ 8.33 (s, 1H), 7.55 (d, J=1.28 Hz, 1H), 7.30-7.39 (m, 4H), 7.25-7.29 (m, 5H), 6.79-6.91 (m, 4H), 6.43 (dd, J=5.50, 8.83 Hz, 1H), 5.34-5.43 (m, 1H), 4.32 (d, J=2.05 Hz, 1H), 3.80 (s, 6H), 3.51-3.59 (m, 1H), 3.40-3.48 (m, 1H), 3.03 (s, 3H), 2.67 (ddd, J=1.54, 5.47, 14.24 Hz, 1H), 2.47 (ddd, J=6.14, 8.64, 14.40 Hz, 1H), 1.45 (d, J=1.15 Hz, 3H)

¹³C NMR (75 MHz, CHLOROFORM-d) δ 163.7, 158.9, 150.4, 144.0, 135.0, 135.0, 130.1, 130.0, 128.1, 128.1, 127.3, 113.4, 111.8, 87.4, 84.3, 83.8, 80.0, 63.0, 60.4, 55.3, 38.7, 38.5, 21.1, 14.2, 11.8

LCMS: M+Na=645.2

### Preparation of Compound D11.04

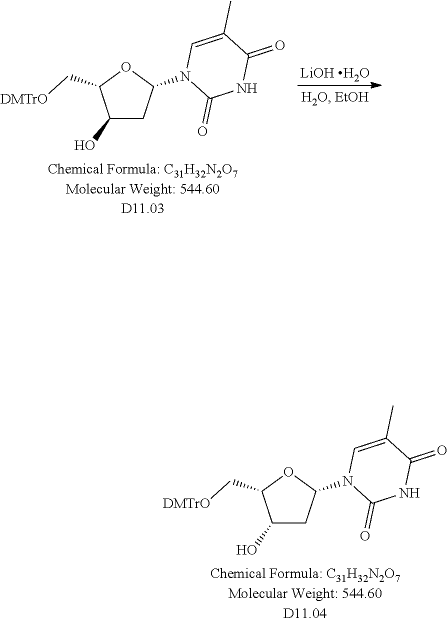

To a solution of (D11.03) (2.0 g, 3.21 mmol) in ethanol (21.4 mL) and distilled water (10.7 mL) was added LiOH (231 mg, 9.63 mmol). After the reaction mixture was heated at 80° C. for 3 h, the ethanol was evaporated and extracted with dichloromethane. The organic residue was evaporated, and purification by Biotage (Si, 25 g col, 0-80% ethyl acetate/hexanes) afforded the desired compound (D11.04) as a white solid (1.4 g, 80%).

¹H NMR (300 MHz, METHANOL-d4) δ 7.74 (d, J=1.15 Hz, 1H), 7.46-7.56 (m, 2H), 7.33-7.44 (m, 4H), 7.14-7.32 (m, 3H), 6.78-6.89 (m, 4H), 6.17 (dd, J=2.18, 7.94 Hz, 1H), 4.24-4.30 (m, 1H), 4.13-4.20 (m, 1H), 3.75 (s, 6H), 3.52-3.64 (m, 1H), 3.34-3.40 (m, 1H), 2.59 (ddd, J=5.25, 8.00, 14.79 Hz, 1H), 1.97 (d, J=1.41 Hz, 1H), 1.74 (d, J=1.02 Hz, 3H)

¹³C NMR (75 MHz, METHANOL-d4) δ 166.6, 160.1, 152.5, 146.5, 139.2, 137.4, 137.3, 131.4, 129.4, 128.8, 127.8, 114.1, 114.1, 110.4, 87.7, 86.8, 85.6, 71.1, 66.9, 64.2, 55.7, 42.5, 15.5, 12.7

LCMS: M+Na=567.2

### Preparation of Compound 12a

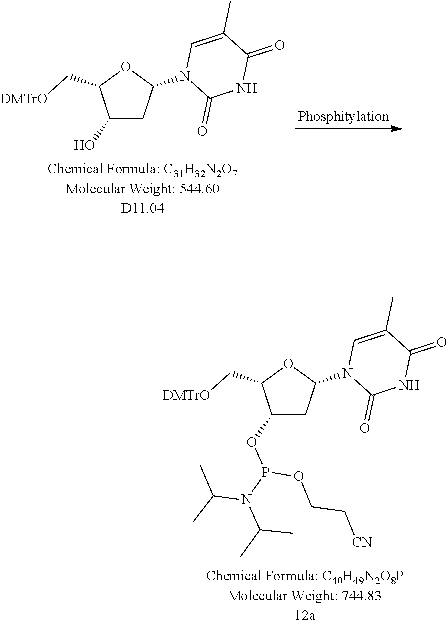

1H-Tetrazole (103 mg, 1.47 mmol) and 1-methylimidazole (0.037 mL, 0.46 mmol) were added to a solution of 1-((2S,4S,5S)-5-((bis(4-methoxyphenyl)(phenyl)methoxy)methyl)-4-hydroxytetrahydrofuran-2-yl)-5-methylpyrimidine-2,4(1H,3H)-dione (D11.04) (1.00 g, 1.84 mmol) in DMF (18.4 mL) at room temperature under an atmosphere of nitrogen. 3-Bis(diisopropylamino)phosphanyloxypropanenitrile (0.908 mL, 2.75 mmol) was then added dropwise and the reaction was stirred at room temperature overnight. Water (0.1 mL) was added to quench the reaction. A 3:1 mixture of toluene/hexanes was added, and the organic layer was washed four times with a 3:2 mixture of DMF/H₂O. The organics were then washed with saturated sodium bicarbonate solution, brine, dried over solid sodium sulfate and concentrated under reduced pressure to a white foam. Purification by Biotage (Si, 50 g col, 0-60% ethyl acetate/hexanes) afforded the desired product (12a) as a white solid (0.743 g, 54%).

¹H NMR (300 MHz, CHLOROFORM-d) δ 8.93-9.09 (m, 1H), 7.43-7.53 (m, 3H), 7.15-7.41 (m, 7H), 6.74-6.91 (m, 4H), 6.10-6.29 (m, 1H), 4.39-4.53 (m, 1H), 4.26 (dt, J=3.65, 7.14 Hz, 1H), 3.78 (d, J=3.33 Hz, 6H), 3.34-3.62 (m, 5H), 2.48-2.80 (m, 2H), 2.16-2.46 (m, 2H), 1.74-1.81 (m, 3H), 1.13-1.15 (m, 1H), 1.05-1.15 (m, 6H), 0.88-0.98 (m, 6H)

¹³C NMR (75 MHz, CHLOROFORM-d) δ 164.1, 163.9, 158.5, 158.5, 158.5, 158.5, 150.4, 150.4, 144.7, 135.9, 136.2, 130.2, 130.1, 130.1, 128.3, 128.2, 127.9, 126.9, 126.8, 117.3, 113.1, 109.7, 109.4, 86.5, 86.4, 85.6, 85.3, 84.1, 84.1, 73.7, 73.5, 72.2, 72.0, 63.3, 63.1, 60.4, 58.3, 58.0, 57.9, 57.6, 55.2, 55.2, 45.6, 45.5, 43.2, 43.1, 43.0, 43.0, 40.8, 40.3, 24.6, 24.5, 24.4, 24.3, 24.2, 23.2, 23.1, 22.3, 22.2, 21.1, 20.3, 20.2, 20.1, 14.2, 12.5

³¹P NMR (121 MHz, CHLOROFORM-d) δ 149.89 (s, 1P), 148.39 (s, 1P)

LCMS: M−1=743.4

1.-245. (canceled)

246. An oligomeric compound comprising a modified oligonucleotide consisting of 12-30 linked nucleosides, wherein at least one nucleoside of the modified oligonucleotide is a stereo-non-standard nucleoside having Formula II; 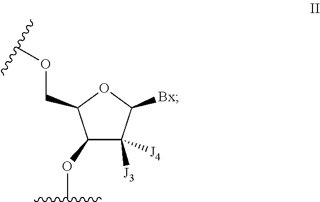
wherein J₃ is H and J₄ is selected from H, OH, F, OCH₃, OCH₂CH₂OCH₃, C₁-C₆ alkoxy, and SCH₃;
Bx is a heterocyclic base moiety;
and wherein the oligomeric compound is an RNAi compound.

247. The oligomeric compound of claim 246, wherein J₄ is H.

248. The oligomeric compound of claim 246, wherein J₄ is F.

249. The oligomeric compound of claim 246, wherein J₄ is OCH₃.

250. The oligomeric compound of claim 246, wherein Bx is selected from uracil, thymine, cytosine, 5-methyl cytosine, adenine and guanine.

251. The oligomeric compound of claim 246, wherein exactly one nucleoside of the modified oligonucleotide is a stereo-non-standard nucleoside.

252. The oligomeric compound of claim 246, wherein exactly two nucleosides of the modified oligonucleotide are stereo-non-standard nucleosides.

253. The oligomeric compound of claim 246, wherein the oligomeric compound is a double-stranded siRNA compound comprising an antisense siRNA oligomeric compound comprising an antisense siRNA oligonucleotide and a sense siRNA oligomeric compound comprising a sense siRNA oligonucleotide, wherein at least one of the antisense siRNA oligomeric compound and the sense siRNA oligomeric compound is an oligomeric compound according to claim 1.

254. The oligomeric compound of claim 253, wherein the antisense siRNA oligonucleotide consists of 17-30 linked nucleosides.

255. The oligomeric compound of claim 253, wherein the 2^{nd} nucleoside of the antisense siRNA oligonucleotide, counting from the 5′-end, is a stereo-non-standard nucleoside having Formula II.

256. The oligomeric compound of claim 253, wherein the 14^{th} nucleoside of the antisense siRNA oligonucleotide, counting from the 5′-end, is a stereo-non-standard nucleoside having Formula II.

257. The oligomeric compound of claim 253, wherein each nucleoside of the antisense siRNA oligonucleotide that is not a stereo-non-standard nucleoside having Formula II is a stereo-standard nucleoside or a bicyclic nucleoside selected from an LNA nucleoside, a cEt nucleoside, a 2′-MOE nucleoside, a 2′-OMe nucleoside, a 2′-F nucleoside, a 5′-Me nucleoside, a DNA nucleoside, and a RNA nucleoside.

258. The oligomeric compound of claim 253, wherein the 5′-end of the antisense siRNA oligonucleotide comprises a stabilized phosphate group.

259. The oligomeric compound of claim 258, wherein the stabilized phosphate group is 5′-vinyl phosphonate.

260. The oligomeric compound of claim 253, wherein the sense siRNA oligonucleotide consists of 17-30 linked nucleosides.

261. The oligomeric compound of claim 253, wherein the 7^{th}, 9^{th}, 10^{th}, or 11^{th} nucleoside of the sense siRNA oligonucleotide, counting from the 5′ end, is a stereo-non-standard nucleoside having Formula II.

262. The oligomeric compound of claim 253, wherein each nucleoside of the sense siRNA oligonucleotide that is not a stereo-non-standard nucleoside having Formula II is a stereo-standard nucleoside or a bicyclic nucleoside selected from an LNA nucleoside, a cEt nucleoside, a 2′-MOE nucleoside, a 2′-OMe nucleoside, a 2′-F nucleoside, a 5′-Me nucleoside, a DNA nucleoside, and a RNA nucleoside.

263. The oligomeric compound of claim 253, wherein the antisense siRNA oligonucleotide has a nucleobase sequence comprising a targeting region comprising at least 15 contiguous nucleobases, wherein the nucleobase sequence of targeting region is at least 85% complementary to an equal length portion of the nucleobase sequence of a target RNA.

264. The oligomeric compound of claim 253, comprising a conjugate.

265. The oligomeric compound of claim 265, wherein a conjugate is attached to the sense siRNA oligonucleotide.

266. The oligomeric compound of claim 265, wherein the conjugate comprises a GalNAc moiety.

267. A pharmaceutical composition, comprising the oligomeric compound of claim 246.

268. The pharmaceutical composition of claim 267, comprising a pharmaceutically acceptable diluent.


SEQUENCE LISTING (16 sequences)

SEQ ID NO: 1 - DNA, 14836 bp, Mus musculus
cctcccccgtgtctccccacacccgggttggggttgttttggttgaccagagtggaacacaacgatctattggcagggctgaacaccaatgggtctatttgtaaagcgccaatgaccactttctgaagcagggttttagggagcggggccttagggaactctttggtcctttttagaacactggactttcttctggaaaggcaggaaacactgaagtttaagaagttgtttccagcttccattaactgaacacacattaaaaccaagcacagagaatcaggacgtttcgcgggagtgagacccagtcatttctcctccgtttccattctgcagggtgagagttgtaatcacccacccactattcgtaccatccacccacccccagtcgagagaataggggtacagaggggaggtggcaaagaaaattcacgatactgagtatctctgggagacctgtttggtctctttgctcggtagcgcagccctacgttagaatgcatcttcccgggaatgactgtagtgagactttggctgggaatccaagttattctaactgtagattggtccacgttgccctaagcctagcagtccactgcggcacagacaccctggacatgaggtgggtcagcttaagttcctggcacgaaagaaagggtactctggcaacttttggatgcggcgaaacagactgtttcgtctctcaggttcttatttcacggcttgtgcctttgacagccccttagtttctctatctgcaggatgggagcattaagctctacgacccagcctctttacaattcaggtccaaagagcccgcccaagttggggactgggaagatcaaaggtctcagcacccagcggagccgcggacactgagggcgccaagaagggggtgggtaggtagggaactggaagggcggctgctccgcaggggatgcgcgtcagagaccccagccacactccaggcccgccccttgatgagccccgccccgccccgcctggttttcgcctctaaagcgcccagcgctcgcctcccgctgccgcactttcactctcggtccacctcggtgtcctcttgctgtccagctctgcagcctccggcgcgccctcccgcccacgccatggacgccaaggtcgtcgccgtgctggccctggtgctggccgcgctctgcatcagtgacggtgagtgcaatccgcggccgggcccgggaaaggctcgcagctctgcgccggagctccttcgggtccgcggttcctctgcccgcgccgaagtcgcggagaaagaactcggtcggcgccgttcactacaagcgaacttggggcagtccactttgcagggcgcactcccaccgggtgccctttcccgtgtcccacgggtcgcaccgaggttttgtgctctgcgaagtgcggccataggacctagagagggctgcaggggaggacccgcaggattgttgggcaagagtgggttcggcgcggaatggaagcgtgggcgattgtgtccggggcttgggccccggagcgcgccagctgcactcagctagtgtctaccggcgcccagatgtttccagaggcgaagggcagcgcggtcccggagttgaccgtgcaagaggttcactcgggtggtgcgtgtgtcagcaaactctcaaagaccggtcaagtagctcgaagtgcatggcttggctataggttcagtggtgaggctgagtttcgtcccctgcgggtgtagcgtgttctcttacagcaccctcgaggggctcagggccaccagcagcgcagcgcagctcttgaactcgcgctgccagccagggccgcgcttctgcacagttcgttggtccgtagcgacgcggacctgagcacgcgtctcttcactgcccctttttcttcttacccgggtcactagacaaaggctcagcagttacccaagctatatgcacacctctccccaacccccaaacacacctgcaaacgggcgcttttgtagccagccccggagtcctcagctctggaatgagagctgcagcggagttcagtctcccagacccagggtggtgtcttctttcactgggaaagggctttcattttgttttctttttttgacactgaagagaaaactctcagcgctgttacaagacaccgttgctgcaaaacaaaacaaaccattgcctctgaacacaaaacaaaatcctactagtcgatcccctgccttcctccgcagtggtgtttcctggagagaactgagggacagtcggggctcttggtgagactgagctctaaatgctgcccaagtacaccaactcgttcgtttgggttctttccctgtgacaacggggtacgggaatggttggagttgcctagtccgagggaaatgttctgtaaaagaatagtcagttgctgatcggagtagtaaaaaaaaagaaatgaaaggcagtttcgatttttttttttttttttttttttttttttgttaccgagaacacccgggaggctgagccttcccactggtcccccagtgccccgtcatggagcacattgatttgggcattaataattgaatgagctggtgatgttgcaagggtcacagcctctggcaagttaggtatggggcaagaatgtaggactcaggtcctcaaggttggagtgcaattatccagagtaaaagttgtctcaccctcaacatattctgaccctaggaagagtcggattgttgacagtgtctggatcagacctgttctctaggcaggaccccattgtgctgcccgaatgaacttttttacctcctagtgcctgtgtgccctctgatcttacacagccctcaagttgcagcacggctaaccttgctgtggttcctgtcttttcccatcagctactccaactcagaagctagatagtagacaccggaggcttctttggttaaacccagagcagcaggcttgccaggcttgttagattgaatggacccctggttccctaagccaagctctctagattcccaagtccagggtggcagcagagctggattagactttggtctgtacctgaagtctggttttcctatgctttagagtctaaagacactacccttcctggggcatgcatcccttagctaaataatgcttgcagaagaagataatcccatcatatatttaattcggtccacttctccagctgcttcccaaaggcagtgaacttcagaatacccagaagtctcctggaactctaaataagcaaacttaaaatcctggggctaactattctcagtcatacttttaaactttggtgaaaagacccataaattgaaacatttggggatgctcagtagagctaggataaaaccctgttgttgggggagcagctacaaatccagcagtcctcagggtttgcaattctagacttaaagggtggttcttaagggggggttctaaaggagccccttgctaatttacactaatgagtgtcaattatagcattttgcaaattggtgaattggcaaacaaagctggtaataggatccaggaggcctaggcatccaggtagtgaccataaaagccacggttgaccccagcttttgggaaaagctggatagaaggtaaatccgggtcctcccctctggattcttttgtgatttccagggcttaggatagggtgagtgggaggagggaaaactgcaggtggtagaagtgaagccccccacctccaggcctgcaccagagggccacaagggagcccagaactctgccaccccacttctcctgggtccttttgtcctttagaggctgagcccagtcagatctcactgtgatccctggccgaggggatggtctttgcaagaaactttctgtaaccattcctgctgatgttcctgagtcttccccacaagagccaccaaaccccctgcaccaggcagataatgactggccccacttttctctctacacctcctctaggtaaaccagtcagcctgagctaccgatgcccctgccggttcttcgagagccacatcgccagagccaacgtcaagcatctgaaaatcctcaacactccaaactgtgcccttcagattgtgtaagtcctagccgccatcccccaaagaggagcatggtatagaagcctcggacttggcataactaggggcagctgttaccaccaccaccacggggacactgatatgccatcagacatgggtttcaaaggatacttttgttccccagagccctgatgtcctcagtgtttctcactcttgctttccaagctgtttcttgcagcacagtgggccgcctctctacagaaaaagccatggacttgatggaggtcagccctcagctgacagttgggtctgtcttgtcagtttcaaggttctggtgtccaaagttaatcctttctcacatagaaaaaaaaattacaagacccggatggcacggggggggggggggttcagttttactcacttgcactcacttgctcagaggtcatttttgttttagagttttagagtttgctggagtgtgatggtagctgccagtatttgatttaaatttacctgggaaataagaaaagcccaaaaaaggtataaatgatgtgaatatctcactcagagtctggtagacttggcagagatgtgtcctgtgctagtctgtcctgctcactgccccccagcaggggttcccatcctcgggagactcaacactaacaacagtataaggatgcagcagctggagcaatgctagcctgacggctttgtcacccaacggtgactgcttcagactttctgtgctcatcagccttcctctccagcctccgctgctgtgttatgtacagtaggctttagagacctagatgatgaatattatttttgctgttttgattaaaatacaatactctcccgagaaagggattttaaagatgatgagtttacgtttgaataggctgtgctggtgcactgtcccgggaagggcccttgaacttagagggtcaaatacaactattgattctgggtgatcactaagttaataaatggcaggatccagactgacacccctgatccctgttgaagttacatccctctgaacgactggtcaactgcagggcagcctgcttgaagagggttacctgtccctaggacactgaacaggcatttgtttttcctagaagacagttcaccagctggagaggagtcgtctcccgtagtttctgtttggttgcttttggtttttgtttggttttggttttttaattatctggcatccaggacttgatggaaaataaccagagctaagctcaccggttcatctgcccattaggaagttctagggatgggagaaagaacacggcgtcaattaacaaatccacaaagctaagaccttgaagcattctgtgaacttgtaaacgcgctcaggcaaccattggacaatttgtctagactgctccttgcccacctgaactgccctgttcctccccttctggactcctgccgtcttcctccagagctacctttaaggttgtcccatgtactatcaaggtgctctgtcaaaagttcttaggctgcttctggcactctccagaattttccaagacctcccccccaccatgatatcagtcatccgcgccttctgggtggttcttcctccacaccctttgggcactttgactcctgtgggatattcgtccttccttttcctttagctttcctcacttgccaagctccaacttggccagaagctcaaatgcctccactgtggtctcttctctgtgtcccctgggagacatccttagcacgtccctaactctgcggtggtggtcccaacacgattcaagtgctatgtcttccaaaactgaagcttccgggagcagcagctgggccctgcagtgaggacctttagctgggtgtgttgggtgagcccacaggatcgctttctcccgcttggctgtacagcgtctctccccttgtgttttggcagtgcacggctgaagaacaacaacagacaagtgtgcattgacccgaaattaaagtggatccaagagtacctggagaaagctttaaacaagtaagcacaacagcccaaaggactttccagtagacccccgaggaaggctgacatccgtgggagatgcaagggcagtggtggggaggagggcctgaaccctggccaggatggccggcgggacagcactgactggggtcatgctaaggtttgccagcataaagacactccgccatagcatatggtacgatattgcagcttatattcatccctgccctcgcccgtgcacaatggagcttttataactggggtttttctaaggaattgtattaccctaaccagttagcttcatccccattctcctcatcctcatcttcattttaaaaagcagtgattacttcaagggctgtattcagtttgctttggagcttctctttgccctggggcctctgggcacagttatagacggtggctttgcagggagccctagagagaaaccttccaccagagcagagtccgaggaacgctgcagggcttgtcctgcagggggcgctcctcgacagatgccttgtcctgagtcaacacaagatccggcagagggaggctcctttatccagttcagtgccagggtcgggaagcttcctttagaagtgatccctgaagctgtgctcagagaccctttcctagccgttcctgctctctgcttgcctccaaacgcatgcttcatctgacttccgcttctcacctctgtagcctgacggaccaatgctgcaatggaagggaggagagtgatgtggggtgccccctccctctcttccctttgctttcctctcacttgggccctttgtgagatttttctttggcctcctgtagaatggagccagaccatcctggataatgtgagaacatgcctagatttacccacaaaacacaagtctgagaattaatcataaacggaagtttaaatgaggatttggactttggtaattgtccctgagtcctatatatttcaacagtggctctatgggctctgatcgaatatcagtgatgaaaataataataataataataataacgaataagccagaatcttgccatgaagccacagtggggattctgggttccaatcagaaatggagacaagataaaacttgcatacattcttatgatcacagacggccctggtggtttttggtaactatttacaaggcatttttttacatatatttttgtgcactttttatgtttctttggaagacaaatgtatttcagaatatatttgtagtcaattcatatatttgaagtggagccatagtaatgccagtagatatctctatgatcttgagctactggcaacttgtaaagaaatatatatgacatataaatgtattgtagctttccggtgtcagccacggtgtatttttccacttggaatgaaattgtatcaactgtgacattatatgcactagcaataaaatgctaattgtttcatgctgtaaacctcctaccgtatgtgggaatttatttacctgaaataaaatctactagttgttagatggagtgcacatacatttctgaagatggagaaaaacaggtgtgcctgctgatcaggtgctgtgggctgccctgcagtcctggtgagcgacagacactgaggcaggcttgtctcatgaacaggctgcctctgcagtgaaagtttttgtgtattttttttaacccaagctagttttctaatgaataatacttgactcactaatttcccctcctcctccttctcctcagttctcctaacatcctcatgtgatccccagactcaactccagtaatatcaagctttcctattttcccatgtaaaaaaatcccatgactctgggccatgttaatatcaggcttttgtgggaacaggtggcctcaccccataaatcattaaataccattcagcttgaatcattttaatgtgacagtcacaaaccagttgctctaataaaaactctgctaaccatccttctccttagctctctagaacaatctcagttatccctagggatgctccccagcatccagaaaagagaagtgggatcaatcatcctgcctttctccccctcctctcttggagggctgcctgagcccgtggcctccacctcccctgctttgtataatttgaaatgcagatttgtagtgaaggcagagttcacctctgcattgaaagggaaggcaggcccagagcttccttccctgccctctgagatgtgcatttatgtctcaggatggatgagctttggtaggaatgctcaaaaccaggaccagccagacaaactggcagtccctgtaagcggttcccgggtcatagggttagggcacccctgtttaactttggggtggggaaagtatctggttttctttgataaattgcttgtgaaccacatttgccaagtggcctccaggcctcaaactcaaagaccgagctaaatcgactcggaaggcaatgctgaatgaagattgtgggaactgagatagatacactcctctatgttgcaatgtgattaatggttctactaattttatctaagggggcgcagagaagaaaaagtggggaaaaaagaaaagataggaaaaaagaagcgacagaagaagagaaaggctgcccagaaaaggaaaaactagttccccgcttcctgccgatggaccgcagtgcgctctgctctggcgctttgtaactcgctcctccctcttcgggggcagaccccacactccgggcaggtgctcaaacctgacggtaaactcttccctcttcgggggcagaccccataccccggggcgggtgcttaggctttcctgccctggtggccacaccagctgctgtatttatgtgcttcataaggccctgctctgtctgctaaagctatgaagaaagatgtgcagagactggggtggagactaagccaaagaggagctgcctagcctggcagcattgccccgagctgagcccccttggccaggacttcacaaggctcacacctacaatcccatgaaggccagggtggtctgcttagccaggaaagggcaagtgccttcccctcggccacactgccccttgtggccttctcgggacatgtggtaactgacttgctctcaggcccacccgcagcttttccaaatacctgcagccttcagccctgctgccctgcctgtgggagcagctttgactccagtccagaagggtttctgcagactgtgttgggtgagacgcagaaaggatgaaatctcagaacacatgtcagctgcttctcaggaaatcttttctttggacaattcactttagagtctttaaacgggtctctcgtggggaggatagatgtgctctggaactttctgaaggaccagcagcttcagggactcttagtctgtccttccccacttttggtcccaacatccctgggatggtgtgctgtctgggcaccacggtctccatcctcactcctgagagatttctgccttctgtgagttgggttaaagctctggaattatctactatcccaatccactaccctcacctggcaatatttgtctgtttttgtttgtttgtttgtttgtttttgtcttttgccagtttgaattagaaggcaaggctctgattttagtagtgttttggaaaaggacttttttcttcaccttcctctttgcctcatgtgtacacacacacacacatcttgtaccccagacctctgggtataattttcataattggtgcagaaagaagaaatgatctgaagatgtgttaaatggattgcaggggaaggaaggcccagggccctgtgtgtcatgccctcttgggttcctaagttctatgttccttagaggttctagcattaaacagataaagcccttcatggtcctggctgaggaagagtcttgctagggggattcagggaagacccgtgttaccagctcttaccctttatctggacagctctcctaccctgtatcttctcctcagatctgaggatagcaggctggactattggtgggcacctttcaagcccagggctactgtttgtcctgtggcagccggctacagtctcgtctgagtggcctcatctggacccttcctgttattaataaaacgcttctggaggccagatctgtgctcaagccatagttctgcttagaaagggatgccccacccttaccggacactgggaagaactgttggcccctagaaaccaaaggccaaactgaggctgccctgagttggaagaccactttctgaaatgcccatggactctgcctcccaaccattcgtctctcactcctagcagagctgtctgtgcagactgtttcttaggaggcacagcaagctccagggaaccctctgtgcttatgaagctcgtctggtgggcaaccccagcccactggacagagtcctcatggaaatgcctgggaagctgatttcatctaaggatgggttgaagtaggatgtgctcctgcgacttctcaggcaggtgagaggggtagtccttacactgtctagcataaacgccttccggaaggacctgcagctccagagaccacctcctgagcaccaagacctcttctggtggtgtggaaccagccaagagatttcaaggaagagtgattatttgatgaatgctatgggaatggcctcttctcttggagttctgaggcctggggatgcccaggaacactgggcacctgctgctgttagggccaatgcatagtctcagcaccggtgtcctaaggttaaggcggtgcgccttgtcatgtgctccttgtaccatgccatctgtgccagtgtgtgtctgcctcaccctgtgcttgacatgttcacccatcttctctgcttcccgccaccatccagatcctcagcggccgccccggctgtgcccttccctgctctcccgctctctcaggcctcggaaggaagatcggtggctgcgagctgaactaaggagtagggcctgtggctcagcgctaggccacgcacgcagcatcccaggcatgtggtgagaaactgccttaatgtgtctcctctgttcttgtcaacaggaggctcaagatgtgagaggtgtgagtcagacgcccgaggaacttacaggaggagcctaggtctgaagtcagtgttagggaagggcccatagccacttcctctgctcctgagcagggctgaagccgtttgcaagggacttgctttgcacagttttgctgtactttcacattttattatgtagcaagatacatggtgatttttttttttttcatttagcctgattttccaacgtcattggtgacaggccaaggccactatgttatttcctttgttctggtatccttcccttggaggaccttctctgagtagtggctccccaggtttgtcctttgagctgaggcaggaggctcacccattcttctgaataggaactgggtgttcccaccccccaaggactgcagggctttcccaagctgaggcaggaacgtgaggccagggaagagtgagcttcaccctcatcccacgctgtcctcctcaacccaccatgctcatcattctgtctcatccatccatccatccatccattcatcgccatgtgtccgcaagactgtctccatgaccctgaaaaaggactctcgagatgaaatcctttattcaaatgggacagcaagaaggaaaagccaatgtctggtgtctctccccccgcccctaccctgcgcgcatctatgtcttgtttggaatattgtctcttcaaccccctgttcatgtccttctcactcatgatcgatgtcttgtctgtgcactgtctctaacccaaatgcaaaggctgagtgtgaggtgatggccccgaggtccaggttgtagtcatggaaagagccctgctgtctcccttctcagggggcccattttagacacacaaagcccaaagaaaggtggtttgcaacagtgcttagctcgagcctccatatttccataactgttagcttaaaactgtggggttttaccttcctggaaccaaatgcattcttctgttgaggagtaacaggtctcaattcttttcaattaattttaaaagtcaatcactaagagcatcggctttgggccctgatgggcaggcatttccctggaaagggggtgaactacctacctctcctcaagacagccgaagggtgggattggtgccgctctgggaagcgtggccccaggagttttgtcctctgcagtttttaatgcaagttcactgccactttgacaaaagcccaattagaagccagtctctagttccttaaacaaaacagacagagtaaggaaaggaaggagggtggcagccagctggctggacactcgagaaagacggggaagtaagctacagaaagatagtcttcaaaaacaggtgtttgagagtgaatactctgtagaattgttagtggggtgtgtgtggtggtggtggggggatttctacaaaatagtcctttaagttgagtttacagcagatgaaaaatccaaccagcaaaattttgatcaaatttgaacaaaaacccaaaaacctaaaactgttgagcaggttgcgatgaggagcacagggctagctgcagagctggatcctcaggaggatagcgaattattttcaaccctggaatagaaaccacacactggcttgctgtgcaccagccactttgcatctaatccaagctttgaagggtgttgcttgggaggaaacaaatacagccttccatcttcactccagttagggatcctttcaaagtctccttcacagtgaggaaaaagagaagggtagaaactttagggagccggatttgtgtatcaattcctccgctgacagtcagtttctagatggagacagcctgcttaaagcaaatccgaatttaaataggacatttacatcggaaaagtctctccctaccttaatcccccattctcttgctttcaaaatacaagcacagcagtccttgaatggctgttgacccagggcacctagctgtccctgctggtcctggggctgccagaattcccttgggcgccaagcaacctgccaggtagccagtccctctgttacaagcctttgcatctggatagggaaaggggtggagacatacagtctgctttgtgttgaaacccagatttgtaccctgtgtttatacactgctgctggctcccgaggacagtgggactttagcaaggaagtgcagccgaggggtaaagagccctctggttcattgcctgatcggctttgagagagggtttggagggcaaggggctgcattcctctgagggacttggcctgaggcctttcgggcctctccagtgggttctgtttatcctctcatgggtgattatctcagtggtgtcaccaggggcttcctcccagaagtcagtcatccccaggccgtgcacccttttcagctggatgagagccagggatgcattctctccaaacagctaccctggcccattttaaggtaatctcattcttcaaaatgttccatagaatcctccaaattcccccagcagacttctaccctcgccaagttcccaaaacccactcagcaaagttgccaacctcgacgggctagcagtgtctaagcagcgatgggttcagtgttgtgtgtggtgaatactgtattttgtttcagttctgtctcccagataatgtgaaaacggtccaggagaaggcagcttcctatatgcagcgtgtgctttcttattcttatttttaatatatgacagttatttgagaagccatttctactttgaagtcattatcgatgaaagtgatgtatcttcacctaccattttcctaataaagttctgtattcaaatatagctgccaagcatcctcagtgaatgttaccatgtggaattttccacacttggttttaccccctcaaacctgactctgaccgtgcagtcttagcagaagagcttagcaggtcctagtgttcactcttggtctaactgctggtgtcagaagatctctacagggagaggtgttccattttctccacatgacctggattgctccttagaggtcagacagccttgcactgtacaaggcaatggcttagggtaaagtcccaggagttttccctacagtcccaagaatttggaagaggaaggcccacactacacatgcaggtcatggtggaaggtgacagaggaaggactctgtccctgtaagacagctggaaaccacaatattctgcatgttcctatcctgggtgaggacgctaatggaagtcaaaggggaatttgctaactgctgttggccagcttcctccaagaatcctgcttccccaacagacagagcctttgtctcttatagtttggtcttcagattctctttatcccacattcagccatttttgtaaaagagaggctagcaccagctccaaatatccaaatctgcagtgtttgagatctcactgcgcctcctccataccaacacatttgccattacttatagggtagttttcatgtgagttctaagttgattaacacacaagaattagaagggtgggaggctctaggaaaggcactgtgggactatttgactgcatgggtgtgaaaatgtaaggaacaggcaagagcttggatcccattctctctgcccacattgtgacttgagatatactaattgctcttgggggtctcagtcatataccatccataacagagttaaactgagagagatacaggatcagctagaatgaaaagcccaccccatgcttccagaaagtcccctctttatacctcctgtgatatgaactagaggaaaagcaattgactttgcttctcaaacagcctacggcaaagccctgtgagtttg

SEQ ID NO: 2 - DNA, 20 bp, Artificial sequence
  Primer
ccagagccaacgtcaagcat

SEQ ID NO: 3 - DNA, 20 bp, Artificial sequence
  Primer
cagccgtgcaacaatctgaa

SEQ ID NO: 4 - DNA, 29 bp, Artificial sequence
  Probe
tgaaaatcctcaacactccaaactgtgcc

SEQ ID NO: 5 - DNA, 16 bp, Artificial sequence
  Synthetic oligonucleotide
gcatgttctcacatta

SEQ ID NO: 6 - RNA, 20 nt, Artificial sequence
  Synthetic oligonucleotide
gauaaugugagaacaugccu

SEQ ID NO: 7 - DNA, 23 bp, Artificial sequence
  Synthetic oligonucleotide
  misc_feature (1)..(21): bases at these positions are RNA
auaaaaucuacagucauaggatt

SEQ ID NO: 8 - DNA, 1435 bp, Homo sapiens
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa

SEQ ID NO: 9 - RNA, 21 nt, Artificial sequence
  Synthetic oligonucleotide
uccuaugacuguagauuuuau

SEQ ID NO: 10 - DNA, 22 bp, Artificial sequence
  Primer
ttgttgtaggatatgcccttga

SEQ ID NO: 11 - DNA, 21 bp, Artificial sequence
  Primer
gcgatgtcaataggactccag

SEQ ID NO: 12 - DNA, 31 bp, Artificial sequence
  Probe
agcctaagatgagagttcaagttgagtttgg

SEQ ID NO: 13 - DNA, 23 bp, Artificial sequence
  Synthetic oligonucleotide
  misc_feature (2)..(21): bases at these positions are RNA
tuaaaaucuacagucauaggatt

SEQ ID NO: 14 - DNA, 23 bp, Artificial sequence
  Synthetic oligonucleotide
  misc_feature (1)..(21): bases at these positions are RNA
uuaaaaucuacagucauaggatt

SEQ ID NO: 15 - RNA, 20 nt, Artificial sequence
  Synthetic oligonucleotide
uaaaaucuacagucauagga

SEQ ID NO: 16 - DNA, 12 bp, Artificial sequence
  Synthetic oligonucleotide
tttttttttttt